(19) United States
(12) Patent Application Publication
(10) Pub. No.: US 2026/0250271 A1
(43) Pub. Date: Aug. 27, 2026

(54) MITOGEN-ACTIVATED PROTEIN KINASE INTERACTING KINASE (MNK) INHIBITORS AND USES THEREOF

(71) Applicant: Northwestern University, Evanston, IL (US)

(72) Inventors: Gary E. Schiltz, Evanston, IL (US); Purav Pankaj Vagadia, Evanston, IL (US)

(21) Appl. No.: 19/548,871

(22) Filed: Feb. 24, 2026

Related U.S. Application Data
(60) Provisional application No. 63/762,610, filed on Feb. 24, 2025.

(51) Int. Cl. C07D 403/14; A61K 31/506; A61K 31/5377; A61K 31/706; A61P 35/02; C07D 401/14; C07D 403/04; C07D 405/14; C07D 471/04; C07D 491/048

(52) U.S. Cl. CPC C07D 403/14; A61K 31/506; A61K 31/5377; A61K 31/706; A61P 35/02; C07D 401/14; C07D 403/04; C07D 405/14; C07D 471/04; C07D 491/048

(57) ABSTRACT

Disclosed are neuronal mitogen-activated protein kinase interacting kinase (MNK) inhibitors and methods of using the same in treating a disease or disorder associated with MNK1 and/or MNK2 activity, such as a cancer or a cell proliferative disorder, or a central nervous system (CNS) disease.

### CROSS-REFERENCE TO RELATED APPLICATIONS

The present application claims the benefit of priority to U.S. Provisional Application No. 63/762,610, filed on Feb. 24, 2025. The contents of which is incorporated herein by reference in its entirety.

### STATEMENT REGARDING FEDERALLY SPONSORED RESEARCH OR DEVELOPMENT

This invention was made with government support under grant numbers NS113425, OD030332, CA060553, OD023681 and CA121192 awarded by the National Institutes of Health, and grant number ECCS2025633 awarded by the National Science Foundation. The government has certain rights in the invention.

### BACKGROUND OF THE INVENTION

Cellular mechanisms that control mRNA translation play essential roles in gene expression.¹ The critical factor for the initiation of mRNA translation is the availability of eIF4E (eukaryotic translation initiation factor 4E) for the initiation complex 4F formation, along with the RNA helicase eIF4A and the scaffolding protein eIF4G.² eIF4E is a key component of this complex because it recognizes and directly binds to the 5′-cap of the mRNA structure, which includes a 7-methylguanosine (m7G) moiety.² The eIF4G scaffolding protein also directly interacts with eIF4E and other components of the complex.² This complex also includes the eIF4B protein that supports the RNA-helicase function of eIF4A, thus controlling the translation of mRNAs that contain 5′-untranslated regions (UTRs).³eIF4E plays key roles in regulating the translation of mRNAs that lead to the expression of oncogenic proteins, such as c-Myc, cyclins D1 and D3, and Mcl-1.^{2, 4, 5}

The oncogenic activity of eIF4E is activated by its phosphorylation at Ser209 by mitogen-activated protein kinase (MAPK) interacting kinase 1 and 2 (MNK1/2).^{1, 2, 4, 6, 7} MNKs are serine-threonine kinases that are activated downstream of MAP kinases (either ERK or p38) in response to stress, growth factors, and cytokines^{1, 2, 4, 6, 7}. There are two known MNK genes: MKNK1 and MKNK2. The MKNK1 gene produces protein isoforms MNK1a and MNK1b, and the MKNK2 gene produces protein isoforms MNK2a and MNK2b, all four of which have a similar N-terminal region (involved in binding to eIF4G), but different C-termini.^{1, 2, 4, 6, 7} The longer MNK1a and MNK2a isoforms have a MAPK binding site at their C-terminal regions that allows for phosphorylation by ERK and p38 MAPK.^{1, 2, 4, 6-8} MNKs are phosphorylated by either p38 or ERK on Thr197/202 within the T-loop domain stimulating kinase activity.^{6, 7} The kinase Pak2 can also phosphorylate MNK1 at Thr22/Ser27, resulting in decreased affinity for eIF4G, while at the same time Pak2 also phosphorylates eIF4G, inhibiting its interaction with eIF4E.⁷ Protein phosphatase 2A (PP2A) negatively regulates MNK activity.^{7, 9}

Elevated eIF4E phosphorylation drives malignant progression in multiple cancers, including glioblastoma (GBM), where it promotes translation of key oncoproteins responsible for tumor growth and therapy resistance. The MNK-eIF4E pathway in glioblastoma is controlled by multiple oncogenic signal pathways, including constitutive activation of receptor tyrosine kinases such as epidermal growth factor receptor (EGFR), loss of tumor suppressors like phosphatase and tensin homolog (PTEN), and activation of the RAS/MAPK signaling cascade.

There are several known MNK inhibitors that have been reported in the literature.^{10, 11} Some of these inhibitors are ATP competitive and bind to the DFD-in active conformation of MNK. MNK inhibitors that bind to the DFD-out inactive form of MNK were also reported. There remains a need for novel MNK inhibitors that are potent, selective, and have high brain exposure and oral bioavailability.

### BRIEF SUMMARY OF THE INVENTION

Disclosed herein are MNK inhibitors and methods of using the same in the treatment of diseases and disorders.

One aspect of the present disclosure provides a compound of formula (I), or a pharmaceutically acceptable salt, hydrate, or solvate thereof,

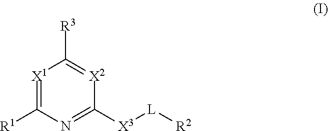

wherein
(i) X³ is NR⁹, X¹ is N, X² is CR⁸;
   R³ is —H;
   R⁸ is —CN, —H, halogen,- or —CF₃;
   R⁹ is —H, or R⁹ together with the nitrogen atom it is attached to, L, and R² form a monocyclic or bicyclic heterocycloalkyl substituted with 0-1 hydroxyl or —NH₂;
   R¹ is benzimidazolyl substituted with 0-1 —NH₂;
   L is absent or an alkylene, wherein L is substituted with 0-1 hydroxyl; and
   R² is heterocycloalkyl, -4-pyridyl, phenyl, or cycloalkyl; wherein R² is substituted with 0-1 hydroxyl, halogen, —NH₂, or alkyl optionally substituted with amino; or
(ii) X³ is O, X¹ is N, X² is CR⁴;
   R³ is —H, alkyl or cycloalkyl;
   R⁴ is halogen, —H, —CN, or haloalkyl;
   R¹ is heteroaryl, wherein R¹ is substituted with 0-1 —NH₂, halogen, —CN, or —NO₂;
   L is absent or an alkylene; and
   R² is pyridyl, heterocycloalkyl, cycloalkyl, or phenyl, wherein R² is substituted with 0-1 amino, alkyl, oxo, halogen, —NH—C(O)-cycloalkyl, —C(O)—O-alkyl, —C(O)—OH, or alkyl optionally substituted with amino;
or
(iii) X³ is O, X¹ is CR⁵, X² is N;
   R³ is —H;
   R⁵ is —H or halogen;
   R¹ is a heteroaryl, wherein R¹ is substituted with 0-1 —NH₂;
   L is absent; and
   R² is piperidinyl, wherein R² is substituted with 0-1 alkyl;
or
(iv) X³ is NR⁶, X¹ is CR⁷, X² is N;
   R³ is —H;
   R⁶ is —H, or R⁶ together with the nitrogen atom it is attached to, L, and R² form a bicyclic heterocycloalkyl;
   R⁷ is —H, —Cl, or —Br;
   R¹ is benzimidazolyl, pyrazolyl, or imidazolyl, wherein R¹ is substituted with 0-1 —NH₂, halogen, —CN, methyl, —C₆H₅, or —C(O)—NH₂; and
   -L-R² is

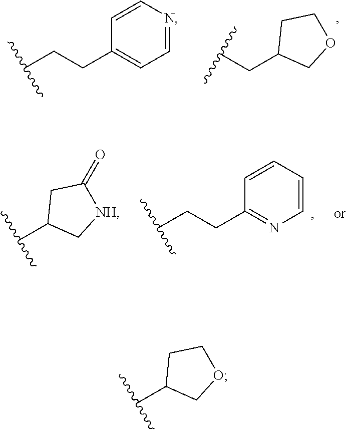 [SMILES: *CCc1ccncc1 ; *CC1CCOC1 ; *C1CNC(=O)C1 ; *CCc1ccccn1 ; *C1CCOC1]

with the proviso that the compound is not

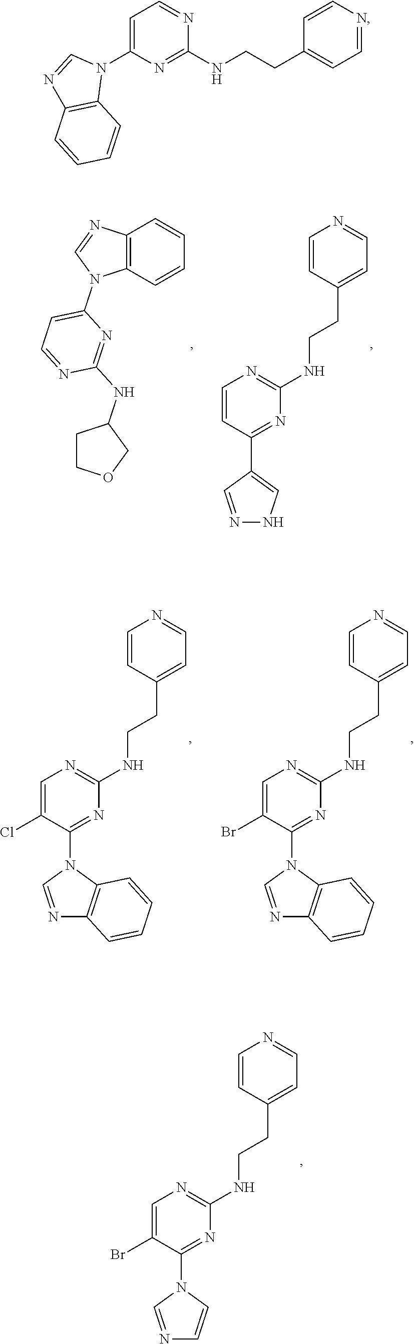 [SMILES: c1ccc2c(c1)ncn2-c1ccnc(NCCc2ccncc2)n1 ; c1ccc2c(c1)ncn2-c1ccnc(NC2CCOC2)n1 ; c1cc(CCNc2nccc(-c3cn[nH]c3)n2)ccn1 ; Clc1cnc(NCCc2ccncc2)nc1-n1cnc2ccccc21 ; Brc1cnc(NCCc2ccncc2)nc1-n1cnc2ccccc21 ; Brc1cnc(NCCc2ccncc2)nc1-n1ccnc1]

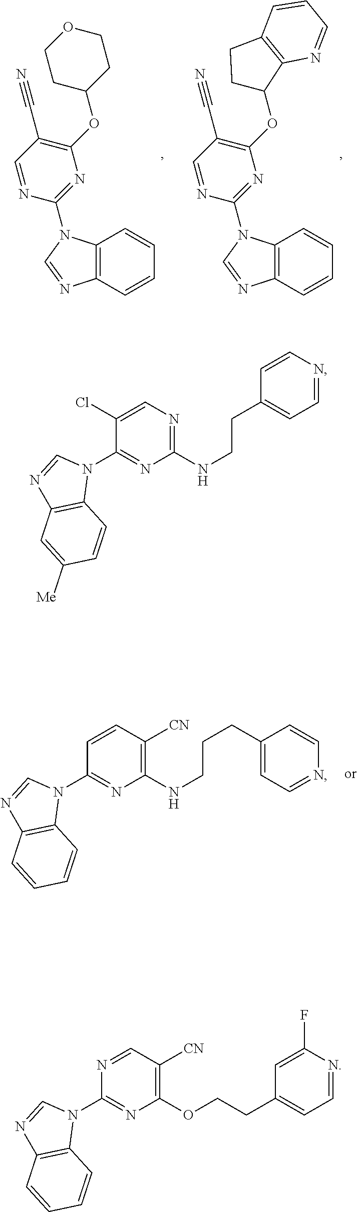 [SMILES: N#Cc1cnc(-n2cnc3ccccc32)nc1OC1CCOCC1 ; N#Cc1cnc(-n2cnc3ccccc32)nc1OC1CCc2cccnc21 ; Cc1ccc2c(c1)ncn2-c1nc(NCCc2ccncc2)ncc1Cl ; N#Cc1ccc(-n2cnc3ccccc32)nc1NCCCc1ccncc1 ; "or" ; N#Cc1cnc(-n2cnc3ccccc32)nc1OCCc1ccnc(F)c1]

Another aspect of the present disclosure provides a method for treating a disease or disorder associated with mitogen-activated protein kinase interacting kinase 1 and/or 2 (MNK1 and/or MNK2) activity in a subject in need thereof, the method comprising administering an effective amount of the compound disclosed herein, or a pharmaceutically acceptable salt, hydrate, or solvate thereof; or the pharmaceutical composition disclosed herein to the subject.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 shows the Kinome plots depicting the greater kinase selectivity of amine-linked inhibitor 8b versus ether-linked inhibitor 12a across 97 kinases (10 μM concentration for both compounds). Spot cutoff value: 35% of control. MNK1 and MNK2 are both highlighted.

FIG. 2 shows the Kinome plot of 12g across 97 kinases (10 μM concentration). Spot cutoff value: 35% of control. MNK1 and MNK2 are both highlighted.

FIG. 3 shows cellular inhibition of eIF4E Ser209 phosphorylation. MV4-11 or U937 cells were treated with the indicated compounds at increasing concentrations and the IC₅₀ values were calculated and are shown in Table 2. Error bars are the mean±S.E.M. from two independent repeats.

FIG. 4 shows the co-crystal structure of 12 h in complex with mutated MNK2-D228G (PDB 9HRC).

FIG. 5 shows that MNK inhibitors inhibit MNK activity, reduce AML cell viability, and enhance 5-azacytidine antineoplastic effects. Phospho-eIF4e (p-eIF4E) at serine 209 is monitored by immunoblotting after treatment with 12a (A) or 12g (B) in U937 and MV4-11 cells at 1 and 4 h at 1 and 10 μM. (C) U937 or MV4-11 cells were treated with 12a or 12g at increasing concentrations and cell viability was determined using the cell viability reagent WST-1. Data represent means±SEM of 3 (U937) or 4 (MV4-11) independent experiments, each done in triplicate. (D) Table of IC₅₀ values for cell viability inhibition for both U937 and MV4-11 cells. IC₅₀ values were calculated in GraphPad Prism using a non-linear regression—[inhibitor] vs. response—analysis. (E) U937 or MV4-11 cells were treated with 12g and/or 5-azacytidine (AZA) at increasing concentrations and subjected to viability assays as in (C). Data represent means SEM of 4 (U937) or 3 (MV4-11) independent experiments, each done in triplicate.

FIG. 6 shows that MNK inhibitor 12g induces apoptosis. (A) Quantification of apoptosis effects, as shown by percentage of annexin positive cells induced by 72-hour treatment with 12g in U937 (n=3) and MV4-11 (n=3) cells. Data represent means±SEM of 3 independent experiments. Ordinary one-way ANOVA with Dunnett's multiple comparison test to compare each experimental group to the control group; **, p=0.0090; ****, p£0.0001. (B, C) Representative flow cytometry dot plots from experiment in (A) for U937 (B) and MV4-11 (C) cells.

FIG. 7 shows the pharmacokinetics of 12g after PO and IV dosing.

FIG. 8 shows a summary of structures.

FIG. 9 shows ORTEP of intermediate 2f.

FIG. 10 shows ORTEP of compound 3a.

FIG. 11 shows ORTEP of compound 3c.

FIG. 12 shows ORTEP of compound 3d.

FIG. 13 shows ORTEP of compound 8a.

FIG. 14 shows ORTEP of compound 8d.

FIG. 15 shows ORTEP of compound 10a.

FIG. 16 shows ORTEP of compound 12h.

FIG. 17 shows superimposed ¹H NMR spectra of compounds in Table 2 in CD₃OD.

FIG. 18 shows (A) Phospho-eIF4e (p-eIF4E) at serine 209 is monitored by immunoblotting after treatment with II-11k (NUCC-0201049), II-9d (NUCC-0231068), II-11f, II-12f, II-14f, IT-11n, II-12n, and eFT508 in LN229 cells at 2 h at 10 and 100 nM. (B) Structures of compounds tested by western blot.

FIG. 19 shows biochemical data of MNK inhibitors. (A) Dose-response curves for MNK1/2 inhibition. (B) MNK1/2 IC₅₀ values of compounds. IC₅₀ values were calculated in GraphPad Prism using a non-linear regression analysis ([inhibitor] vs. response (four parameters). Data represent mean±SEM of two independent repeats. ^{a}The average of the IC₅₀ values from 2 separate assay runs each with 9 dose points conducted in biological duplicate are reported with their 95% CI.

FIG. 20 shows molecular modeling of new MNK inhibitors bound to MNK2-D228G. (A) Docked pose of II-9d (NUCC-0231068) bound to MNK2. (B) Bound structure of II-11k (NUCC-0201049) with MNK2 from X-ray co-crystal structure 9HRC as a comparison. The identified hydrogen-bonds are shown in dotted lines.

FIG. 21 shows the dose-response curves for inhibition of eIF4E Ser209 phosphorylation in LN229 cells for the amine hits shown in Table 15.

FIG. 22 shows the dose-response curves for inhibition of eIF4E Ser209 phosphorylation in LN229 cells for the ether hits shown in Table 16.

### DETAILED DESCRIPTION OF THE INVENTION

Disclosed herein are compounds and derivative thereof for use as mitogen-activated protein kinase interacting kinase (MNK) inhibitors. The compounds and compositions disclosed herein may be used in methods of treating diseases or disorders, such as a cancer or a cell proliferative disorder, or a central nervous system (CNS) disease.

### Chemical Entities

The term “alkyl” refers to a straight-chain, branched, or cyclized alkyl radical in all of its isomeric forms, such as a straight or branched group of 1-12, 1-10, or 1-6 carbon atoms, referred to herein as C₁-C₁₂-alkyl, C₁-C₁₀-alkyl, and C₁-C₆-alkyl, respectively.

The term “alkylene” refers to a diradical of straight-chain or branched alkyl group (e.g., a diradical of straight-chain or branched C₁-C₁₂ alkyl group). Exemplary alkylene groups include, but are not limited to —CH₂—, —CH₂CH₂—, —CH₂CH₂CH₂—, —CH(CH₃)CH₂—, —CH₂CH(CH₃)CH₂—, —CH(CH₂CH₃)CH₂—, and the like.

The term “alkenyl” refers to an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively.

The terms “alkoxy” or “alkoxyl” refers to an alkyl group, as defined above, having an oxygen radical attached thereto. Representative alkoxy groups include methoxy, ethoxy, tert-butoxy and the like.

The term “cycloalkyl” refers to a monovalent saturated or partially saturated cyclic, bicyclic, or polycyclic hydrocarbon group of 3-12, 3-8, 4-8, or 4-6 carbons, referred to herein, e.g., as “C₄₋₈-cycloalkyl,” derived from a cycloalkane. Unless specified otherwise, the cycloalkyl group is not substituted, i.e., it is unsubstituted. The bicyclic or polycyclic cycloalkyl may contain a fused, a bridged (e.g., adamantyl), or a spiro cycloalkyl.

The term “heterocycloalkyl” (or “heterocyclyl”) refers to a monovalent saturated or partially saturated cyclic, bicyclic, or polycyclic hydrocarbon group of 3-12, 3-8, 4-8, or 4-6 carbons in which at least one carbon of the cycloalkane is replaced with a heteroatom such as, for example, N, O, and/or S. The bicyclic or polycyclic heterocycloalkyl may contain a fused, a bridged, or a spiro heterocycloalkyl.

The terms “amine” and “amino” refer to both unsubstituted (e.g., —NH₂) and substituted amines (e.g., mono-substituted amines or di-substituted amines), wherein substituents may include, for example, alkyl, haloalkyl, cycloalkyl, heterocyclyl, alkenyl, and aryl.

The term “oxo” refers to the ═O substituent.

The terms “halo” and “halogen” refer to a halogen atom or halogen radical (e.g., —F, —Cl, —Br, or —I).

The term “haloalkyl” refers to an alkyl group that is substituted with at least one halogen.

For example, —CH₂F, —CHF₂, —CF₃, —CH₂CF₃, —CF₂CF₃, and the like.

The term “cyano” refers to —CN.

The term “hydroxy” refers to —OH.

The term “aryl” refers to a carbocyclic aromatic group. The term “aryl” includes monocyclic ring systems, and polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aryl ring is unsubstituted. In certain embodiments, the aryl group is a 6-10 membered ring structure. Representative aryl groups include phenyl, naphthyl, anthracenyl, 1,3-benzodioxolyl and the like.

The substituents on the aryl (e.g., phenyl) rings in the compounds of the disclosure may be at ortho-, meta-, or para-positions.

The term “heteroaryl” refers to an aromatic 5- to 12-membered ring structure, alternatively 6- to 10-membered rings, whose ring structures include one to four heteroatoms, such as nitrogen, oxygen, and sulfur. The number of ring atoms in the heteroaryl group can be specified using Cx-Cx nomenclature where x is an integer specifying the number of ring atoms. For example, a C₃-C₇ heteroaryl group refers to an aromatic 3- to 7-membered ring structure containing one to four heteroatoms, such as nitrogen, oxygen, and sulfur. The term “heteroaryl” includes monocyclic ring systems, and polycyclic ring systems having two or more heterocyclic rings in which two or more carbon or heteroatom are common to two adjoining rings (the rings are “fused rings”) wherein at least one of the rings is a heterocyclic aromatic group and, e.g., the other ring(s) may be cycloalkyls, cycloalkenyls, cycloalkynyls, and/or aryls. Representative heteroaryl groups include benzimidazolyl, imidazopyridinyl, pyrazolyl, imidazolyl, pyridinyl (pyridyl), quinolinyl, furanyl, thionyl, indolyl, and the like.

The term “optionally substituted” refers to one or more carbon atoms in the group being independently substituted with one or more functional groups described herein.

If a group is described as being “substituted”, a non-hydrogen substituent group is in the place of hydrogen on a carbon or nitrogen of that group. Thus, for example, a substituted alkyl is an alkyl in which at least one non-hydrogen group is in the place of a hydrogen on the alkyl. To illustrate, monofluoroalkyl is alkyl substituted with a fluoro group, and difluoroalkyl is alkyl substituted with two fluoro groups. It should be recognized that if there is more than one substitution on a substituent, each non-hydrogen group may be identical or different (unless otherwise stated). Substituent groups include, but are not limited to, halogen, ═O, ═S, cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” or “+” or “−” depending on the configuration of substituents around the stereogenic carbon atom and or the optical rotation observed. The present invention encompasses various stereo isomers of these compounds and mixtures thereof. Stereoisomers include enantiomers and diastereomers. Mixtures of enantiomers or diastereomers may be designated (±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise. Also contemplated herein are compositions comprising, consisting essentially of, or consisting of an enantiopure compound, which composition may comprise, consist essentially of, or consist of at least about 50%, 60%, 70%, 80%, 90%, 95%, 96%, 97%, 98%, 99%, or 100% of a single enantiomer of a given compound (e.g., at least about 99% of an R enantiomer of a given compound).

As used herein, “salt” refers to acid addition salts and basic addition salts. It may also refer to those salts that may be prepared in situ during the final isolation and purification of the compounds of the invention.

Examples of acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, malate, maleate, methanesulfonate, nicotinate, 2-naphthalenesulfonate, oxalate, palmitate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid, and citric acid.

Basic addition salts may be prepared in situ during the final isolation and purification of compounds of this invention by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other examples of organic amines useful for the formation of base addition salts include ethylenediamine, ethanolamine, diethanolamine, piperidine, piperazine and the like.

Compounds described herein may exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

### Compounds

One aspect of the present disclosure provides a compound of formula (I), or a pharmaceutically acceptable salt, hydrate, or solvate thereof,

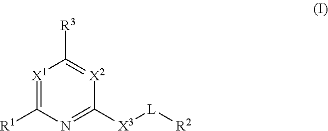

wherein
(i) X³ is NR⁹, X¹ is N, X² is CR⁸;
   R³ is —H;
   R⁸ is —CN, —H, halogen,- or —CF₃;
   R⁹ is —H, or R⁹ together with the nitrogen atom it is attached to, L, and R² form a monocyclic or bicyclic heterocycloalkyl substituted with 0-1 hydroxyl or —NH₂;
   R¹ is benzimidazolyl substituted with 0-1 —NH₂;
   L is absent or an alkylene, wherein L is substituted with 0-1 hydroxyl; and
   R² is heterocycloalkyl, -4-pyridyl, phenyl, or cycloalkyl; wherein R² is substituted with 0-1 hydroxyl, halogen, —NH₂, or alkyl optionally substituted with amino; or
(ii) X³ is O, X¹ is N, X² is CR⁴;
   R³ is —H, alkyl or cycloalkyl;
   R⁴ is halogen, —H, —CN, or haloalkyl;
   R¹ is heteroaryl, wherein R¹ is substituted with 0-1 —NH₂, halogen, —CN, or —NO₂;
   L is absent or an alkylene; and
   R² is pyridyl, heterocycloalkyl, cycloalkyl, or phenyl, wherein R² is substituted with 0-1 amino, alkyl, oxo, halogen, —NH—C(O)-cycloalkyl, —C(O)—O-alkyl, —C(O)—OH, or alkyl optionally substituted with amino;
or
(iii) X³ is O, X¹ is CR⁵, X² is N;
   R³ is —H;
   R⁵ is —H or halogen;
   R¹ is a heteroaryl, wherein R¹ is substituted with 0-1 —NH₂;
   L is absent; and
   R² is piperidinyl, wherein R² is substituted with 0-1 alkyl;
or
(iv) X³ is NR⁶, X¹ is CR⁷, X² is N;
   R³ is —H;
   R⁶ is —H, or R⁶ together with the nitrogen atom it is attached to, L, and R² form a bicyclic heterocycloalkyl;
   R⁷ is —H, —Cl, or —Br;
   R¹ is benzimidazolyl, pyrazolyl, or imidazolyl, wherein R¹ is substituted with 0-1 —NH₂, halogen, —CN, methyl, —C₆H₅, or —C(O)—NH₂; and
   -L-R² is

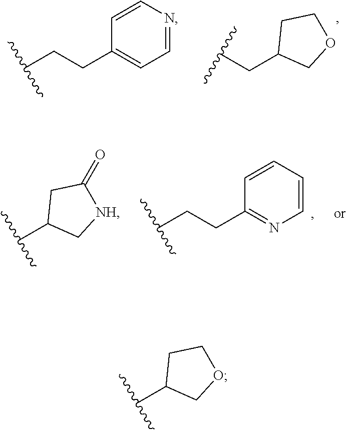 [SMILES: *CCc1ccncc1 ; *CC1CCOC1 ; *C1CNC(=O)C1 ; *CCc1ccccn1 ; *C1CCOC1]

with the proviso that the compound is not

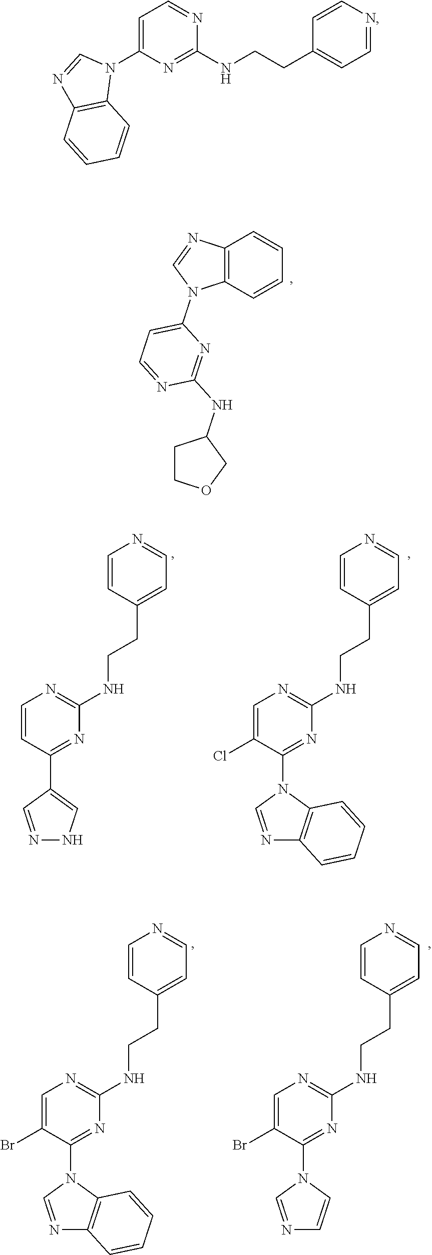 [SMILES: c1ccc2c(c1)ncn2-c1ccnc(NCCc2ccncc2)n1 ; c1ccc2c(c1)ncn2-c1ccnc(NC2CCOC2)n1 ; c1cc(CCNc2nccc(-c3cn[nH]c3)n2)ccn1 ; Clc1cnc(NCCc2ccncc2)nc1-n1cnc2ccccc21 ; Brc1cnc(NCCc2ccncc2)nc1-n1cnc2ccccc21 ; Brc1cnc(NCCc2ccncc2)nc1-n1ccnc1]

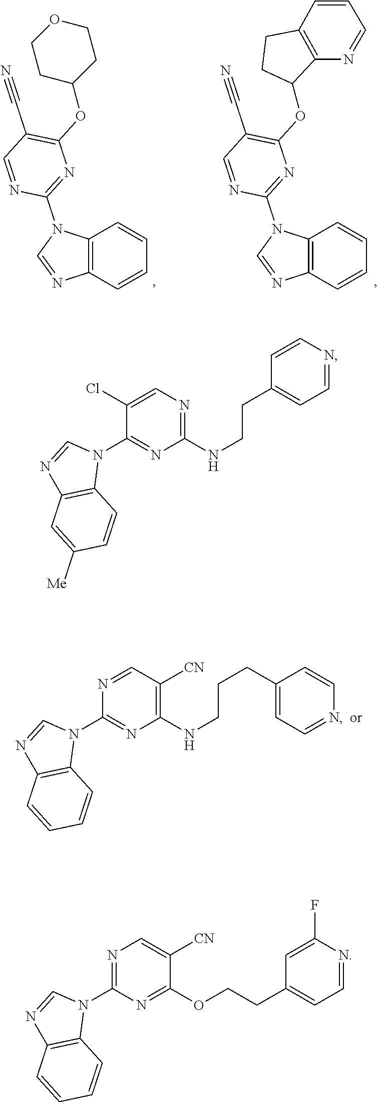

In some embodiments, (i) X³ is NR, X¹ is N, and X² is CR⁸.

In some embodiments of scenario (i), R⁸ is —CN, —H, —Cl, —Br, or —CF₃.

In some embodiments of scenario (i), L is absent. In some other embodiments of scenario (iv), L is ethylene substituted with 0-1 hydroxyl, and R² is-4-pyridyl or heterocycloalkyl.

In some embodiments of scenario (i), R⁹ is —H. In some other embodiments of scenario (i), R⁹ together with the nitrogen atom it is attached to, L, and R² form

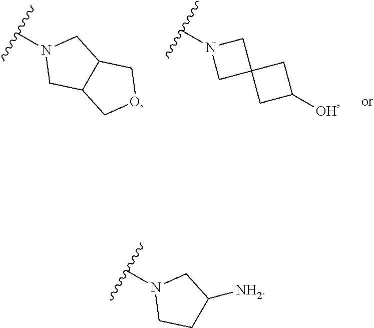 [SMILES: *N1CC2COCC2C1 ; *N1CC2(CC(O)C2)C1 ; *N1CCC(N)C1]

In some embodiments of scenario (i), R² is heterocycloalkyl or cycloalkyl, wherein R² is substituted with 0-1 hydroxyl, —NH₂, or —CH₂—NH₂.

In some embodiments of scenario (i), R² is

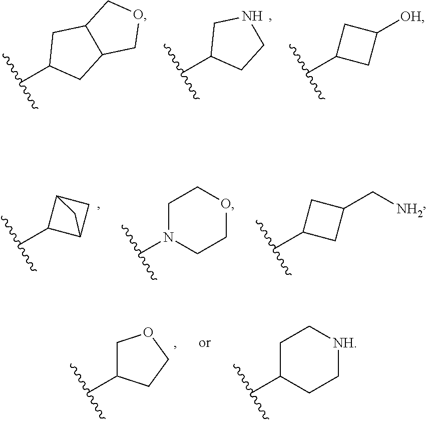

In some embodiments of scenario (i), R² is pyridyl or phenyl substituted with 0-1 —F.

In some embodiments, (ii) X³ is O, X¹ is N, and X² is CR⁴.

In some embodiments of scenario (ii), R¹ is benzimidazolyl or imidazopyridinyl substituted with 0-1 —NH₂, halogen, —CN, or —NO₂. In some such embodiments, R¹ may be benzimidazolyl substituted with 0-1 —NH₂, —CN, or —Br. In some other such embodiments, R¹ may be imidazopyridinyl substituted with 0-1 —NH₂ or —F.

In some embodiments of scenario (ii), R³ is H.

In some embodiments of scenario (ii), R³ is methyl or cyclopropyl.

In some embodiment of scenario (ii), R⁴ is —Cl, —F, —Br, —CF₃, or —CN.

In some embodiments of scenario (ii), L is C₁-C₆ alkylene. In some such embodiments, L may be ethylene.

In some embodiments of scenario (ii), L is absent.

In some embodiments of scenario (ii), R² is heterocycloalkyl substituted with 0-1 methyl, oxo, or —C(O)—O-tBu. In some such embodiments, R² may be

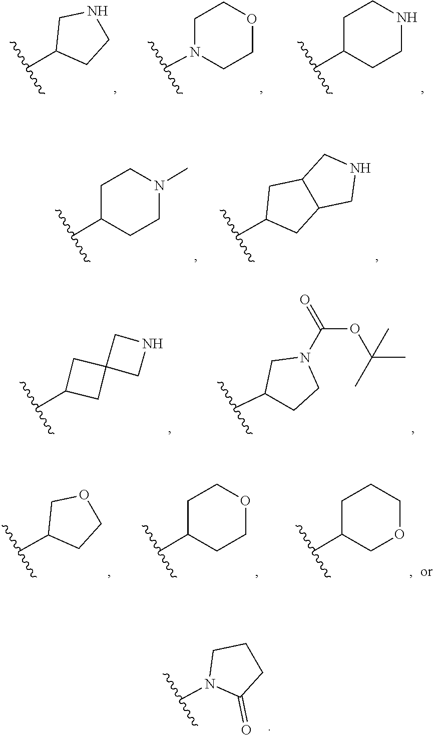

In some embodiments of scenario (ii), R² is pyridyl substituted with 0-1 —F.

In some embodiments of scenario (ii), R² is cycloalkyl substituted with 0-1 —NH₂, —NH—CH₃, —NH—C(O)-cyclopropyl, —CH₂—NH₂, or —C(O)—OH. In some such embodiments, R² may be

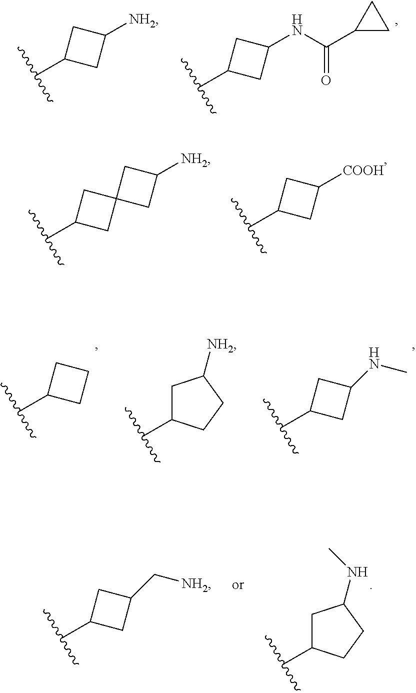

In some embodiments of scenario (ii), R² is phenyl substituted with 0-1 —F, —Cl, or —NH₂.

In some embodiments, (iii) X³ is O, X¹ is CR⁵, and X² is N.

In some embodiments of scenario (iii), R⁵ is —Cl.

In some embodiments of scenario (iii), R¹ is benzimidazolyl substituted with 0-1 —NH₂.

In some embodiments of scenario (iii), R² is piperidinyl substituted with 0-1 methyl. In some such embodiments, R² may be

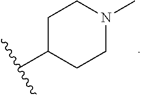 [SMILES: *C1CCN(C)CC1]

In some embodiments, (iv) X³ is NR⁶, X¹ is CR⁷, and X² is N.

In some embodiments of scenario (iv), R⁶ is H.

In some embodiments of scenario (iv), R⁶ together with X³, L, and R² form a bicyclic heterocycloalkyl, such as

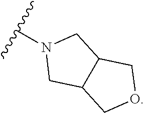 [SMILES: *N1CC2COCC2C1]

In some embodiments of scenario (iv), R¹ is pyrazolyl substituted with 0-1 substituent independently selected from methyl and —C₆H₅.

In some embodiments of scenario (iv), R¹ is benzimidazolyl substituted with 0-1 substituent independently selected from —C(O)—NH₂, —CN, —NH₂, and —Br.

In some embodiments of scenario (iv), R¹ is imidazolyl.

In some embodiments, the compound is

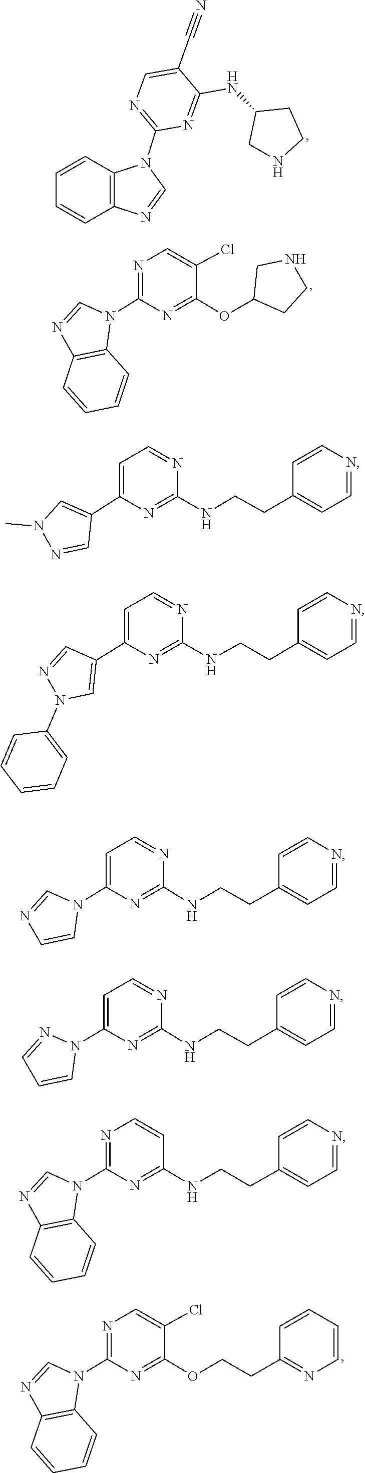

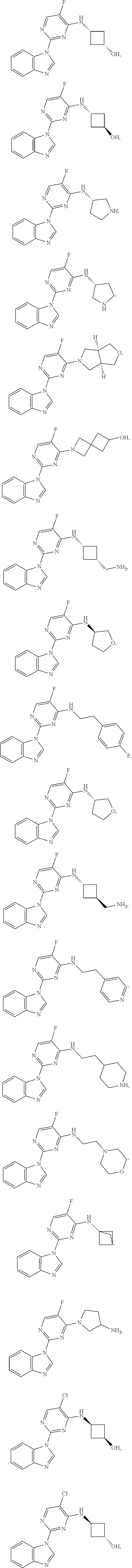

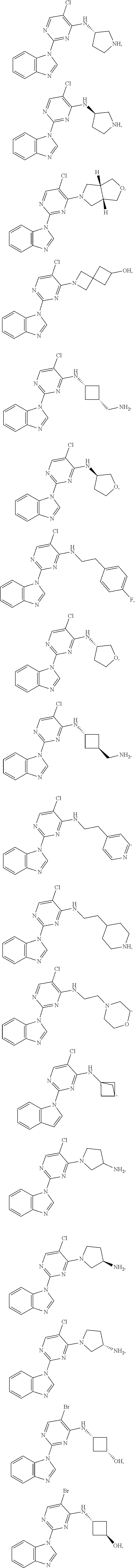

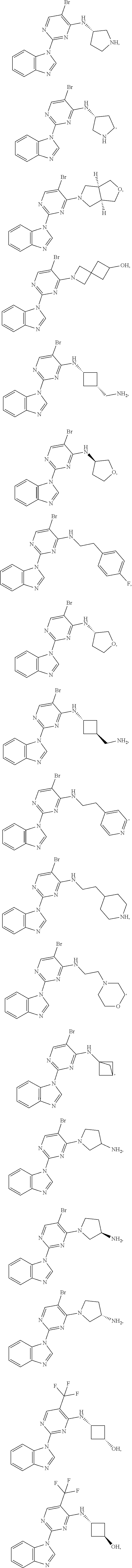

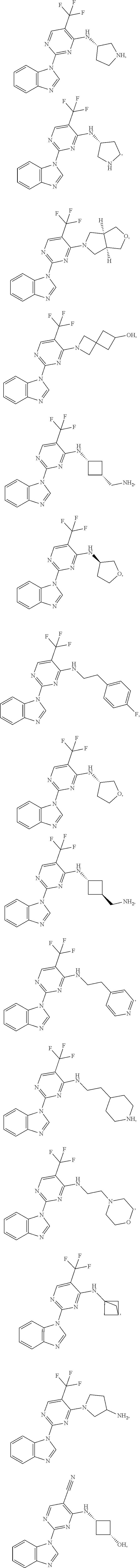

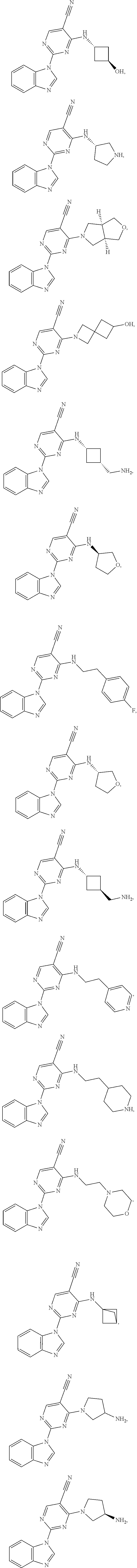

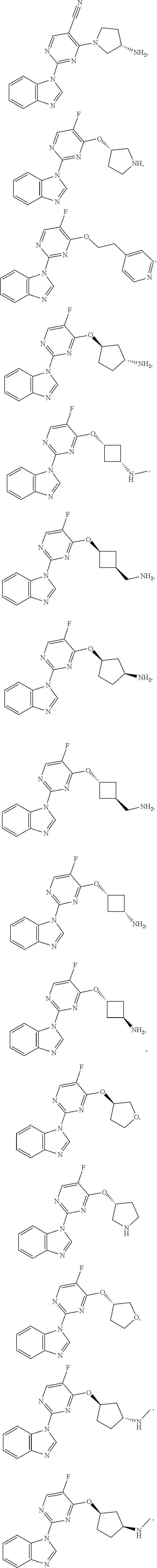

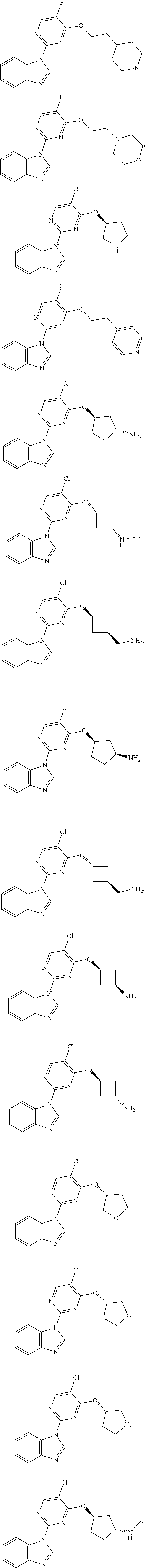

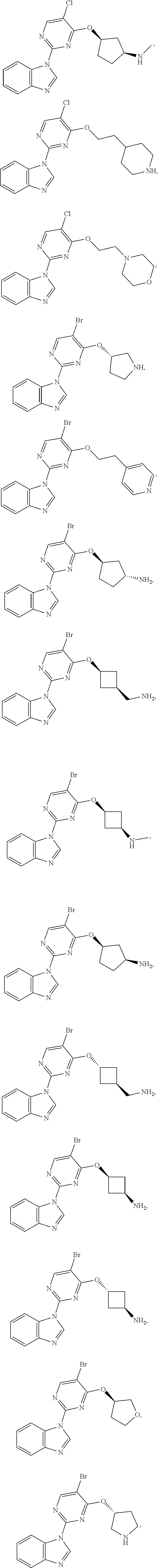

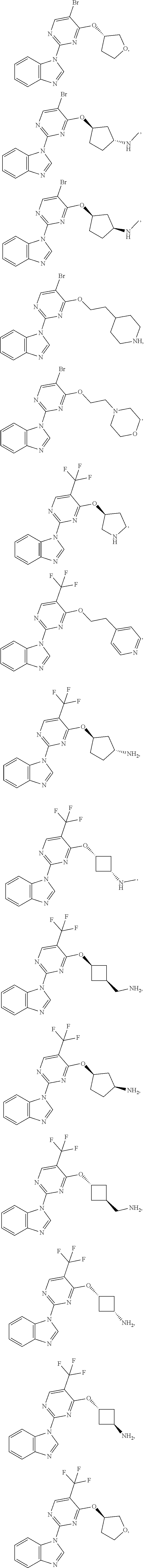

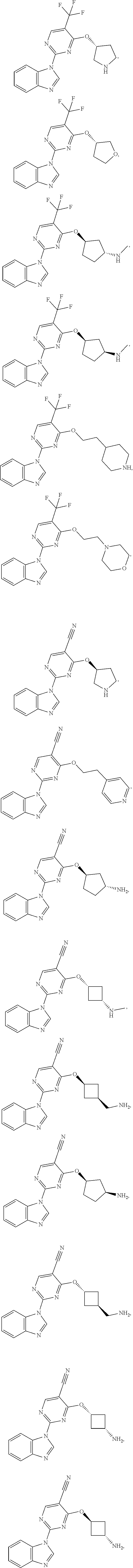

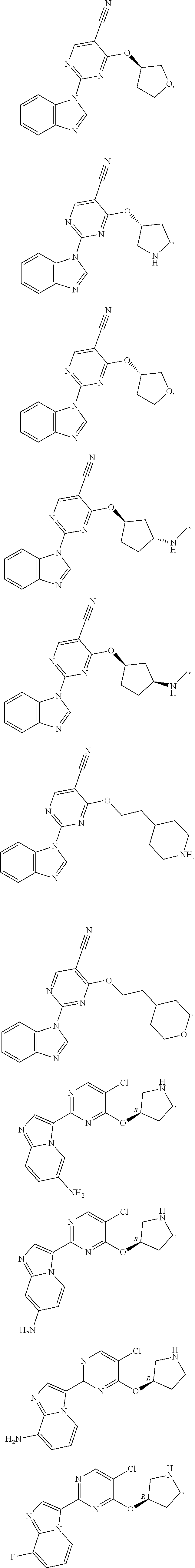

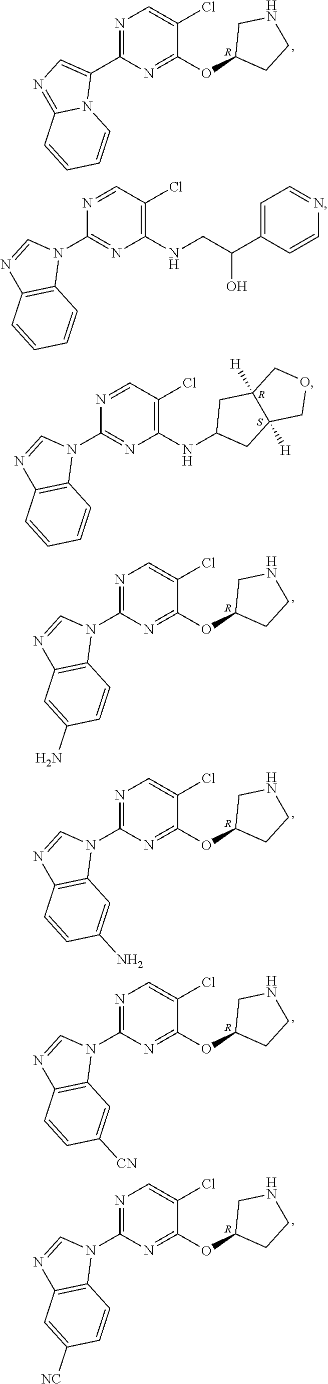

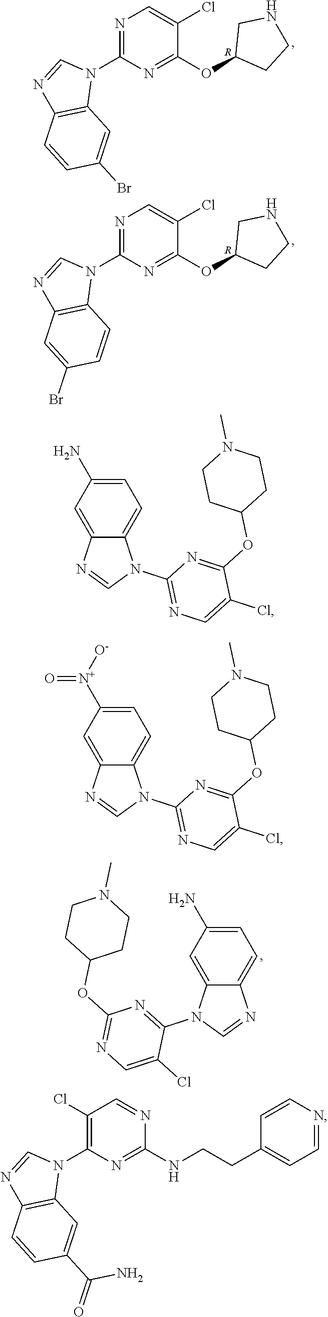

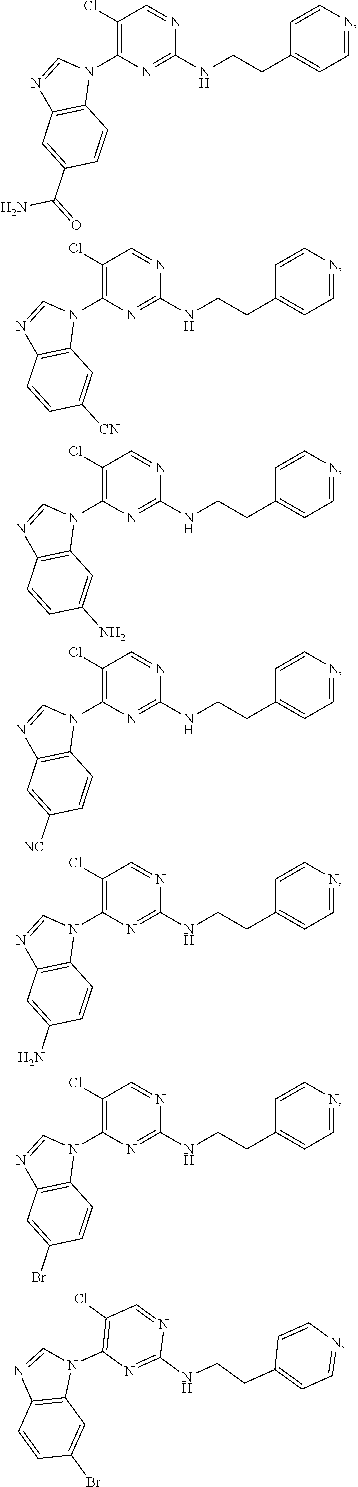

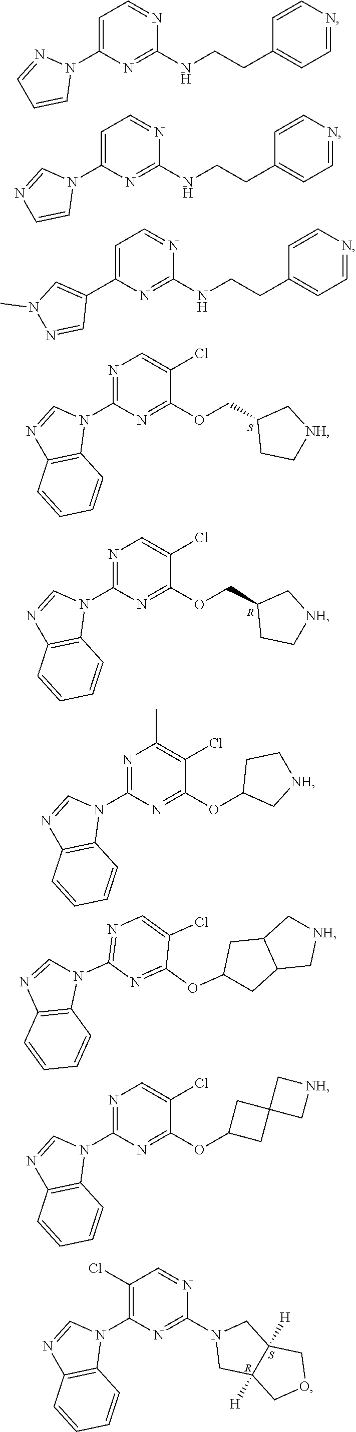

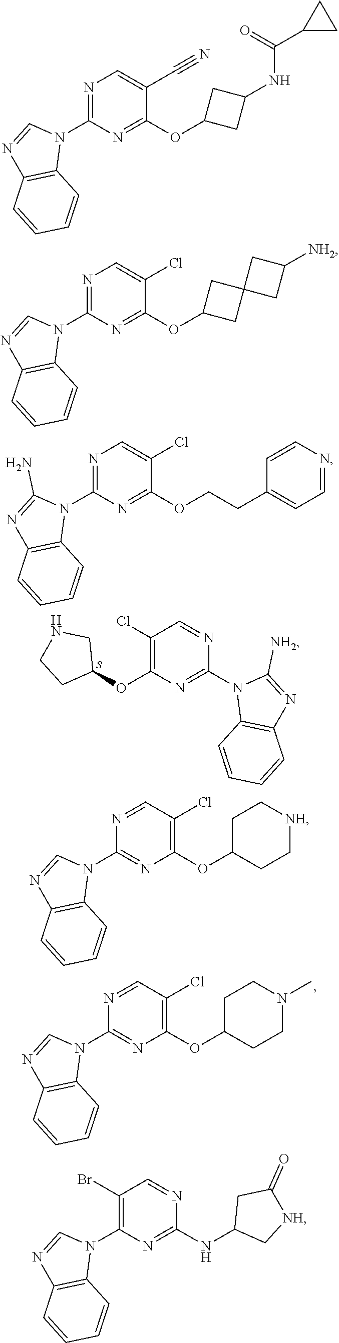

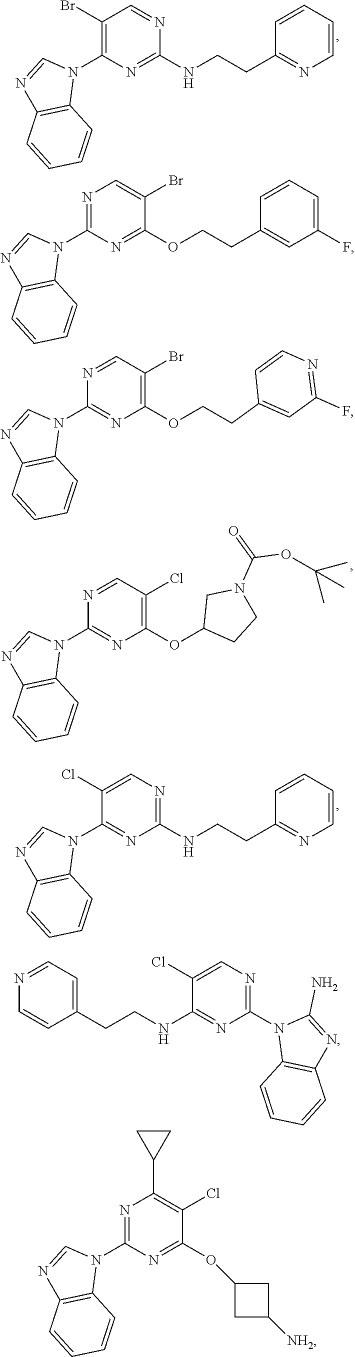

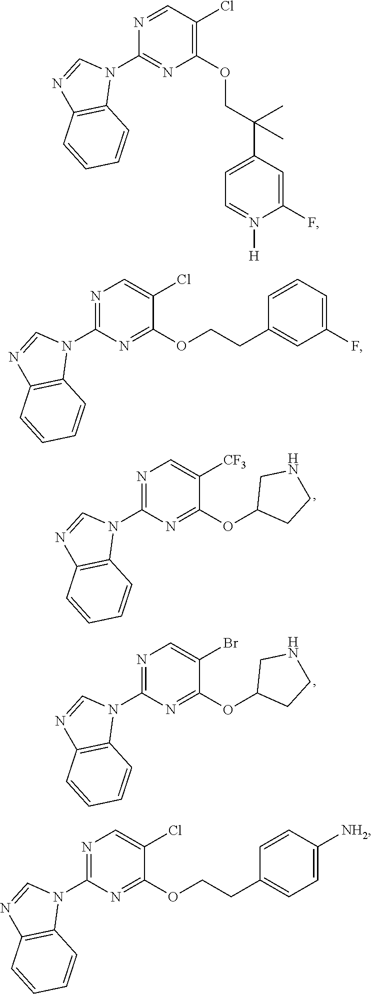

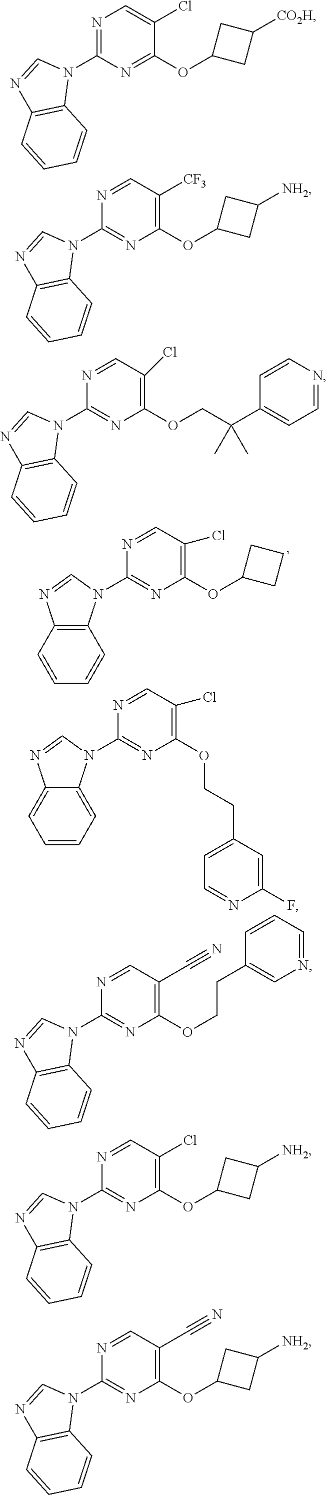

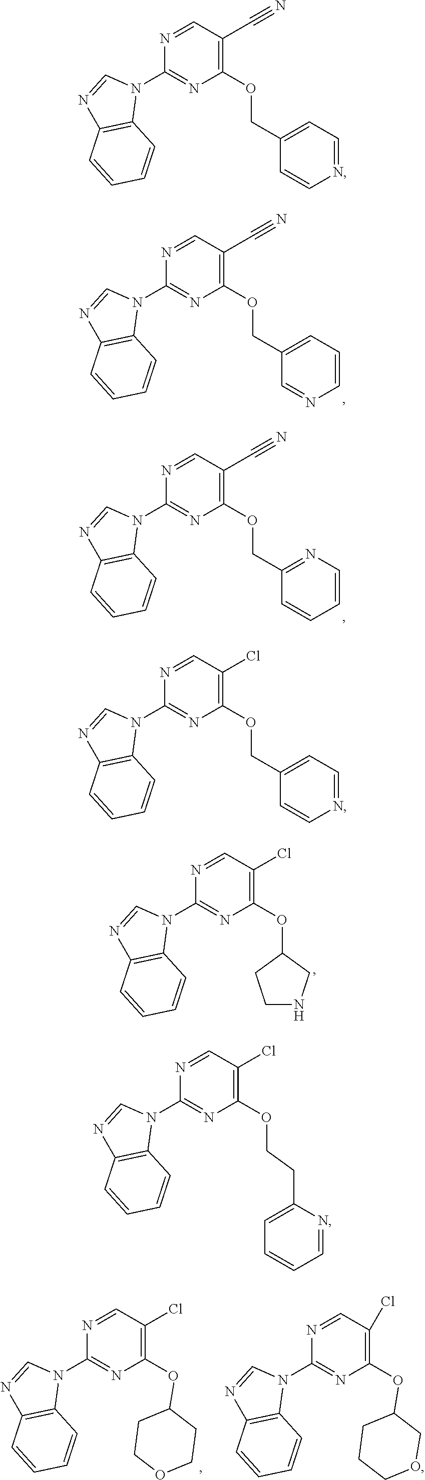

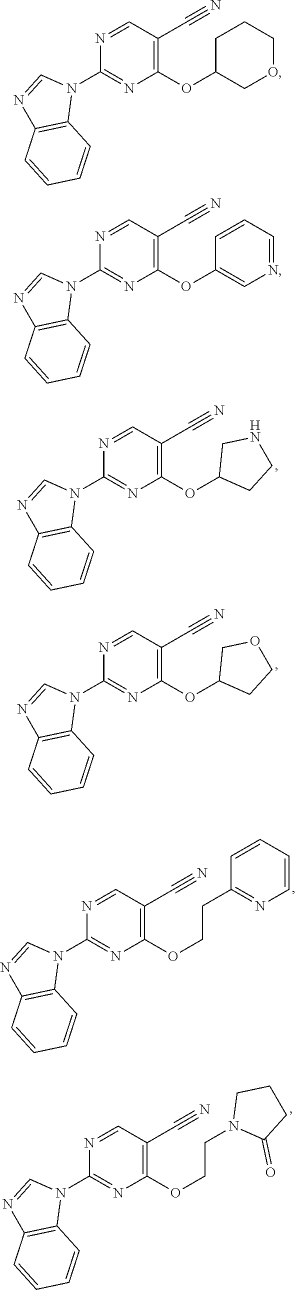 [SMILES: N#Cc1cnc(-n2cnc3ccccc32)nc1OC1CCCOC1 ; N#Cc1cnc(-n2cnc3ccccc32)nc1Oc1cccnc1 ; N#Cc1cnc(-n2cnc3ccccc32)nc1OC1CCNC1 ; N#Cc1cnc(-n2cnc3ccccc32)nc1OC1CCOC1 ; N#Cc1cnc(-n2cnc3ccccc32)nc1OCCc1ccccn1 ; N#Cc1cnc(-n2cnc3ccccc32)nc1OCCN1CCCC1=O]

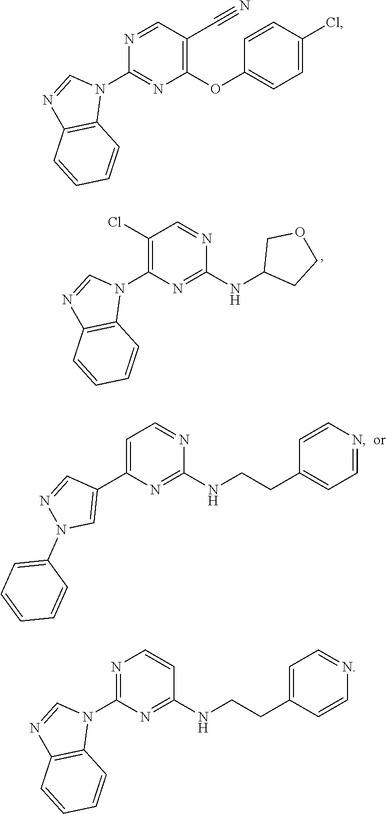

or a pharmaceutically acceptable salt, hydrate, or solvate thereof.

In some embodiments, the compound is

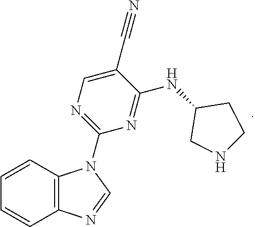 [SMILES: N#Cc1cnc(-n2cnc3ccccc32)nc1N[C@@H]1CCNC1]

or a pharmaceutically acceptable salt, hydrate, or solvate thereof.

In some embodiments, the compound is

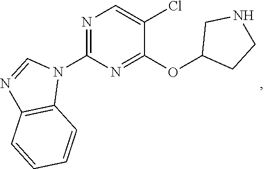 [SMILES: Clc1cnc(-n2cnc3ccccc32)nc1OC1CCNC1]

or a pharmaceutically acceptable salt, hydrate, or solvate thereof.

The disclosed compounds may be formulated as pharmaceutical compositions comprising any of the compounds disclosed herein and a pharmaceutically acceptable excipient, carrier, or diluent.

The disclosed compounds and compositions comprising the same may exhibit one or more biological activities. In some embodiments, the disclosed compounds inhibit the activity of mitogen-activated protein kinase interacting kinase 1 and/or 2 (MNK1 and/or MNK2). In some embodiments, the disclosed compounds inhibit the activity of MNK1 and/or MNK2 by at least 50%, 60%, 70%, 80%, 90%, 95%, 96%, 97%, 98%, 99%, or more, at a concentration of less than 100 μM, 50 μM, 10 μM, 1 μM, 0.1 μM, 0.05 μM, 0.01 μM, 0.005 μM, 0.001 μM, or less.

Concentration ranges also are contemplated herein, for example, a concentration range bounded by end-point concentrations selected from 0.001 μM, 0.005 μM, 0.01 μM, 0.5 μM, 0.1 μM, 1.0 μM, 10 μM, and 100 μM.

Cell proliferation and inhibition thereof by the disclosed compounds may be assessed by methods disclosed in the art, including an ADP-Glo universal kinase assay and radiometric biochemical assays, according to methods described herein. In some embodiments, the disclosed compounds have an IC₅₀ against MNK1 and/or MNK2 from greater than 0 μM to 150 μM, from greater than 0 μM to 100 μM, from greater than 0 μM to 50 μM, from greater than 0 μM to 40 μM, from greater than 0 μM to 30 μM, from greater than 0 μM to 20 μM, from greater than 0 μM to 10 μM, from greater than 0 μM to 5 μM, from greater than 0 μM to 1 μM, from greater than 0 μM to 0.5 μM, from greater than 0 μM to 0.1 μM, from greater than 0 μM to 0.01 μM, or less than 0.01 μM, in ADP-Glo kinase assay or biochemical assay.

In some embodiments, the disclosed compounds are selective MNK1/2 inhibitors.

In some embodiments, kinase selectivity may be assessed through a kinase binding assay in a kinase panel and selectivity scores (S scores). In short, compounds that bind the kinase active site and directly (sterically) or indirectly (allosterically) prevent kinase binding to the immobilized ligand, will reduce the amount of kinase captured on the solid support. Conversely, test molecules that do not bind the kinase have no effect on the amount of kinase captured on the solid support. Screening “hits” are identified by measuring the amount of kinase captured in test versus control samples.

In some embodiments, selective MNK1/2 inhibitors exhibit the strongest inhibition of MNK (e.g., MNK1 and/or MNK2) among all the kinases screened, such as shown in Tables 4 and 5.

Percent Control (% Ctrl). The compounds may be screened at any suitable concentration.

For example, the compounds may be screened at a 10 μM concentration but other concentrations may also be used. Results for primary screen binding interactions are reported as “% Ctrl” or “POC”, where lower numbers indicate stronger hits in the matrix. % Ctrl is defined as (eqn 1):

$$\% Ctrl = 100 \times ( TS - CPOS ) / ( CNEG - CPOS ) \qquad ( eqn 1 )$$

where TS is the test compound signal, CPOS is the positive control signal (0% Ctrl), CNEG is the DMSO negative control (100% Ctrl).

Selectivity Score (S scores). Selectivity Score or S-score is a quantitative measure of compound selectivity. The S-score is calculated by dividing the number of kinases that compounds bind to by the total number of distinct kinases tested, excluding mutant variants (eqn 2):

$$S = Number of hits / Number of assays \qquad ( eqn 2 )$$

This value can be calculated using % Ctrl as a potency threshold and provides a quantitative method of describing compound selectivity to facilitate comparison of different compounds:

$$S ( 35 ) = ( \# of non - mutant kinases with \% Ctrl < 35 ) / ( \# of non - mutant kinases tested ) \qquad ( eqn 3 )$$

$$S ( 10 ) = ( \# of non - mutant kinases with \% Ctrl < 10 ) / ( \# of non - mutant kinases tested ) \qquad ( eqn 4 )$$

$$S ( 1 ) = ( \# of non - mutant kinases with \% Ctrl < 1 ) / ( \# of non - mutant kinases tested ) \qquad ( eqn 5 )$$

The disclosed compounds may have a % Ctrl less than 5% for MNK1, MNK2, or both MNK1 and MNK2. In some instances, the compound may have a % Ctrl less than 4%, 3%, 2%, 1%, 0.5%, or 0.1% and greater than 0% for MNK1, MNK2, or both MNK1 and MNK2.

The disclosed compounds may have an S(35) value less than 0.25. In some instances, the disclosed compounds may have an S(35) value less than 0.20 or 0.15. The disclosed compounds may have an S(10) value less than 0.15. In some instances, the disclosed compounds may have an S(10) value less than 0.10, 0.08, or 0.06. The disclosed compounds may have an S(1) value less than 0.1. In some instances, the disclosed compounds may have an S(1) value less than 0.08, 0.05, or 0.03.

In some embodiments, the disclosed compounds decrease phosphorylation of eIF4E at Ser209 in AML cells, such as in MV4-11 cells and/or in U937 cells; or in glioblastoma (GBM) cells, such as in LN229 cells. In some embodiments, the disclosed compounds decrease phosphorylation of eIF4E at Ser209 in AML and/or GBM cells by at least 30%, 40%, 50%, 60%, 70%, 80%, 90%, 95%, 96%, 97%, 98%, 99%, or more, at a concentration of 100 μM, 50 μM, 10 μM, 1 μM, 0.1 μM, 0.05 μM, 0.01 μM, 0.005 μM, 0.001 μM, or less. In some embodiments, phosphorylation of eIF4E may be assessed using cellular flow cytometric eIF4E phosphorylation assays, Western blotting, or in-cell Western assays, according to methods described herein. In some embodiments, the disclosed compounds have an IC₅₀ in inhibiting phosphorylation of eIF4E at Ser209 from greater than 0 μM to 150 μM, from greater than 0 μM to 100 μM, from greater than 0 μM to 50 μM, from greater than 0 μM to 40 μM, from greater than 0 μM to 30 μM, from greater than 0 μM to 20 μM, from greater than 0 μM to 10 μM, from greater than 0 μM to 5 μM, from greater than 0 μM to 1 μM, from greater than 0 μM to 0.5 μM, from greater than 0 μM to 0.1 μM, from greater than 0 μM to 0.01 μM, or less than 0.01 μM, in cellular flow cytometric eIF4E phosphorylation assay, Western blotting, or in-cell Western assay.

In some embodiments, the disclosed compounds decrease the viability of AML cells, such as MV4-11 cells and/or U937 cells. In some embodiments, the disclosed compounds decrease viability of AML cells by at least 30%, 40%, 50%, 60%, 70%, 80%, 90%, 95%, 96%, 97%, 98%, 99%, or more, at a concentration of 100 μM, 50 μM, 10 μM, 1 μM, 0.1 μM, 0.05 μM, 0.01 μM, 0.005 μM, 0.001 μM, or less.

In some embodiments, the disclosed compounds enhance antineoplastic effects of a hypomethylating agent, such as 5-azacytidine. In some embodiments, the disclosed compounds, in combination with a hypomethylating agent, decrease viability of AML cells by at least 30%, 40%, 50%, 60%, 70%, 80%, 90%, 95%, 96%, 97%, 98%, 99%, or more, at a concentration of 100 μM, 50 μM, 10 μM, 1 μM, 0.1 μM, 0.05 μM, 0.01 μM, 0.005 μM, 0.001 μM, or less.

In some embodiments, the disclosed compounds induce apoptosis in AML cells, such as in MV4-11 cells and/or in U937 cells. In some embodiments, the disclosed compounds induce apoptosis of at least least 30%, 40%, 50%, 60%, 70%, 80%, 90%, 95%, 96%, 97%, 98%, 99%, or more, of the AML cells at a concentration of 100 μM, 50 μM, 10 μM, 1 μM, 0.1 μM, 0.05 μM, 0.01 μM, 0.005 μM, 0.001 μM, or less. In some embodiments, apoptosis of AML cells is analyzed through flow cytometry analysis using Annexin V FITC and 4′-6-diamidino-2-phenylindole (DAPI)-DNA staining, according to methods described herein.

In some embodiments, the disclosed compounds may penetrate the blood-brain barrier (BBB) and achieve a desirable drug level in the brain. In some embodiments, the disclosed compounds achieve a total brain/plasma (B/P) ratio of at least 2.5, 3, 3.5, 4, 4.5, or 5, in a subject after administering the compound to the subject. Methods of administration include, but are not limited to, oral administration and intravenous administration.

### Pharmaceutical Compositions

Another aspect of the present disclosure provides a pharmaceutical composition comprising the compound disclosed herein and a pharmaceutically acceptable excipient, carrier, or diluent.

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

In some embodiments, the compounds disclosed herein may be formulated as pharmaceutical compositions that include: (a) a therapeutically effective amount of one or more compounds as disclosed herein; and (b) one or more pharmaceutically acceptable carriers, excipients, or diluents. The pharmaceutical composition may include the compound in a range of about 0.1 to 2000 mg (preferably about 0.5 to 500 mg, and more preferably about 1 to 100 mg).

The pharmaceutical composition may be administered to provide the compound at a daily dose of about 0.1 to about 1000 mg/kg body weight (preferably about 0.5 to about 500 mg/kg body weight, more preferably about 50 to about 100 mg/kg body weight). In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.001 nM, 0.005 nM, 0.01 nM, 0.5 nM, 0.1 nM, 1.0 nM, 10 nM, and 100 nM (e.g., 0.1 nM-1.0 nM).

It is understood by those skilled in the art that dosage amount will vary with the activity of a particular inhibitor compound, disease state, route of administration, duration of treatment, and like factors well-known in the medical and pharmaceutical arts. In general, a suitable dose will be an amount which is the lowest dose effective to produce a therapeutic or prophylactic effect. If desired, an effective dose of such a compound, pharmaceutically acceptable salt thereof, or related composition may be administered in two or more sub-doses, administered separately over an appropriate period of time.

The compounds for use according to the methods of disclosed herein may be administered as a single compound or a combination of compounds. For example, a compound that inhibits MNK activity (e.g., MNK1 and/or MNK2 activity) may be administered as a single compound or in combination with another compound that inhibits MNK activity, or that has a different pharmacological activity. In some embodiments, one or more additional therapeutic agents may be administered with the disclosed compounds or with pharmaceutical compositions comprising the disclosed compounds, where the additional therapeutic agent is administered prior to, concurrently with, or after administering the disclosed compounds or the pharmaceutical compositions comprising the disclosed compounds. In some embodiments, the disclosed pharmaceutical composition is formulated to comprise the disclosed compounds and further to comprise one or more additional therapeutic agents, for example, one or more additional therapeutic agents for treating diseases and disorders.

As indicated above, pharmaceutically acceptable salts of the compounds are contemplated and also may be utilized in the disclosed methods. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein may not be critical to the activity of the compound, so long as the salt as a whole is pharmacologically acceptable and as long as the counterion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

The pharmaceutical composition may include the compound in a range of about 0.1 to 2000 mg. In some embodiments, the pharmaceutical composition may include the compound in a range of from about 0.5 to 500 mg. In some embodiments, the pharmaceutical composition may include the compound in a range from about 1 to 100 mg. The pharmaceutical composition may be administered to provide the compound at a daily dose of about 0.1 to about 1000 mg/kg body weight. In some embodiments, the pharmaceutical composition may be administered to provide the compound at a daily dose of about 0.5 to about 500 mg/kg body weight. In some embodiments, the pharmaceutical composition may be administered to provide the compound at a daily dose of about 50 to about 100 mg/kg body weight. A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment. In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.001 nM, 0.005 nM, 0.01 nM, 0.5 nM, 0.1 nM, 1.0 nM, 10 nM, and 100 nM (e.g., 0.1 nM-1.0 nM).

The compounds utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The compounds utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™) Suitable lubricants, including agents that act on the flowability of the powder to be compressed, may include colloidal silicon dioxide, such as Aerosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame.

Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

The compounds utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are in some embodiments applied as a topical ointment or cream.

When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use.

Extemporaneous injection solutions and suspensions may be prepared from sterile powders, granules and tablets.

Oral administration is an illustrative route of administering the compounds employed in the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

Methods of preparing pharmaceutical formulations or compositions include the step of bringing a disclosed compound into association with a carrier and, optionally, one or more additional adjuvants or ingredients. For example, standard pharmaceutical formulation techniques can be employed, such as those described in Remington's Pharmaceutical Sciences, Mack Publishing Company, Easton, PA.

Regardless of composition or formulation, those skilled in the art will recognize various avenues for medicament administration, together with corresponding factors and parameters to be considered in rendering such a medicament suitable for administration.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

### Methods

The disclosed compounds and pharmaceutical compositions comprising the disclosed compounds may be administered in methods of treating a disease or a disorder in a subject in need thereof, such as in methods of treating a disease or disorder associated with MNK1 and/or MNK2 activity.

Another aspect of the present disclosure provides a method for treating a disease or disorder associated with mitogen-activated protein kinase interacting kinase 1 and/or 2 (MNK1 and/or MNK2) activity in a subject in need thereof, the method comprising administering an effective amount of the compound disclosed herein, or a pharmaceutically acceptable salt, hydrate, or solvate thereof; or the pharmaceutical composition disclosed herein to the subject.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

A “subject in need thereof” as utilized herein refers to a subject in need of prevention and/or treatment for a disease or disorder associated with MNK activity, such as MNK1 and/or MNK2 activity. The term “subject” may be used interchangeably with the terms “individual” and “patient” and includes human and non-human mammalian subjects. In some embodiments, the treated subject may be a mammalian subject. Although the methods disclosed herein are particularly intended for the treatment of humans, other mammals are included. By way of non-limiting examples, mammalian subjects include monkeys, equines, cattle, canines, felines, mice, rats and pigs.

As used herein, the term “disorder” refers to a condition in which there is a disturbance of normal functioning. A “disease” is any abnormal condition of the body or mind that causes discomfort, dysfunction, or distress to the person affected or those in contact with the person. Sometimes the term is used broadly to include injuries, disabilities, syndromes, symptoms, deviant behaviors, and atypical variations of structure and function, while in other contexts these may be considered distinguishable categories. It should be noted that the terms “disease”, “disorder”, “condition” and “illness”, are equally used herein.

In some embodiments, the disease or disorder is a cancer or a cell proliferative disorder. In some embodiments, the cancer or the cell proliferative disorder includes, but is not limited to, leukemia, brain cancer (e.g., glioblastoma (GBM)), breast cancer, colon cancer, prostate cancer, bladder cancer, lung cancer, and pancreatic cancer. In some embodiments, the cancer or the cell proliferative disorder is a hematological malignancy, such as acute myeloid leukemia (AML).

In some embodiments, the disease or disorder is a central nervous system (CNS) disease. In some embodiments, the CNS disease includes, but is not limited to, Alzheimer's disease (AD), autism, and amyotrophic lateral sclerosis (ALS). In some embodiments, the disease or disorder is glioblastoma.

In some embodiments, the disease or disorder is a viral infection, inflammation, or depression.

In some embodiments, the method further comprises administering a hypomethylating agent to the subject. The hypomethylating agent includes, but is not limited to, 5-azacytidine and decitabine. In some embodiments, the hypomethylating agent is 5-azacytidine.

In some embodiments of the disclosed treatment methods, the subject may be administered a dose of a compound as low as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg once daily, twice daily, three times daily, four times daily, once weekly, twice weekly, or three times per week in order to treat the disease or disorder in the subject. In some embodiments, the subject may be administered a dose of a compound as high as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg, once daily, twice daily, three times daily, four times daily, once weekly, twice weekly, or three times per week in order to treat the disease or disorder in the subject. Minimal and/or maximal doses of the compounds may include doses falling within dose ranges having as end-points any of these disclosed doses (e.g., 2.5 mg-200 mg).

In some embodiments of the disclosed treatment methods, a minimal dose level of a compound for achieving therapy in the disclosed methods of treatment may be at least about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. In some embodiments, a maximal dose level of a compound for achieving therapy in the disclosed methods of treatment may not exceed about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. Minimal and/or maximal dose levels of the compounds for achieving therapy in the disclosed methods of treatment may include dose levels falling within ranges having as end-points any of these disclosed dose levels (e.g., 500-2000 ng/kg body weight of the subject).

As used herein the term “effective amount” refers to the amount or dose of the compound or composition, upon single or multiple dose administration to the subject, which provides the desired effect in the subject under diagnosis or treatment. The disclosed methods may include administering an effective amount of the disclosed compounds (e.g., as present in a pharmaceutical composition) for treating and/or preventing a disease or disorder associated with MNK1 and/or MNK2 activity.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

The compounds and compositions disclosed herein may be administered in methods of treatment as known in the art. Accordingly, various such compounds and compositions can be administered in conjunction with such a method in any suitable way. For example, administration may comprise oral, intravenous, intraarterial, intramuscular, subcutaneous, intraperitoneal, parenteral, transdermal, intravaginal, intranasal, mucosal, sublingual, topical, rectal or subcutaneous administration, or any combination thereof.

### Miscellaneous

Unless otherwise specified or indicated by context, the terms “a”, “an”, and “the” mean “one or more.” For example, “a molecule” should be interpreted to mean “one or more molecules.” As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus <10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1

### Results and Discussion

### Design of New MNK Inhibitors

In our effort to develop new MNK inhibitor chemotypes that were potent, selective, and had good pharmacokinetics properties, we incorporated a Mutation-based Induced-Fit Docking (M-IFD) in silico screening platform that enabled high-throughput virtual screening.²⁵

### Structure-Activity Relationships of the Hinge-Binding Group

We prepared heterocycle-containing hydrogen-bond acceptors (Table 1). Based on our studies of docking compounds into MNK2 kinase, the pyrazole nitrogen served as a hydrogen-bond acceptor and formed the key interaction with the hinge region Met162 backbone N—H. We have explored different variations to the pyrazole ring. Compound 3f displayed single-digit micromolar inhibition of both MNK1 and MNK2. Compound 3c without an methoxy group showed similar potency for MNK1 or MNK2 compared to 3f. When the pyrazole phenyl substituent was replaced by a methyl group (3b), potency decreased appreciably. The free N—H (3a) without the pyrazole nitrogen substituent resulted in potency that was very similar to compound 3f. Compound with the pyrazole hydrogen-bond acceptor nitrogen at the 2-position and directly attached the pyrazole to the pyrimidine (3e) showed significantly diminished potency for both MNK isoforms. Since having the hydrogen-bond acceptor group distal to the pyrimidine appeared beneficial, we prepared the imidazole derivative, 3d, which showed an improvement in MNK1 inhibition (1.3 μM). We then combined our observations that the hydrophobic aryl group and the imidazole hydrogen-bond acceptor nitrogen were both beneficial and synthesized the benzimidazole compounds. 8c showed a significant improvement in both MNK1 (IC₅₀=0.33 μM) and MNK2 (0.03 μM) inhibition potency. To confirm the importance of the benzimidazole N-3, the indole version of this compound (8e) was prepared and found to be significantly less potent against both MNK isoforms.

We also examined the role of the pyrimidine nitrogens. We prepared 8a, in which the benzimidazole is attached to the pyrimidine 2-position. This compound, 8a, showed an approximately 2-fold improvement in potency versus the benzimidazole-4-pyrimidine inhibitor 8c. As we continued to evaluate our molecular modeling, we appreciated that there existed a small hydrophobic pocket facing the pyrimidine 5-position of inhibitor 8a. To fill this pocket, we synthesized the chloro derivative 8b which further improved potency, with a MNK1 IC₅₀=70 nM and MNK2 IC₅₀=14 nM. To verify that the chloro substitution still preferred the benzimidazole in the 2-position of the pyrimidine, we synthesized regioisomeric compound 8d. This was approximately 100-fold less potent than 8b, further confirming the importance of having the benzimidazole in the 2-position of the pyrimidine ring going forward.

TABLE 1
SAR studies leading to benzimidazole as the preferred substituent in the
pyrimidine 2-position.

| | | IC₅₀ (μM) | |
|---|---|---|---|
| Compound | Structure | MNK1^{a} | MNK2^{a} |
| 3a | 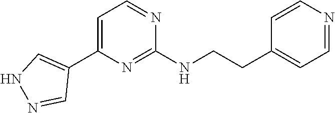 [SMILES: c1cc(CCNc2nccc(-c3cn[nH]c3)n2)ccn1] | 3.4 ± 1.3 | 5.6 ± 1.3 |
| 3b | 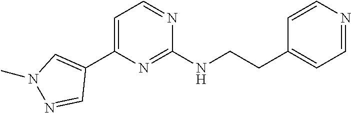 [SMILES: Cn1cc(-c2ccnc(NCCc3ccncc3)n2)cn1] | >20 | >20 |
| 3c | 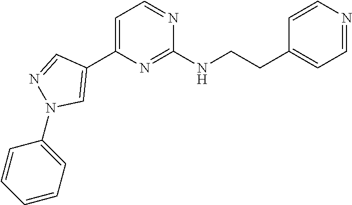 [SMILES: c1ccc(-n2cc(-c3ccnc(NCCc4ccncc4)n3)cn2)cc1] | 4.0 ± 1.8 | 2.6 ± 0.53 |
| 3d | 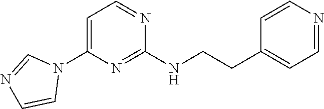 [SMILES: c1cc(CCNc2nccc(-n3ccnc3)n2)ccn1] | 1.3 ± 0.07^{b} | 8.8 ± 0.6^{b} |
| 3e | 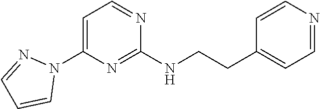 [SMILES: c1cnn(-c2ccnc(NCCc3ccncc3)n2)c1] | 15.5 ± 0.7^{b} | >20 |
| 3f | 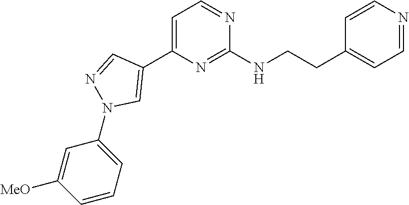 [SMILES: COc1cccc(-n2cc(-c3ccnc(NCCc4ccncc4)n3)cn2)c1] | 6.0^{c} | 2.1^{c} |
| 8a | 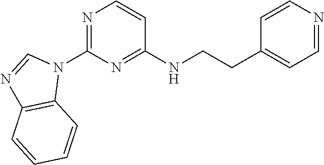 [SMILES: c1ccc2c(c1)ncn2-c1nccc(NCCc2ccncc2)n1] | 0.14 ± 0.09^{b} | 0.02 ± 0.027^{b} |
| 8b | 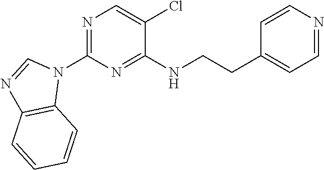 [SMILES: Clc1cnc(-n2cnc3ccccc32)nc1NCCc1ccncc1] | 0.070 ± 0.015^{b} | 0.014 ± 0.005^{b} |
| 8c | 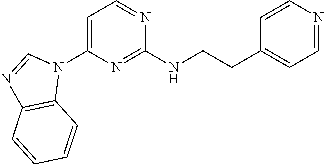 [SMILES: c1ccc2c(c1)ncn2-c1ccnc(NCCc2ccncc2)n1] | 0.33 ± 0.10 | 0.030 ± 0.005 |
| 8d | 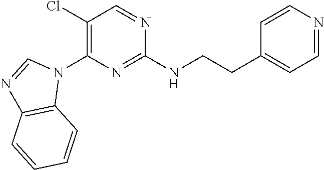 [SMILES: Clc1cnc(NCCc2ccncc2)nc1-n1cnc2ccccc21] | 10.3 ± 3.8^{b} | 0.90 ± 0.17^{b} |
| 8e | 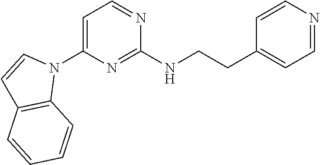 [SMILES: c1ccc2c(c1)ccn2-c1ccnc(NCCc2ccncc2)n1] | 5.7 ± 2.4 | 4.8 ± 1.4 |

^{a}IC₅₀ estimates were calculated using the variable Hill slope model with 12 dose points conducted in biological duplicate unless otherwise noted; the 95% confidence interval of the IC₅₀ value was converted to the ± Standard deviation (SD) assuming normal distribution (see statistical analysis).
^{b}The average of the IC₅₀ values from at least 2 separate assay runs each with 12 dose points conducted in biological duplicate are reported with their ± Standard deviation (SD).
^{c}Compound 3f was tested in singlicate.

### Ether Analogs

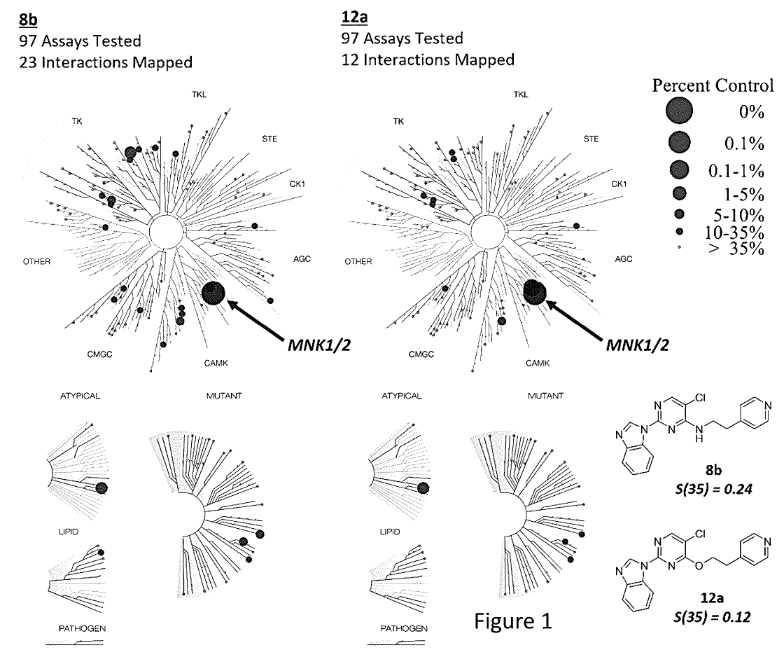

We next turned our attention to modifying the right-hand side pyridylethylamine moiety. Based on our molecular modeling of this series, the aminopyrimidine was surprisingly not involved in hinge binding. As this is a common motif among kinase inhibitors,²⁶ we surmised that the presence of this aminopyrimidine group might impart inhibitory activity against other kinases and make it difficult to achieve good MNK selectivity. We first synthesized the ether analog (12a) to eliminate the N—H hydrogen-bond donor and were gratified to see that it showed comparable biochemical activity (MNK1 IC₅₀=48 nM; MNK2 IC₅₀=13 nM) to that of its amine counterpart, 8b (Table 2). As the potency between compounds with the ether and amine linkers was essentially the same, we then wanted to see if this change impacted the broader kinome selectivity as we hypothesized. Compounds 8b and 12a were screened in a 97-kinase panel to assess their selectivity. In this panel of diverse kinases from across the kinome, inhibitor 12a with an ether linker was found to only have 12 interactions, whereas amine 8b had 23 (FIG. 1 and Tables 4 and 5). Based on this significantly improved selectivity, we maintained the ether linker for subsequent analogs.

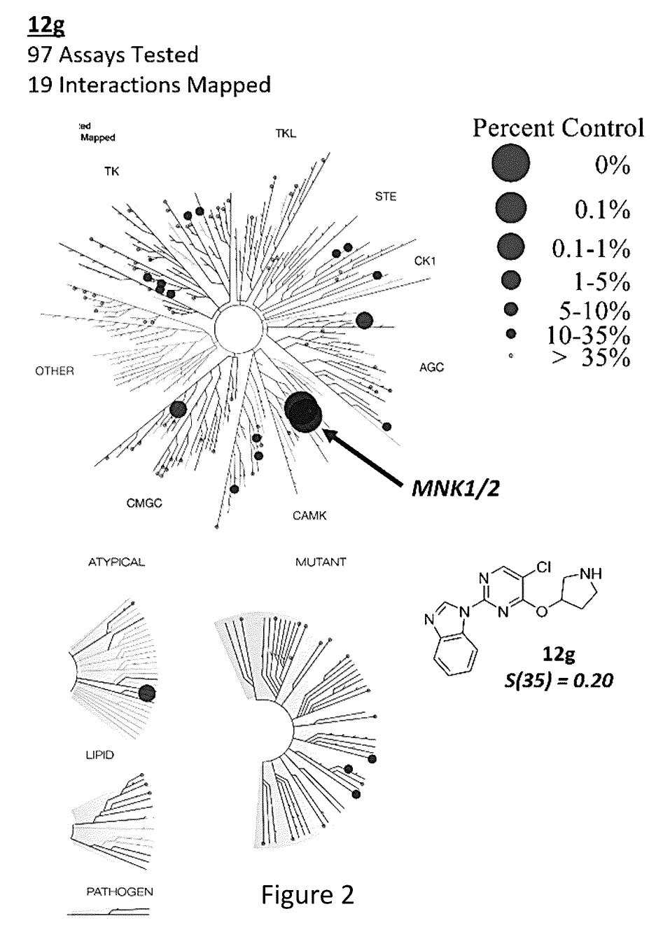

Synthesis of the 2-pyridyl analog (12b) resulted in essentially no change in potency relative to 12a. We next explored non-aromatic replacements that might be more favorably positioned in the solvent-exposed orientation, including the morpholine derivative 12c, which resulted in a significant drop in potency. The piperidine derivative, 12d, was significantly more potent than 12c (MNK1 IC₅₀=0.34 μM versus 1.4 μM) but not as potent as the pyridyl derivative. Reducing the size of the ring system and shortening the linker to engage Asp226 led us to synthesize the aminoazetidine compounds 12e and 12f. While the trans-derivative 12e had a MNK2 IC₅₀=0.12 μM, the cis-analog was approximately twice as potent (MNK1 IC₅₀=61 nM). We then synthesized the racemic pyrrolidine compound 12g which inhibited MNK1 with an IC₅₀=42 nM. Following up on this result, we synthesized the individual enantiomers and found that the (R)-pyrrolidine (12h) was significantly more potent than the (S)-pyrrolidine 12i, although comparable to the racemic compound. To confirm that the ether-linked pyrrolidine retained a preference for attachment to the pyrimidine 4-position, we synthesized and tested the regioisomer where the ether was attached to the 2-position (12j) and found this compound had no significant MNK inhibitory activity. Compound 12g also had a similar kinome profile to 12a, although it engaged several more kinases than 12a (FIG. 2). Since both compounds were tested at the same concentration in the kinome screen, we postulate that this apparent decrease in selectivity is due to the greater potency of 12g vs 12a which was observed both in our biochemical assay and cellular assays.

TABLE 2
SAR of ether substituents.

| | | IC₅₀ (μM) | | | |
|---|---|---|---|---|---|
| | | | | p-eIF4E | p-eIF4E |
| Compound | Structure | MNK1^{a} | MNK2^{a} | MV4-11^{c} | U937^{c} |
| 12a | 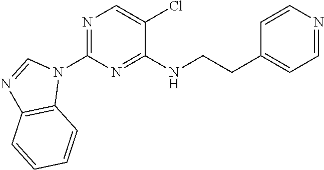 [SMILES: Clc1cnc(-n2cnc3ccccc32)nc1NCCc1ccncc1] | 0.048 ± 0.008^{b} | 0.013 ± 0.005^{b} | 0.046 ± 0.016 | 0.070 ± 0.034 |
| 12b | 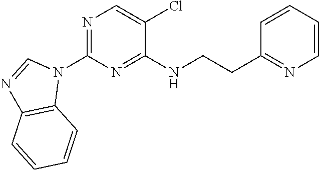 [SMILES: Clc1cnc(-n2cnc3ccccc32)nc1NCCc1ccccn1] | 0.058 ± 0.045^{b} | 0.010 ± 0.006^{b} | 0.038 ± 0.020 | 0.054 ± 0.013 |
| 12c | 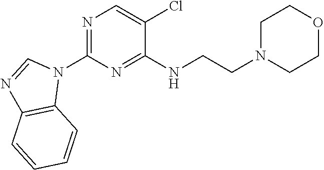 [SMILES: Clc1cnc(-n2cnc3ccccc32)nc1NCCN1CCOCC1] | 1.4 ± 1.2 | 0.69 ± 0.51 | ND^{d} | ND^{d} |
| 12d | 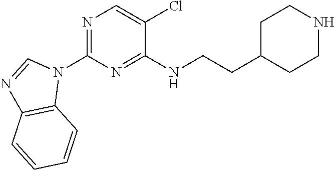 [SMILES: Clc1cnc(-n2cnc3ccccc32)nc1NCCC1CCNCC1] | 0.34 ± 0.38 | 0.18 ± 0.13 | ND^{d} | ND^{d} |
| 12e | 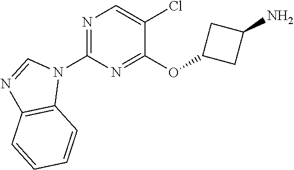 | 0.12 ± 0.031 | 0.051 ± 0.022 | ND^{d} | ND^{d} |
| 12f | 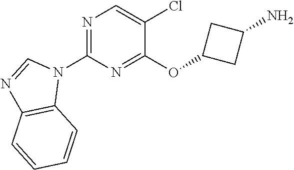 | 0.061 ± 0.019 | 0.026 ± 0.014 | 0.026 ± 0.012 | 0.406 ± 0.237 |
| 12g (NUCC- 0200808) | 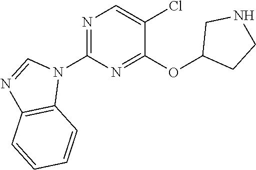 [SMILES: Clc1cnc(-n2cnc3ccccc32)nc1OC1CCNC1] | 0.042 ± 0.024^{b} | 0.029 ± 0.028^{b} | 0.021 ± 0.005 | 0.060 ± 0.100 |
| 12h (NUCC- 0201049) | 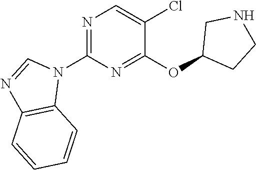 [SMILES: Clc1cnc(-n2cnc3ccccc32)nc1O[C@@H]1CCNC1] | 0.034 ± 0.028^{b} | 0.010 ± 0.009^{b} | 0.045 ± 0.033 | 0.637 ± 0.621 |
| 12i | 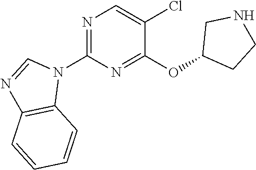 [SMILES: Clc1cnc(-n2cnc3ccccc32)nc1O[C@H]1CCNC1] | 0.10 ± 0.012 | 0.098 ± 0.035 | ND^{d} | ND^{d} |
| 12j | 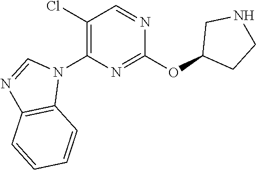 [SMILES: Clc1cnc(O[C@@H]2CCNC2)nc1-n1cnc2ccccc21] | >20 | >20 | ND^{d} | ND^{d} |

^{a}IC₅₀ estimates were calculated using the variable Hill slope model with 12 dose points conducted in biological duplicate unless otherwise noted; the 95% confidence interval was converted to the ± Standard deviation (SD) assuming normal distribution (see statistical analysis).
^{b}The average of the IC₅₀ values from at least 2 separate assay runs each with 12 dose points conducted in biological duplicate are reported with their ± Standard deviation (SD).
^{c}IC₅₀ estimates were calculated using non-linear regression curve fitting with 8 dose points conducted in biological duplicate; the 95% confidence interval was converted to the ± Standard deviation (SD) assuming normal distribution (see statistical analysis).
^{d}Not Determined.

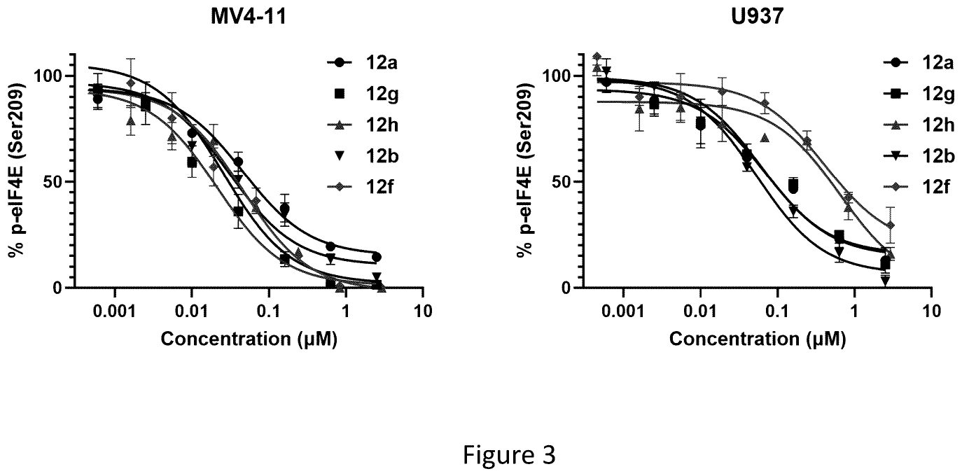

Compounds that had a MNK1 IC₅₀<100 nM were also assessed in our cellular assay²⁵ with MV4-11 and U937 cells to measure their ability to inhibit phosphorylation of Ser209 on eIF4E, which is known to be phosphorylated by MNKs.²⁷ The observed potency of the compounds tended to be better in MV4-11 cells than U937 cells (FIG. 3 and Table 2).

### Synthesis of MNK Inhibitors

Compounds in Table 1 were synthesized via several different routes as shown in Schemes 1-3. Compounds 3c, 3a, and 3b in Table 1 were synthesized by subjecting the appropriate heteroaryl boronic acid to a Suzuki coupling reaction with 2,4-dichloropyrimidine, followed by displacement of the chlorine in the 2-position with 2-(pyridin-4-yl)ethanamine and DIPEA (Scheme 1). The regioselectivity of the Suzuki reaction was confirmed by obtaining small molecule X-ray structures of compounds 3c, 3d, and 3a (see SI). For compound 3a, Boc-protected pyrazole boronic acid was used in the first step to afford intermediate 2a. The Boc-protecting group from 2a was eliminated during reaction with 2-(pyridin-4-yl)ethanamine to give compound 3a. In the cases of 3d and 3e, the 2-chloropyrimidines with the desired imidazole or pyrazole 4-position substituent, respectively, were commercially available. These commercially available materials were used in the final SNAr reaction to provide 3d and 3e.

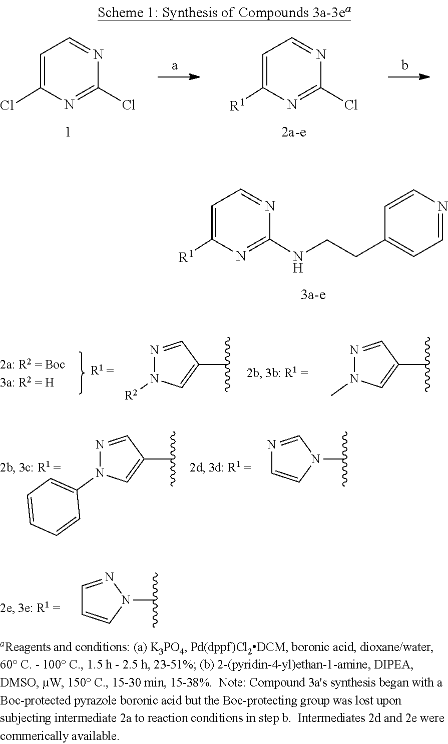

To synthesize compound 8c, we initially pursued a route in which 2,4-dichloropyrimidine (1 equiv) was stirred with benzimidazole (1 equiv) in the presence of Hunig's base (1.5 equiv) at room temperature. The reaction did not go to completion and two isomers were observed by LCMS. We attempted this reaction using sodium hydride (1.2 equiv) instead of DIPEA and one equivalent each of 2,4-dichloropyrimidine and benzimidazole at 0° C. in DMF. The reaction went to completion with a 3:1 mixture of product isomers. To our surprise, the major isomer isolated was that with the benzimidazole in the 2-position rather than the 4-position. See SI (Scheme 5) for further details and confirmation of the structure by X-ray crystallography. To overcome these issues and enable a convergent route for efficient synthesis of analogs, compounds 8a-8d with benzimidazole substituents were synthesized via a route similar to that reported by Islam Al-Khawaldeh et al.²⁸ in which 2-chloropyrimidin-4-amine (4c) was reacted with sodium hydride in the presence of 1-fluoro-2-nitrobenzene (Scheme 2). The resulting intermediate was then treated with 2-(pyridin-4-yl)ethan-1-amine and DIPEA with heat to displace the chlorine in the 2-position. Reduction of the nitro group followed by cyclization with trimethyl orthoformate and catalytic p-TsOH gave the desired cyclized product. Compounds 8a, 8b, and 8d were synthesized in a similar manner starting with 4-chloropyrimidin-2-amine (4a), 4,5-dichloropyrimidin-2-amine (4b), and 2,5-dichloropyrimidin-4-amine (4d), respectively (Scheme 2). Analog 8d was also synthesized via a shorter route in which 2,4,5-trichloropyrimidine was reacted with benzimidazole in the presence of Hunig's base at room temperature to give intermediate 10a whose NMR matched that reported previously²⁹ and was also confirmed by small molecule X-ray crystallography (see SI). This was followed by displacement of the chlorine in the 2-position with 2-(pyridin-4-yl)ethan-1-amine and DIPEA at room temperature to give the desired final compound whose NMR spectrum matched that of 8d made via the longer route from 2,5-dichloropyrimidin-4-amine described above (Scheme 3). Lastly, analog 8e was synthesized by reacting 2-chloro-4-fluoropyrimidine with sodium hydride in the presence of indole, followed by displacement of the chlorine in the 2-position with 2-(pyridin-4-yl)ethan-1-amine and DIPEA at elevated temperature (see Scheme 6 for more details).

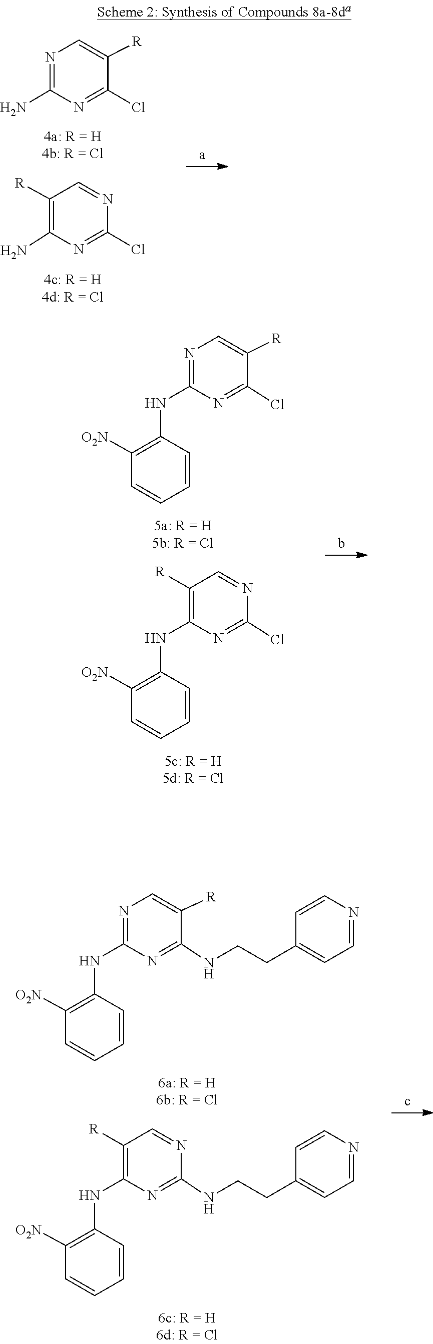

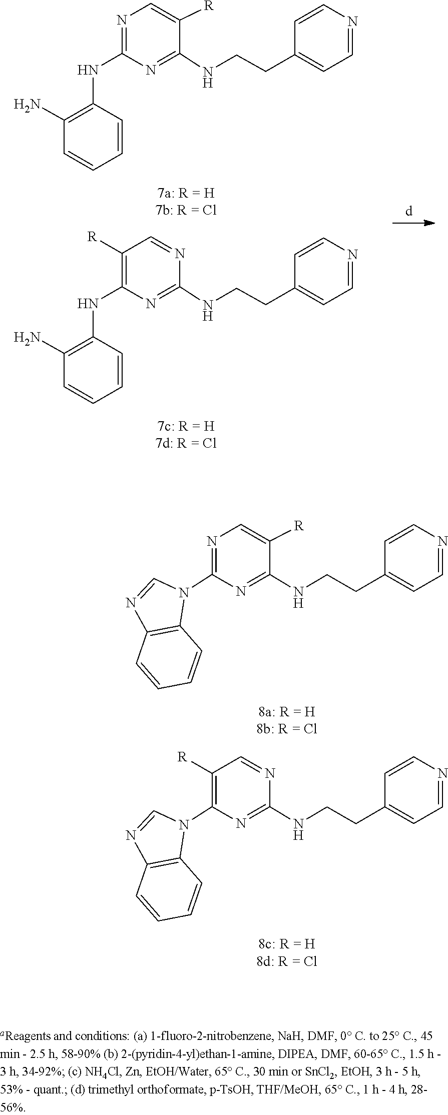

To synthesize the ether-containing compounds in Table 2, we initially attempted a route (Scheme 3) in which benzimidazole intermediate 10a was treated with the appropriate alcohol and sodium hydride. Surprisingly, we found that the benzimidazole migrated from the 4-position in intermediate 10a to the 2-position in the final compound in the presence of sodium hydride. We confirmed this intriguing result by synthesizing a representative ether, 12h, via two different routes, a shorter route with sodium hydride, and a longer route starting with 2-amino-4,5-dichloropyrimidine (See Scheme 4). The product resulting from these 2 different routes had identical NMR spectra, and we confirmed the structure of 12 h by X-ray crystallography (see SI). The rest of the ether analogs 12a-i in Table 2 were prepared using the more convenient and shorter synthetic route in which intermediate 10a was reacted with the appropriate alcohol in the presence of sodium hydride. If a Boc group was present in the alcohol, the appropriate boc-protected intermediates (11d-11i) were subjected to TFA/DCM conditions overnight to give final compounds. The characteristic singlets in the ¹H-NMR spectra of final compounds were used to confirm that the benzimidazole was indeed in the 2-position (see SI). Compound 12j in which the benzimidazole is in the 4-position was synthesized by following a slightly modified route beginning with 2,5-dichloropyrimidin-4-amine, 4d (Scheme 7). Attempts to react 2,5-dichloropyrimidin-4-amine (4d) with 1-fluoro-2-nitrobenzene in the presence of sodium hydride were unsuccessful using the same conditions as those used to synthesize intermediate 5b in Scheme 4. Conversion was seen at elevated temperature (60° C.) to afford 2,5-dichloro-N-(2-nitrophenyl)pyrimidin-4-amine (not shown). However, this compound was unreactive towards subsequent SNAr reaction with tert-butyl (R)-3-hydroxypyrrolidine-1-carboxylate despite using two equivalents of sodium hydride at elevated temperature (65° C.). Under these conditions, only starting material remained, presumably because of the difference in reactivity between the chlorine in the 2-position of this intermediate and the chlorine in the 4-position of intermediate 5b. To overcome this obstacle, the order of the first two steps was therefore reversed by first displacing the chlorine in the 2-position of 4d with tert-butyl (R)-3-hydroxypyrrolidine-1-carboxylate to give compound 16 which then reacted smoothly with 1-fluoro-2-nitrobenzene in the presence of sodium hydride (see Scheme 7 for details). The NMR spectrum and biochemical activity of 12j was markedly different compared to that of 12h, further confirming our structural assignments.

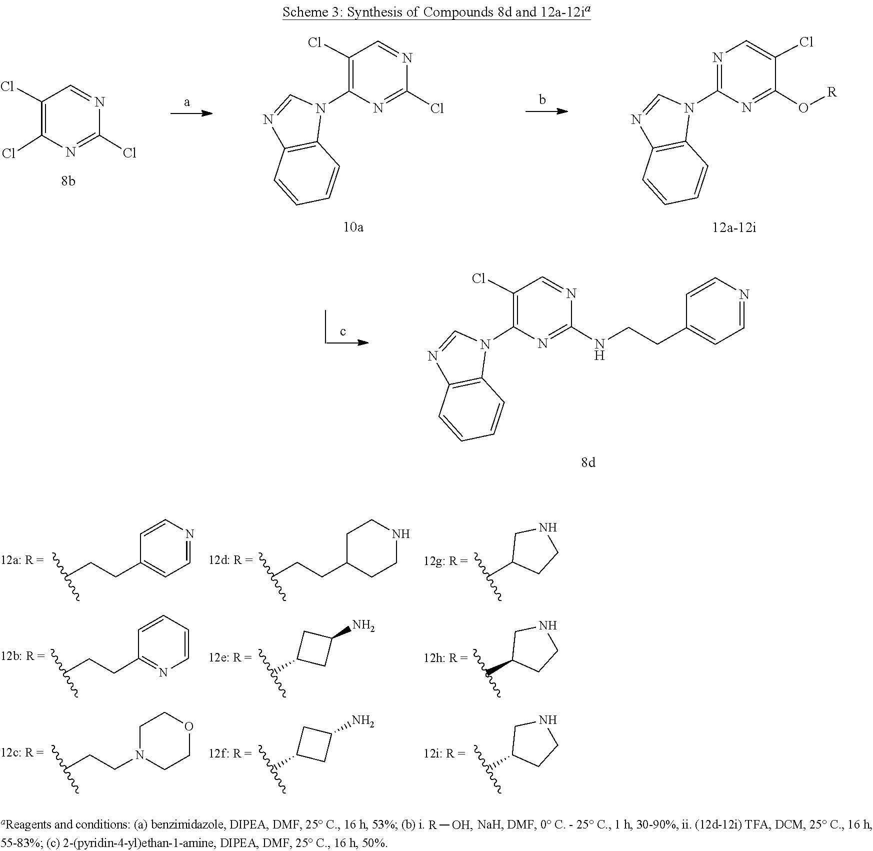

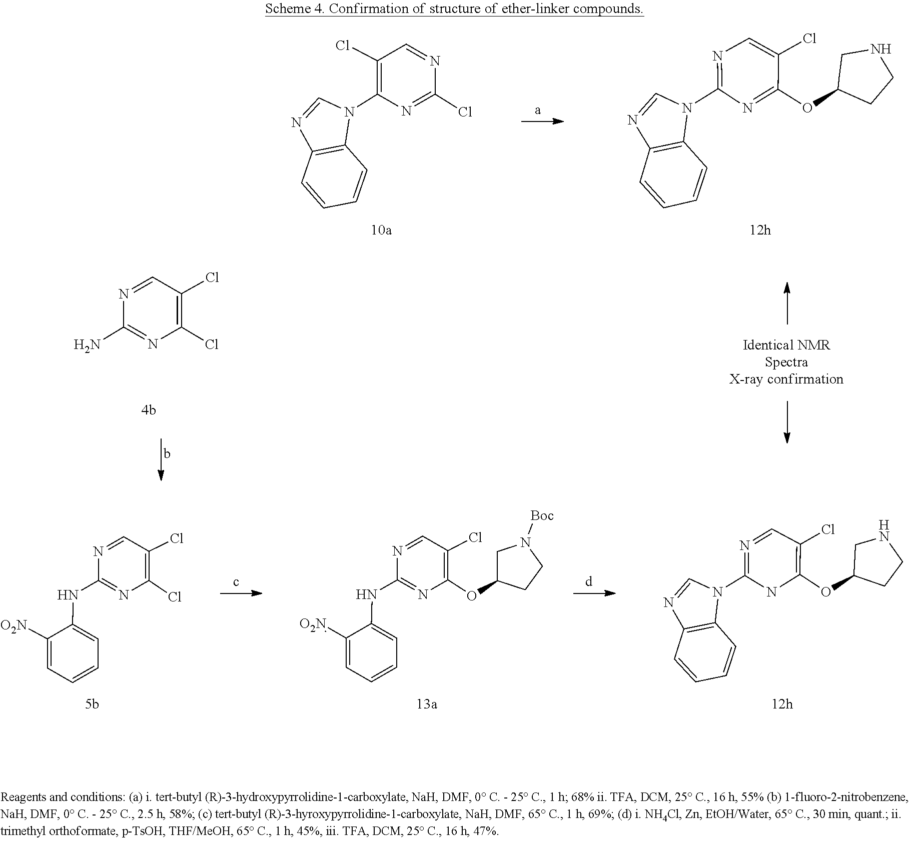

### X-Ray Crystal Structure of Bound Inhibitor

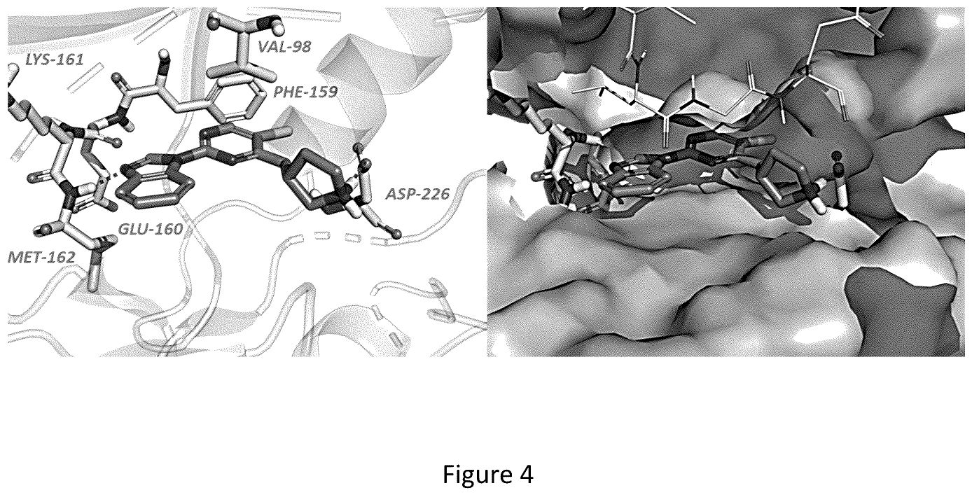

To confirm the binding mode of our inhibitor series and enable future optimization, we obtained a crystal structure of 12h (NUCC-0201049) in complex with MNK2-D228G (FIG. 4, PDB 9HRC). We elected to use MNK2-D228G rather than wild-type protein as this construct has been found to be more readily crystallizable with ligands.^{21, 30} As shown in FIG. 4, our scaffold assumes a unique binding pose in which the benzimidazole interacts with the hinge residues. The benzimidazole N-3 nitrogen forms a key hydrogen bond interaction with the backbone amide of hinge residue Met162, explaining the greater potency of benzimidazole versus indole. The chloropyrimidine ring is surrounded by several residues including Phe159 which may have hydrophobic interactions with the C-5 chlorine on the pyrimidine ring. Additionally, there may also be a halogen-pi interaction between our C-5 chlorine and the adjacent Phe159. In the protein surface view, it is evident that the binding pocket is very tight and large substituents would likely not be tolerated in the C-5 and C-6 positions, which is consistent with our observed SAR. The pyrrolidine ring points away from the binding pocket and its nitrogen atom forms a hydrogen bond with the side chain of Asp226.

Reduction of eIF4E Phosphorylation and Cell Viability in AML Cells

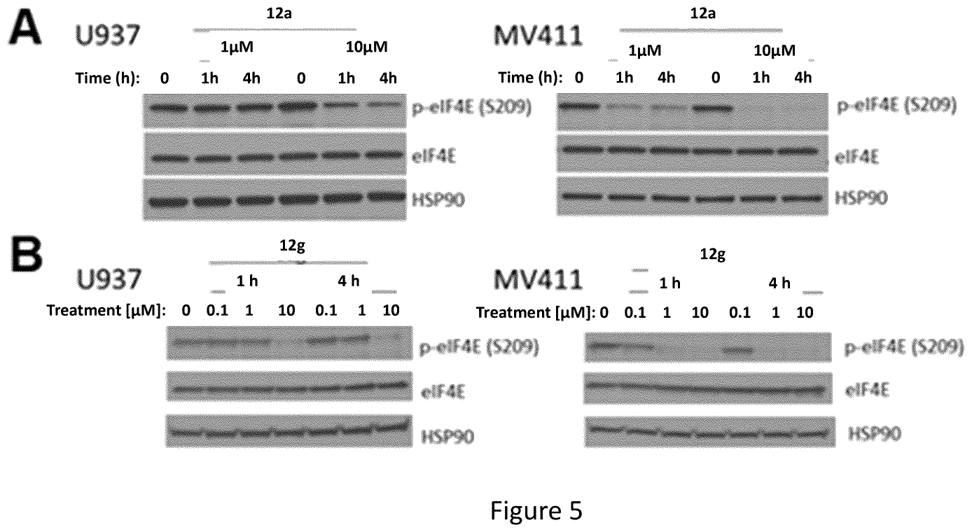

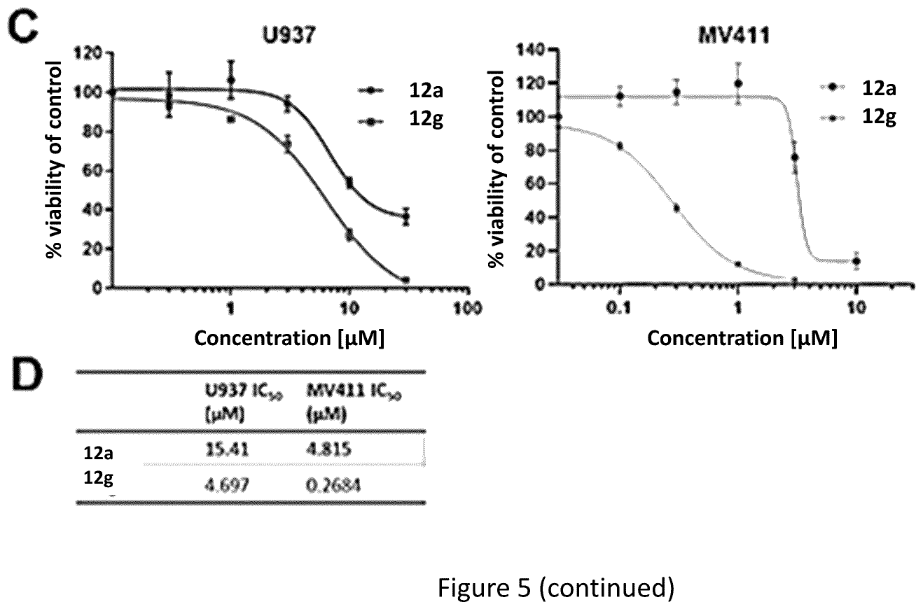

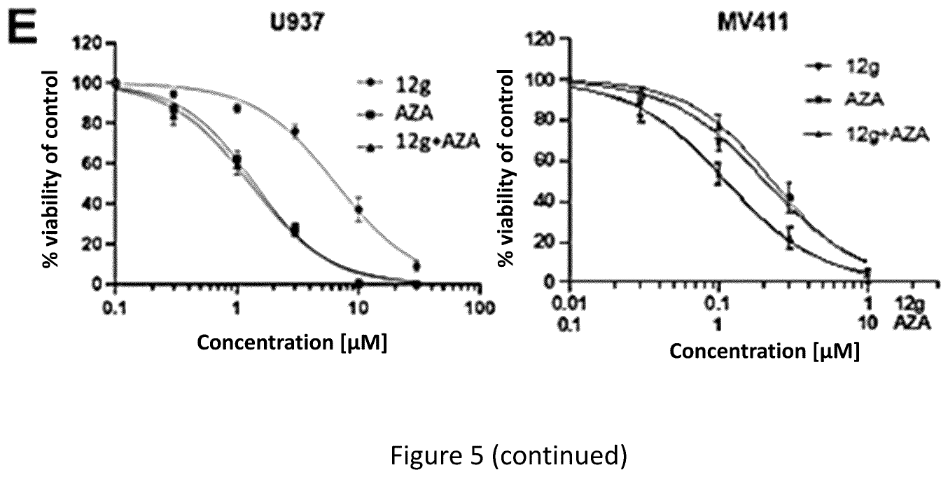

Compound 12a produced a decrease in phosphorylation of eIF4E at Ser209 after 1 and 4 h at 10 μM in U937 cells and at 1 μM for MV4-11 cells (FIG. 5, panel A). Similarly, for compound 12g (NUCC-0200808) in MV4-11 cells we observed near total reduction of phosphorylation of eIF4E at Ser209 at 1 and 10 μM at 1 and 4h, while clear reduction was seen at 10 μM in U937 cells (FIG. 5, panel B). This higher selectivity for MV4-11 cells over U937 cells is in line with our previous results (see FIG. 3).

Both compounds 12a and 12g decreased the viability of U937 and MV4-11 cells (FIG. 5, panel C). Compound 12g had the lowest IC₅₀ in both U937 and MV4-11 cells with 4.7 and 0.27 μM, respectively (FIG. 5, panel D). Also, in the context of cell viability, both compounds were more potent in MV4-11 cells than in U937 cells (FIG. 5, panels C, and D). Additionally, 12g was tested in combination with 5-azacytidine, a hypomethylating agent with major clinical activity in AML, in U937 and MV4-11 cells and this combination showed enhanced effects on viability reduction in MV4-11 cells (FIG. 5, panel E).

### Compound 12g (NUCC-0200808) Induces Apoptosis in AML Cells

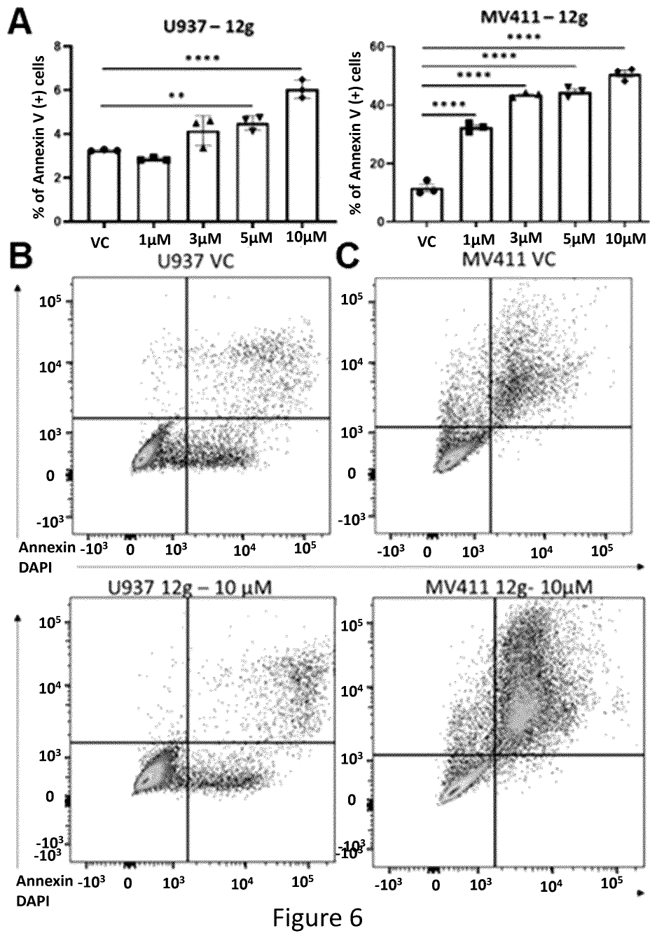

In MV4-11 cells, inhibitor 12g significantly induced apoptosis after 72 h compared to the DMSO vehicle control starting as low as 1 μM (FIG. 6, panel A). In U937 cells, the induction was significant at 5 and 10 μM. Representative dot plots are shown for the vehicle control and 10 μM in both cell lines which demonstrate the more substantial increase in apoptotic cells in MV4-11 cells by 12g (FIG. 6, panels B and C).

### Pharmacokinetics of MNK Inhibitor 12g (NUCC-0200808)

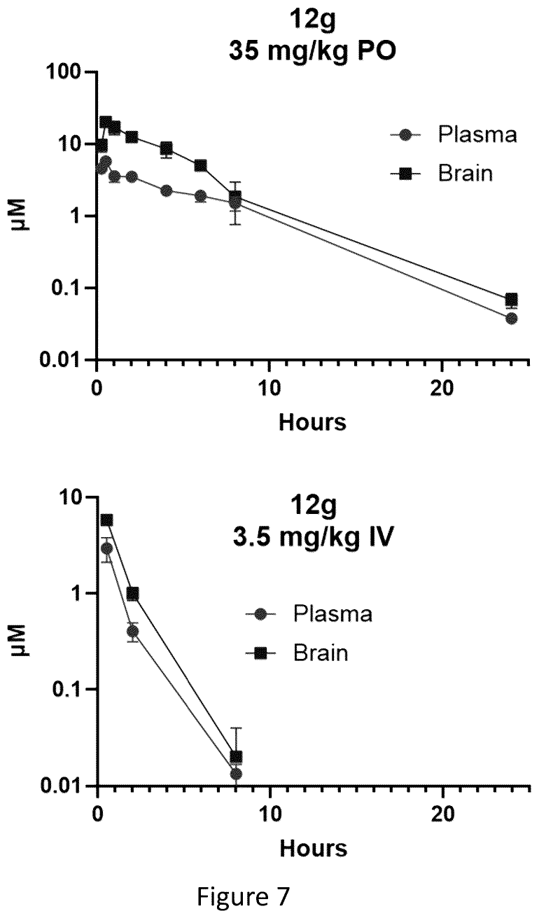

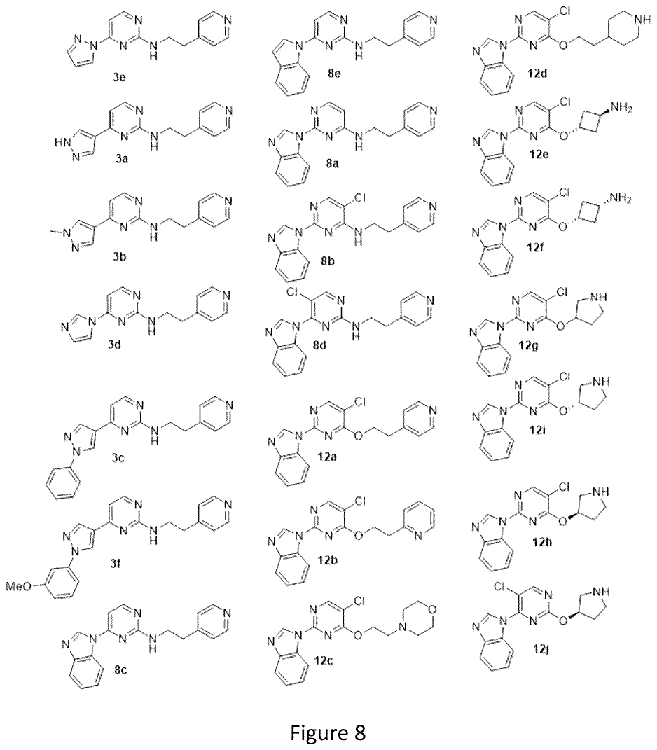

To assess the potential for in vivo use of 12g, its PK parameters were determined in C58Bl/6 male mice (FIG. 7). As shown in Table 3, after oral gavage of a 35 mg/kg dose, significant concentrations of drug were achieved in the plasma. Notably, we also found high levels of 12g in brain homogenate (Cₘₐₓ=20.0 μM). This also translated to total exposure, where high drug exposures were observed in both plasma and brain. The calculated total brain/plasma (B/P) ratio was 4.5, indicating high brain penetration of 12g. While half-life was moderate, 2.9 hrs and 7.0 hrs in brain and plasma, respectively, we found that this compound was able to achieve high drug levels in the brain. Based on I.V. dosing at 3.5 mg/kg, we found 12g to have excellent oral bioavailability as well, with oral bioavailability of 8700 based on plasma exposure and 9700 based on brain exposure. Plasma protein binding of 12g was 81.0% in mouse plasma at 6 hours and 82.2% in mouse brain homogenate after 6 hours.

TABLE 3
Pharmacokinetic parameters of 12g.

| | |
|---|---|
| PO dosing | IV dosing |
| (35 mg/kg) | (3.5 mg/kg) |

| Parameter | Plasma | Brain | Plasma | Brain |
|---|---|---|---|---|
| Cₘₐₓ (μM) | 5.7 | 20.0 | 1.3 | 5.8 |
| Tₘₐₓ (h) | 0.5 | 0.5 | 0.25 | 0.5 |
| T_{1/2} (h) | 7.0 | 2.9 | 1.3 | 1.0 |
| AUC₀₋ₗₐₛₜ (μM · h) | 20.9 | 93.5 | 2.4 | 9.6 |
| % F | 87.1 | 97.4 | N/A | N/A |

Tables 4 and 5 show kinase panel screening results. Specifically, Table 4 shows the percent control (0% Ctrl) values of compounds 8b, 12a, and 12g. Table 5 shows the selectivity score (S score) of compounds 8b, 12a, and 12g.

TABLE 4

| | | Percent control | | |
|---|---|---|---|---|
| DiscoveRx | Entrez Gene | 8b | 12a | 12g |
| Gene Symbol | Symbol | (10 μM) | (10 μM) | (10 μM) |
| ABL1(E255K)- | ABL1 | 100 | 91 | 95 |
| phosphorylated | | | | |
| ABL1(T315I)- | ABL1 | 92 | 74 | 56 |
| phosphorylated | | | | |
| ABL1-nonphosphorylated | ABL1 | 78 | 68 | 88 |
| ABL1-phosphorylated | ABL1 | 76 | 77 | 88 |
| ACVR1B | ACVR1B | 88 | 96 | 99 |
| ADCK3 | CABC1 | 85 | 94 | 95 |
| AKT1 | AKT1 | 100 | 100 | 68 |
| AKT2 | AKT2 | 100 | 83 | 99 |
| ALK | ALK | 46 | 54 | 48 |
| AURKA | AURKA | 51 | 95 | 90 |
| AURKB | AURKB | 91 | 78 | 59 |
| AXL | AXL | 18 | 39 | 40 |
| BMPR2 | BMPR2 | 18 | 47 | 49 |
| BRAF | BRAF | 73 | 73 | 76 |
| BRAF(V600E) | BRAF | 78 | 82 | 78 |
| BTK | BTK | 100 | 100 | 100 |
| CDK11 | CDK19 | 18 | 69 | 59 |
| CDK2 | CDK2 | 57 | 76 | 80 |
| CDK3 | CDK3 | 89 | 70 | 77 |
| CDK7 | CDK7 | 16 | 26 | 2.5 |
| CDK9 | CDK9 | 88 | 96 | 86 |
| CHEK1 | CHEK1 | 100 | 98 | 31 |
| CSF1R | CSF1R | 65 | 71 | 70 |
| CSNK1D | CSNK1D | 46 | 60 | 22 |
| CSNK1G2 | CSNK1G2 | 63 | 62 | 75 |
| DCAMKL1 | DCLK1 | 83 | 87 | 84 |
| DYRK1B | DYRK1B | 14 | 44 | 67 |
| EGFR | EGFR | 58 | 76 | 100 |
| EGFR(L858R) | EGFR | 52 | 85 | 100 |
| EPHA2 | EPHA2 | 100 | 96 | 99 |
| ERBB2 | ERBB2 | 61 | 92 | 100 |
| ERBB4 | ERBB4 | 69 | 80 | 66 |
| ERK1 | MAPK3 | 95 | 90 | 99 |
| FAK | PTK2 | 78 | 83 | 83 |
| FGFR2 | FGFR2 | 100 | 99 | 100 |
| FGFR3 | FGFR3 | 98 | 86 | 93 |
| FLT3 | FLT3 | 5.7 | 18 | 10 |
| GSK3B | GSK3B | 86 | 99 | 98 |
| IGF1R | IGF1R | 82 | 80 | 93 |
| IKK-alpha | CHUK | 24 | 52 | 55 |
| IKK-beta | IKBKB | 48 | 63 | 75 |
| INSR | INSR | 77 | 83 | 67 |
| JAK2(JH1domain- | JAK2 | 37 | 25 | 83 |
| catalytic) | | | | |
| JAK3(JH1 domain- | JAK3 | 2.8 | 12 | 28 |
| catalytic) | | | | |
| JNK1 | MAPK8 | 89 | 100 | 100 |
| JNK2 | MAPK9 | 44 | 53 | 73 |
| JNK3 | MAPK10 | 41 | 71 | 74 |
| KIT | KIT | 11 | 20 | 29 |
| KIT(D816V) | KIT | 9.2 | 30 | 17 |
| KIT(V559D, T670I) | KIT | 48 | 39 | 53 |
| LKB1 | STK11 | 7.4 | 7.3 | 19 |
| MAP3K4 | MAP3K4 | 100 | 100 | 100 |
| MAPKAPK2 | MAPKAPK2 | 100 | 94 | 100 |
| MARK3 | MARK3 | 72 | 62 | 92 |
| MEK1 | MAP2K1 | 43 | 59 | 21 |
| MEK2 | MAP2K2 | 50 | 67 | 24 |
| MET | MET | 48 | 57 | 93 |
| MKNK1 | MKNK1 | 8.7 | 0.3 | 0.05 |
| MKNK2 | MKNK2 | 0.05 | 0 | 0 |
| MLK1 | MAP3K9 | 65 | 65 | 80 |
| p38-alpha | MAPK14 | 91 | 98 | 87 |
| p38-beta | MAPK11 | 100 | 100 | 98 |
| PAK1 | PAK1 | 70 | 88 | 77 |
| PAK2 | PAK2 | 36 | 66 | 70 |
| PAK4 | PAK4 | 60 | 79 | 76 |
| PCTK1 | CDK16 | 100 | 100 | 100 |
| PDGFRA | PDGFRA | 37 | 51 | 29 |
| PDGFRB | PDGFRB | 18 | 26 | 13 |
| PDPK1 | PDPK1 | 86 | 60 | 90 |
| PIK3C2B | PIK3C2B | 57 | 73 | 74 |
| PIK3CA | PIK3CA | 77 | 80 | 68 |
| PIK3CG | PIK3CG | 11 | 39 | 50 |
| PIM1 | PIM1 | 28 | 68 | 84 |
| PIM2 | PIM2 | 100 | 100 | 100 |
| PIM3 | PIM3 | 24 | 69 | 53 |
| PKAC-alpha | PRKACA | 100 | 100 | 50 |
| PLK1 | PLK1 | 81 | 82 | 83 |
| PLK3 | PLK3 | 90 | 91 | 86 |
| PLK4 | PLK4 | 39 | 63 | 68 |
| PRKCE | PRKCE | 23 | 61 | 14 |
| RAF1 | RAF1 | 100 | 93 | 100 |
| RET | RET | 60 | 74 | 67 |
| RIOK2 | RIOK2 | 2.2 | 1.2 | 1.6 |
| ROCK2 | ROCK2 | 26 | 33 | 4.3 |
| RSK2(Kin.Dom.1-N- | RPS6KA3 | 74 | 60 | 66 |
| terminal) | | | | |
| SNARK | NUAK2 | 25 | 49 | 17 |
| SRC | SRC | 98 | 86 | 100 |
| SRPK3 | SRPK3 | 86 | 73 | 66 |
| TGFBR1 | TGFBR1 | 81 | 77 | 88 |
| TIE2 | TEK | 97 | 89 | 100 |
| TRKA | NTRK1 | 16 | 40 | 29 |
| TSSK1B | TSSK1B | 71 | 50 | 73 |
| TYK2(JH1 domain- | TYK2 | 31 | 61 | 59 |
| catalytic) | | | | |
| ULK2 | ULK2 | 55 | 48 | 45 |
| VEGFR2 | KDR | 69 | 74 | 41 |
| YANK3 | STK32C | 100 | 76 | 90 |
| ZAP70 | ZAP70 | 75 | 57 | 71 |

TABLE 5

| | | | Number | Screening | |
|---|---|---|---|---|---|
| | Selectivity | Number | of Non- | Concen- | |
| Compound | Score | of | Mutant | tration | Selectivity |
| Name | Type | Hits | Kinases | (nM) | Score |
| 8b | S(35) | 22 | 90 | 10000 | 0.244 |
| 8b | S(10) | 6 | 90 | 10000 | 0.067 |
| 8b | S(1) | 1 | 90 | 10000 | 0.011 |
| 12a | S(35) | 11 | 90 | 10000 | 0.122 |
| 12a | S(10) | 4 | 90 | 10000 | 0.044 |
| 12a | S(1) | 2 | 90 | 10000 | 0.022 |
| 12g | S(35) | 18 | 90 | 10000 | 0.2 |
| 12g | S(10) | 5 | 90 | 10000 | 0.056 |
| 12g | S(1) | 2 | 90 | 10000 | 0.022 |

### Conclusion

Translation of mRNA into oncogenic proteins is a key step in the transformation and proliferation of many types of leukemia cells. This process is facilitated by the essential phosphorylation of Ser209 on eIIF4E. Phosphorylated p-eIF4E activates binding of the RNA 5′-cap and subsequent translation of many oncogenic proteins. The only kinases known to phosphorylate Ser209 on eIF4E are the MVNKs, MNK1 and M/NK2. Because of the essentiality of the kinase activity of NVINK in the activation of eIF4E and subsequent translation of oncogenic proteins, significant effort has been made to develop drug-like MNK, inhibitors.

To date, a number of MNK inhibitors have been described in the literature. Among them is eFT508 (Tomivosertib), a potent MNK inhibitor that has been studied in clinical trials for several types of solid tumors, including prostate, breast, and lung. Efficacy in these trials has been modest and Tomivosertib has not been approved for use in any indications. Evaluation of Tomivosertib and other potent MNK inhibitors has focused mostly on their effects in solid tumors, with relatively little attention given to their potential use for leukemia and other hematological cancers. Other MNK inhibitors described have been shown to possess good potency, but selectivity for MNK1/2 has been either modest or not fully characterized, making it difficult to interpret their biological data. Additionally, the use of most published MNK inhibitors in animal models has not been established due in part to the lack of supporting pharmacokinetic data. Given these limitations, we therefore set out to discover new potent and drug-like MNK inhibitors that could serve as tool compounds to study MNK inhibition in vivo.

As we previously described, our work initially utilized a new mutation-based induced-fit in silico screen to identify potential MNK inhibitor chemotypes.²⁵ Using an inhibitor with a unique pyrimidine core, we initiated a medicinal chemistry project to improve the potency and selectivity of our scaffold. Based on an X-ray crystal structure of our inhibitor bound to MNK2, we used structural insights to guide the optimization of our lead series. We found that a benzimidazole could serve as an effective hinge-binding motif and our central pyrimidine core was not directly engaged in binding to the kinase hinge. To increase selectivity, our SAR led to the discovery that a unique ether linkage to the pyrimidine produced greater MNK1/2 selectivity than an amine linker. With a potent and selective inhibitor in-hand, we tested its ability to reduce cell viability of leukemia cells, induce apoptosis in those cells, and reduce leukemic progenitor colony formation. Finally, based on these results, the pharmacokinetics of our MNK inhibitor 12g (NUCC-0200808) were evaluated. These data showed that inhibitor 12g was highly orally bioavailable, possessed reasonable half-life, and interestingly, was highly brain penetrant. These results indicate that MNK inhibitor 12g is an effective compound for use in future animal studies to examine the role of MNK1/2 in AML and other diseases. Given the high brain penetration of compound 12g, future work can focus on further improving its half-life to expand its therapeutic potential for CNS disorders. While the pyrrolidine ring on the right-hand side of the molecule resulted in favorable biological activity, we hypothesize that it might be prone to metabolism by CYP450 oxidation. One potential strategy to reduce metabolism would be to functionalize the pyrrolidine ring to reduce the electron density and/or increase steric bulk on the ring.

### Experimental Section

General Chemical Methods. All chemical reagents were obtained from commercial suppliers and used without further purification unless otherwise stated. Anhydrous solvents were purchased from Sigma-Aldrich and dried over 3 Å molecular sieves when necessary. Normal-phase flash column chromatography was performed using Biotage KP-Sil 50 μm silica gel columns and ACS grade solvents on a Biotage Isolera flash purification system. Reverse phase prep conditions are detailed if relevant. Analytical thin layer chromatography (TLC) was performed on EM Reagent 0.25 mm silica gel 60 F254 plates and visualized by UV light. Proton (¹H), and carbon (¹³C) NMR spectra were recorded on a 500 MHz Bruker Avance III with direct cryoprobe spectrometer. Chemical shifts were reported in ppm (δ) and were referenced using residual nondeuterated solvent as an internal standard (CDCl₃ at 7.24 ppm for ¹H-NMR and 77.0 for ¹³C-NMR; CD₃OD at 3.33 ppm for ¹H-NMR and 47.6 for ¹³C-NMR; DMSO-d₆ at 2.52 ppm for ¹H-NMR and 39.9 ppm for ¹³C-NMR). Proton coupling constants are expressed in hertz (Hz). The following abbreviations were used to denote spin multiplicity for proton NMR: s=singlet, d=doublet, t=triplet, q=quartet, m=multiplet, brs=broad singlet, dd=doublet of doublets, dt=doublet of triplets, quin=quintet, tt=triplet of triplets. Low resolution liquid chromatography/mass spectrometry (LCMS) was performed on a Waters Acquity-H UPLC/MS system with a 2.1 mm×50 mm, 1.7 μm, reversed phase BEH C18 column and LCMS grade solvents. A gradient elution from 95% water+0.1% TFA/5% acetonitrile+0.1% TFA to 95% acetonitrile+0.1% TFA/5% water+0.1% TFA over 2 min plus a further minute continuing this mixture at a flow rate of 0.85 mL/min was used as the eluent. Total ion current traces were obtained for electrospray positive and negative ionization (ESI+/ESI−). All compounds are >95% pure by HPLC analysis. High-resolution mass spectra were obtained using an Agilent 6210 μLC-TOF spectrometer in the positive ion mode using electrospray ionization with an Agilent G1312A HPLC pump and an Agilent G1368B autoinjector at the Integrated Molecular Structure Education and Research Center (IMSERC), Northwestern University.

### Synthesis of Benzimidazole Intermediate 10a

To a solution of 2,4,5-trichloropyrimidine, 8b (6.3 mL, 54.5 mmol, 1 equiv) in DMF (250 mL) was added 1H-benzo[d]imidazole (6.44 g, 54.5 mmol, 1 equiv) followed by DIPEA (14.2 mL, 81.8 mmol, 1.5 equiv) and the reaction mixture was stirred for 16 h after water (600 mL) was added to the reaction mixture and solids formed. The mixture was stirred vigorously for 10 min and filtered to give 1-(2,5-dichloropyrimidin-4-yl)-1H-benzo[d]imidazole, 10a (7.7 g, 53%) as a light yellow solid whose NMR matches that reported previously.²² The structure of the product was also confirmed by x-ray crystallography (see SI). ¹H NMR (500 MHz, CDCl₃) δ 8.77 (s, 1H), 8.75 (s, 1H), 8.04-7.96 (m, 1H), 7.87-7.84 (m, 1H), 7.48-7.35 (m, 2H). ¹³C NMR (126 MHz, CDCl₃) δ 161.56, 158.89, 153.75, 143.50, 140.95, 131.92, 125.34, 124.84, 120.89, 119.83, 114.27.

### 4-(1H-pyrazol-1-yl)-N-(2-(pyridin-4-yl)ethyl)pyrimidin-2-amine (3e)

To a vial containing 2-chloro-4-(1H-pyrazol-1-yl)pyrimidine, 2e (200.0 mg, 1.11 mmol, 1 equiv, source: enamine) was added 2-(pyridin-4-yl)ethan-1-amine (388 μL, 3.322 mmol, 3 equiv) after which DMSO (2 mL) was added followed by DIPEA (579 μL, 3.322 mmol, 3 equiv). The reaction was heated to 150° C. for 15 min after which it was concentrated under a stream of nitrogen. The residue was purified by silica gel chromatography eluting with 0 to 5% MeOH in DCM. Relevant fractions were concentrated to give a solid which was further triturated in ether to give 4-(1H-pyrazol-1-yl)-N-(2-(pyridin-4-yl)ethyl)pyrimidin-2-amine, 3e (43.5 mg, 14.7%). ¹H NMR (500 MHz, CD₃OD) δ 8.57 (d, J=2.7 Hz, 1H), 8.43-8.38 (m, 2H), 8.30 (d, J=5.5 Hz, 1H), 7.79 (d, J=1.7 Hz, 1H), 7.39-7.34 (m, 2H), 7.10 (d, J=5.5 Hz, 1H), 6.58-6.53 (m, 1H), 3.75 (t, J=7.0 Hz, 2H), 3.01 (t, J=7.0 Hz, 2H); ¹³C NMR (126 MHz, CD₃OD) δ 162.03, 159.58, 157.74, 150.42, 148.43, 143.02, 127.19, 124.77, 108.13, 96.85, 41.18, 34.71. HRMS (ESI+): m/z calcd for C₁₄H₁₄N₆Na: 289.1172 [M+Na]⁺; found: 289.1178 [M+Na]⁺.

### 4-(1H-pyrazol-4-yl)-N-(2-(pyridin-4-yl)ethyl)pyrimidin-2-amine (3a)

To (1-(tert-butoxycarbonyl)-1H-pyrazol-4-yl)boronic acid (711.6 mg, 3.356 mmol, 1 equiv) in dioxane/water (5:1, 12 mL) was added PdCl2(dppf)·DCM (137 mg, 167.8 mol, 0.05 equiv), 2,4-dichloropyrimidine, 1 (500.0 mg, 3.4 mmol, 1 equiv), and K₃PO₄ (713 mg, 3.4 mmol, 1 equiv) after which the reaction mixture was degassed under nitrogen for 5 min then heated at 60° C. for 3.5 h after which the mixture was allowed to cool to room temperature. The mixture was diluted with dichloromethane and water. The organic layer was washed with brine, filtered through an isolute phase separator, and concentrated after which it was purified by silica gel chromatography eluting with 0 to 50% ethyl acetate in hexanes to give tert-butyl 4-(2-chloropyrimidin-4-yl)-1H-pyrazole-1-carboxylate, 2a (218 mg, 23.1%) as a white solid. ¹H NMR (500 MHz, CDCl₃) δ 8.73 (d, J=0.8 Hz, 1H), 8.58 (d, J=5.2 Hz, 1H), 8.23 (d, J=0.8 Hz, 1H), 7.39 (d, J=5.2 Hz, 1H), 1.66 (s, 9H).

To tert-butyl 4-(2-chloropyrimidin-4-yl)-1H-pyrazole-1-carboxylate, 2a (158 mg, 563 mol, 1 equiv) in DMSO (2 mL) was added 2-(pyridin-4-yl)ethan-1-amine (269 μL, 2.25 mmol, 4 equiv) and DIPEA (392 μL, 2.25 mmol, 4 equiv) after which the reaction was heated in the microwave at 150° C. for 1 h. The mixture was concentrated under a stream of nitrogen after which it was purified by silica gel chromatography with 0 to 10% MeOH in DCM. Relevant fractions concentrated to a solid which was further triturated from ether to give 4-(1H-pyrazol-4-yl)-N-(2-(pyridin-4-yl)ethyl)pyrimidin-2-amine, 3a (32 mg, 21%) as a tan solid. The structure of the product was confirmed by X-ray crystallography (see SI). ¹H NMR (500 MHz, CD₃OD) δ 8.45-8.38 (m, 2H), 8.33-8.03 (m, 3H), 7.39-7.34 (m, 2H), 6.88 (d, J=5.3 Hz, 1H), 3.74 (t, J=7.0 Hz, 2H), 3.01 (t, J=7.0 Hz, 2H).; ¹³C NMR (126 MHz, CD₃OD) δ 162.19, 160.12, 157.41, 150.59, 148.40, 137.93, 128.48, 124.77, 121.18, 105.52, 41.15, 34.79. HRMS (ESI+): m/z calcd for C₁₄H₁₄N₆Na: 289.1172 [M+Na]⁺; found: 289.1176 [M+Na]⁺.

### 4-(1-methyl-1H-pyrazol-4-yl)-N-(2-(pyridin-4-yl)ethyl)pyrimidin-2-amine (3b)

To (1-methyl-1H-pyrazol-4-yl)boronic acid (422.6 mg, 3.4 mmol, 1 equiv) in dioxane/water (5:1, 12 mL) was added K₃PO₄ (712.5 mg, 3.356 mmol, 1 equiv), 2,4-dichloropyrimidine, 1 (500.0 mg, 3.4 mmol, 1 equiv), and PdCl₂(dppf)·DCM (137.0 mg, 167.8 mol, 0.05 equiv). The reaction was degassed under nitrogen for 5 min after which it was heated at 100° C. for 1.5 h. The reaction was allowed to cool after which it was diluted with DCM and water. The organic layer was washed with brine, filtered through an isolute phase separator, and concentrated. The residue was purified by silica gel chromatography eluting with 0 to 100% ethyl acetate in hexanes to give 2-chloro-4-(1-methyl-1H-pyrazol-4-yl)pyrimidine, 2b (333 mg, 51.0%) as a yellow solid. ¹H NMR (500 MHz, CDCl₃) δ 8.47 (d, J=5.2 Hz, 1H), 8.08 (d, J=0.8 Hz, 1H), 7.99 (d, J=0.8 Hz, 1H), 7.28 (d, J=5.2 Hz, 1H), 3.95 (s, 3H).

To 2-chloro-4-(1-methyl-1H-pyrazol-4-yl)pyrimidine, 2b (150.0 mg, 770.7 mol, 1 equiv) was added DMSO (2 mL) after which 2-(pyridin-4-yl)ethan-1-amine (275.9 μL, 2.312 mmol, 3 equiv) and DIPEA (403 μL, 2.312 mmol, 3 equiv) were added. The reaction was heated to 150° C. for 15 min after which it was allowed to cool to room temperature. The mixture was diluted with ethyl acetate and water after which it was filtered to remove insoluble solids. The filtrate was transferred to a separatory funnel, and the organic layer was washed with water, 10% LiCl aqueous solution, brine, filtered through an isolute phase separator, and concentrated. The residue was purified by silica gel chromatography eluting with 0 to 10% methanol in dichloromethane to give to give 4-(1-methyl-1H-pyrazol-4-yl)-N-(2-(pyridin-4-yl)ethyl)pyrimidin-2-amine, 3b (58 mg, 27%). ¹H NMR (500 MHz, CDCl₃) δ 8.52-8.47 (m, 2H), 8.19 (d, J=5.2 Hz, 1H), 7.96-7.91 (m, 1H), 7.88 (s, 1H), 7.17-7.13 (m, 2H), 6.66 (d, J=5.2 Hz, 1H), 5.12 (t, J=6.3 Hz, 1H), 3.93 (s, 3H), 3.72 (q, J=6.9 Hz, 2H), 2.92 (t, J=7.0 Hz, 2H); ¹³C NMR (126 MHz, CDCl₃) δ 162.28, 159.45, 158.09, 149.87, 148.44, 138.38, 129.80, 124.23, 122.10, 106.23, 41.68, 39.27, 35.27. HRMS (ESI+): m/z calcd for C₁₅H₁₆N₆Na: 303.1329 [M+Na]⁺; found: 303.1332 [M+Na]⁺.

### 4-(1H-imidazol-1-yl)-N-(2-(pyridin-4-yl)ethyl)pyrimidin-2-amine (3d)

To 2-chloro-4-(1H-imidazol-1-yl)pyrimidine, 2d (200 mg, 1.11 mmol, 1 equiv, source: CombiBlocks) in DMSO (2 mL) was added 2-(pyridin-4-yl)ethan-1-amine (388 μL, 3.32 mmol, 3 equiv), and DIPEA (579 μL, 3.32 mmol, 3 equiv). The reaction was heated in the microwave at 150° C. for 15 min after it was concentrated under a stream of nitrogen. The residue was purified by silica gel chromatography eluting with 0 to 20% methanol in dichloromethane. Relevant fractions were concentrated to give a solid which was further triturated from ether to give 4-(1H-imidazol-1-yl)-N-(2-(pyridin-4-yl)ethyl)pyrimidin-2-amine, 3d (70 mg, 24%). The structure was confirmed by X-ray crystallography (see SI). ¹H NMR (500 MHz, CDCl₃) δ 8.51-8.45 (m, 2H), 8.31 (m, 2H), 7.55 (s, 1H), 7.14 (dd, J=5.5, 3.9 Hz, 3H), 6.54 (d, J=5.4 Hz, 1H), 5.55 (s, 1H), 3.73 (q, J=6.7 Hz, 2H), 2.92 (t, J=7.0 Hz, 2H); ¹³C NMR (126 MHz, CDCl₃) δ 162.18, 160.45, 155.52, 149.93, 147.98, 135.00, 131.02, 124.15, 115.57, 97.67, 41.74, 35.05. HRMS (ESI+): m/z calcd for C₁₄H₁₄N₆Na: 289.1172 [M+Na]⁺; found: 289.1180 [M+Na]⁺.

### 4-(1-phenyl-1H-pyrazol-4-yl)-N-(2-(pyridin-4-yl)ethyl)pyrimidin-2-amine (3c)

To a vial containing (1-phenyl-1H-pyrazol-4-yl)boronic acid (252.4 mg, 1.3 mmol, 1 equiv) in dioxane/water (5:1, 6 mL) was added K₃PO₄ (285.0 mg, 1.343 mmol, 1 equiv), 2,4-dichloropyrimidine, 1 (20.0 mg, 134 μmol, 1 equiv), and PdCl₂(dppf)·DCM (55 mg, 67.13 μmol, 0.05 equiv). The reaction was degassed under nitrogen for 5 min after which it was heated at 100° C. for 2.5 h. The reaction was allowed to cool to room temperature after which it was diluted with dichloromethane and water. The organic layer was washed with brine, filtered through an isolute phase separator, and concentrated. The residue was purified by silica gel chromatography eluting with 0 to 50% ethyl acetate in hexanes to give 2-chloro-4-(1-phenyl-1H-pyrazol-4-yl)pyrimidine, 2c (160 mg, 46.4%). ¹H NMR (500 MHz, CDCl₃) δ 8.64 (br s, 1H), 8.54 (d, J=5.2 Hz, 1H), 8.23-8.19 (m, 1H), 7.77-7.71 (m, 2H), 7.53-7.45 (m, 2H), 7.40-7.34 (m, 2H).

To 2-chloro-4-(1-phenyl-1H-pyrazol-4-yl)pyrimidine, 2c (100.0 mg, 389.6 mol, 1 equiv) in DMSO (1.5 mL) was added 2-(pyridin-4-yl)ethan-1-amine (139.4 μL, 1.169 mmol, 3 equiv) and DIPEA (204 μL, 1.169 mmol, 3 equiv) and the reaction was heated in the microwave at 150° C. for 15 min and the reaction was allowed to cool to room temperature after which it was diluted with ethyl acetate and water. The organic layer was washed with water, 10% LiCl aqueous solution, brine, filtered through an isolute phase separator, and concentrated to a residue which was purified by silica gel chromatography eluting with 0 to 5% methanol in dichloromethane give 4-(1-phenyl-1H-pyrazol-4-yl)-N-(2-(pyridin-4-yl)ethyl)pyrimidin-2-amine, 3c (50.0 mg, 37.5%) as an off white solid. The structure of the product was confirmed by X-ray crystallography (see SI). ¹H NMR (500 MHz, CDCl3) δ 8.54-8.49 (m, 2H), 8.45 (s, 1H), 8.26 (d, J=5.1 Hz, 1H), 8.16 (s, 1H), 7.75-7.69 (m, 2H), 7.51-7.43 (m, 2H), 7.36-7.29 (m, 1H), 7.20-7.15 (m, 2H), 6.77 (d, J=5.1 Hz, 1H), 5.15 (t, J=6.1 Hz, 1H), 3.76 (q, J=6.7 Hz, 2H), 2.95 (t, J=7.0 Hz, 2H); ¹³C NMR (126 MHz, CDCl3) δ 162.29, 159.02, 158.35, 149.92, 148.41, 139.89, 139.66, 129.57, 127.18, 126.33, 124.28, 123.68, 119.35, 106.48, 41.72, 35.26. HRMS (ESI+): m/z calcd for C₂₀H₁₈N₆Na: 365.1485 [M+Na]⁺; found: 365.1493 [M+Na]⁺.

### 4-(1H-benzo[d]imidazol-1-yl)-N-(2-(pyridin-4-yl)ethyl)pyrimidin-2-amine (8c)

To 2-chloropyrimidin-4-amine, 4c (1.0 g, 7.7 mmol, 1 equiv) was added DMF (10 mL) and the mixture was cooled in an ice/water bath subjected to an atmosphere of nitrogen. NaH (0.62 g, 60% Wt, 15 mmol, 2 equiv) was added slowly and the mixture was stirred for 5 min after which 1-fluoro-2-nitrobenzene (0.81 mL, 7.7 mmol, 1 equiv) was added. After stirring for 1.5 h, the reaction was quenched with water and the suspension was stirred vigorously for 30 min then filtered to isolate 2-chloro-N-(2-nitrophenyl)pyrimidin-4-amine, 5c (1.75 g, 90%). ¹H NMR (500 MHz, CDCl3) δ 10.20 (s, 1H), 8.76 (dd, J=8.6, 1.3 Hz, 1H), 8.30 (d, J=5.7 Hz, 1H), 8.24 (dd, J=8.4, 1.6 Hz, 1H), 7.70 (ddd, J=8.7, 7.1, 1.6 Hz, 1H), 7.18 (ddd, J=8.5, 7.2, 1.3 Hz, 1H), 6.74 (d, J=5.7 Hz, 1H).

To 2-chloro-N-(2-nitrophenyl)pyrimidin-4-amine, 5c (1.75 g, 6.98 mmol, 1 equiv) was added DMF (10 mL), DIPEA (2.43 mL, 14.0 mmol, 2 equiv) and 2-(pyridin-4-yl)ethan-1-amine (1.67 mL, 14.0 mmol, 2 equiv) after which the reaction was heated to 65° C. for 1.5 h. Additional DIPEA (365 μL, 2.09 mmol, 0.3 equiv) and 2-(4-Pyridinyl)ethylamine (249 μL, 2.09 mmol, 0.3 equiv) were added and the reaction was heated to 70° C. for 3 h. The reaction was quenched with water and stirred vigorously for 10 min then filtered to isolate solids which were purified by silica gel chromatography eluting with 0 to 10% dichloromethane in methanol to give N4-(2-nitrophenyl)-N2-(2-(pyridin-4-yl)ethyl)pyrimidine-2,4-diamine, 6c (793 mg, 33.8%). ¹H NMR (500 MHz, CDCl3) δ 10.00 (s, 1H), 8.65 (d, J=8.6 Hz, 1H), 8.53-8.48 (m, 2H), 8.29-8.19 (m, 1H), 7.96 (s, 1H), 7.56 (ddd, J=8.7, 7.2, 1.6 Hz, 1H), 7.16-7.09 (m, 3H), 6.17 (d, J=6.0 Hz, 1H), 3.75-3.64 (m, 2H), 2.93 (t, J=7.2 Hz, 2H).

To N4-(2-nitrophenyl)-N2-(2-(pyridin-4-yl)ethyl)pyrimidine-2,4-diamine, 6c (793.0 mg, 2.4 mmol, 1 equiv) was added ethanol:water (3:1, 13.3 mL) after which ammonium chloride (504 mg, 9.4 mmol, 4 equiv) and zinc (617 mg, 9.4 mmol, 4 equiv) were added and the reaction was heated to 65° C. for 30 min after which the reaction was allowed to cool to room temperature. The mixture was filtered through a pad of celite, washing with ethyl acetate to give N4-(2-aminophenyl)-N2-(2-(pyridin-4-yl)ethyl)pyrimidine-2,4-diamine, 7c (722 mg, quant.). ¹H NMR (500 MHz, CD3OD) δ 8.37 (d, J=5.5 Hz, 2H), 7.73 (d, J=6.7 Hz, 1H), 7.22 (s, 2H), 7.18-7.10 (m, 2H), 6.91 (dd, J=7.8, 1.4 Hz, 1H), 6.76 (td, J=7.6, 1.4 Hz, 1H), 6.04 (s, 1H), 3.60 (t, J=6.9 Hz, 2H), 2.91 (t, J=7.3 Hz, 2H).

To N4-(2-nitrophenyl)-N2-(2-(pyridin-4-yl)ethyl)pyrimidine-2,4-diamine, 7c (722 mg, 2.15 mmol, 1 equiv) was added THF/MeOH (4:1, 9 mL) and the suspension was sonicated after which trimethyl orthoformate (4.7 mL, 42.9 mmol, 20 equiv) and p-Toluenesulfonic acid (37.0 mg, 215 mol, 0.1 equiv) were added. The reaction was heated to 65° C. for 2 h after which additional trimethyl orthoformate (3.05 mL, 27.9 mmol, 13 equiv) and p-Toluenesulfonic acid (37.0 mg, 215 mol, 0.1 equiv) were added and heating was continued for 2 h. The reaction was neutralized with saturated aqueous sodium bicarbonate solution and it was diluted with dichloromethane. Combined organic extracts were washed with brine, filtered through an isolute phase separator, and concentrated to a residue which was purified by silica gel chromatography eluting with 0 to 10% methanol in dichloromethane. Relevant fractions were concentrated to give a solid which was further triturated from ether to give 4-(1H-benzo[d]imidazol-1-yl)-N-(2-(pyridin-4-yl)ethyl)pyrimidin-2-amine, 8c (188 mg, 27.7%). ¹H NMR (500 MHz, CD₃OD) δ 8.90 (s, 1H), 8.43-8.35 (m, 4H), 7.80-7.73 (m, 1H), 7.45-7.35 (m, 4H), 7.03 (d, J=5.6 Hz, 1H), 3.79 (s, 2H), 3.05 (t, J=7.1 Hz, 2H); ¹³C NMR (126 MHz, CD₃OD) δ 162.43, 159.91, 157.12, 150.29, 148.49, 143.74, 141.48, 131.48, 124.75, 124.54, 123.82, 119.29, 114.96, 97.89, 41.35, 34.65. HRMS (ESI+): m/z calcd for C₁₈H₁₆N₆Na: 339.1329 [M+Na]⁺; found: 339.1334 [M+Na]⁺.

### 2-(1H-benzo[d]imidazol-1-yl)-N-(2-(pyridin-4-yl)ethyl)pyrimidin-4-amine (8a)

To 4-chloropyrimidin-2-amine, 4a (1.0 g, 7.7 mmol, 1 equiv) was added DMF (10 mL) after which NaH (0.62 g, 60% Wt, 15 mmol, 2 equiv) was added slowly. After stirring for 5 min in an ice water bath and under nitrogen, 1-fluoro-2-nitrobenzene (0.81 mL, 7.7 mmol, 1 equiv) was added. After 1.5 h, the reaction was quenched with water, stirred for 30 min, then filtered to isolate 4-chloro-N-(2-nitrophenyl)pyrimidin-2-amine, 5a (1.29 g, 67%). ¹H NMR (500 MHz, CDCl3) δ 10.47 (s, 1H), 8.88 (dd, J=8.7, 1.4 Hz, 1H), 8.38 (d, J=5.2 Hz, 1H), 8.24 (dd, J=8.5, 1.6 Hz, 1H), 7.65 (ddd, J=8.8, 7.1, 1.6 Hz, 1H), 7.10 (ddd, J=8.5, 7.1, 1.3 Hz, 1H), 6.91 (d, J=5.2 Hz, 1H).

To 4-chloro-N-(2-nitrophenyl)pyrimidin-2-amine, 5a (1.29 g, 5.15 mmol, 1 equiv) was added DMF (10 mL) after which DIPEA (1.79 mL, 10.3 mmol, 2 equiv) and 2-(pyridin-4-yl)ethan-1-amine (1.23 mL, 10.3 mmol, 2 equiv) were added and the reaction was heated at 65° C. for 1.5 h after it was allowed to cool to room temperature then diluted with ethyl acetate and water. The organic layer was washed with aqueous 10% LiCl solution, brine, filtered through an isolute phase separator and concentrated to a residue which was purified by silica gel chromatography eluting with 0 to 10% methanol in dichloromethane to give N2-(2-nitrophenyl)-N4-(2-(pyridin-4-yl)ethyl)pyrimidine-2,4-diamine, 6a (1.08 g, 62.4%). ¹H NMR (500 MHz, CDCl3) δ 10.19 (s, 1H), 8.91 (dd, J=8.7, 1.3 Hz, 1H), 8.54-8.48 (m, 2H), 8.19 (dd, J=8.5, 1.6 Hz, 1H), 7.97 (dd, J=7.9, 4.6 Hz, 1H), 7.53 (ddd, J=8.6, 7.1, 1.6 Hz, 1H), 7.16-7.11 (m, 2H), 6.99 (ddd, J=8.4, 7.1, 1.3 Hz, 1H), 5.98 (d, J=5.9 Hz, 1H), 5.18 (t, J=6.1 Hz, 1H), 3.68 (q, J=6.5 Hz, 2H), 2.93 (t, J=7.0 Hz, 2H).

To a vial containing N2-(2-nitrophenyl)-N4-(2-(pyridin-4-yl)ethyl)pyrimidine-2,4-diamine, 6a (1.08 g, 3.21 mmol, 1 equiv) in EtOH:water (3:1, 13 mL) was added ammonium chloride (687 mg, 12.8 mmol, 4 equiv) and zinc (3.9 mg, 59.5 mol, 2 equiv) after which the reaction was heated at 65° C. for 30 min. The reaction was allowed to cool to room temperature after which it was filtered through a pad of celite, washing with ethyl acetate. The filtrate was concentrated to give N2-(2-aminophenyl)-N4-(2-(pyridin-4-yl)ethyl)pyrimidine-2,4-diamine, 7a (984 mg, quant) which was taken on to the next step without further purification. ¹H NMR (500 MHz, CD₃OD) δ 8.48-8.43 (m, 2H), 7.56 (d, J=7.1 Hz, 1H), 7.28 (s, 2H), 7.22-7.13 (m, 2H), 6.93 (dd, J=8.1, 1.4 Hz, 1H), 6.77 (td, J=7.6, 1.4 Hz, 1H), 6.10 (d, J=7.1 Hz, 1H), 3.70 (t, J=7.1 Hz, 2H), 2.96 (t, J=7.2 Hz, 2H).

To N2-(2-aminophenyl)-N4-(2-(pyridin-4-yl)ethyl)pyrimidine-2,4-diamine, 7a (984 mg, 3.21 mmol, 1 equiv) was added THF:MeOH (5:1, 10 mL) after which trimethyl orthoformate (7.0 mL, 64.2 mmol, 20 equiv) and p-TsOH (55.3 mg, 321 mol, 0.1 equiv) were added. The reaction was heated at 65° C. for 2 h after which it was allowed to cool to room temperature then neutralized with saturated aqueous sodium bicarbonate solution and diluted with dichloromethane. Organic extracts were washed with brine, filtered through an isolute phase separator, and concentrated to a residue which was purified by silica gel chromatography eluting with 0 to 10% methanol in dichloromethane to give a solid which was further purified by trituration from ether to give 2-(1H-benzo[d]imidazol-1-yl)-N-(2-(pyridin-4-yl)ethyl)pyrimidin-4-amine, 8a (314 mg, 30.9%). The structure of this compound was confirmed by x-ray crystallography (see SI). ¹H NMR (500 MHz, DMSO) δ 9.06 (s, 1H), 8.56-8.47 (m, 3H), 8.13 (d, J=5.9 Hz, 1H), 8.03 (d, J=5.9 Hz, 1H), 7.80-7.74 (m, 1H), 7.36 (dd, J=8.9, 6.6 Hz, 4H), 6.45 (d, J=6.0 Hz, 1H), 3.79 (q, J=6.7 Hz, 2H), 2.99 (t, J=7.2 Hz, 2H). ¹³C NMR (126 MHz, DMSO) δ 163.26, 155.74, 155.08, 149.86, 148.68, 144.90, 142.50, 132.05, 124.70, 124.33, 123.48, 120.20, 115.80, 104.01, 40.84, 34.27. HRMS (ESI+): m/z calcd for C₁₈H₁₆N₆Na: 339.1329 [M+Na]⁺; found: 339.1335 [M+Na]⁺.

### 2-(1H-benzo[d]imidazol-1-yl)-5-chloro-N-(2-(pyridin-4-yl)ethyl)pyrimidin-4-amine (8b)

To 4,5-dichloropyrimidin-2-amine, 4b (2.5 g, 15 mmol, 1 equiv) in DMF (45 mL) was added 1-fluoro-2-nitrobenzene (1.6 mL, 15 mmol, 1 equiv) and the mixture was cooled in an ice/water bath after which NaH (2.4 g, 60% Wt, 61 mmol, 4 equiv) was added slowly over 20 min. The reaction was stirred for 2.5 h under nitrogen as the ice bath expired after which the reaction was quenched with water. The mixture was stirred vigorously then filtered to isolate solids which were triturated from ether and filtered to give 4,5-dichloro-N-(2-nitrophenyl)pyrimidin-2-amine, 5b (2.5 g, 58%). ¹H NMR (500 MHz, CDCl3) δ 10.52 (s, 1H), 8.80 (d, J=8.6 Hz, 1H), 8.44 (s, 1H), 8.24 (dd, J=8.5, 1.6 Hz, 1H), 7.69-7.62 (m, 1H), 7.12 (t, J=7.8 Hz, 1H).

To a vial containing 4,5-dichloro-N-(2-nitrophenyl)pyrimidin-2-amine, 5b (1.0 g, 3.5 mmol, 1 equiv) in DMF (7 mL) was added 2-(pyridin-4-yl)ethan-1-amine (0.84 mL, 7.0 mmol, 2 equiv) and DIPEA (1.2 mL, 7.0 mmol, 2 equiv) and the reaction was heated at 60° C. for 2 h after the reaction was allowed to cool to room temperature. Water was added and the mixture was stirred then filtered to isolate 5-chloro-N2-(2-nitrophenyl)-N4-(2-(pyridin-4-yl)ethyl)pyrimidine-2,4-diamine, 6b (1.2 g, 92%). ¹H NMR (500 MHz, CDCl3) δ 10.25 (s, 1H), 8.87 (dd, J=8.6, 1.3 Hz, 1H), 8.62-8.57 (m, 2H), 8.22 (dd, J=8.5, 1.6 Hz, 1H), 8.01 (s, 1H), 7.56 (ddd, J=8.7, 7.1, 1.6 Hz, 1H), 7.43 (d, J=5.4 Hz, 2H), 7.03 (ddd, J=8.4, 7.2, 1.3 Hz, 1H), 5.48 (t, J=5.9 Hz, 1H), 3.85 (q, J=6.8 Hz, 2H), 3.10 (t, J=7.1 Hz, 2H).

To 5-chloro-N2-(2-nitrophenyl)-N4-(2-(pyridin-4-yl)ethyl)pyrimidine-2,4-diamine, 6b (1.20 g, 3.24 mmol, 1 equiv) and EtOH (8 mL) was added tin(II) chloride (1.23 g, 6.47 mmol, 2 equiv) and the reaction was heated to 65 degrees for 3 h after which additional tin(II) chloride (0.61 g, 3.23 mmol, 1 equiv) was added and the reaction was heated at 70° C. for 2 h. The material was allowed to cool to room temperature after which it basified with saturated aqueous sodium carbonate solution. The mixture was further diluted with ethyl acetate and Rochelle's salt and the mixture was stirred vigorously for 45 min after which it was filtered. The filtrate was transferred to a separatory funnel, and the organic layer was washed with brine, filtered through an isolute phase separator, and concentrated to give crude material which was triturated from ether and filtered to give N2-(2-aminophenyl)-5-chloro-N4-(2-(pyridin-4-yl)ethyl)pyrimidine-2,4-diamine, 7b (580 mg, 53%). ¹H NMR (500 MHz, DMSO) δ 8.43 (q, J=1.9 Hz, 2H), 8.13 (s, 1H), 7.85 (d, J=1.7 Hz, 1H), 7.32 (d, J=7.9 Hz, 1H), 7.19 (t, J=6.0 Hz, 1H), 7.15-7.09 (m, 2H), 6.88 (t, J=7.6 Hz, 1H), 6.73 (d, J=7.9 Hz, 1H), 6.53 (t, J=7.6 Hz, 1H), 4.81 (s, 2H), 3.50 (q, J=7.0 Hz, 2H), 2.81 (t, J=7.8 Hz, 2H).

To N2-(2-aminophenyl)-5-chloro-N4-(2-(pyridin-4-yl)ethyl)pyrimidine-2,4-diamine, 7b (580 mg, 1.70 mmol, 1 equiv) was added THF:MeOH (3:1, 8 mL) and trimethyl orthoformate (3.7 mL, 34.0 mmol, 20 equiv) followed by p-TsOH (29.3 mg, 170 mol, 0.1 equiv) and the reaction was heated to 65° C. for 1 h after which it was allowed to cool to room temperature then basified with saturated aqueous sodium bicarbonate solution and diluted with dichloromethane. The desired product began to precipitate out of solution. The mixture was filtered to isolate these solids which were further purified by trituration from ether to give 2-(1H-benzo[d]imidazol-1-yl)-5-chloro-N-(2-(pyridin-4-yl)ethyl)pyrimidin-4-amine, 8b (190 mg, 31.8%). ¹H NMR (500 MHz, CD3OD) δ 8.94 (s, 1H), 8.48-8.41 (m, 1H), 8.45-8.38 (m, 2H), 8.15 (s, 1H), 7.75-7.69 (m, 1H), 7.35 (td, J=6.6, 6.1, 2.7 Hz, 4H), 3.88 (t, J=7.2 Hz, 2H), 3.06 (t, J=7.2 Hz, 2H). ¹³C NMR (126 MHz, CD3OD) δ 158.59, 153.52, 152.75, 149.82, 148.56, 143.70, 141.94, 131.46, 124.73, 124.26, 123.50, 119.05, 115.39, 110.78, 41.12, 34.32. HRMS (ESI+): m/z calcd for C₁₈H₁₅ClN₆Na: 373.0939 [M+Na]⁺; found: 373.0947 [M+Na]⁺.

### 4-(1H-benzo[d]imidazol-1-yl)-5-chloro-N-(2-(pyridin-4-yl)ethyl)pyrimidin-2-amine (8d)

### Using Longer Route (Scheme 2):

To a round bottom flask containing 2,5-dichloropyrimidin-4-amine, 4d (2.0 g, 12 mmol, 1 equiv) in DMF (20 mL) in an ice/water bath was added NaH (0.98 g, 60% Wt, 24 mmol, 2 equiv) portionwise. After addition of NaH, the flask was heated to 60° C. and stirred for 5 min after which 1-fluoro-2-nitrobenzene (1.3 mL, 12 mmol, 1 equiv) was added. After stirring for 45 min, the reaction was allowed to cool to room temperature after which it was quenched with water. The mixture was stirred vigorously then filtered to isolate 2,5-dichloro-N-(2-nitrophenyl)pyrimidin-4-amine, 5d (2.76 g, 9.68 mmol, 79%). ¹H NMR (500 MHz, CD₃OD) δ 8.55 (dd, J=8.4, 1.4 Hz, 1H), 8.44 (s, 1H), 8.27 (dd, J=8.3, 1.6 Hz, 1H), 7.81 (ddd, J=8.6, 7.3, 1.6 Hz, 1H), 7.41 (ddd, J=8.6, 7.3, 1.3 Hz, 1H).

To 2,5-dichloro-N-(2-nitrophenyl)pyrimidin-4-amine, 5d (1.0 g, 3.5 mmol, 1 equiv) in DMF (7 mL) was added 2-(pyridin-4-yl)ethan-1-amine (0.84 mL, 7.0 mmol, 2 equiv) and DIPEA (1.2 mL, 7.0 mmol, 2 equiv) and the reaction was heated at 60° C. for 3 h after which the reaction was allowed to cool to room temperature. Water was added and the suspension was stirred then filtered to isolate 5-chloro-N4-(2-nitrophenyl)-N2-(2-(pyridin-4-yl)ethyl)pyrimidine-2,4-diamine, 6d (1.1 g, 85%). ¹H NMR (500 MHz, CDCl3) δ 10.65 (s, 1H), 8.84 (d, J=8.6 Hz, 1H), 8.54 (d, J=5.2 Hz, 2H), 8.26 (d, J=8.5 Hz, 1H), 8.05 (s, 1H), 7.65-7.54 (m, 1H), 7.32 (s, 2H), 7.21-7.14 (m, 1H), 3.72 (q, J=6.8 Hz, 2H), 3.02 (t, J=7.1 Hz, 2H).

To a 5-chloro-N4-(2-nitrophenyl)-N2-(2-(pyridin-4-yl)ethyl)pyrimidine-2,4-diamine, 6d (1.1 g, 3.0 mmol, 1 equiv) and Ethanol (8 mL) was added tin(II) chloride (1.1 g, 5.9 mmol, 2 equiv) and the reaction was heated to 65° C. for 3 h. Additional tin (II) chloride (0.55 g, 3 mmol, 1 equiv) was added and after stirring at 70° C. for 2 h, the reaction was allowed to cool to room temperature after which it was basified by adding saturated aqueous sodium carbonate solution. It was further diluted with ethyl acetate and Rochelle's salt and stirred vigorously for 45 min then filtered to remove insoluble solids. The filtrate was transferred to a separatory funnel and the organic layer was washed with brine, filtered through an isolute phase separator, and concentrated to give crude material which was triturated from ether and filtered to give N4-(2-aminophenyl)-5-chloro-N2-(2-(pyridin-4-yl)ethyl)pyrimidine-2,4-diamine, 7d (340 mg, 998 mol, 34%) which was taken on to the next step without further purification. ¹H NMR (500 MHz, DMSO) δ 8.47-8.30 (m, 2H), 8.03 (d, J=16.5 Hz, 1H), 7.89 (s, 1H), 7.16 (d, J=7.8 Hz, 1H), 7.06-6.90 (m, 2H), 6.82-6.74 (m, 1H), 6.59 (t, J=7.5 Hz, 1H), 4.78 (br s, 2H), 3.20 (br s, 2H), 2.79-2.56 (m, 2H).

To N4-(2-aminophenyl)-5-chloro-N2-(2-(pyridin-4-yl)ethyl)pyrimidine-2,4-diamine, 7d (340 mg, 998 mol, 1 equiv) was added THF:MeOH (4:1, 6 mL) and trimethyl orthoformate (2.2 mL, 20.0 mmol, 20 equiv) followed by p-TsOH (17.2 mg, 99.8 mol, 0.1 equiv) after which the reaction was heated to 65° C. After 1 h of stirring, additional trimethyl orthoformate (1.1 mL, 10.0 mmol, 10 equiv) and p-TsOH (17.2 mg, 99.8 mol, 0.1 equiv) were added and the reaction was stirred at 65° C. for another 1.5 h after which it was allowed to cool to room temperature and it was basified with saturated aqueous sodium bicarbonate solution, and diluted with dichloromethane. The organic layer was washed with water, brine, filtered through an isolute phase separator, concentrated and purified by silica gel chromatography eluting with 0 to 10% methanol in dichloromethane with 0.1% TEA to give 4-(1H-benzo[d]imidazol-1-yl)-5-chloro-N-(2-(pyridin-4-yl)ethyl)pyrimidin-2-amine, 8d (195 mg, 55.7%). The structure was confirmed by x-ray crystallography (see SI). ¹H NMR (500 MHz, CD₃OD) δ 8.72 (s, 1H), 8.51 (s, 1H), 8.40 (d, J=28.2 Hz, 2H), 7.86-7.76 (m, 2H), 7.52-7.15 (m, 4H), 3.72 (t, J=7.0 Hz, 2H), 2.99 (t, J=6.8 Hz, 2H). ¹³C NMR (126 MHz, CD₃OD) δ 161.01, 160.51, 152.24, 150.11, 148.45, 142.46, 142.40, 132.09, 124.70, 124.21, 123.65, 119.16, 113.47, 108.52, 41.60, 34.47. HRMS (ESI+): m/z calcd for C₁₈H₁₅ClN₆Na: 373.0939 [M+Na]⁺; found: 373.0950 [M+Na]⁺.

### Using Shorter Route (Scheme 3):

To 1-(2,5-dichloropyrimidin-4-yl)-1H-benzo[d]imidazole, 10a (300.0 mg, 1.132 mmol, 1 equiv) was added acetonitrile (4 mL) after which 2-(pyridin-4-yl)ethan-1-amine (135.0 μL, 1.132 mmol, 1 equiv) and DIPEA (296 μL, 1.7 mmol, 1.5 equiv) were added. The reaction was stirred at room temperature for 16 after which it was diluted with ethyl acetate and water. The organic layer was washed with saturated aqueous ammonium chloride solution, brine, filtered through an isolute phase separator, filtered, and concentrated to a yellow oil which was triturated from ether to give 4-(1H-benzo[d]imidazol-1-yl)-5-chloro-N-(2-(pyridin-4-yl)ethyl)pyrimidin-2-amine, 8d (200 mg, 50.4%). ¹H NMR (500 MHz, CD₃OD) δ 8.73 (s, 1H), 8.52 (dt, J=8.4, 2.9 Hz, 1H), 8.44-8.29 (m, 2H), 7.86-7.72 (m, 2H), 7.50-7.22 (m, 4H), 3.72 (t, J=6.9 Hz, 2H), 3.00 (t, J=7.2 Hz, 2H); ¹³C NMR (126 MHz, CD₃OD) δ 161.07, 160.51, 152.31, 150.22, 148.43, 142.49, 142.42, 132.12, 124.75, 124.22, 123.65, 119.15, 113.46, 108.63, 41.61, 34.50. NMR matches that of compound 8d obtained via the longer route above.

### 1-(5-chloro-4-(2-(pyridin-4-yl)ethoxy)pyrimidin-2-yl)-1H-benzo[d]imidazole (12a)

To 1-(2,5-dichloropyrimidin-4-yl)-1H-benzo[d]imidazole, 10a (500 mg, 1.89 mmol, 1 equiv) and 2-(pyridin-4-yl)ethan-1-ol (232 mg, 1.89 mmol, 1 equiv) in DMF (8 mL) in an ice/water bath was added NaH (91 mg, 60% Wt, 2.26 mmol, 1.2 equiv) and the reaction mixture was stirred under nitrogen for 1 h after which water (~10 mL) was added. The suspension was stirred vigorously for 20 minutes then filtered to isolate 1-(5-chloro-4-(2-(pyridin-4-yl)ethoxy)pyrimidin-2-yl)-1H-benzo[d]imidazole, 12a (540 mg, 81.4%) as a tan solid. ¹H NMR (500 MHz, Chloroform-d) δ 8.93 (s, 1H), 8.59-8.54 (m, 2H), 8.46 (d, J=1.5 Hz, 1H), 8.45-8.39 (m, 1H), 7.88-7.76 (m, 1H), 7.38 (tt, J=7.5, 6.0 Hz, 2H), 7.33-7.28 (m, 2H), 4.81 (td, J=6.5, 1.6 Hz, 2H), 3.23 (t, J=6.2 Hz, 2H). ¹³C NMR (126 MHz, CDCl₃) δ 164.91, 156.69, 153.31, 149.68, 144.95, 141.72, 131.63, 124.78, 124.53, 124.51, 124.02, 120.69, 115.07, 113.58, 67.58, 34.42. HRMS (ESI+): m/z calcd for C₁₈H₁₄ClN₅NaO: 374.0779 [M+Na]⁺; found: 374.0783 [M+Na]⁺.

### 1-(5-chloro-4-(2-(pyridin-2-yl)ethoxy)pyrimidin-2-yl)-1H-benzo[d]imidazole (12b)

To a vial containing 1-(2,5-dichloropyrimidin-4-yl)-1H-benzo[d]imidazole, 10a (0.1 g, 0.377 mmol, 1 equiv) and 2-(pyridin-2-yl)ethanol (0.043 ml, 0.377 mmol, 1 equiv) in DMF (2 mL) immersed in an ice/water bath was added NaH (0.018 g, 60% Wt, 0.453 mmol, 1.2 equiv) and the reaction was stirred for 90 min after which it was quenched with saturated aqueous ammonium chloride solution. The suspension was filtered to isolate solids which were washed extensively with water to give 1-(5-chloro-4-(2-(pyridin-2-yl)ethoxy)pyrimidin-2-yl)-1H-benzo[d]imidazole, 12b (40 mg, 30.1% yield) as a white solid. ¹H NMR (500 MHz, CD₃OD) δ 9.11 (d, J=3.2 Hz, 1H), 8.61-8.48 (m, 3H), 7.82-7.73 (m, 2H), 7.54-7.22 (m, 4H), 5.08-5.01 (m, 2H), 3.42 (tt, J=6.1, 2.5 Hz, 2H); ¹³C NMR (126 MHz, CD₃OD) δ 165.21, 157.65, 156.58, 153.18, 148.60, 143.82, 142.11, 137.31, 131.39, 124.73, 124.27, 123.88, 122.08, 119.20, 115.35, 113.74, 67.49, 36.47. HRMS (ESI+): m/z calcd for C₁₈H₁₄ClN₅NaO: 374.0779 [M+Na]⁺; found: 374.0783 [M+Na]⁺.

### 4-(2-((2-(1H-benzo[d]imidazol-1-yl)-5-chloropyrimidin-4-yl)oxy)ethyl)morpholine (12c)

To a vial containing 1-(2,5-dichloropyrimidin-4-yl)-1H-benzo[d]imidazole, 10a (200.0 mg, 754 mol, 1 equiv) was added DMF (6 mL) after which the mixture was cooled in an ice water bath and placed under nitrogen. 2-morpholinoethan-1-ol (91 μL, 754 mol, 1 equiv) was added after which NaH (39 mg, 60% Wt, 981 mol, 1.3 equiv) was added. The reaction mixture was stirred for 1 h after which it was quenched with water, stirred vigorously, and filtered to isolate solids which were further purified by silica gel chromatography eluting with 0 to 10% MeOH in DCM. Relevant fractions were combined and concentrated to give 4-(2-((2-(1H-benzo[d]imidazol-1-yl)-5-chloropyrimidin-4-yl)oxy)ethyl)morpholine, 12c (181 mg, 67%). ¹H NMR (500 MHz, CDCl3) δ 8.93 (s, 1H), 8.46-8.41 (m, 2H), 7.84-7.78 (m, 1H), 7.42-7.32 (m, 2H), 4.71 (t, J=5.7 Hz, 2H), 3.74-3.68 (m, 4H), 2.91 (t, J=5.7 Hz, 2H), 2.65-2.59 (m, 4H); ¹³C NMR (126 MHz, CDCl3) δ 165.13, 156.50, 153.32, 144.95, 141.74, 131.64, 124.73, 123.93, 120.62, 115.13, 113.63, 66.96, 66.67, 56.67, 54.12. HRMS (ESI+): m/z calcd for C₁₇H₁₈ClN₅NaO₂: 382.1041 [M+Na]⁺; found: 382.1047 [M+Na]⁺.

### 2-(1H-benzo[d]imidazol-1-yl)-5-chloro-N-(2-(piperidin-4-yl)ethyl)pyrimidin-4-amine (12d)

To 1-(2,5-dichloropyrimidin-4-yl)-1H-benzo[d]imidazole, 10a (200.0 mg, 754.4 mol, 1 equiv) in DMF (5 mL) in an ice/water bath and under nitrogen was added tert-butyl 4-(2-hydroxyethyl)piperidine-1-carboxylate (173.0 mg, 754.4 mol, 1 equiv) was added after which NaH (39.23 mg, 60% Wt, 980.8 mol, 1.3 equiv) was added. The reaction was stirred for 30 min then quenched with water and diluted with ethyl acetate. Combined organic extracts were washed with 10% aqueous LiCl solution, brine, filtered through an isolute phase separator, concentrated then purified by silica gel chromatography eluting with 0 to 100% EtOAc in hexanes to give tert-butyl 4-(2-((2-(1H-benzo[d]imidazol-1-yl)-5-chloropyrimidin-4-yl)oxy)ethyl)piperidine-1-carboxylate, 11d (310 mg, 90%). ¹H NMR (500 MHz, CD₃OD) δ 9.08 (d, J=1.6 Hz, 1H), 8.60 (d, J=1.6 Hz, 1H), 8.53 (d, J=8.1 Hz, 1H), 7.76 (d, J=7.9 Hz, 1H), 7.43 (dt, J=22.7, 7.5 Hz, 2H), 4.75-4.69 (m, 2H), 4.14-4.07 (m, 2H), 2.81 (s, 2H), 1.95-1.77 (m, 5H), 1.47 (m, 9H), 1.32-1.18 (m, 2H).

To a solution of tert-butyl 4-(2-((2-(1H-benzo[d]imidazol-1-yl)-5-chloropyrimidin-4-yl)oxy)ethyl)piperidine-1-carboxylate, 11d (310 mg, 0.12 mmol, 1 equiv) in dichloromethane (4 mL) was added TFA (1.4 mL, 18.1 mmol, 20 equiv) and the reaction was stirred for 16 h after which it was concentrated and purified by silica gel chromatography eluting with 0 to 25% methanol in dichloromethane to give 1-(5-chloro-4-(2-(piperidin-4-yl)ethoxy)pyrimidin-2-yl)-1H-benzo[d]imidazole trifluoroacetate, 12d (194 mg, 61%). ¹H NMR (500 MHz, MeOD) δ 9.09 (s, 1H), 8.62 (s, 1H), 8.56-8.51 (m, 1H), 7.79-7.74 (m, 1H), 7.44 (dtd, J=23.5, 7.4, 1.3 Hz, 2H), 4.74 (t, J=5.9 Hz, 2H), 3.44 (dt, J=12.7, 3.3 Hz, 2H), 3.05 (td, J=12.9, 3.0 Hz, 2H), 2.16-2.08 (m, 2H), 1.98 (dq, J=4.6, 2.5 Hz, 3H), 1.62-1.50 (m, 2H); ¹³C NMR (126 MHz, MeOD) δ 165.25, 156.52, 153.23, 143.83, 142.00, 131.38, 124.74, 123.92, 119.26, 115.27, 113.76, 66.26, 43.79, 34.11, 31.07, 28.61. HRMS (ESI+): m/z calcd for C₁₈H₂₀ClN₅NaO: 380.1249 [M+Na]⁺; found: 380.1255 [M+Na]⁺.

### (1r,3r)-3-((2-(1H-benzo[d]imidazol-1-yl)-5-chloropyrimidin-4-yl)oxy)cyclobutan-1-amine (12e)

To 1-(2,5-dichloropyrimidin-4-yl)-1H-benzo[d]imidazole, 10a (300.0 mg, 1.13 mmol, 1 equiv) was added DMF (6 mL) after which the mixture was cooled in an ice water bath and placed under nitrogen. tert-butyl ((1r,3r)-3-hydroxycyclobutyl)carbamate (211.9 mg, 1.13 mmol, 1 equiv) was added after which NaH (58.9 mg, 60% Wt, 1.47 mmol, 1.3 equiv) was added. The reaction mixture was taken out of the ice bath since a suspension formed after 5 min. The reaction was stirred for 1 h after which it was quenched with water, stirred vigorously, and filtered to isolate solids which were triturated from dichloromethane and filtered to isolate tert-butyl ((1r,3r)-3-((2-(1H-benzo[d]imidazol-1-yl)-5-chloropyrimidin-4-yl)oxy)cyclobutyl)carbamate, lie (344 mg, 73%) as a white solid. ¹H NMR (500 MHz, CDCl₃) δ 8.90 (s, 1H), 8.46 (s, 1H), 8.45-8.39 (m, 1H), 7.85-7.80 (m, 1H), 7.43-7.32 (m, 2H), 5.54 (tt, J=7.2, 3.8 Hz, 1H), 4.82 (s, 1H), 4.41 (s, 1H), 2.72 (ddd, J=14.3, 8.2, 3.7 Hz, 2H), 2.58 (dt, J=13.8, 6.6 Hz, 2H), 1.45 (s, 9H).

To tert-butyl ((1r,3r)-3-((2-(1H-benzo[d]imidazol-1-yl)-5-chloropyrimidin-4-yl)oxy)cyclobutyl)carbamate, lie (315 mg, 757 mol, 1 equiv) in dichloromethane (3 mL) was added TFA (1.17 mL, 15.1 mmol, 20 equiv) and the reaction was stirred overnight at room temperature after which it was concentrated to a residue which was purified by silica gel chromatography eluting with 0 to 25% methanol in dichloromethane to give (1r,3r)-3-((2-(1H-benzo[d]imidazol-1-yl)-5-chloropyrimidin-4-yl)oxy)cyclobutan-1-amine trifluoroacetate, 12e (269 mg, 83%) as a white solid. ¹H NMR (500 MHz, CD₃OD) δ 9.06 (s, 1H), 8.70 (s, 1H), 8.54 (dt, J=8.3, 0.9 Hz, 1H), 7.78 (dt, J=7.8, 0.9 Hz, 1H), 7.49 (ddd, J=8.3, 7.2, 1.3 Hz, 1H), 7.43 (td, J=7.7, 1.3 Hz, 1H), 5.79-5.71 (m, 1H), 4.16-4.06 (m, 1H), 2.88 (dd, J=7.4, 5.5 Hz, 4H); 13C NMR (126 MHz, CD₃OD) δ 164.47, 157.03, 153.16, 143.85, 141.89, 131.40, 124.78, 124.00, 119.33, 115.17, 113.95, 70.36, 42.28, 33.91. HRMS (ESI+): m/z calcd for C₁₅H₁₅ClN₅O: 316.0960 [M+H]⁺; found: 316.0977 [M+H]⁺.

### (1s,3s)-3-((2-(1H-benzo[d]imidazol-1-yl)-5-chloropyrimidin-4-yl)oxy)cyclobutan-1-amine (12f)

To 1-(2,5-dichloropyrimidin-4-yl)-1H-benzo[d]imidazole, 10a (300.0 mg, 1.13 mmol, 1 equiv) was added DMF (5 mL) after which the mixture was cooled in an ice water bath and placed under nitrogen. tert-butyl ((1s,3s)-3-hydroxycyclobutyl)carbamate (211.9 mg, 1.13 mmol, 1 equiv) was added after which NaH (58.9 mg, 60% Wt, 1.471 mmol, 1.3 equiv) was added. The reaction was stirred for 1 h after which it was quenched with water, stirred vigorously, then filtered to isolate the solids which were purified by silica gel chromatography eluting with 0 to 10% MeOH in DCM to give tert-butyl ((1s,3s)-3-((2-(1H-benzo[d]imidazol-1-yl)-5-chloropyrimidin-4-yl)oxy)cyclobutyl)carbamate, 11f (403 mg, 86%). ¹H NMR (500 MHz, CDCl₃) δ 8.90 (s, 1H), 8.47-8.40 (m, 2H), 7.82 (d, J=7.9 Hz, 1H), 7.38 (dt, J=23.3, 7.5 Hz, 2H), 5.16 (p, J=6.9 Hz, 1H), 4.88-4.64 (m, 1H), 4.15-3.96 (m, 1H), 3.10 (q, J=10.1, 8.8 Hz, 2H), 2.19 (q, J=9.5 Hz, 2H), 1.43 (s, 9H).

To tert-butyl ((1s,3s)-3-((2-(1H-benzo[d]imidazol-1-yl)-5-chloropyrimidin-4-yl)oxy)cyclobutyl)carbamate, 11f (253.0 mg, 608.3 mol, 1 equiv) in dichloromethane (3 mL) was added TFA (937.4 μL, 12.17 mmol, 20 equiv) and the reaction was stirred overnight at room temperature after which it was concentrated. The residue was purified by silica gel chromatography eluting with 0 to 25% methanol in dichloromethane to give (1s,3s)-3-((2-(1H-benzo[d]imidazol-1-yl)-5-chloropyrimidin-4-yl)oxy)cyclobutan-1-amine trifluoroacetate, 12f (170 mg, 65.2%). ¹H NMR (500 MHz, CD₃OD) δ 9.07 (s, 1H), 8.67 (s, 1H), 8.54 (d, J=8.1 Hz, 1H), 7.77 (d, J=7.9 Hz, 1H), 7.51-7.45 (m, 1H), 7.42 (td, J=7.6, 1.3 Hz, 1H), 5.43 (p, J=7.1 Hz, 1H), 3.78-3.68 (m, 1H), 3.18 (dtt, J=9.3, 7.0, 2.1 Hz, 2H), 2.51 (dddd, J=13.4, 10.6, 6.2, 2.6 Hz, 2H); ¹³C NMR (126 MHz, CD₃OD) δ 164.17, 157.02, 153.15, 143.83, 141.96, 131.37, 124.81, 123.98, 119.28, 115.23, 113.67, 66.02, 38.20, 35.39. HRMS (ESI+): m/z calcd for C₁₅H₁₅ClN₅O: 316.0960 [M+H]⁺; found: 316.0973 [M+H]⁺.

### 1-(5-chloro-4-(pyrrolidin-3-yloxy)pyrimidin-2-yl)-1H-benzo[d]imidazole (12g)

To 1-(2,5-dichloropyrimidin-4-yl)-1H-benzo[d]imidazole, 10a (3.0 g, 11 mmol, 1 equiv) in DMF (50 mL) cooled in an ice/water bath was added tert-butyl 3-hydroxypyrrolidine-1-carboxylate (2.1 g, 11 mmol, 1 equiv) after which the mixture was stirred under nitrogen. After all solids dissolved, NaH (0.54 g, 60% Wt, 14 mmol, 1.2 equiv) was added portionwise. The reaction mixture was stirred for 1 h after which water (~100-150 mL) was added to quench the reaction. The suspension was stirred vigorously then filtered to isolate crude solids which were purified by silica gel chromatography eluting with 0 to 50% EtOAc in hexanes to give tert-butyl 3-((2-(1H-benzo[d]imidazol-1-yl)-5-chloropyrimidin-4-yl)oxy)pyrrolidine-1-carboxylate, 11g (3.0 g, 64%) as a white solid. ¹H NMR (500 MHz, CDCl₃) δ 8.99 (s, 1H), 8.48 (s, 1H), 8.43 (d, J=6.7 Hz, 1H), 7.85 (d, J=7.7 Hz, 1H), 7.40 (p, J=7.4 Hz, 2H), 5.76 (s, 1H), 3.81 (s, 1H), 3.73-3.50 (m, 3H), 2.32 (s, 2H), 1.46 (m, 9H).

To tert-butyl 3-((2-(1H-benzo[d]imidazol-1-yl)-5-chloropyrimidin-4-yl)oxy)pyrrolidine-1-carboxylate, 11g (3.0 g, 7.2 mmol, 1 equiv) was added dichloromethane (50 mL) and the mixture was cooled in an ice/water bath after which TFA (5.6 mL, 72 mmol, 10 equiv) was added and the reaction was stirred for 16 h as the ice bath expired after which it was concentrated. The residue was purified by silica gel chromatography eluting with 0 to 30% MeOH in DCM to give 1-(5-chloro-4-(pyrrolidin-3-yloxy)pyrimidin-2-yl)-1H-benzo[d]imidazole trifluoroacetate, 12g (2.5 g, 81%). Determined to be a 1.0 TFA salt by following a protocol reported in the literature.^{31 1}H NMR (500 MHz, CD₃OD) δ 9.13 (s, 1H), 8.71 (s, 1H), 8.54 (dt, J=8.3, 1.0 Hz, 1H), 7.78 (dt, J=7.8, 1.0 Hz, 1H), 7.49 (ddd, J=8.3, 7.3, 1.3 Hz, 1H), 7.43 (td, J=7.7, 1.3 Hz, 1H), 6.07 (tt, J=4.5, 1.9 Hz, 1H), 3.87-3.75 (m, 2H), 3.66-3.54 (m, 3H), 2.65-2.52 (m, 2H); ¹³C NMR (126 MHz, CD₃OD) δ 164.08, 157.37, 152.97, 143.86, 142.06, 131.36, 124.86, 124.03, 119.32, 115.23, 114.05, 77.06, 50.51, 44.16, 30.57. HRMS (ESI+): m/z calcd for C₁₅H₁₅ClN₅O: 316.0960 [M+H]⁺; found: 316.0979 [M+H]⁺.

### (S)-1-(5-chloro-4-(pyrrolidin-3-yloxy)pyrimidin-2-yl)-1H-benzo[d]imidazole (12i)

To 1-(2,5-dichloropyrimidin-4-yl)-1H-benzo[d]imidazole, 10a (300.0 mg, 1.13 mmol, 1 equiv) was added DMF (5 mL) after which the mixture was cooled in an ice water bath and placed under nitrogen. Tert-butyl (S)-3-hydroxypyrrolidine-1-carboxylate (211.9 mg, 1.13 mmol, 1 equiv) was added after which NaH (58.85 mg, 60% Wt, 1.471 mmol, 1.3 equiv) was added. After stirring for 1 h, the reaction was quenched with water, stirred vigorously, then filtered to isolate solids which were purified by silica gel chromatography eluting with 0 to 10% MeOH in DCM to give tert-butyl (S)-3-((2-(1H-benzo[d]imidazol-1-yl)-5-chloropyrimidin-4-yl)oxy)pyrrolidine-1-carboxylate, 11i (410 mg, 87%). ¹H NMR (500 MHz, CDCl₃) δ 8.92 (s, 1H), 8.48 (s, 1H), 8.41 (d, J=7.7 Hz, 1H), 7.83 (d, J=7.7 Hz, 1H), 7.37 (q, J=8.4, 7.4 Hz, 2H), 5.78-5.74 (m, 1H), 3.82 (br s, 2H), 3.75-3.46 (m, 3H), 2.32 (s, 2H), 1.49-1.43 (m, 9H).

To tert-butyl (S)-3-((2-(1H-benzo[d]imidazol-1-yl)-5-chloropyrimidin-4-yl)oxy)pyrrolidine-1-carboxylate, 11i (246 mg, 592 mol, 1 equiv) in dichloromethane (3 mL) was added TFA (911 μL, 11.8 mmol, 20 equiv) and the reaction was stirred overnight at room temperature after which it was concentrated and purified by silica gel chromatography eluting with 0 to 25% methanol in dichloromethane to give (S)-1-(5-chloro-4-(pyrrolidin-3-yloxy)pyrimidin-2-yl)-1H-benzo[d]imidazole trifluoroacetate, 12i (179 mg, 71%). ¹H NMR (500 MHz, CD₃OD) δ 9.11 (s, 1H), 8.68 (d, J=1.4 Hz, 1H), 8.51 (d, J=8.1 Hz, 1H), 7.77 (d, J=7.8 Hz, 1H), 7.50-7.45 (m, 1H), 7.42 (td, J=7.7, 1.3 Hz, 1H), 6.08-6.02 (m, 1H), 3.87-3.75 (m, 2H), 3.66-3.53 (m, 2H), 2.65-2.51 (m, 2H); ¹³C NMR (126 MHz, CD₃OD) δ 164.04, 157.32, 152.92, 143.83, 142.05, 131.33, 124.85, 124.02, 119.31, 115.23, 114.04, 77.05, 50.48, 44.14, 30.57. HRMS (ESI+): m/z calcd for C₁₅H₁₅ClN₅O: 316.0960 [M+H]⁺; found: 316.0977 [M+H]⁺.

### (R)-1-(5-chloro-4-(pyrrolidin-3-yloxy)pyrimidin-2-yl)-1H-benzo[d]imidazole (12h)

Made via two different routes as discussed below. The NMR spectra of the final compound resulting from both routes were identical. Furthermore, the structure of the final compound was confirmed by X-ray crystallography (see SI).

### Synthesis of 12 h Using the Shorter Route (Scheme 3):

To a vial containing 1-(2,5-dichloropyrimidin-4-yl)-1H-benzo[d]imidazole, 10a (300 mg, 1.13 mmol, 1 equiv) was added anhydrous DMF (4 mL) and tert-butyl (R)-3-hydroxypyrrolidine-1-carboxylate (212 mg, 1.13 mmol, 1 equiv) after which the mixture was cooled in an ice/water bath and NaH (54.3 mg, 60% Wt, 1.36 mmol, 1.2 equiv) was added. The mixture was stirred under nitrogen for 45 min after which it was quenched with water. The mixture was stirred vigorously for 5 min then filtered to isolate the solids which were purified by silica gel chromatography eluting with 0 to 50% EtOAc in hexanes to give tert-butyl (R)-3-((2-(1H-benzo[d]imidazol-1-yl)-5-chloropyrimidin-4-yl)oxy)pyrrolidine-1-carboxylate, 11h (321 mg, 68.2%). ¹H NMR (500 MHz, CDCl₃) δ 8.91 (s, 1H), 8.46 (s, 1H), 8.39 (d, J=7.8 Hz, 1H), 7.82 (d, J=7.6 Hz, 1H), 7.37 (p, J=7.5 Hz, 2H), 5.75 (s, 1H), 3.81 (br s, 1H), 3.63 (dtd, J=34.3, 17.5, 16.0, 8.9 Hz, 3H), 2.32 (br s, 2H), 1.46 (m, 9H).

To tert-butyl (R)-3-((2-(1H-benzo[d]imidazol-1-yl)-5-chloropyrimidin-4-yl)oxy)pyrrolidine-1-carboxylate, 11h (300 mg, 721 mol, 1 equiv) was added DCM (2 mL) after which TFA (1.10 mL, 14.4 mmol, 20 equiv) was added and the reaction was stirred for 16 h after which it was concentrated and purified by silica gel chromatography eluting with 0 to 30% MeOH in DCM to give (R)-1-(5-chloro-4-(pyrrolidin-3-yloxy)pyrimidin-2-yl)-1H-benzo[d]imidazole trifluoroacetate, 12h (170 mg, 55%). ¹H NMR (500 MHz, CD₃OD) δ 9.11 (s, 1H), 8.69 (s, 1H), 8.51 (d, J=8.1 Hz, 1H), 7.77 (d, J=7.9 Hz, 1H), 7.48 (t, J=7.7 Hz, 1H), 7.42 (t, J=7.7 Hz, 1H), 6.05 (d, J=4.3 Hz, 1H), 3.87-3.75 (m, 2H), 3.60 (q, J=9.7, 7.7 Hz, 2H), 2.62-2.53 (m, 2H); ^{1 13}C NMR (126 MHz, CD₃OD) δ 164.05, 157.33, 152.92, 143.83, 142.05, 131.33, 124.85, 124.02, 119.31, 115.23, 114.04, 77.05, 50.49, 44.14, 30.57.

### Synthesis of 12 h Using the Longer Route (Scheme 4):

To 4,5-dichloro-N-(2-nitrophenyl)pyrimidin-2-amine (made previously above), 5b (500.0 mg, 1.8 mmol, 1 equiv) was added DMF (10 mL) and the reaction was cooled in an ice/water bath after which tert-butyl (R)-3-hydroxypyrrolidine-1-carboxylate (328 mg, 1.8 mmol, 1 equiv) was added. While stirring, NaH (210.5 mg, 60% Wt, 5.3 mmol, 3 equiv) was added and the reaction was stirred at room temperature for 5 min after which it was heated at 65° C. under nitrogen for 1 h. The reaction was allowed to cool to room temperature after which it was immersed in an ice/water bath and quenched with water. The mixture was stirred vigorously then filtered to isolate solids which were purified by silica gel chromatography eluting with 0 to 30% ethyl acetate in hexanes to give tert-butyl (R)-3-((5-chloro-2-((2-nitrophenyl)amino)pyrimidin-4-yl)oxy)pyrrolidine-1-carboxylate, 13a (530 mg, 69%). ¹H NMR (500 MHz, CD₃OD) δ 8.62 (d, J=8.5 Hz, 1H), 8.31 (s, 1H), 8.21 (dd, J=8.4, 1.5 Hz, 1H), 7.72 (t, J=7.9 Hz, 1H), 7.21 (t, J=7.8 Hz, 1H), 5.68 (s, 1H), 3.71 (dt, J=12.6, 6.2 Hz, 1H), 3.67-3.58 (m, 2H), 3.52 (q, J=9.5 Hz, 1H), 2.29 (d, J=9.5 Hz, 2H), 1.49 (m, 9H).

To tert-butyl (R)-3-((5-chloro-2-((2-nitrophenyl)amino)pyrimidin-4-yl)oxy)pyrrolidine-1-carboxylate, 13a (497.0 mg, 1.1 mmol, 1 equiv) was added EtOH:water (5:1, 6 mL) and the mixture was sonicated to dissolve the solids after which zinc (373 mg, 5.7 mmol, 5 equiv) and ammonium chloride (305 mg, 5.7 mmol, 5 equiv) were added. The reaction was stirred at 65° C. for 30 min after which it was allowed to cool to room temperature and diluted with ethyl acetate. The solution was filtered through a pad of celite and the filtrate was washed with water, brine, filtered through an isolute phase separator and concentrated to give tert-butyl (R)-3-((2-((2-aminophenyl)amino)-5-chloropyrimidin-4-yl)oxy)pyrrolidine-1-carboxylate, 14a (462 mg, quant). This material was taken on to the next step without further purification. ¹H NMR (500 MHz, CD₃OD) δ 8.06 (s, 1H), 7.24 (dd, J=7.9, 1.6 Hz, 1H), 7.02 (t, J=7.7 Hz, 1H), 6.87 (dd, J=8.1, 1.5 Hz, 1H), 6.74 (td, J=7.6, 1.5 Hz, 1H), 5.51 (s, 1H), 3.62-3.42 (m, 4H), 2.19 (s, 2H), 1.49 (m, 9H) (the material appears to exist as a mixture of amide rotamers).

To tert-butyl (R)-3-((2-((2-aminophenyl)amino)-5-chloropyrimidin-4-yl)oxy)pyrrolidine-1-carboxylate, 14a (460 mg, 1.13 mmol, 1 equiv) was added THF:MeOH (3:1, 4 mL) and trimethyl orthoformate (3.72 mL, 34.0 mmol, 30 equiv) after which p-TsOH (19.5 mg, 113 mol, 0.1 equiv) was added and the reaction was heated to 65° C. for 1 h. After allowing the reaction to cool to room temperature, it was basified with saturated aqueous sodium bicarbonate solution. The organic layer was washed with brine, filtered through an isolute phase separator and concentrated to a residue which was purified by silica gel chromatography eluting with 0 to 50% ethyl acetate in hexanes to give tert-butyl (R)-3-((2-(1H-benzo[d]imidazol-1-yl)-5-chloropyrimidin-4-yl)oxy)pyrrolidine-1-carboxylate, 11h (214 mg, 45%) as a yellow oil. ¹H NMR (500 MHz, CDCl₃) δ 8.91 (s, 1H), 8.46 (s, 1H), 8.39 (d, J=7.8 Hz, 1H), 7.82 (d, J=7.6 Hz, 1H), 7.37 (p, J=7.5 Hz, 2H), 5.75 (s, 1H), 3.81 (br s, 1H), 3.62 (ddt, J=34.6, 16.6, 8.1 Hz, 3H), 2.32 (br s, 2H), 1.46 (m, 9H).

To tert-butyl (R)-3-((2-(1H-benzo[d]imidazol-1-yl)-5-chloropyrimidin-4-yl)oxy)pyrrolidine-1-carboxylate, 11h (190 mg, 457 mol, 1 equiv) was added DCM (3 mL) and TFA (0.7 mL, 9.1 mmol, 20 equiv) and the reaction was stirred for 16 h after which overnight after which it was concentrated and purified by silica gel chromatography eluting with 0 to 30% methanol in dichloromethane to give (R)-1-(5-chloro-4-(pyrrolidin-3-yloxy)pyrimidin-2-yl)-1H-benzo[d]imidazole trifluoroacetate, 12h (93 mg, 47%). ¹H NMR (500 MHz, CD₃OD) δ 9.12 (s, 1H), 8.69 (s, 1H), 8.52 (d, J=8.1 Hz, 1H), 7.77 (d, J=7.9 Hz, 1H), 7.48 (t, J=7.7 Hz, 1H), 7.42 (t, J=7.6 Hz, 1H), 6.05 (tt, J=4.4, 1.8 Hz, 1H), 3.87-3.75 (m, 2H), 3.66-3.53 (m, 2H), 2.57 (tt, J=7.0, 3.6 Hz, 2H); ¹³C NMR (126 MHz, CD₃OD) δ 164.06, 157.34, 152.94, 143.84, 142.07, 131.35, 124.86, 124.03, 119.31, 115.23, 114.05, 77.06, 50.49, 44.15, 30.57. HRMS (ESI+): m/z calcd for C₁₅H₁₅ClN₅O: 316.0960 [M+H]⁺; found: 316.0974 [M+H]⁺.

Small molecule X-ray crystallography. Small molecule x-ray crystallography was obtained on a Rigaku XtaLAB Synergy, Single source at home/near, HyPix diffractometer. See the SI for ORTEPs of X-ray structures.

MKNK2^{D228G} in Complex with Compound 12h: Materials and Methods

Protein Production. The MKNK2^{D228G} construct used for crystallization was produced as described previously (PDB entry 2AC5).³⁰

Crystallization. MKNK2 in complex with the compound 12 h was crystallized by hanging-drop vapor-diffusion at 20° C. MKNK2 (10 mg/mL in 50 mM NaCl, 10 mM TRIS-HCl pH 7.5 and 1 mM DTT) was incubated with 1.5 mM of the compound 12 h and 5 mM DTT for 1 hour at 4° C. 2.0 μL of the sample was mixed with 1.0 μL of crystallization solution (0.02 M Magnesium Chloride, 0.1 M HEPES-NaOH pH 7.1 and 29% w/v Sodium Polyacrylate 5100) and equilibrated against a reservoir containing 0.2 mL crystallization solution. The crystals were mounted after three weeks. Crystals were cryo-protected in crystallization solution supplemented with 20% v/v Glycerol (final concentration) and cooled in liquid nitrogen. Data were collected at the BioMAX beamline of the MAX IV Laboratory (Lund, Sweden).

Structure determination. Diffraction data were integrated, analyzed and scaled with XDS, POINTLESS, AIMLESS.^{32, 33} The structure was determined by molecular replacement with PHASER using a previously determined model of the complex (without any ligands) as a starting model. The models were improved through manual rebuilding of the model in COOT and restrained refinement with REFMAC5.^{34, 35} Atomic displacement factors were modelled with a single isotropic B-factor per atom. The backbone geometry was analyzed with MOLPROBITY.³⁶ The restraints for the modelled compounds were generated with GRADE.³⁷ See the SI for further crystallization parameters.

Biochemical MNK1 and MNK2 assays. This assay was run using the same conditions that we have reported before.²⁵ The ADP-Glo universal kinase assay (Promega) was used to evaluate the compounds. Active recombinant Mnk1 or Mnk2 (Thermo Scientific) was added to 1× kinase buffer (Cell Signaling Technology) at a final concentration of 4 ng/l for Mnk1 or 2 ng/l for Mnk2. RS domain derived peptide (GRSRSRSRSR, AnaSpec) was included at a saturating concentration of 250 M as a kinase substrate. The kinase/substrate mixture was dispensed into a 384-well white ProxiPlate (PerkinElmer) using a Mantis liquid handler (Formulatrix), and then appropriate nanoliter volumes of compounds were added using an Echo550 (Labcyte). Compounds were incubated for 30 min at ambient temperature before adding ATP to a final concentration of M with the Mantis. The ATP concentration was well below the KM values determined with recent lots of Mnk1 (>500 μM) and Mnk2 (105 M). The kinase reaction proceeded for 1 h at ambient temperature after which the kinase reaction was stopped by addition of 4 μL of ADP-Glo reagent and incubating for 45 min at ambient temperature. Kinase detection agent (9 μL) was added and after incubating for another 45 min at ambient temperature, luminescence was detected on an EnSpire plate reader (PerkinElmer). Graphing analysis and regression analysis were performed in prism (GraphPad Software) or CDD vault.

Cellular flow cytometric eIF4E phosphorylation assays. U937 and MV4-11 cells were maintained in standard growth media. Compounds were tested by first transferring appropriate nanoliter volumes into empty 96-well flat bottom non-treated plate (Greiner) using an Echo550. Cells grown to a density of 0.35-0.7 million cells per mL were pipetted onto the compounds, mixed, and incubated for 2-3 hours at 37° C. with 5% CO₂. Compounds 12f and 12 h were incubated for 2 hours; compounds 12a, 12b, and 12g were incubated for 3 hours. Cells were fixed by adding 32% paraformaldehyde (Electron Microscopy Sciences) to a final concentration of 3% and incubating 30 minutes at ambient temperature. Cells were washed twice in 1× Perm/Wash Buffer I (BD Biosciences, Cat. No. 557885) and suspended in the same buffer with phospho-Ser209 specific eIF4E antibody conjugated to Alexa 647 (BD Biosciences, clone J77-925). Cells were stained overnight at 4° C. Cells were washed twice in 1× Perm/Wash Buffer I and analyzed on a BD LSRII flow cytometer equipped with a high throughput sampler. Median fluorescence intensity values in the eIF4E pSer209 channel were obtained from singlet- and size-gated cells using Cytobank software.

Cell culture. AML cell lines were obtained from ATCC. U937 were grown in RPMI 1640 medium and MV411 in IMDM medium, both with 10% fetal bovine serum (FBS) and antibiotics at 37° C. and 5% CO₂. Cells were tested through STR at least once per year.

Immunoblotting. Cells were treated for 1 or 4 h at 1 or 10 μM of the MNK inhibitors compared to a DMSO vehicle control. Cell lysis and western blots were executed as previously described³⁸. The antibody against phosphorylated-eIF4E at Ser209 (Cat. No. 9741) was purchased from Cell Signaling Technology. Antibodies against eIF4E (Cat. No. sc-9976) and HSP-90 (Cat. No. sc-7947) were bought from Santa Cruz Biotechnology.

Cell viability. Cell viability was determined as previously described with treatments of DMSO as the vehicle control and increasing concentrations of MNK inhibitors, ranging from 0.1 to 30 μM.³⁸ After 4 days, plate absorbance was read after incubating with WST-1 Reagent as indicated by the manufacturer (Sigma-Aldrich) and viability was determined based on the DMSO treated control. GraphPad Prism was used to calculate IC₅₀ values. Combinations of 5-azacytidine (Sigma-Aldrich, St. Louis, MO, USA) with MNK inhibitors were tested in the same format.

Apoptosis. Annexin V FITC and 4′-6-diamidino-2-phenylindole (DAPI)-DNA staining were used to determine apoptosis induction through flow cytometry analysis in MV411 and U937 cells. Cells were treated with 12g at increasing concentrations from 1 to 10 μM for 72 h. Using the BD Pharmingen kit (BD Biosciences), manufacturer's instructions were followed for collecting cells and adding Annexin V FITC and DAPI stains. An LSRFortessa (BD Biosciences) was used to perform the flow analysis and 10,000 cells were read. FlowJo software (Tree Star, Ashland, OR) was used for gating, quantification, and generating dot plots.

Clonogenic leukemic progenitor assays in methylcellulose. Assays were performed based on a previously reported protocol 39. Briefly, 300-500 cells were plated in methylcellulose (MethoCult™ H4434 Classic w/o EPO, Stem Cell Technologies) and treated with DMSO or indicated MNK inhibitors at 1, 5, or 10 μM. After 7 days, colonies were counted and compared to the vehicle control.

Pharmacokinetics. PK studies used C57Bl/6 male mice. Compound was formulated for both PO and IV arms as a 7 mg/mL DMSO/10% Captisol; 1:9 colorless solution. Compounds were administered at 35 mg/kg PO and 3.5 mg/kg IV and plasma and brain were isolated at the indicated time points. 5 μL plasma samples were directly loaded to a 96-well Millipore Multiscreen Solvinter 0.45 m low-binding PTFE hydrophilic filter plate. Brain samples were homogenized with water (×3 dilution) then 10 μL was loaded to the filter plate. All plasma/brain samples were treated with 75 μL 90/10 acetonitrile/water with Carbamazepine as IS to extract the analyte and precipitate protein. The plates were agitated on ice for approximately ten minutes prior to centrifugation into a collection plate. Separate standard curves were prepared in blank mouse plasma and brain homogenate and processed in parallel with the samples. The filtrate was directly analyzed by LC-MS/MS analysis. Data were processed using PKSolver 2.0.

Statistical analysis. For the biochemical assay measuring MNK1/2 inhibition, data shown in Table 1 and Table 2 was processed using Collaborative Drug Discovery's (CDD) built-in algorithm using a 4-parameter Hill slope model. The resulting 95% confidence intervals of the IC₅₀ values were converted to the ±Standard deviation (SD) assuming normal distribution using the equation below:

$$\pm Standard deviation ( SD ) = \frac{Upper bound {IC}_{50} - Lower bound {IC}_{50}}{1.96 * 2}$$

All in vitro experiments in AML cells were performed in two biological replicates and statistical analyses were performed using GraphPad Prism 8.1.2 software or higher. The resulting 95% confidence intervals of the IC₅₀ values were converted to the ±Standard deviation (SD) assuming normal distribution using the equation above.

### REFERENCES

(1) Kosciuczuk, E. M.; Saleiro, D.; Platanias, L. C. Dual targeting of eIF4E by blocking MNK and mTOR pathways in leukemia. Cytokine 2017, 89, 116-121. DOI: 10.1016/j.cyto.2016.01.024
(2) Siddiqui, N.; Sonenberg, N. Signalling to eIF4E in cancer. Biochem. Soc. Trans. 2015, 43 (5), 763-772. DOI: 10.1042/BST20150126
(3) Quintas, A.; Harvey, R. F.; Horvilleur, E.; Garland, G. D.; Schmidt, T.; Kalmar, L.; Dezi, V.; Marini, A.; Fulton, A. M.; Poyry, T. A. A.; Cole, C. H.; Turner, M.; Sawarkar, R.; Chapman, M. A.; Bushell, M.; Willis, A. E. Eukaryotic initiation factor 4B is a multi-functional RNA binding protein that regulates histone mRNAs. Nucleic Acids Res. 2024. DOI: 10.1093/nar/gkae767
(4) Proud, C. G. Mnks, eIF4E phosphorylation and cancer. Biochim. Biophys. Acta. 2015, 1849 (7), 766-773. DOI: 10.1016/j.bbagrm.2014.10.003
(5) Yang, X.; Zhong, W.; Cao, R. Phosphorylation of the mRNA cap-binding protein eIF4E and cancer. Cell. Signal. 2020, 73, 109689. DOI: 10.1016/j.cellsig.2020.109689
(6) Joshi, S.; Platanias, L. C. Mnk Kinases in Cytokine Signaling and Regulation of Cytokine Responses. Biomol. Concepts 2012, 3 (2), 127-139. DOI: 10.1515/bmc-2011-0057
(7) Joshi, S.; Platanias, L. C. Mnk kinase pathway: Cellular functions and biological outcomes. World J. Biol. Chem. 2014, 5 (3), 321-333. DOI: 10.4331/wjbc.v5.i3.321
(8) Parra, J. L.; Buxade, M.; Proud, C. G. Features of the catalytic domains and C termini of the MAPK signal-integrating kinases Mnk1 and Mnk2 determine their differing activities and regulatory properties. J. Biol. Chem. 2005, 280 (45), 37623-37633. DOI: 10.1074/jbc.M508356200
(9) Li, Y.; Yue, P.; Deng, X.; Ueda, T.; Fukunaga, R.; Khuri, F. R.; Sun, S. Y. Protein phosphatase 2A negatively regulates eukaryotic initiation factor 4E phosphorylation and eIF4F assembly through direct dephosphorylation of Mnk and eIF4E. Neoplasia 2010, 12 (10), 848-855. DOI: 10.1593/neo.10704
(10) Xu, W.; Kannan, S.; Verma, C. S.; Nacro, K. Update on the Development of MNK Inhibitors as Therapeutic Agents. J. Med. Chem. 2022, 65 (2), 983-1007. DOI: 10.1021/acs.jmedchem.lc00368
(11) Fernandez, A.; Monsen, P. J.; Platanias, L. C.; Schiltz, G. E. Medicinal chemistry approaches to target the MNK-eIF4E axis in cancer. RSC Med. Chem. 2023, 14 (6), 1060-1087. DOI: 10.1039/d3md00121k
(12) Tschopp, C.; Knauf, U.; Brauchle, M.; Zurini, M.; Ramage, P.; Glueck, D.; New, L.; Han, J.; Gram, H. Phosphorylation of eIF-4E on Ser 209 in response to mitogenic and inflammatory stimuli is faithfully detected by specific antibodies. Mol. Cell. Biol. Res. Commun. 2000, 3 (4), 205-211. DOI: 10.1006/mcbr.2000.0217
(13) Beggs, J. E.; Tian, S.; Jones, G. G.; Xie, J.; Iadevaia, V.; Jenei, V.; Thomas, G.; Proud, C. G. The MAP kinase-interacting kinases regulate cell migration, vimentin expression and eIF4E/CYFIP1 binding. Biochem. J. 2015, 467 (1), 63-76. DOI: 10.1042/BJ20141066
(14) Bain, J.; Plater, L.; Elliott, M.; Shpiro, N.; Hastie, C. J.; McLauchlan, H.; Klevernic, I.; Arthur, J. S.; Alessi, D. R.; Cohen, P. The selectivity of protein kinase inhibitors: a further update. Biochem. J. 2007, 408 (3), 297-315. DOI: 10.1042/BJ20070797
(15) Kwiatkowski, J.; Liu, B.; Pang, S.; Ahmad, N. H. B.; Wang, G.; Poulsen, A.; Yang, H.; Poh, Y. R.; Tee, D. H. Y.; Ong, E.; Retna, P.; Dinie, N.; Kwek, P.; Wee, J. L. K.; Manoharan, V.; Low, C. B.; Seah, P. G.; Pendharkar, V.; Sangthongpitag, K.; Joy, J.; Baburajendran, N.; Jansson, A. E.; Nacro, K.; Hill, J.; Keller, T. H.; Hung, A. W. Stepwise Evolution of Fragment Hits against MAPK Interacting Kinases 1 and 2. J. Med. Chem. 2020, 63 (2), 621-637. DOI: 10.1021/acs.jmedchem.9b01582
(16) Jin, X.; Merrett, J.; Tong, S.; Flower, B.; Xie, J.; Yu, R.; Tian, S.; Gao, L.; Zhao, J.; Wang, X.; Jiang, T.; Proud, C. G. Design, synthesis and activity of Mnk1 and Mnk2 selective inhibitors containing thieno[2,3-d]pyrimidine scaffold. Eur. J Med. Chem. 2019, 162, 735-751. DOI: 10.1016/j.ejmech.2018.10.070
(17) Li, Q.; Ke, L.; Yu, D.; Xu, H.; Zhang, Z.; Yu, R.; Jiang, T.; Guo, Y. W.; Su, M.; Jin, X. Discovery of D25, a Potent and Selective MNK Inhibitor for Sepsis-Associated Acute Spleen Injury. J. Med. Chem. 2024, 67 (4), 3167-3189. DOI: 10.1021/acs.jmedchem.3c02441
(18) Cherian, J.; Nacro, K.; Poh, Z. Y.; Guo, S.; Jeyaraj, D. A.; Wong, Y. X.; Ho, M.; Yang, H. Y.; Joy, J. K.; Kwek, Z. P.; Liu, B.; Wee, J. L.; Ong, E. H.; Choong, M. L.; Poulsen, A.; Lee, M. A.; Pendharkar, V.; Ding, L. J.; Manoharan, V.; Chew, Y. S.; Sangthongpitag, K.; Lim, S.; Ong, S. T.; Hill, J.; Keller, T. H. Structure-Activity Relationship Studies of Mitogen Activated Protein Kinase Interacting Kinase (MNK) 1 and 2 and BCR-ABL1 Inhibitors Targeting Chronic Myeloid Leukemic Cells. J. Med. Chem. 2016, 59 (7), 3063-3078. DOI: 10.1021/acs.jmedchem.5b01712
(19) Kannan, S.; Pradhan, M. R.; Cherian, J.; Joseph, T. L.; Poh, Z. Y.; Hai Yan, Y.; Melvyn, H.; Boping, L.; Jeffrey, H.; Nacro, K.; Verma, C. S. Small Molecules Targeting the Inactive Form of the Mnk1/2 Kinases. ACS Omega 2017, 2 (11), 7881-7891. DOI: 10.1021/acsomega.7b01403
(20) Bou-Petit, E.; Hummer, S.; Alarcon, H.; Slobodnyuk, K.; Cano-Galietero, M.; Fuentes, P.; Guijarro, P. J.; Munoz, M. J.; Suarez-Cabrera, L.; Santamaria, A.; Estrada-Tejedor, R.; Borrell, J. I.; Ramon, Y. C. S. Overcoming Paradoxical Kinase Priming by a Novel MNK1 Inhibitor. J. Med. Chem. 2022, 65 (8), 6070-6087. DOI: 10.1021/acs.jmedchem.1c01941
(21) Reich, S. H.; Sprengeler, P. A.; Chiang, G. G.; Appleman, J. R.; Chen, J.; Clarine, J.; Eam, B.; Ernst, J. T.; Han, Q.; Goel, V. K.; Han, E. Z. R.; Huang, V.; Hung, I. N. J.; Jemison, A.; Jessen, K. A.; Molter, J.; Murphy, D.; Neal, M.; Parker, G. S.; Shaghafi, M.; Sperry, S.; Staunton, J.; Stumpf, C. R.; Thompson, P. A.; Tran, C.; Webber, S. E.; Wegerski, C. J.; Zheng, H.; Webster, K. R. Structure-based Design of Pyridone-Aminal eFT508 Targeting Dysregulated Translation by Selective Mitogen-activated Protein Kinase Interacting Kinases 1 and 2 (MNK1/2) Inhibition. J. Med. Chem. 2018, 61 (8), 3516-3540. DOI: 10.1021/acs.jmedchem.7b01795
(22) Yang, H.; Chennamaneni, L. R.; Ho, M. W. T.; Ang, S. H.; Tan, E. S. W.; Jeyaraj, D. A.; Yeap, Y. S.; Liu, B.; Ong, E. H.; Joy, J. K.; Wee, J. L. K.; Kwek, P.; Retna, P.; Dinie, N.; Nguyen, T. T. H.; Tai, S. J.; Manoharan, V.; Pendharkar, V.; Low, C. B.; Chew, Y. S.; Vuddagiri, S.; Sangthongpitag, K.; Choong, M. L.; Lee, M. A.; Kannan, S.; Verma, C. S.; Poulsen, A.; Lim, S.; Chuah, C.; Ong, T. S.; Hill, J.; Matter, A.; Nacro, K. Optimization of Selective Mitogen-Activated Protein Kinase Interacting Kinases 1 and 2 Inhibitors for the Treatment of Blast Crisis Leukemia. J. Med. Chem. 2018, 61 (10), 4348-4369. DOI: 10.1021/acs.jmedchem.7b01714
(23) Santag, S.; Siegel, F.; Wengner, A. M.; Lange, C.; Bomer, U.; Eis, K.; Puhler, F.; Lienau, P.; Bergemann, L.; Michels, M.; von Nussbaum, F.; Mumberg, D.; Petersen, K. BAY 1143269, a novel MNK1 inhibitor, targets oncogenic protein expression and shows potent anti-tumor activity. Cancer Lett. 2017, 390, 21-29. DOI: 10.1016/j.canlet.2016.12.029
(24) Yuan, X.; Guan, D.; Chen, C.; Guo, S.; Wu, H.; Bu, H.; Yang, C. Y.; Wang, M.; Zhou, J.; Zhang, H. Development of an Imidazopyridazine-Based MNK1/2 Inhibitor for the Treatment of Lymphoma. J. Med. Chem. 2024. DOI: 10.1021/acs.jmedchem.3c02008
(25) Mishra, R. K.; Clutter, M. R.; Blyth, G. T.; Kosciuczuk, E. M.; Blackburn, A. Z.; Beauchamp, E. M.; Schiltz, G. E.; Platanias, L. C. Discovery of novel Mnk inhibitors using mutation-based induced-fit virtual high-throughput screening. Chem. Biol. Drug Des. 2019, 94 (4), 1813-1823. DOI: 10.1111/cbdd.13585
(26) Xing, L.; Klug-Mcleod, J.; Rai, B.; Lunney, E. A. Kinase hinge binding scaffolds and their hydrogen bond patterns. Bioorg. Med. Chem. 2015, 23 (19), 6520-6527. DOI: 10.1016/j.bmc.2015.08.006
(27) Shveygert, M.; Kaiser, C.; Bradrick, S. S.; Gromeier, M. Regulation of eukaryotic initiation factor 4E (eIF4E) phosphorylation by mitogen-activated protein kinase occurs through modulation of Mnk1-eIF4G interaction. Mol. Cell. Biol. 2010, 30 (21), 5160-5167. DOI: 10.1128/MCB.00448-10
(28) Al-Khawaldeh, I.; Al Yasiri, M. J.; Aldred, G. G.; Basmadjian, C.; Bordoni, C.; Harnor, S. J.; Heptinstall, A. B.; Hobson, S. J.; Jennings, C. E.; Khalifa, S.; Lebraud, H.; Martin, M. P.; Miller, D. C.; Shrives, H. J.; de Souza, J. V.; Stewart, H. L.; Temple, M.; Thomas, H. D.; Totobenazara, J.; Tucker, J. A.; Tudhope, S. J.; Wang, L. Z.; Bronowska, A. K.; Cano, C.; Endicott, J. A.; Golding, B. T.; Hardcastle, I. R.; Hickson, I.; Wedge, S. R.; Willmore, E.; Noble, M. E. M.; Waring, M. J. An Alkynylpyrimidine-Based Covalent Inhibitor That Targets a Unique Cysteine in NF-kappaB-Inducing Kinase. J. Med. Chem. 2021, 64 (14), 10001-10018. DOI: 10.1021/acs.jmedchem.0c01249
(29) Gabriel, C. M.; Lee, N. R.; Bigorne, F.; Klumphu, P.; Parmentier, M.; Gallou, F.; Lipshutz, B. H. Effects of Co-solvents on Reactions Run under Micellar Catalysis Conditions. Org. Lett. 2017, 19 (1), 194-197. DOI: 10.1021/acs.orglett.6b03468
(30) Jauch, R.; Jakel, S.; Netter, C.; Schreiter, K.; Aicher, B.; Jackle, H.; Wahl, M. C. Crystal structures of the Mnk2 kinase domain reveal an inhibitory conformation and a zinc binding site. Structure 2005, 13 (10), 1559-1568. DOI: 10.1016/j.str.2005.07.013
(31) Little, M. J.; Aubry, N.; Beaudoin, M. E.; Goudreau, N.; LaPlante, S. R. Quantifying trifluoroacetic acid as a counterion in drug discovery by 19F NMR and capillary electrophoresis. J. Pharm. Biomed. Anal. 2007, 43 (4), 1324-1330. DOI: 10.1016/j.jpba.2006.10.039
(32) Evans, P. Scaling and assessment of data quality. Acta Crystallogr. D 2006, 62 (Pt 1), 72-82. DOI: 10.1107/S0907444905036693
(33) Kabsch, W. Xds. Acta Crystallogr. D 2010, 66 (Pt 2), 125-132. DOI: 10.1107/S0907444909047337
(34) Emsley, P.; Lohkamp, B.; Scott, W. G.; Cowtan, K. Features and development of Coot. Acta Crystallogr. D 2010, 66 (Pt 4), 486-501. DOI: 10.1107/S0907444910007493
(35) Murshudov, G. N.; Skubak, P.; Lebedev, A. A.; Pannu, N. S.; Steiner, R. A.; Nicholls, R. A.; Winn, M. D.; Long, F.; Vagin, A. A. REFMAC5 for the refinement of macromolecular crystal structures. Acta Crystallogr. D 2011, 67 (Pt 4), 355-367. DOI: 10.1107/S0907444911001314
(36) Williams, C. J.; Headd, J. J.; Moriarty, N. W.; Prisant, M. G.; Videau, L. L.; Deis, L. N.; Verma, V.; Keedy, D. A.; Hintze, B. J.; Chen, V. B.; Jain, S.; Lewis, S. M.; Arendall, W. B., 3rd; Snoeyink, J.; Adams, P. D.; Lovell, S. C.; Richardson, J. S.; Richardson, D. C. MolProbity: More and better reference data for improved all-atom structure validation. Protein Sci. 2018, 27 (1), 293-315. DOI: 10.1002/pro.3330
(37) Smart, O. S.; Bricogne, G. Achieving High Quality Ligand Chemistry in Protein-Ligand Crystal Structures for Drug Design. Dordrecht, 2015; Springer Netherlands: pp 165-181.
(38) Beauchamp, E. M.; Kosciuczuk, E. M.; Serrano, R.; Nanavati, D.; Swindell, E. P.; Viollet, B.; O'Halloran, T. V.; Altman, J. K.; Platanias, L. C. Direct binding of arsenic trioxide to AMPK and generation of inhibitory effects on acute myeloid leukemia precursors. Mol. Cancer Ther. 2015, 14 (1), 202-212. DOI: 10.1158/1535-7163.MCT-14-0665-T
(39) Colamonici, M.; Blyth, G.; Saleiro, D.; Szilard, A.; Bliss-Moreau, M.; Giles, F. J.; Altman, J. K.; Beauchamp, E. M.; Platanias, L. C. Dual targeting of acute myeloid leukemia progenitors by catalytic mTOR inhibition and blockade of the p110alpha subunit of PI3 kinase. Oncotarget 2015, 6 (10), 8062-8070. DOI: 10.18632/oncotarget.3509

### Supporting Information (SI)

X-ray quality crystals of 2f were obtained by slow diffusion of pentane into dichloromethane. See Table 6 for more detailed crystal data and structure refinement for this compound. CCDC 2386091 contains supplementary crystallographic data for compound 2f.

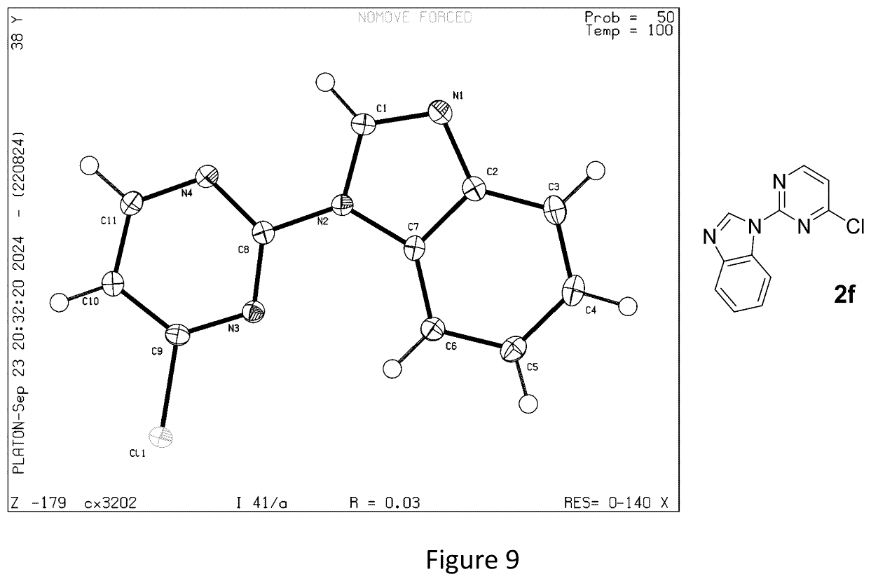

FIG. 9 shows ORTEP of intermediate 2f.

Experimental. Single crystals of C₁₁H₇ClN₄ [cx3202] were supplied. A suitable crystal was selected and the crystal was mounted on a MITIGEN holder in paratone oil on a XtaLAB Synergy R, DW system, HyPix diffractometer. The crystal was kept at 99.99(10) K during data collection. Using Olex2¹, the structure was solved with the SHELXT² structure solution program using Intrinsic Phasing and refined with the XL³ refinement package using Least Squares minimization.

Refinement Details. No special refinement necessary.

Solvent Treatment Details. N/A

X-ray quality crystals of 3a were obtained by slow diffusion of pentane into dichloromethane. Table 7 for more detailed crystal data and structure refinement for this compound. CCDC 2386090 contains supplementary crystallographic data for compound 3a.

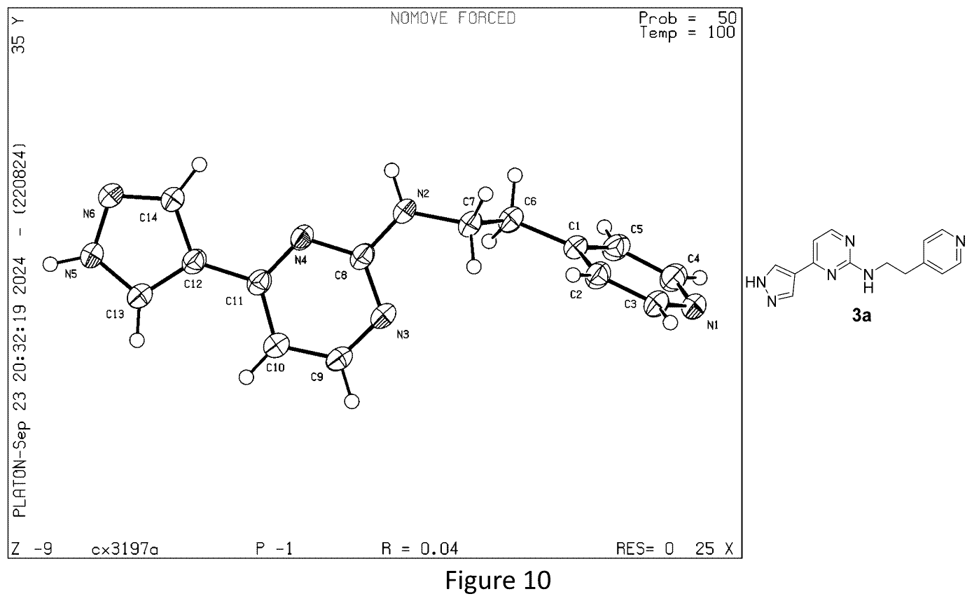

FIG. 10 shows ORTEP of compound 3a.

Experimental. Single crystals of C₁₄H₁₄N₆[cx3197a] were supplied. A suitable crystal was selected and the crystal was mounted on a MITIGEN holder in paratone oil on a XtaLAB Synergy R, DW system, HyPix diffractometer. The crystal was kept at 100.03(19) K during data collection. Using Olex2¹, the structure was solved with the SHELXT² structure solution program using Intrinsic Phasing and refined with the XL³ refinement package using Least Squares minimisation.

Refinement Details. No special refinement necessary.

Solvent Treatment Details. N/A

X-ray quality crystals of 3c were obtained by slow diffusion of pentane into dichloromethane. See Table 8 for more detailed crystal data and structure refinement for this compound. CCDC 2386088 contains supplementary crystallographic data for compound 3c.

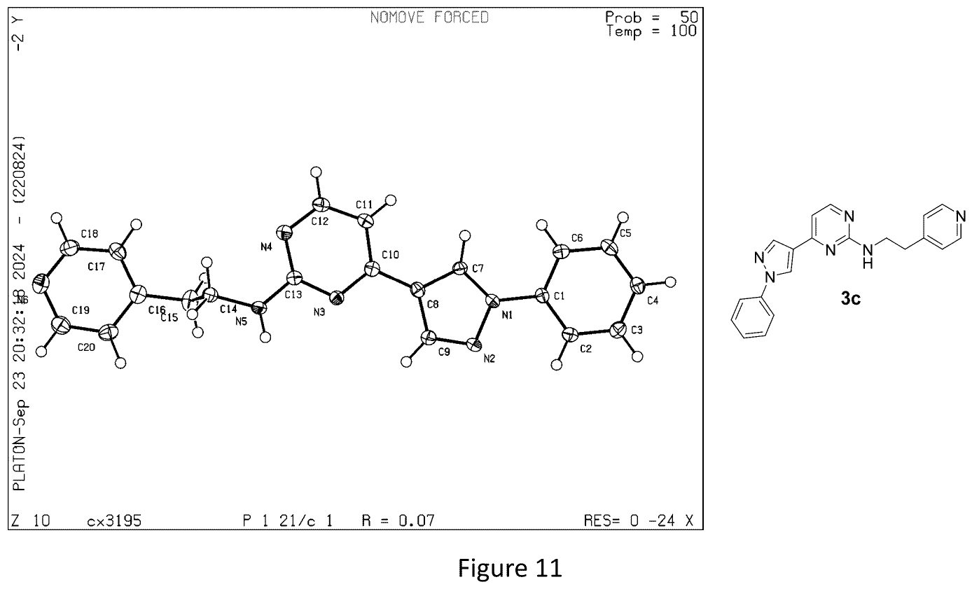

FIG. 11 shows ORTEP of compound 3c.

Experimental. Single crystals of C₂₀H₁₈N₆[cx3195] were supplied. A suitable crystal was selected and the crystal was mounted on a MITIGEN holder in paratone oil on a XtaLAB Synergy R, DW system, HyPix diffractometer. The crystal was kept at 100.00(16) K during data collection. Using Olex2¹, the structure was solved with the SHELXT² structure solution program using Intrinsic Phasing and refined with the XL³ refinement package using Least Squares minimisation.

Refinement Details. No special refinement necessary.

Solvent Treatment Details. N/A

X-ray quality crystals of 3d were obtained by slow diffusion of pentane into dichloromethane. See Table 9 for more detailed crystal data and structure refinement for this compound. CCDC 2386089 contains supplementary crystallographic data for compound 3d.

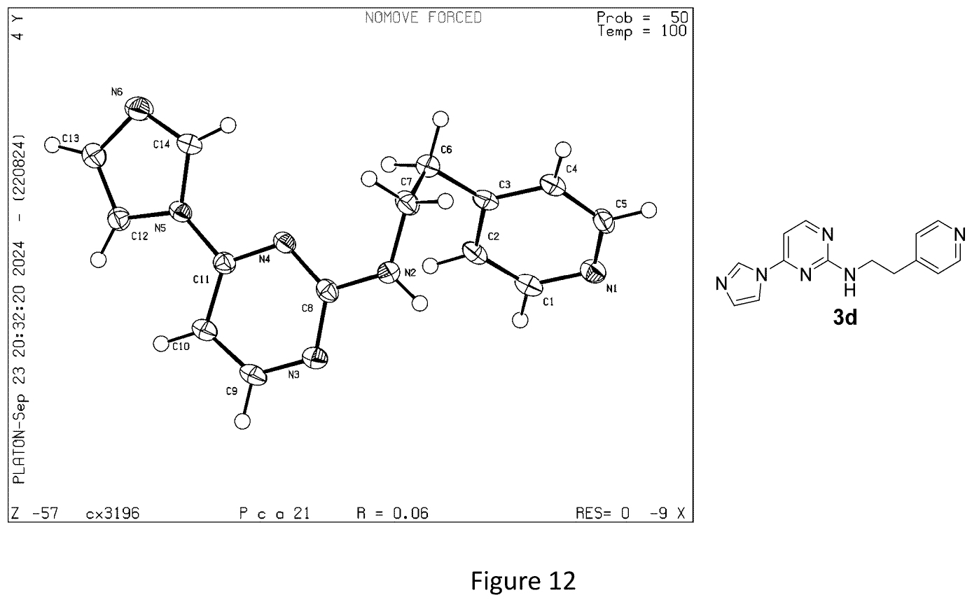

FIG. 12 shows ORTEP of compound 3d.

Experimental. Single crystals of C₁₄H₁₄N₆[cx3196] were supplied. A suitable crystal was selected and the crystal was mounted on a MITIGEN holder in paratone oil on a XtaLAB Synergy R, DW system, HyPix diffractometer. The crystal was kept at 100.01(10) K during data collection. Using Olex2¹, the structure was solved with the SHELXT² structure solution program using Intrinsic Phasing and refined with the XL³ refinement package using Least Squares minimisation.

Refinement Details. No special refinement necessary.

Solvent Treatment Details. N/A

X-ray quality crystals of 8a were obtained by slow diffusion of pentane into dichloromethane. See Table 10 for more detailed crystal data and structure refinement for this compound. CCDC 2386092 contains supplementary crystallographic data for compound 8a.

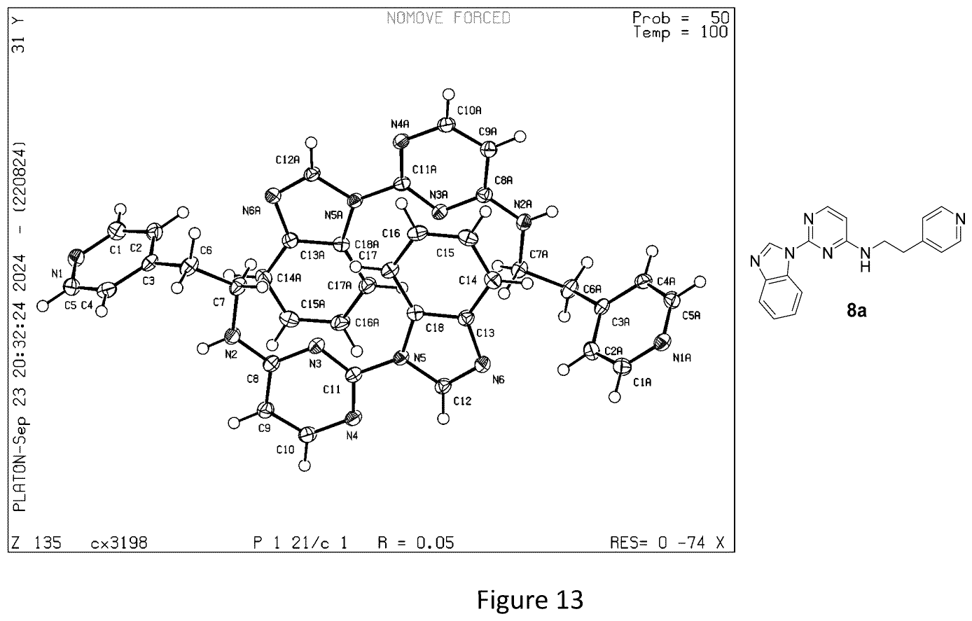

FIG. 13 shows ORTEP of compound 8a.

Experimental. Single crystals of C₁₈H₁₆N₆[cx3198] were supplied. A suitable crystal was selected and the crystal was mounted on a MITIGEN holder in paratone oil on a XtaLAB Synergy R, DW system, HyPix diffractometer. The crystal was kept at 100.02(15) K during data collection. Using Olex2¹, the structure was solved with the SHELXT² structure solution program using Intrinsic Phasing and refined with the XL³ refinement package using Least Squares minimisation.

Refinement Details. No special refinement necessary.

Solvent Treatment Details. N/A

X-ray quality crystals of 8d were obtained by slow diffusion of pentane into dichloromethane. See Table 11 for more detailed crystal data and structure refinement for this compound. CCDC 2386094 contains supplementary crystallographic data for compound 8d.

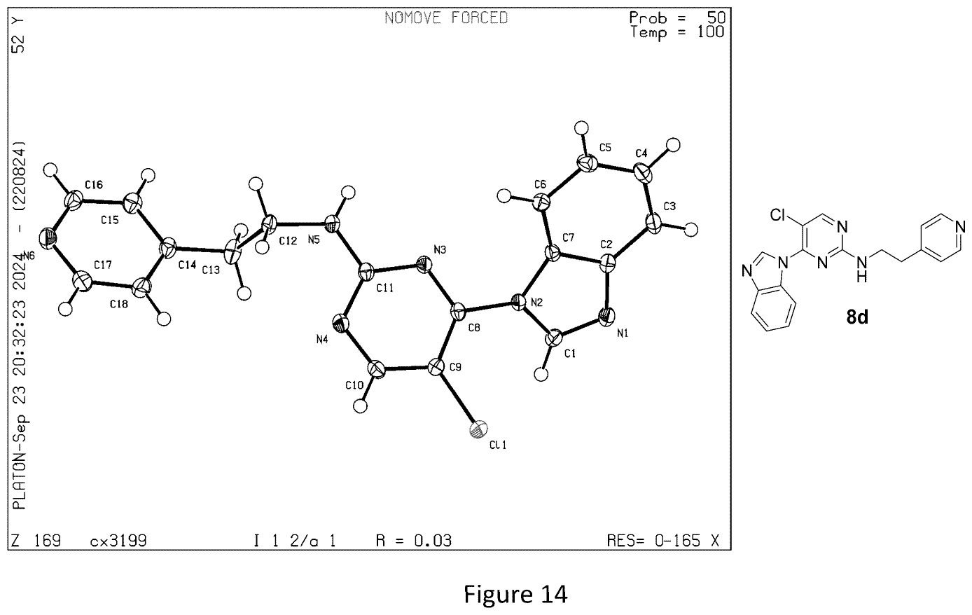

FIG. 14 shows ORTEP of compound 8d.

Experimental. Single crystals of C₁₈H₁₅ClN₆ [cx3199] were supplied. A suitable crystal was selected and the crystal was mounted on a MITIGEN holder in paratone oil on a XtaLAB Synergy R, DW system, HyPix diffractometer. The crystal was kept at 100.00(10) K during data collection. Using Olex2¹, the structure was solved with the SHELXT² structure solution program using Intrinsic Phasing and refined with the XL³ refinement package using Least Squares minimisation.

Refinement Details. No special refinement necessary.

Solvent Treatment Details. N/A

X-ray quality crystals of 10a were obtained by slow diffusion of pentane into dichloromethane. See Table 12 for more detailed crystal data and structure refinement for this compound. CCDC 2386095 contains supplementary crystallographic data for compound 10a.

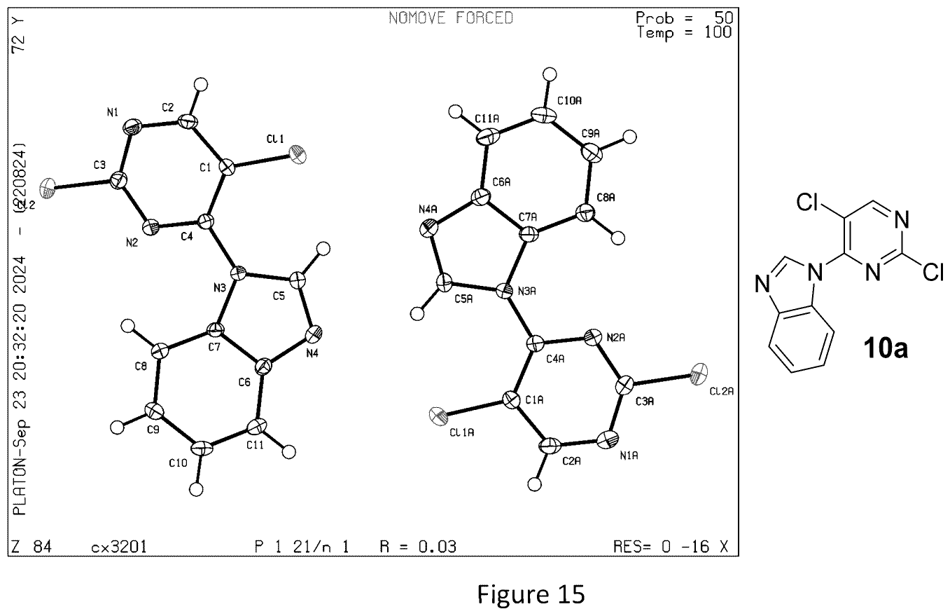

FIG. 15 shows ORTEP of compound 10a.

Experimental. Single crystals of C₁₁H₆Cl₂N₄[cx3201] were supplied. A suitable crystal was selected and the crystal was mounted on a MITIGEN holder in paratone oil on a XtaLAB Synergy R, DW system, HyPix diffractometer. The crystal was kept at 99.99(10) K during data collection. Using Olex2¹, the structure was solved with the SHELXT² structure solution program using Intrinsic Phasing and refined with the XL³ refinement package using Least Squares minimisation.

Refinement Details. The crystal under investigation was found to be pseudo-merohedrally twinned. The exact twin matrix was found to be (−1.0, 0.0, 0.0, 0.0, -1.0, 0.0, 1.0, 0.0, 1.0). The twin fraction refined to a value of 0.7136(8).

Solvent Treatment Details. N/A

X-ray quality crystals of 12 h were obtained by slow diffusion of pentane into dichloromethane. See Table 13 for more detailed crystal data and structure refinement for this compound. CCDC 2386093 contains supplementary crystallographic data for compound 12h.

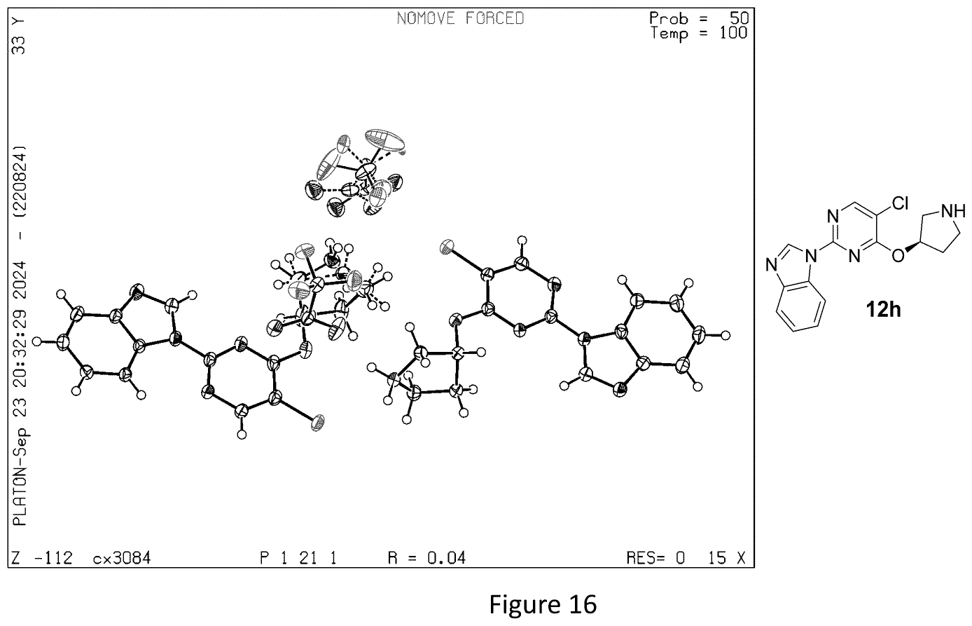

FIG. 16 shows ORTEP of compound 12h.

Experimental. Single crystals of C₁₇H₁₅ClF₃N₅O₃ [cx3084] were supplied. A suitable crystal was selected and the crystal was mounted on a MITIGEN holder in paratone oil on a XtaLAB Synergy, Single source at home/near, HyPix diffractometer. The crystal was kept at 100.0(9) K during data collection. Using Olex2¹, the structure was solved with the SHELXT² structure solution program using Intrinsic Phasing and refined with the SHELXL³ refinement package using Least Squares minimisation.

Refinement Details. The enhanced rigid-bond restraint (SHELX keyword RIGU) as well as restraints on similar amplitudes separated by less than 1.7 Ang. (SIMU) were applied on the disordered atoms.⁴

Solvent Treatment Details. N/A

TABLE 6
Crystal data and structure refinement for 2f

| Identification code | cx3202 |
|---|---|
| Empirical formula | C₁₁H₇ClN₄ |
| Formula weight | 230.66 |
| Temperature/K | 99.99(10) |
| Crystal system | tetragonal |
| Space group | I4₁/a |
| a/Å | 27.0155(2) |
| b/Å | 27.0155(2) |
| c/Å | 5.3348(5) |
| α/° | 90 |
| β/° | 90 |
| γ/° | 90 |
| Volume/Å³ | 3893.5(4) |
| Z | 16 |
| ρ_{calc}/mg mm⁻³ | 1.574 |
| μ/mm⁻¹ | 3.257 |
| F(000) | 1888 |
| Crystal size/mm³ | 0.451 × 0.056 × 0.055 |
| 2Θ range for | 6.544 to 155.81° |
| data collection | |
| Index ranges | −32 ≤ h ≤ 33, −25 ≤ |
| | k ≤ 33, −6 ≤ 1 ≤ 6 |
| Reflections collected | 17884 |
| Independent reflections | 2045[R(int) = 0.0244] |
| Data/restraints/parameters | 2045/0/146 |
| Goodness-of-fit on F² | 1.075 |
| Final R indexes [I > 2σ (I)] | R₁ = 0.0259, wR₂ = 0.0683 |
| Final R indexes [all data] | R₁ = 0.0265, wR₂ = 0.0686 |
| Largest diff. peak/hole/e Å⁻³ | 0.301/−0.206 |

TABLE 7
Crystal data and structure refinement for 3a

| Identification code | cx3197a |
|---|---|
| Empirical formula | C₁₄H₁₄N₆ |
| Formula weight | 266.31 |
| Temperature/K | 100.03(19) |
| Crystal system | triclinic |
| Space group | P-1 |
| a/Å | 8.1656(4) |
| b/Å | 8.4397(3) |
| c/Å | 10.1002(4) |
| α/° | 88.624(3) |
| β/° | 68.377(4) |
| γ/° | 84.710(3) |
| Volume/Å³ | 644.28(5) |
| Z | 2 |
| ρ_{calc}/mg mm⁻³ | 1.373 |
| μ/mm⁻¹ | 0.716 |
| F(000) | 280 |
| Crystal size/mm³ | 0.041 × 0.034 × 0.015 |
| 2Θ range for | 9.42 to 154.914° |
| data collection | |
| Index ranges | −10 ≤ h ≤ 10, −10 ≤ |
| | k ≤ 10, −12 ≤ 1 ≤ 12 |
| Reflections collected | 24224 |
| Independent reflections | 2624[R(int) = 0.0503] |
| Data/restraints/parameters | 2624/0/189 |
| Goodness-of-fit on F² | 1.049 |
| Final R indexes [I > 2σ (I)] | R₁ = 0.0428, wR₂ = 0.1092 |
| Final R indexes [all data] | R₁ = 0.0592, wR₂ = 0.1184 |
| Largest diff. peak/hole/e Å⁻³ | 0.163/−0.189 |

TABLE 8
Crystal data and structure refinement for 3c

| Identification code | cx3195 |
|---|---|
| Empirical formula | C₂₀H₁₈N₆ |
| Formula weight | 342.40 |
| Temperature/K | 100.00(16) |
| Crystal system | monoclinic |
| Space group | P2₁/c |
| a/Å | 19.5283(9) |
| b/Å | 5.5463(2) |
| c/Å | 16.7134(7) |
| α/° | 90, |
| β/° | 112.649(5) |
| γ/° | 90 |
| Volume/Å³ | 1670.62(14) |
| Z | 4 |
| ρ_{calc}/mg mm⁻³ | 1.361 |
| μ/mm⁻¹ | 0.682 |
| F(000) | 720 |
| Crystal size/mm³ | 0.078 × 0.026 × 0.005 |
| 2Θ range for | 4.904 to 148.018° |
| data collection | |
| Index ranges | −19 ≤ h ≤ 24, −6 ≤ |
| | k ≤ 6, −20 ≤ 1 ≤ 15 |
| Reflections collected | 11031 |
| Independent reflections | 3162[R(int) = 0.0473] |
| Data/restraints/parameters | 3162/0/239 |
| Goodness-of-fit on F² | 1.132 |
| Final R indexes [I > 2σ (I)] | R₁ = 0.0708, wR₂ = 0.1561 |
| Final R indexes [all data] | R₁ = 0.0957, wR₂ = 0.1647 |
| Largest diff. peak/hole/e Å⁻³ | 0.264/−0.319 |

TABLE 9
Crystal data and structure refinement for 3d

| Identification code | cx3196 |
|---|---|
| Empirical formula | C₁₄H₁₄N₆ |
| Formula weight | 266.31 |
| Temperature/K | 100.01(10) |
| Crystal system | orthorhombic |
| Space group | Pca2₁ |
| a/Å | 10.7590(2) |
| b/Å | 16.2690(5) |
| c/Å | 7.6690(2) |
| α/° | 90 |
| β/° | 90 |
| γ/° | 90 |
| Volume/Å³ | 1342.36(6) |
| Z | 4 |
| ρ_{calc}/mg mm⁻³ | 1.318 |
| μ/mm⁻¹ | 0.687 |
| F(000) | 560 |
| Crystal size/mm³ | 0.13 × 0.019 × 0.004 |
| 2Θ range for | 5.432 to 156.36° |
| data collection | |
| Index ranges | −13 ≤ h ≤ 13, −18 ≤ |
| | k ≤ 20, −9 ≤ 1 ≤ 9 |
| Reflections collected | 10736 |
| Independent reflections | 2550[R(int) = 0.0811] |
| Data/restraints/parameters | 2550/1/184 |
| Goodness-of-fit on F² | 1.087 |
| Final R indexes [I > 2σ (I)] | R₁ = 0.0555, wR₂ = 0.1334 |
| Final R indexes [all data] | R₁ = 0.0603, wR₂ = 0.1361 |
| Largest diff. peak/hole/e Å⁻³ | 0.247/−0.332 |

TABLE 10
Crystal data and structure refinement for 8a

| Identification code | cx3198 |
|---|---|
| Empirical formula | C₁₈H₁₆N₆ |
| Formula weight | 316.37 |
| Temperature/K | 100.02(15) |
| Crystal system | monoclinic |
| Space group | P2₁/c |
| a/Å | 9.19700(10) |
| b/Å | 38.0201(3) |
| c/Å | 9.90690(10) |
| α/° | 90 |
| β/° | 116.815(2) |
| γ/° | 90 |
| Volume/Å³ | 3091.64(7) |
| Z | 8 |
| ρ_{calc}/mg mm⁻³ | 1.359 |
| μ/mm⁻¹ | 0.690 |
| F(000) | 1328 |
| Crystal size/mm³ | 0.07 × 0.032 × 0.008 |
| 2Θ range for | 4.648 to 156.092° |
| data collection | |
| Index ranges | −11 ≤ h ≤ 11, −48 ≤ |
| | k ≤ 47, −12 ≤ 1 ≤ 11 |
| Reflections collected | 30422 |
| Independent reflections | 6409[R(int) = 0.0396] |
| Data/restraints/parameters | 6409/0/441 |
| Goodness-of-fit on F² | 1.083 |
| Final R indexes [I > 2σ (I)] | R₁ = 0.0463, wR₂ = 0.1219 |
| Final R indexes [all data] | R₁ = 0.0507, wR₂ = 0.1252 |
| Largest diff. peak/hole/e Å⁻³ | 0.319/−0.260 |

TABLE 11
Crystal data and structure refinement for 8d

| Identification code | cx3199 |
|---|---|
| Empirical formula | C₁₈H₁₅ClN₆ |
| Formula weight | 350.81 |
| Temperature/K | 100.00(10) |
| Crystal system | monoclinic |
| Space group | I2/a |
| a/Å | 16.20660(10) |
| b/Å | 8.89500(10) |
| c/Å | 24.3522(2) |
| α/° | 90 |
| β/° | 103.6870(10) |
| γ/° | 90 |
| Volume/Å³ | 3410.87(5) |
| Z | 8 |
| ρ_{calc}/mg mm⁻³ | 1.366 |
| μ/mm⁻¹ | 2.089 |
| F(000) | 1456 |
| Crystal size/mm³ | 0.437 × 0.184 × 0.02 |
| 2Θ range for | 7.472 to 155.828° |
| data collection | |
| Index ranges | −20 ≤ h ≤ 20, −11 ≤ |
| | k ≤ 11, −30 ≤ 1 ≤ 30 |
| Reflections collected | 31791 |
| Independent reflections | 3594[R(int) = 0.0232] |
| Data/restraints/parameters | 3594/0/231 |
| Goodness-of-fit on F² | 1.068 |
| Final R indexes [I > 2σ (I)] | R₁ = 0.0282, wR₂ = 0.0762 |
| Final R indexes [all data] | R₁ = 0.0288, wR₂ = 0.0766 |
| Largest diff. peak/hole/e Å⁻³ | 0.306/−0.246 |

TABLE 12
Crystal data and structure refinement for 10a

| Identification code | cx3201 |
|---|---|
| Empirical formula | C₁₁H₆Cl₂N₄ |
| Formula weight | 265.10 |
| Temperature/K | 99.99(10) |
| Crystal system | monoclinic |
| Space group | P2₁/n |
| a/Å | 7.06340(10) |
| b/Å | 29.2195(3) |
| c/Å | 10.85370(10) |
| α/° | 90 |
| β/° | 109.011(2) |
| γ/° | 90 |
| Volume/Å³ | 2117.90(5) |
| Z | 8 |
| ρ_{calc}/mg mm⁻³ | 1.663 |
| μ/mm⁻¹ | 5.351 |
| F(000) | 1072 |
| Crystal size/mm³ | 0.145 × 0.025 × 0.015 |
| 2Θ range for | 6.05 to 156.102° |
| data collection | |
| Index ranges | −8 ≤ h ≤ 8, −31 ≤ |
| | k ≤ 36, −13 ≤ 1 ≤ 10 |
| Reflections collected | 20764 |
| Independent reflections | 4357[R(int) = 0.0392] |
| Data/restraints/parameters | 4357/0/308 |
| Goodness-of-fit on F² | 1.039 |
| Final R indexes [I > 2σ (I)] | R₁ = 0.0291, wR₂ = 0.0724 |
| Final R indexes [all data] | R₁ = 0.0302, wR₂ = 0.0730 |
| Largest diff. peak/hole/e Å⁻³ | 0.341/−0.273 |

TABLE 13
Crystal data and structure refinement for 12h

| Identification code | cx3084 |
|---|---|
| Empirical formula | C₁₇H₁₅ClF₃N₅O₃ |
| Formula weight | 429.79 |
| Temperature/K | 100.0(9) |
| Crystal system | monoclinic |
| Space group | P2₁ |
| a/Å | 8.93521(10) |
| b/Å | 14.41163(16) |
| c/Å | 13.84451(15) |
| α/° | 90 |
| β/° | 96.1834(10) |
| γ/° | 90 |
| Volume/Å³ | 1772.40(3) |
| Z | 4 |
| ρ_{calc}/mg mm⁻³ | 1.611 |
| μ/mm⁻¹ | 2.494 |
| F(000) | 880 |
| Crystal size/mm³ | 0.202 × 0.114 × 0.052 |
| 2Θ range for | 6.422 to 159.968° |
| data collection | |
| Index ranges | −11 ≤ h ≤ 11, −17 ≤ |
| | k ≤ 18, −17 ≤ 1 ≤ 17 |
| Reflections collected | 35163 |
| Independent reflections | 7437[R(int) = 0.0665] |
| Data/restraints/parameters | 7437/97/597 |
| Goodness-of-fit on F² | 1.125 |
| Final R indexes [I > 2σ (I)] | R₁ = 0.0427, wR₂ = 0.1169 |
| Final R indexes [all data] | R₁ = 0.0442, wR₂ = 0.1188 |
| Largest diff. peak/hole/e Å⁻³ | 0.269/−0.328 |

TABLE 14
Crystallization Parameters of MKNK2D228G
in complex with Compound 12h

| PDB entry | 9HRC |
|---|---|
| Data processing | |
| Space group | P3₂21 |
| Unit cell dimensions | |
| a, b, c/Å | 106.9 106.9 70.0 |
| a, b, g/° | 90.0 90.0 120.0 |
| Resolution^{a} | 55.8-3.16 (3.38-3.16) |
| Number of reflections | |
| Total | 166441 (30507) |
| Unique | 8187 (1461) |
| Rₘₑₐₛ | 0.150 (1.241) |
| Rₚᵢₘ | 0.045 (0.374) |
| Mean I/sI | 15.2 (2.8) |
| CC_{1/2} | 0.999 (0.864) |
| Multiplicity | 20.3 (20.9) |
| Completeness | 100.0 (100.0) |
| Wilson B-factor/Å² | 77.3 |
| Refinement | |
| Resolution | 35.0-3.16 (3.24-3.16) |
| R_{work} | 0.183 (0.306) |
| R_{free} | 0.243 (0.344) |
| Number of atoms | 2297 |
| Average B-factor | 99.4 |
| R.M.S. deviations | |
| Bond lengths/Å | 0.002 |
| Bond angles/° | 0.768 |
| Ramachandran plot/% | |
| Favored | 94.2 |
| Allowed | 5.4 |
| Outlier | 0.4 |
| Clashscore | 4.0 |

^{a}Figures in parentheses refer to the highest resolution shell

MKNK2 protein crystallography was performed by CRELUX GmbH—a Wuxi AppTec company. CRELUX acknowledges MAX IV Laboratory for time on Beamline BioMAX. Research conducted at MAX IV, a Swedish national user facility, is supported by the Swedish Research council under contract 2018-07152, the Swedish Governmental Agency for Innovation Systems under contract 2018-04969, and Formas under contract 2019-02496.

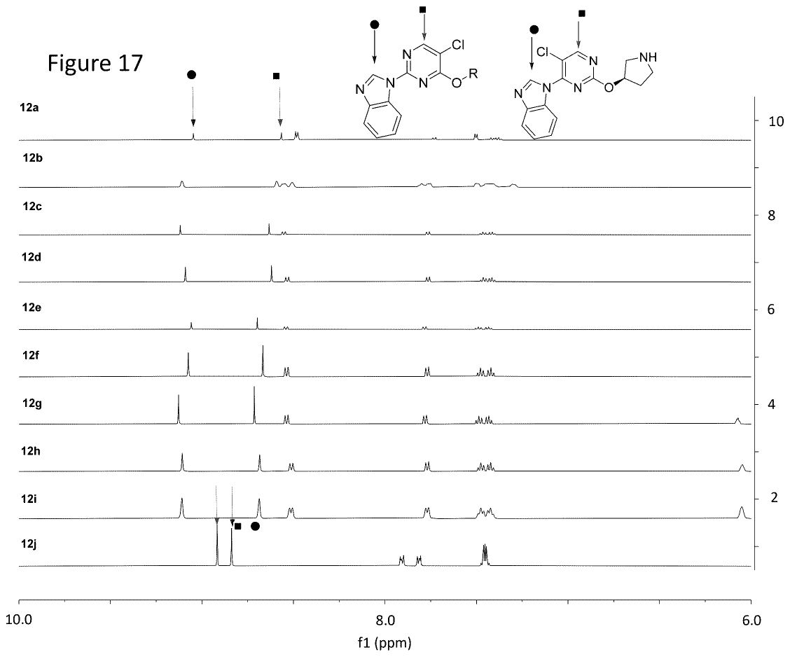

FIG. 17 shows superimposed ¹H NMR spectra of compounds in Table 2 in CD₃OD. All the protons/carbons for compounds 12h (benzimidazole in 2-position) and 12j (benzimidazole in 4-position) were fully assigned by ¹H NMR, ¹³C NMR, COSY, HMBC, and HSQC experiments (data not shown) to determine where the C6 pyrimidine hydrogen and C2 benzimidazole hydrogen appears on ¹H NMR. As can be seen in the stacked ¹H NMR spectra, protons indicated by the blue and red arrow in compounds 12a-12i show up similarly vs those same protons in compound 12j.

Note: for compound 12j, the NMR spectrum used here and for 2D experiments is obtained by silica gel chromatography and before prep HPLC to maintain consistency vs the rest of the ethers.

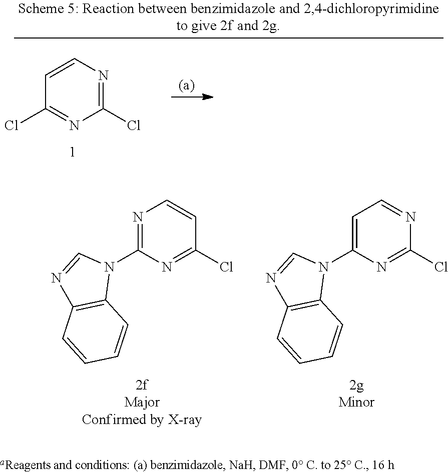 [SMILES: Clc1ccnc(Cl)n1 ; Clc1ccnc(-n2cnc3ccccc32)n1 ; Clc1nccc(-n2cnc3ccccc32)n1]

To a flask containing 2,4-dichloropyrimidine, 1 (1.00 g, 6.71 mmol, 1 equiv) immersed in an ice/water bath in anhydrous DMF (20 mL) was added 1H-benzo[d]imidazole (793 mg, 6.71 mmol, 1 equiv) after which sodium hydride (322 mg, 60.0% Wt, 8.06 mmol, 1.2 equiv) was added and the reaction was stirred as the ice bath expired under nitrogen for 16 h after which the reaction was quenched with saturated aqueous ammonium chloride solution and diluted with EtOAc. The organic layer was washed with 10% LiCl aqueous solution, brine, filtered through an isolute phase separator, and concentrated. The residue was adsorbed onto silica and purified by silica gel chromatography eluting with 0 to 100% ethyl acetate in hexanes. Fractions from the first major peak were combined and concentrated to give 1-(4-chloropyrimidin-2-yl)-1H-benzo[d]imidazole, 2f (340 mg, 22.0%). This structure was confirmed by X-ray crystallography (see above). ¹H NMR (500 MHz, CDCl₃) δ 9.01 (s, 1H), 8.60 (d, J=5.2 Hz, 1H), 8.52 (dd, J=7.9, 1.3 Hz, 1H), 7.82 (dd, J=7.9, 1.3 Hz, 1H), 7.39 (dtd, J=22.9, 7.4, 1.3 Hz, 2H), 7.21 (d, J=5.2 Hz, 1H); ¹³C NMR (126 MHz, CDCl₃) δ 162.24, 159.43, 155.85, 144.89, 141.67, 131.47, 124.96, 124.19, 120.52, 118.06, 115.60.

Fractions from the last peak were combined and concentrated to give 1-(2-chloropyrimidin-4-yl)-1H-benzo[d]imidazole, 2g (109 mg, 7.04%). ¹H NMR (500 MHz, CDCl₃) δ 8.69 (d, J=5.6 Hz, 1H), 8.64 (s, 1H), 8.26-8.20 (m, 1H), 7.87-7.81 (m, 1H), 7.48 (d, J=5.6 Hz, 1H), 7.42 (dtd, J=22.1, 7.4, 1.3 Hz, 2H); ¹³C NMR (126 MHz, CDCl₃) δ 161.58, 160.96, 157.43, 144.88, 140.18, 131.17, 125.55, 124.80, 121.12, 114.37, 107.13.

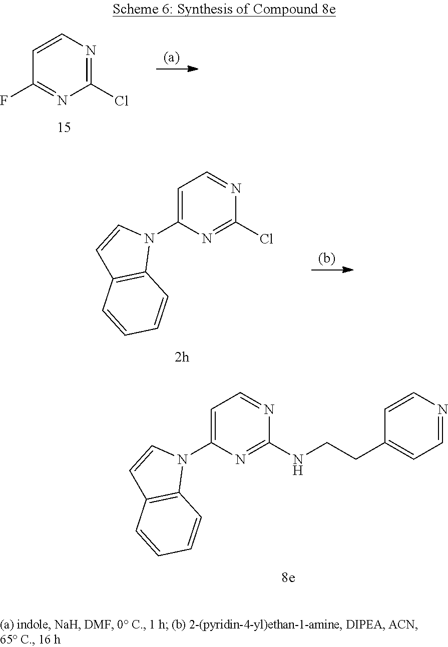 [SMILES: Fc1ccnc(Cl)n1 ; Clc1nccc(-n2ccc3ccccc32)n1 ; c1ccc2c(c1)ccn2-c1ccnc(NCCc2ccncc2)n1]

### 4-(1H-indol-1-yl)-N-(2-(pyridin-4-yl)ethyl)pyrimidin-2-amine (8e)

To 2-chloro-4-fluoropyrimidine, 15 (225.0 mg, 1.7 mmol, 1 equiv) in DMF (4 mL) in an ice/water bath was added NaH (101.9 mg, 60% Wt, 2.547 mmol, 1.5 equiv) and the mixture was stirred as the ice bath expired. After 1 h, the reaction was quenched with water and diluted with ethyl acetate. The organic layer was washed with 10% LiCl aqueous solution, brine, filtered through an isolute phase separator, concentrated and purified by silica gel chromatography eluting with 0 to 50% ethyl acetate in hexanes to give 1-(2-chloropyrimidin-4-yl)-1H-indole, 2h (174 mg, 44.6%). ¹H NMR (500 MHz, CDCl3) δ 8.57-8.48 (m, 2H), 7.68 (d, J=3.7 Hz, 1H), 7.61 (dt, J=7.8, 1.1 Hz, 1H), 7.38 (ddd, J=8.4, 7.2, 1.3 Hz, 1H), 7.32-7.24 (m, 2H), 6.78 (dd, J=3.8, 0.8 Hz, 1H).

To 1-(2-chloropyrimidin-4-yl)-1H-indole, 2h (100.0 mg, 435.4 mol, 1 equiv) in acetonitrile (2 mL) was added DIPEA (114 μL, 653.1 mol, 1.5 equiv) and 2-(pyridin-4-yl)ethan-1-amine (51.95 μL, 435.4 μmol, 1 equiv). The reaction was heated at 65° C. for 16 h after which it was allowed to cool to room temperature and diluted with ethyl acetate and water. Combined organic extracts were washed with brine, filtered through an isolute phase separator, and concentrated to a residue which was purified by silica gel chromatography eluting with 10% methanol in dichloromethane to give 4-(1H-indol-1-yl)-N-(2-(pyridin-4-yl)ethyl)pyrimidin-2-amine, 8e (104 mg, 75.7%)¹H NMR (500 MHz, CD₃OD) δ 8.61 (d, J=8.3 Hz, 1H), 8.43-8.37 (m, 2H), 8.21 (dd, J=7.7, 5.8 Hz, 1H), 7.85 (d, J=3.6 Hz, 1H), 7.63-7.59 (m, 1H), 7.38-7.34 (m, 2H), 7.23 (dtd, J=22.2, 7.2, 1.3 Hz, 2H), 6.84 (d, J=5.8 Hz, 1H), 6.73 (d, J=3.7 Hz, 1H), 3.77 (t, J=7.2 Hz, 2H), 3.04 (t, J=7.2 Hz, 2H).; ¹³C NMR (126 MHz, CD₃OD) δ 162.17, 159.34, 158.50, 150.33, 148.46, 135.26, 131.16, 124.75, 123.23, 121.86, 120.60, 115.53, 107.12, 97.66, 41.39, 34.77. Possible overlap of 1 peak on ¹³C NMR. HRMS (ESI+): m/z calcd for C₁₉H₁₈N₅: 316.1557 [M+H]⁺; found: 316.1567 [M+H]⁺.

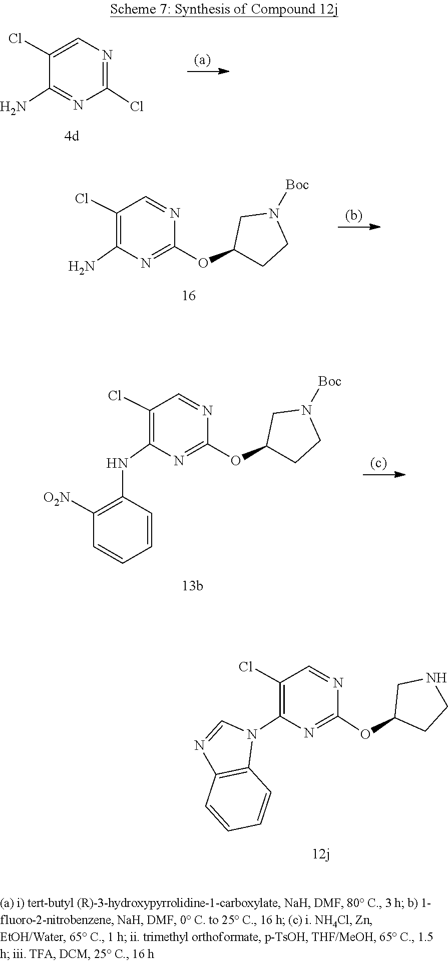 [SMILES: Nc1nc(Cl)ncc1Cl ; CC(C)(C)OC(=O)N1CC[C@@H](Oc2ncc(Cl)c(N)n2)C1 ; CC(C)(C)OC(=O)N1CC[C@@H](Oc2ncc(Cl)c(Nc3ccccc3[N+](=O)[O-])n2)C1 ; Clc1cnc(O[C@@H]2CCNC2)nc1-n1cnc2ccccc21]

### (R)-1-(5-chloro-2-(pyrrolidin-3-yloxy)pyrimidin-4-yl)-1H-benzo[d]imidazole (12j)

To tert-butyl (R)-3-hydroxypyrrolidine-1-carboxylate (1.0 g, 5.3 mmol, 1 equiv) was added DMF (10 mL) after which NaH (1.1 g, 60% Wt, 27 mmol, 5 equiv) was added slowly and portionwise. After stirring for 5 min, this material was added portion wise to a solution of 2,5-dichloropyrimidin-4-amine, 4d (0.88 g, 5.3 mmol, 1 equiv) in DMF (1 mL) at 80° C. The reaction was heated for 16 h at 80° C. after which it was allowed to cool to room temperature. The reaction was quenched by slow addition of water after which it was diluted with ethyl acetate. The organic layer was washed with 10% LiCl aqueous solution (×2), brine, filtered through an isolute phase separator, concentrated, and purified by silica gel chromatography eluting with 0 to 50% ethyl acetate in hexanes to give tert-butyl (R)-3-((4-amino-5-chloropyrimidin-2-yl)oxy)pyrrolidine-1-carboxylate, 16 (870 mg, 48%) as a white foam. ¹H NMR (500 MHz, CDCl3) δ 8.00 (d, J=4.9 Hz, 1H), 5.42-5.35 (m, 1H), 5.25 (d, J=9.1 Hz, 2H), 3.62 (d, J=3.6 Hz, 1H), 3.51 (ddd, J=17.2, 11.8, 7.1 Hz, 2H), 2.22-2.01 (m, 2H), 1.43 (s, 9H).

To a solution tert-butyl (R)-3-((4-amino-5-chloropyrimidin-2-yl)oxy)pyrrolidine-1-carboxylate, 16 (850 mg, 2.7 mmol, 1 equiv) in DMF (5 mL) in an ice/water bath was added NaH (172.8 mg, 60% Wt, 3.7 mmol, 1.6 equiv) and the reaction was stirred under nitrogen for 10 min after which 1-fluoro-2-nitrobenzene (341.7 μL, 3.24 mmol, 1.2 equiv) was added and the reaction was stirred for 3 h as the ice bath expired. The reaction was quenched with water and diluted with ethyl acetate. The organic layer was washed with 10% aqueous LiCl solution, brine, filtered through an isolute phase separator, and concentrated to an oil which was purified by silica gel chromatography eluting with 0 to 50% ethyl acetate in hexanes to give tert-butyl (R)-3-((5-chloro-4-((2-nitrophenyl)amino)pyrimidin-2-yl)oxy)pyrrolidine-1-carboxylate, 13b (650 mg, 55.2%). ¹H NMR (500 MHz, CD3OD) δ 8.62 (t, J=7.3 Hz, 1H), 8.32 (s, 1H), 8.27 (dd, J=8.5, 1.5 Hz, 1H), 7.81 (t, J=7.5 Hz, 1H), 7.35 (dt, J=14.8, 8.1 Hz, 1H), 5.43 (t, J=3.3 Hz, 1H), 3.69-3.44 (m, 4H), 2.22 (s, 2H), 1.48 (m, 9H).

To a solution of tert-butyl (R)-3-((5-chloro-4-((2-nitrophenyl)amino)pyrimidin-2-yl)oxy)pyrrolidine-1-carboxylate, 13b (609 mg, 1.40 mmol, 1 equiv) in EtOH:water (4:1, 5 mL) was added zinc (457 mg, 6.99 mmol, 5 equiv) and ammonium chloride (374 mg, 6.99 mmol, 5 equiv) and the reaction was heated at 65° C. for 1 h after which it was allowed to cool to room temperature. The solution was filtered through a pad of celite, washing with ethyl acetate and dichloromethane, and the filtrate was concentrated to a yellow oil which was purified by silica gel chromatography eluting with 0 to 20% MeOH in dichloromethane to give (R)-3-((4-((2-aminophenyl)amino)-5-chloropyrimidin-2-yl)oxy)pyrrolidine-1-carboxylate, 14b (528 mg, 93.1%). ¹H NMR (500 MHz, CD3OD) δ 8.06 (s, 1H), 7.18 (dd, J=7.8, 1.4 Hz, 1H), 7.10 (t, J=7.8 Hz, 1H), 6.89 (dd, J=8.0, 1.3 Hz, 1H), 6.78-6.74 (m, 1H), 5.13 (dq, J=4.5, 2.4 Hz, 1H), 3.50-3.37 (m, 4H), 2.14-1.96 (m, 2H), 1.46 (d, J=3.7 Hz, 9H).

To a solution tert-butyl (R)-3-((4-((2-aminophenyl)amino)-5-chloropyrimidin-2-yl)oxy)pyrrolidine-1-carboxylate, 14b (479 mg, 1.18 mmol, 1 equiv) was added THF:MeOH (3:1, 4 mL) after which trimethyl orthoformate (3.84 mL, 35.4 mmol, 30 equiv) and p-TsOH (81.3 mg, 472 mol, 0.4 equiv) were added and the reaction was heated to 65 degrees for 1 h. The reaction was allowed to cool to room temperature and it was basified with saturated aqueous bicarbonate solution. The organic layer was washed with brine, filtered through an isolute phase separator, concentrated, and purified by silica gel chromatography eluting with 0 to 50% ethyl acetate in hexanes (each solvent contained 1% triethylamine) to give tert-butyl (R)-3-((4-(1H-benzo[d]imidazol-1-yl)-5-chloropyrimidin-2-yl)oxy)pyrrolidine-1-carboxylate, 11j (393 mg, 80.1%). ¹H NMR (500 MHz, CDCl3) δ 8.68 (d, J=3.7 Hz, 1H), 8.63 (d, J=6.2 Hz, 1H), 7.88-7.83 (m, 2H), 7.39 (hept, J=6.0 Hz, 2H), 5.52 (tt, J=4.5, 2.1 Hz, 1H), 3.80-3.38 (m, 4H), 2.33-2.09 (m, 2H), 1.44 (s, 9H).

To tert-butyl (R)-3-((4-(1H-benzo[d]imidazol-1-yl)-5-chloropyrimidin-2-yl)oxy)pyrrolidine-1-carboxylate, 11j (333 mg, 801 mol, 1 equiv) was added DCM (3 mL) followed by TFA (1.23 mL, 16.0 mmol, 20 equiv) and the reaction was stirred for 16 h at room temperature after which the reaction was concentrated and triturated from ether to give solids which were isolated by filtration. These solids were further purified by silica gel chromatography eluting with 0 to 30% methanol in dichloromethane to give (R)-1-(5-chloro-2-(pyrrolidin-3-yloxy)pyrimidin-4-yl)-1H-benzo[d]imidazole trifluoracetate, 12j (150 mg, 43.7%). This material was around 95% pure. Approximately 30 mg of material was taken up in 1:1 acetonitrile:water and purified by prep HPLC to give (R)-1-(5-chloro-2-(pyrrolidin-3-yloxy)pyrimidin-4-yl)-1H-benzo[d]imidazole, 2.0 trifluoracetate (20 mg). This material was determined to be a 2.0 TFA salt by adding a known amount of internal standard 1,3,5-trimethylbenzene to an NMR sample containing a known amount of compound and performing a relative integration.

¹H NMR (500 MHz, CD₃OD) δ 8.95 (s, 1H), 8.93 (s, 1H), 7.95-7.89 (m, 1H), 7.87-7.79 (m, 1H), 7.48 (td, J=5.9, 5.1, 3.3 Hz, 2H), 5.79 (tt, J=4.4, 1.8 Hz, 1H), 3.76-3.61 (m, 2H), 3.62-3.50 (m, 2H), 2.55-2.39 (m, 2H); ¹³C NMR (126 MHz, CD₃OD) δ 162.28, 162.07, 153.57, 142.37, 141.57, 131.81, 124.88, 124.40, 119.05, 116.10, 113.50, 76.93, 50.59, 43.94, 30.47.

HRMS (ESI+): m/z calcd for C₁₅H₁₅ClN₅O: 316.0960 [M+H]⁺; found: 316.0975 [M+H]⁺. Prep HPLC conditions: Eluting with a gradient of 20 to 60% acetonitrile in water (both containing 0.1% TFA) over 7 min, then 1 min at 100% acetonitrile and a flow rate of 50 mL/min. Column: Phenomenex Kinetex C18 50×30 mm; 5 μm.

### REFERENCES (SI)

(1) Dolomanov, O. V.; Bourhis, L. J.; Gildea, R. J.; Howard, J. A. K.; Puschmann, H. OLEX2: a complete structure solution, refinement and analysis program. Journal of Applied Crystallography 2009, 42 (2), 339-341
(2) Sheldrick, G. M. SHELXT—integrated space-group and crystal-structure determination. Acta Crystallogr A Found Adv 2015, 71 (Pt 1), 3-8
(3) Sheldrick, G. M. Crystal structure refinement with SHELXL. Acta Crystallogr C Struct Chem 2015, 71 (Pt 1), 3-8
(4) Thorn, A.; Dittrich, B.; Sheldrick, G. M. Enhanced rigid-bond restraints; Acta Crystallogr A. 2012 Jul. 1; 68(Pt 4):448-51. doi: 10.1107/S0108767312014535. Epub 2012 May 1.

### Example 2

Amine-containing compounds II-5a-II-5p, II-6a-II-6p, II-7a-II-7p, II-8a-II-8p, and II-9a-II-9p and ether-containing compounds II-10a-II-10p, II-11a-II-11p, II-12a-II-12p, II-13a-II-13p, and II-14a-II-14p were prepared and evaluated for their inhibitory activity (Schemes 8 and 9).

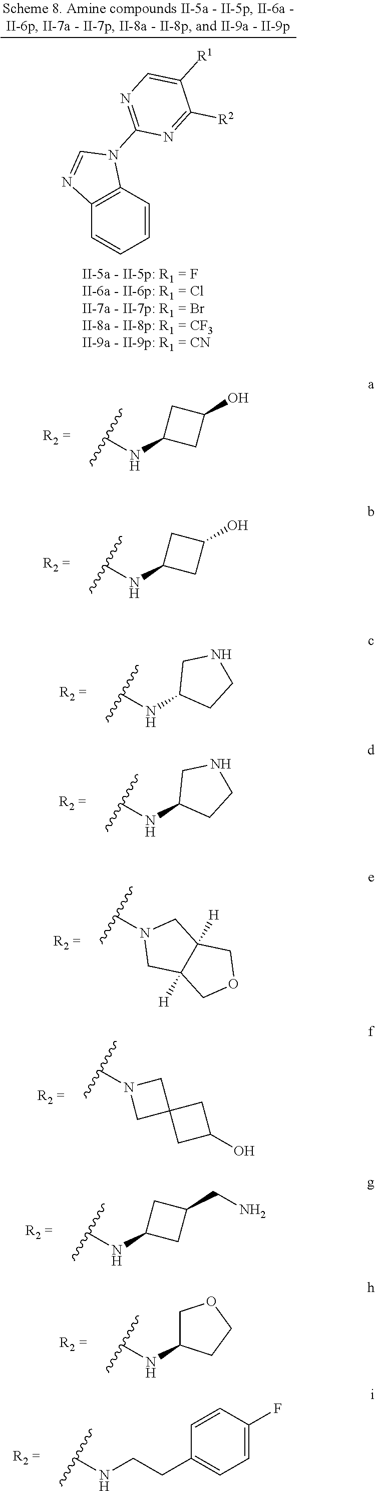

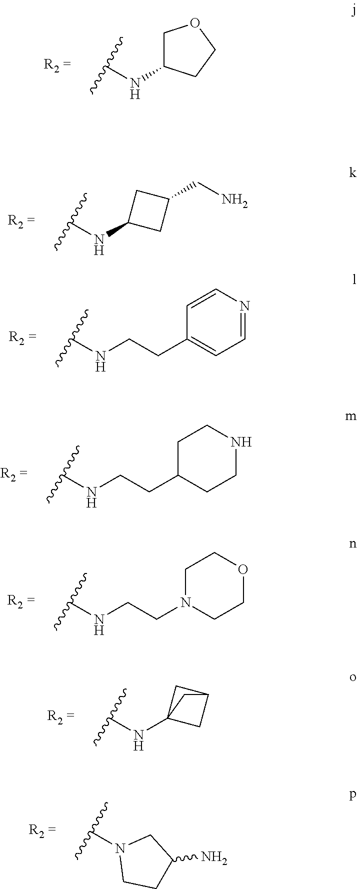

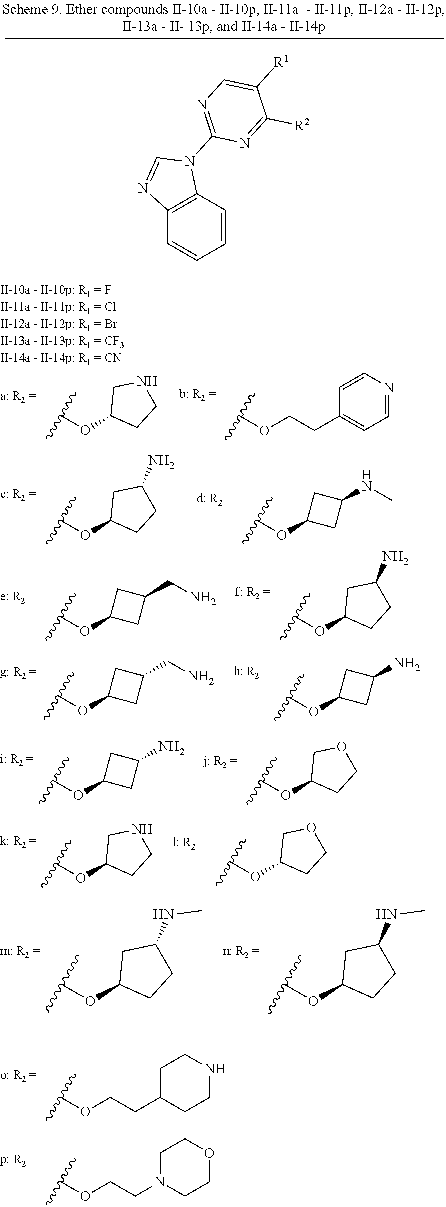

These compounds were screened for Ser209 e-IF4E phosphorylation using an in-cell Western protocol using LN229 cells.

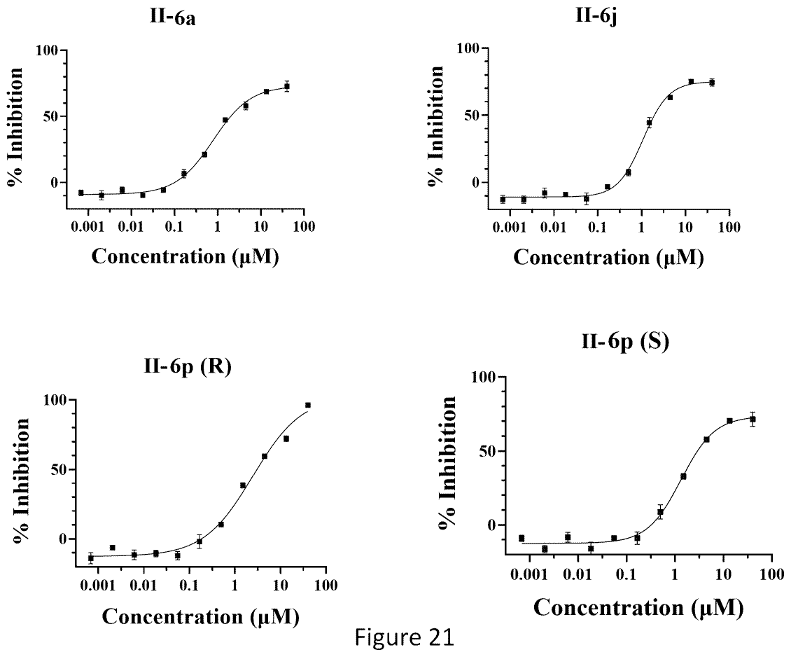

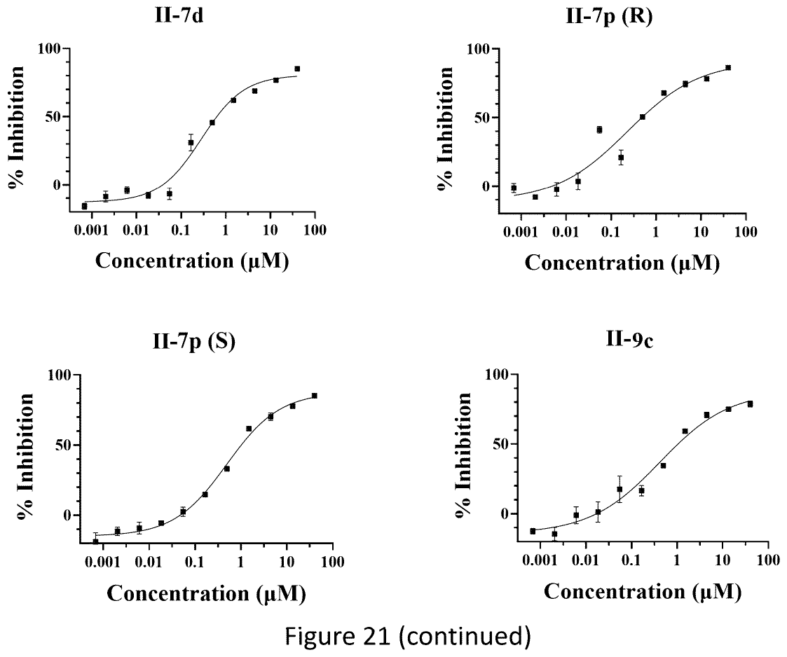

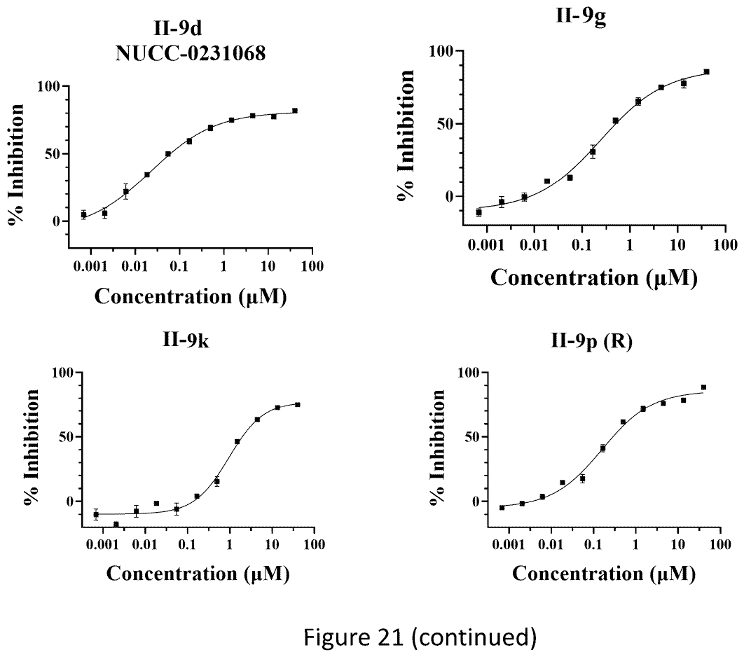

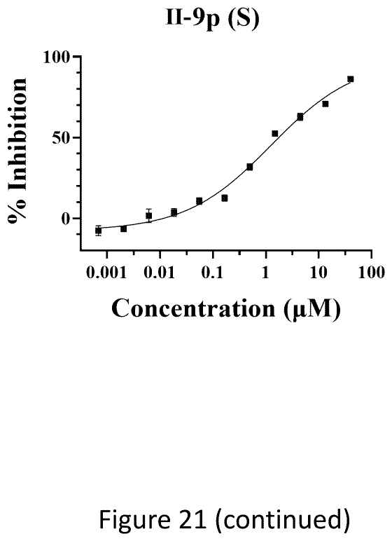

For the amine-containing compounds, the most potent ~15% of hits from the screen were re-synthesized and purified as single compounds according to general procedure D and tested in pure form to confirm their activity in LN229 cells. The results are shown in Table 15 and FIG. 21.

TABLE 15
IC₅₀ Values for the Top 15% amine inhibitors

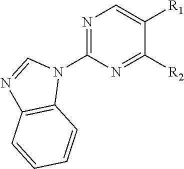

| | | | p-eIF4E IC₅₀ |
|---|---|---|---|
| Compound | R₁ | R₂ | (μM) |
| II-6a | Cl | 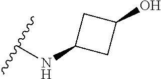 [SMILES: *N[C@H]1C[C@@H](O)C1] | 0.79 (0.62-1.0) |
| II-6j | Cl | 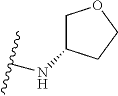 [SMILES: *N[C@H]1CCOC1] | 1.1 (0.90-1.3) |
| II-6p-(R) | Cl | 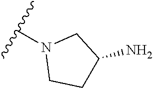 [SMILES: *N1CC[C@@H](N)C1] | 2.5 (1.6-4.8) |
| II-6p-(S) | Cl | 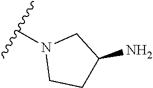 [SMILES: *N1CC[C@H](N)C1] | 1.3 (1.0 to 1.7) |
| II-7d | Br | 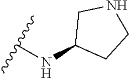 [SMILES: *N[C@@H]1CCNC1] | 0.28 (0.20-0.44) |
| II-7p-(R) | Br | 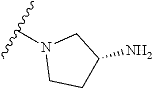 [SMILES: *N1CC[C@@H](N)C1] | 0.23 (0.089-0.87) |
| II-7p-(S) | Br | 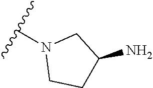 [SMILES: *N1CC[C@H](N)C1] | 0.49 (0.36 to 0.70) |
| II-9c | CN | 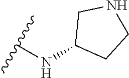 [SMILES: *N[C@H]1CCNC1] | 0.41 (0.20-1.2) |
| II-9d (NUCC- 0231068) | CN | 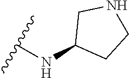 [SMILES: *N[C@@H]1CCNC1] | 0.023 (0.011-0.036) |
| II-9g | CN | 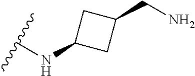 [SMILES: *N[C@H]1C[C@@H](CN)C1] | 0.24 (0.17-0.35) |
| II-9k | CN | 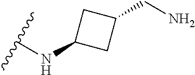 [SMILES: *N[C@H]1C[C@H](CN)C1] | 0.94 (0.71-1.3) |
| II-9p-(R) | CN | 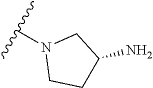 [SMILES: *N1CC[C@@H](N)C1] | 0.16 (0.12-0.21) |
| II-9p-(S) | CN | 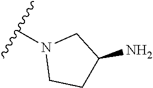 [SMILES: *N1CC[C@H](N)C1] | 1.3 (0.72-4.2) |
| eFT508 | N/A | N/A | 0.018 (0.015-0.022) |

^{a}IC₅₀ values were calculated in GraphPad Prism using a non-linear regression analysis ([inhibitor] vs. response (four parameters). Data represent mean ± SEM of three independent repeats. The 95% Cl of the IC₅₀ value is shown in parentheses.
^{b}D2B IC₅₀ is for the racemate.
eFT508 is included as a control.

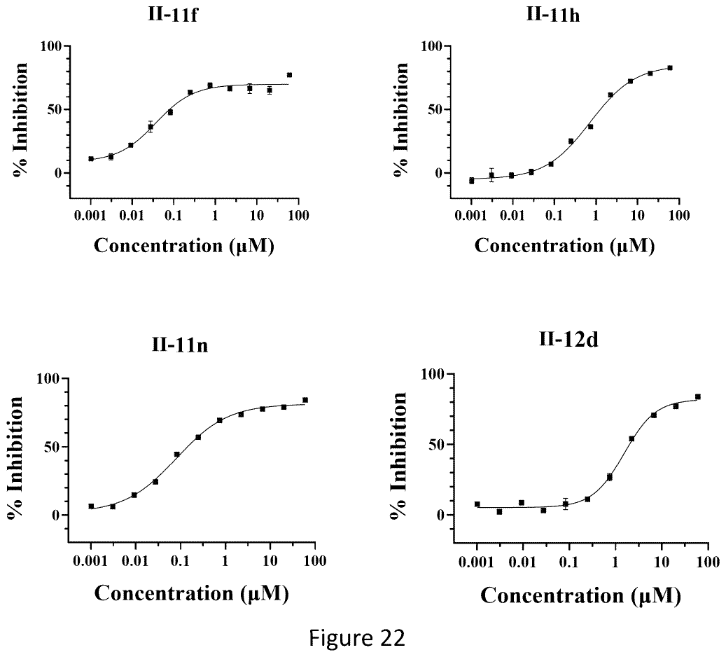

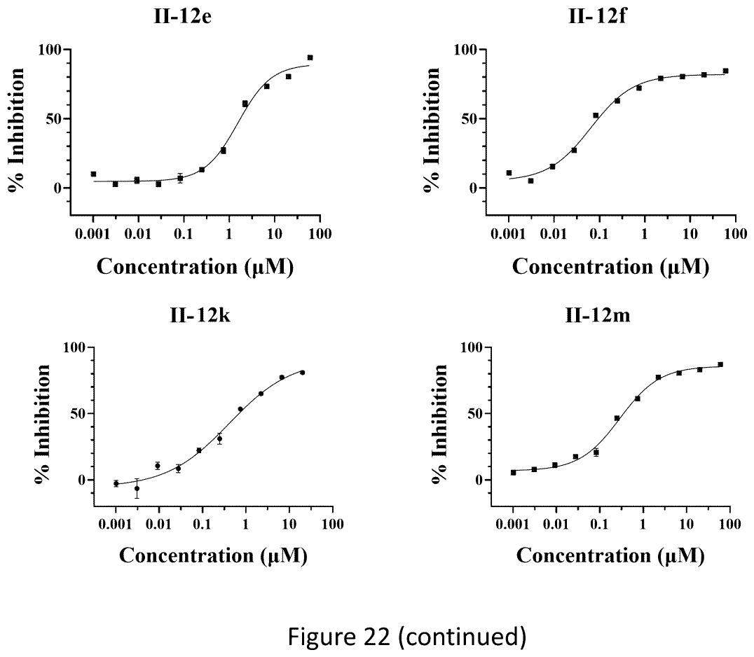

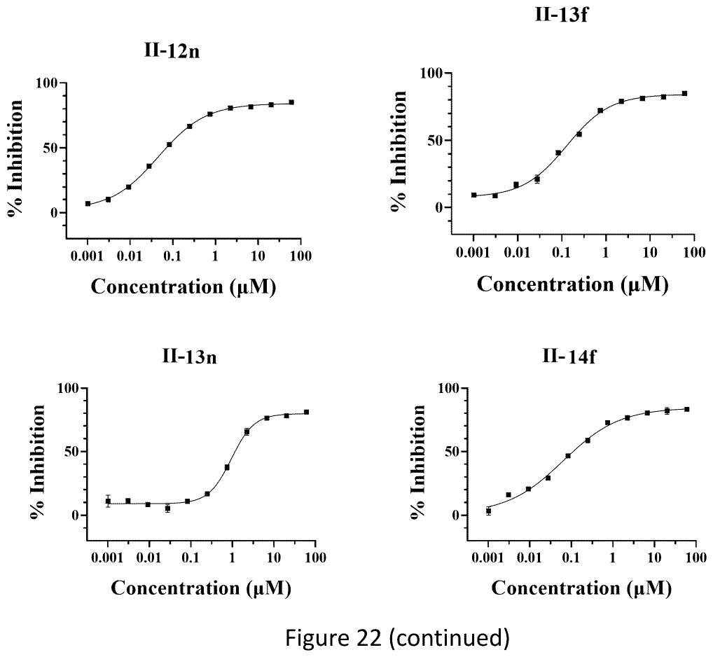

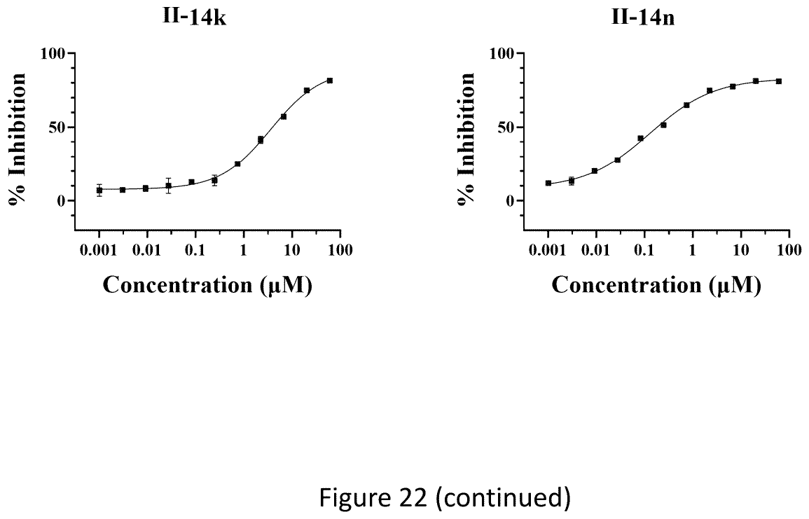

For the ether-containing compounds, the top ~15% of hits from the screen were re-synthesized using either general procedure E or F. General procedure F which employed BTPP as a base was used to synthesize compounds where the C-5 substituent was CN since Cs₂CO₃ conditions led to rapid formation of unwanted dialkylated side product (rapid displacement of both the benzimidazole at C-2 and the chlorine in C-4 with the corresponding alcohol) or conversion of the C-4 chlorine to —OH. As seen from Table 16 and FIG. 22, compound II-14k's activity was confirmed in Table 16 given its unexpected potency difference vs corresponding amine compound II-9d.

TABLE 16
IC₅₀ Values for the Top 15% ether inhibitors

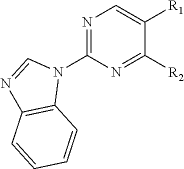

| | | | p-eIF4E IC₅₀ |
|---|---|---|---|
| Compound | R₁ | R₂ | (μM) |
| II-11f | Cl | 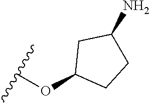 [SMILES: *O[C@@H]1CC[C@H](N)C1] | 0.037 (0.022-0.055) |
| II-11h | Cl | 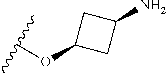 [SMILES: *O[C@H]1C[C@@H](N)C1] | 0.72 (0.58-0.91) |
| II-11n | Cl | 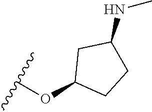 [SMILES: *O[C@@H]1CC[C@H](NC)C1] | 0.080 (0.066-0.097) |
| II-12d | Br | 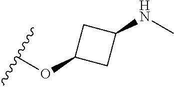 [SMILES: *O[C@H]1C[C@@H](NC)C1] | 1.5 (1.3-1.8) |
| II-12e | Br | 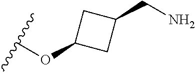 [SMILES: *O[C@H]1C[C@@H](CN)C1] | 1.6 (1.2-2.0) |
| II-12f | Br | 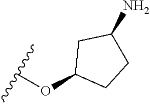 [SMILES: *O[C@@H]1CC[C@H](N)C1] | 0.065 (0.052-0.079) |
| II-12k | Br | 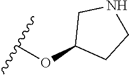 [SMILES: *O[C@@H]1CCNC1] | 0.41 (0.23-1.1) |
| II-12m | Br | 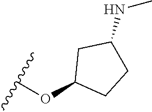 [SMILES: *O[C@@H]1CC[C@@H](NC)C1] | 0.29 (0.24 to 0.35) |
| II-12n | Br | 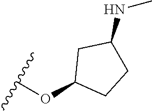 [SMILES: *O[C@@H]1CC[C@H](NC)C1] | 0.046 (0.039-0.052) |
| II-13f | CF₃ | 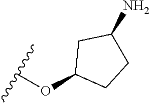 [SMILES: *O[C@@H]1CC[C@H](N)C1] | 0.13 (0.11-0.15) |
| II-13n | CF₃ | 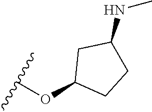 [SMILES: *O[C@@H]1CC[C@H](NC)C1] | 0.95 (0.80-1.1) |
| II-14f | CN | 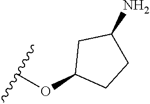 [SMILES: *O[C@@H]1CC[C@H](N)C1] | 0.064 (0.040-0.090) |
| II-14k | CN | 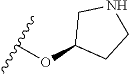 [SMILES: *O[C@@H]1CCNC1] | 3.6 (2.5-6.1) |
| II-14n | CN | 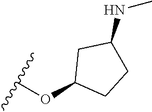 [SMILES: *O[C@@H]1CC[C@H](NC)C1] | 0.13 (0.10-0.16) |
| II-11k (NUCC- 0201049) | Cl | 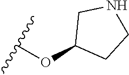 [SMILES: *O[C@@H]1CCNC1] | 1.1 (0.79-1.6) |
| eFT508 | N/A | N/A | 0.018 (0.015-0.022) |

^{a}IC₅₀ values were calculated in GraphPad Prism using a non-linear regression analysis ([inhibitor] vs. response (four parameters). Data represent mean ± SEM of three independent repeats. The 95% Cl of the IC₅₀ value is shown in parentheses.
bUtilized the 3-parameter model since prism identified at least one unstable parameter with the 4-parameter model. Compounds 11k (NUCC-0201049) and eFT508 are included as controls.

Confirmation of Ser209 p-eIF4E Reduction in LN229 Cells

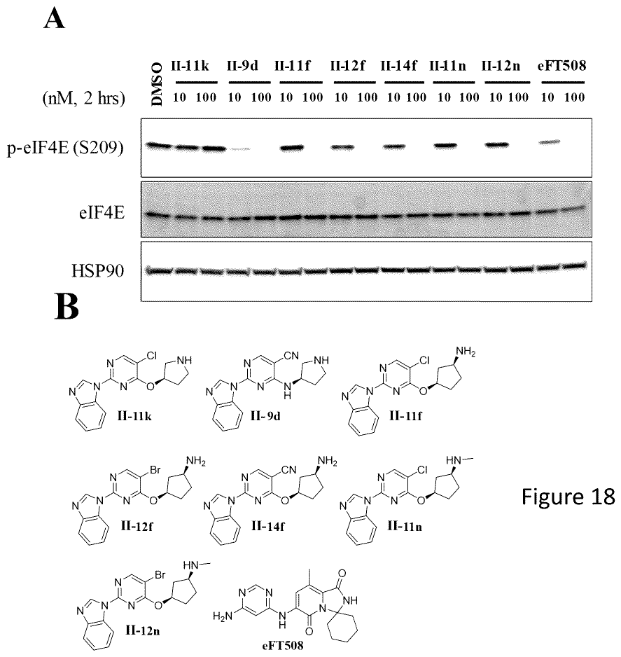

We selected the six most potent compounds and tested their ability to block phosphorylation of eIF4E at Ser209 by Western blotting. We included compound II-11k (NUCC-0201049, corresponding to compound 12 h in Example 1) and eFT508 as controls. As seen in FIG. 18, in alignment with our results from the in-cell Western (ICW) assay in LN229 cells, all compounds had a pronounced effect on p-eIF4E at 100 nM and compound II-9d's activity was comparable to eFT508 at both 10 nM and 100 nM. Notably, compound II-11k (NUCC-0201049), is a much weaker inhibitor of Ser209 eIF4E phosphorylation as compared to the rest of the compounds based on Western blotting, which is in agreement with our in-cell Western (ICW) results.

### MNK1 and MNK2 Biochemical Kinase Inhibitory Activity

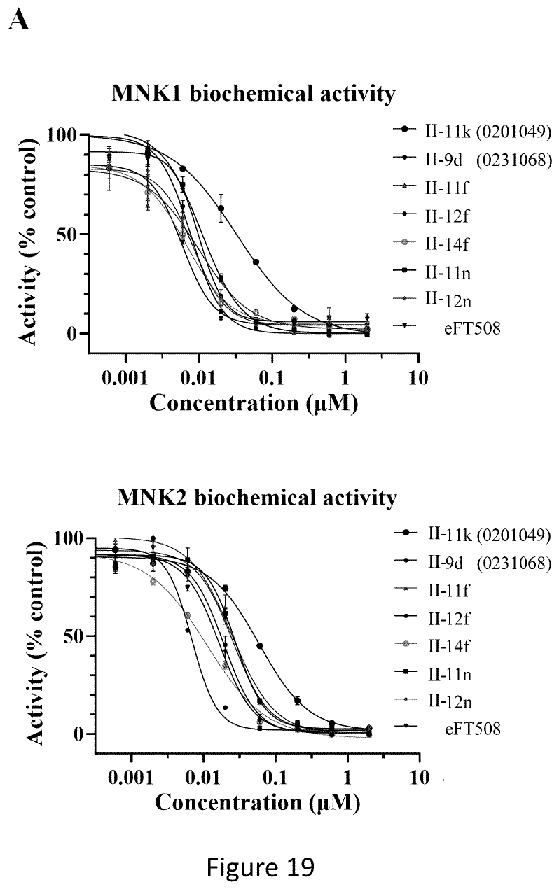

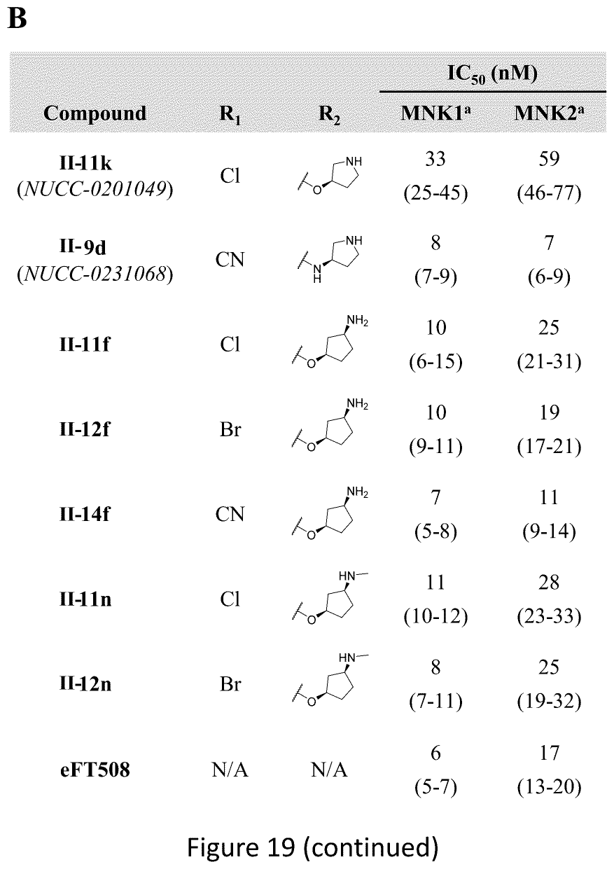

Compounds II-11k (NUCC-0201049),²⁴ II-9d, II-11f, II-12f, II-14f, IT-11n, II-12n, and eFT508 were tested to confirm their inhibition of MNK1 and MNK2 in a cell-free assay. All compounds had similar IC₅₀ values for MNK1 except for II-11k (NUCC-0201049) which was slightly weaker and was in line with its weaker cellular potency in the in-cell Western (ICW) assay (FIG. 19). Most of the compounds had slightly lower IC₅₀ values for MNK1 than for MNK2. Notably, most of these compounds had biochemical potencies comparable to that of the clinically-tested MNK inhibitor eFT508.

### Molecular Modeling of NUCC-0231068 (II-9d) in MNK2

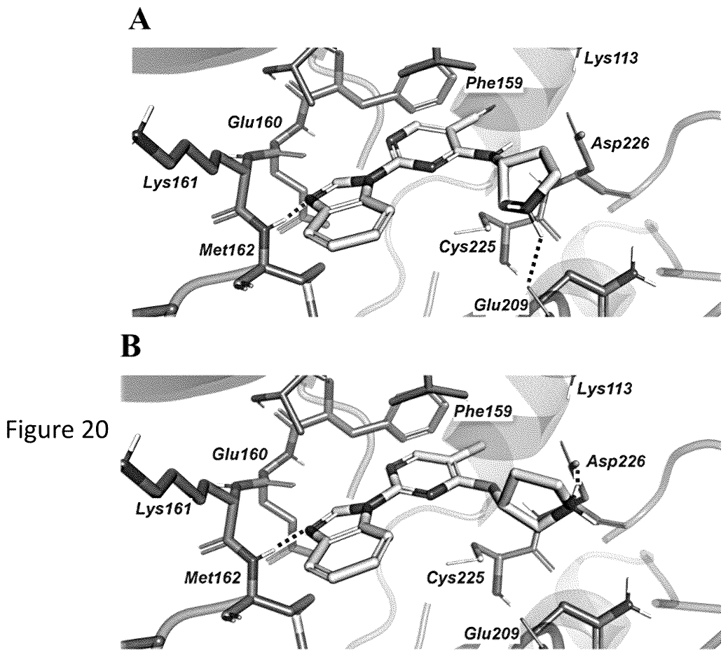

To rationalize the potency of compound II-9d (NUCC-0231068), we docked it into MNK2 D228G (pdb 9HRC) and compared the binding pose and interactions with those of bound ligand II-11k (NUCC-0201049). As shown in FIG. 20, both compounds maintain the same hydrogen bond with the kinase hinge residue Met162 N—H via their benzimidazole nitrogen. The pyrrolidine ring in II-9d appears to have a new hydrogen bonding interaction with residue Glu209. There may also be electrostatic complementarity between the nitrile group of NUCC-0231068 and Lys113 which could contribute to its increased potency. The molecular modeling indicates that the hydrogen bond present with NUCC-0201049 between the pyrrolidine and Asp226 is absent in NUCC-0231068; however, the new amine linker in NUCC-0231068 is 3.6 Å away from Asp226, which could help offset this loss.

### Discussion

Our systematic exploration of C-5 substituents on the pyrimidine core revealed that bromo-chloro-, and cyano- groups produce more potent inhibitors, while fluoro- and trifluoromethyl- are generally less effective substituents. The cyano- group, in particular, emerged as the optimal substituent among this set, with multiple compounds containing this moiety achieving sub-100 nM potencies, including compound II-9d (NUCC-0231068) which had a cellular Ser209 eIF4E IC₅₀=23 nM. The SAR of analogs varying at the pyrimidine C-4 position revealed a preference for small heterocyclic rings with short linkers, consistent with our molecular modeling that showed these features optimize engagement with Asp226. Aromatic substituents were generally not well tolerated, as exemplified by 2-(4-fluorophenyl)ethan-1-amine derivatives showing IC₅₀ values exceeding 50 M across all C-5 variants. Notably, ether-linked compounds generally showed comparable or slightly reduced potency relative to their amine counterparts. The dramatic 100-fold potency improvement observed for amine compound II-9d (containing a cyano- group at C-5) compared to ether compound II-14k highlights how subtle structural changes can profoundly impact cellular activity. This observation warrants further investigation into the molecular basis for this enhanced potency through co-crystallization studies.

The identification of highly potent MNK inhibitors with cellular IC₅₀ values comparable to or better than eFT508 represents a promising step toward developing new therapeutic options for glioblastoma. GBM remains one of the most lethal cancers, with median survival of only 15-18 months despite aggressive therapy. The MNK-eIF4E pathway plays a central role in GBM by selectively enhancing translation of key oncogenic drivers. By potently inhibiting Ser209 eIF4E phosphorylation in LN229 GBM cells, our MNK inhibitors may suppress translation of these oncoproteins and effect tumor growth.

In this work, we demonstrate the development of potent MNK inhibitors targeting eIF4E phosphorylation in glioblastoma cells. Our work yielded several novel MNK inhibitors with cellular potencies below 100 nM, including compound II-9d (NUCC-0231068) with an IC₅₀ for Ser209 eIF4E phosphorylation of 23 nM, which is similar to the clinically-tested compound eFT508 (tomivosertib).

### Experimental

General Chemical Methods. All chemical reagents were obtained from commercial suppliers and used without further purification unless otherwise stated. Anhydrous solvents were purchased from Sigma-Aldrich and dried over 3 Å molecular sieves when necessary. Normal-phase flash column chromatography was performed using Biotage KP-Sil 50 μm silica gel columns and ACS grade solvents on a Biotage Isolera flash purification system. Reverse phase prep conditions are detailed for each particular compound if relevant. Analytical thin layer chromatography (TLC) was performed on EM Reagent 0.25 mm silica gel 60 F254 plates and visualized by UV light. Proton (¹H), and carbon (¹³C) NMR spectra were recorded on a 500 MHz Bruker Avance III with direct cryoprobe spectrometer. Chemical shifts were reported in ppm (δ) and were referenced using residual nondeuterated solvent as an internal standard (CDCl₃ at 7.24 ppm for ¹H-NMR and 77.0 for ¹³C-NMR; CD₃OD at 3.33 ppm for ¹H-NMR and 47.6 for ¹³C-NMR; DMSO-d₆ at 2.52 ppm for ¹H-NMR and 39.9 ppm for ¹³C-NMR). Proton coupling constants are expressed in hertz (Hz). The following abbreviations were used to denote spin multiplicity for proton NMR: s=singlet, d=doublet, t=triplet, q=quartet, m=multiplet, brs=broad singlet, dd=doublet of doublets, dt=doublet of triplets, quin=quintet, tt=triplet of triplets. Low resolution liquid chromatography/mass spectrometry (LCMS) was performed on a Waters Acquity-H UPLC/MS system with a 2.1 mm×50 mm, 1.7 μm, reversed phase BEH C18 column and LCMS grade solvents. A gradient elution from 95% water+0.1% TFA/5% acetonitrile+0.1% TFA to 95% acetonitrile+0.1% TFA/5% water+0.1% TFA over 2 min plus a further minute continuing this mixture at a flow rate of 0.85 mL/min was used as the eluent. Total ion current traces were obtained for electrospray positive and negative ionization (ESI+/ESI−). All compounds are >95% pure by HPLC analysis. High-resolution mass spectra were obtained using an Agilent 6210 LC-TOF spectrometer in the positive ion mode using electrospray ionization with an Agilent G1312A HPLC pump and an Agilent G1368B autoinjector at the Integrated Molecular Structure Education and Research Center (IMSERC), Northwestern University.

### Synthetic Methods

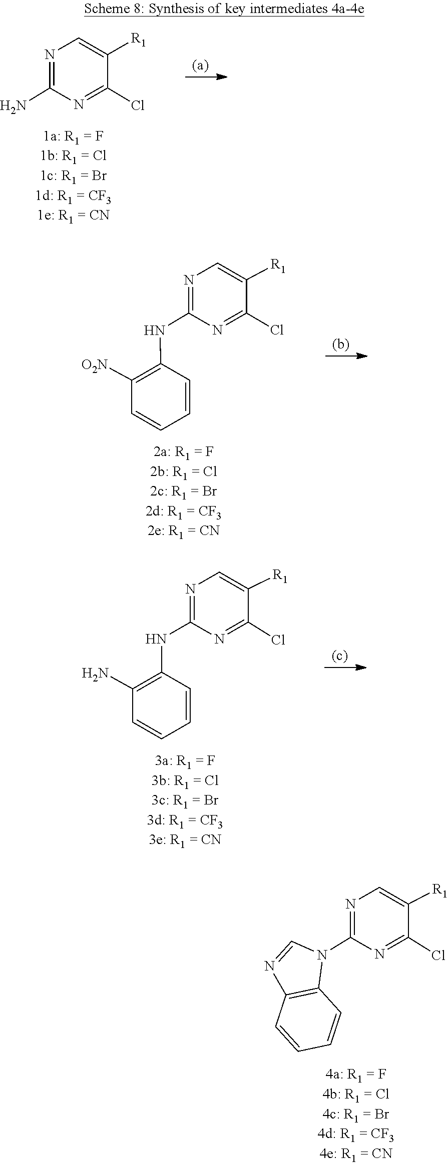

### General Procedure A: SNAr (Scheme 8)

The appropriate 5-substituted 2-amino-4-chloropyrimidine was dissolved in anhydrous DMF (0.2 M), subjected to an atmosphere of nitrogen, then cooled in an ice/water bath after which 1-fluoro-2-nitrobenzene (1.2 equiv.) was added followed by sodium hydride (60% Wt, 2 equiv.). The reaction was stirred in the ice bath for 5 minutes after which it was transferred to a heating block at 60° C. for 25 min-1.5 h depending on the substituent in the 5-position of the pyrimidine ring. Upon completion of the reaction by LCMS, the reaction mixture was allowed to cool to room temperature then transferred again to an ice/water bath. The reaction was quenched slowly by adding water while stirring vigorously until solids precipitated from solution. The mixture was filtered under vacuum to isolate the desired 5-substituted 4-chloro-N-(2-nitrophenyl)pyrimidin-2-amine which was washed generously with water.

### General Procedure B: Nitro Reduction (Scheme 8)

To a solution of the appropriate 5-substituted 4-chloro-N-(2-nitrophenyl)pyrimidin-2-amine in isopropanol:water (3:1, 0.2 M) was added iron powder (10 equiv.) and ammonium chloride (10 equiv.) and the reaction was heated at the indicated temperature for 1 h-1.5 h depending on the substituent in the 5-position of the pyrimidine ring. After the reaction was complete by LCMS, it was allowed to cool to room temperature then filtered through a pad of celite, washing with dichloromethane and methanol. The filtrate was concentrated to give solids which were stirred vigorously in water to dissolve inorganics. This mixture was filtered, the filtrate was discarded, and the collected solids were triturated with petroleum ether to isolate the reduced intermediates 3a-3e.

### General Procedure C: Cyclization (Scheme 8)

To a solution of intermediates 3a-3e in THF:water (4:1, 0.6 M) was added trimethyl orthoformate (20 equiv.) and p-toluenesulfonic acid (0.2 equiv.) and the reaction was heated at 60° C. for 15 min-1 h depending on the substituent in the 5-position of the pyrimidine ring. Upon completion of the reaction, the product had precipitated out of solution. The reaction was allowed to cool to room temperature, then it was filtered to isolate the solids which were washed with methanol and dried under vacuum to give key intermediates 4a-4e.

### 4-chloro-5-fluoro-N-(2-nitrophenyl)pyrimidin-2-amine (2a)

4-chloro-5-fluoropyrimidin-2-amine 1a (1.0 g, 6.8 mmol), 1-fluoro-2-nitrobenzene (1.1 g, 0.86 mL, 8.1 mmol, 1.2 equiv.), and NaH (0.54 g, 60% Wt, 14 mmol, 2 equiv.) were reacted according to general procedure A for 5 min in the ice/water bath followed by 20 minutes at 60° C. to yield 2a (1.36 g, 5.1 mmol, 75%). ¹H NMR (500 MHz, CDCl₃) δ 10.54 (s, 1H), 8.81 (dd, J=8.7, 1.3 Hz, 1H), 8.35 (s, 1H), 8.24 (dd, J=8.5, 1.7 Hz, 1H), 7.65 (ddd, J=8.8, 7.2, 1.7 Hz, 1H), 7.14-7.04 (m, 1H).

### N1-(4-chloro-5-fluoropyrimidin-2-yl)benzene-1,2-diamine (3a)

4-chloro-5-fluoro-N-(2-nitrophenyl)pyrimidin-2-amine 2a (1.36 g, 5.1 mmol), iron (2.83 g, 50.6 mmol, 10 equiv.) and ammonium chloride (2.71 g, 50.6 mmol, 10 equiv.) were reacted according to general procedure B at 50° C. for 1.5 h to yield 3a (872 mg, 3.65 mmol, 72%). ¹H NMR (500 MHz, CD₃OD) δ 8.26 (s, 1H), 7.25 (d, J=7.9 Hz, 1H), 7.03 (t, J=7.6 Hz, 1H), 6.87 (d, J=7.9 Hz, 1H), 6.74 (t, J=7.6 Hz, 1H).

### 1-(4-chloro-5-fluoropyrimidin-2-yl)-1H-benzo[d]imidazole (4a)

N1-(4-chloro-5-fluoropyrimidin-2-yl)benzene-1,2-diamine 3a (872 mg, 3.65 mmol), trimethyl orthoformate (7.76 g, 7.99 mL, 73.1 mmol, 20 equiv.) and p-TsOH (139 mg, 0.731 mmol, 0.2 equiv.) were reacted according to general procedure C at 60° C. for 1 h. For this intermediate, solids did not form spontaneously upon completion of the reaction therefore a work-up was performed by diluting the reaction mixture with dichloromethane and saturated aqueous sodium bicarbonate solution. Organics were washed with brine, filtered through an isolute phase separator, and concentrated to give solids which were further triturated from hexanes to give 4a (682 mg, 2.74 mmol, 75.1%). ¹H NMR (500 MHz, CDCl₃) δ 8.94 (s, 1H), 8.59 (s, 1H), 8.47 (dd, J=8.2, 1.2 Hz, 1H), 7.83 (dd, J=8.0, 1.2 Hz, 1H), 7.46-7.35 (m, 2H).

### 4,5-dichloro-N-(2-nitrophenyl)pyrimidin-2-amine (2b)

4,5-dichloropyrimidin-2-amine 1b (1.0 g, 6.1 mmol), 1-fluoro-2-nitrobenzene (1.0 g, 0.77 mL, 7.3 mmol, 1.2 equiv.), and NaH (0.49 g, 60% Wt, 12 mmol, 2 equiv.) were reacted according to general procedure A for 5 min in the ice/water bath followed by 20 minutes at 60° C. to yield 2b (1.3 g, 4.6 mmol, 75%). ¹H NMR (500 MHz, CDCl₃) δ 10.52 (s, 1H), 8.80 (dd, J=8.7, 1.3 Hz, 1H), 8.44 (s, 1H), 8.24 (dd, J=8.4, 1.6 Hz, 1H), 7.65 (ddd, J=8.7, 7.2, 1.6 Hz, 1H), 7.12 (ddd, J=8.5, 7.4, 1.3 Hz, 1H).

### N1-(4,5-dichloropyrimidin-2-yl)benzene-1,2-diamine (3b)

4,5-dichloro-N-(2-nitrophenyl)pyrimidin-2-amine 2b (1.3 g, 4.6 mmol), iron (2.5 g, 46 mmol, 10 equiv.) and ammonium chloride (2.4 g, 46 mmol, 10 equiv.) were reacted according to general procedure B at 80° C. for 1 h to yield 3b (1 g, 4 mmol, 90%). ¹H NMR (500 MHz, CDCl₃) δ 8.28 (s, 1H), 7.36 (d, J=7.8 Hz, 1H), 7.07 (t, J=7.6 Hz, 1H), 6.82 (d, J=7.8 Hz, 2H), 6.72 (s, 1H).

### 1-(4,5-dichloropyrimidin-2-yl)-1H-benzo[d]imidazole (4b)

N1-(4,5-dichloropyrimidin-2-yl)benzene-1,2-diamine 3b (1.0 g, 3.9 mmol), trimethyl orthoformate (8.3 g, 8.6 mL, 78 mmol, 20 equiv.), and p-TsOH (0.15 g, 0.78 mmol, 0.2 equiv.) were reacted according to general procedure C at 60° C. for 1 h to give 4b (757 mg, 2.86 mmol, 73%). ¹H NMR (500 MHz, CDCl₃) δ 8.96 (s, 1H), 8.69 (d, J=0.7 Hz, 1H), 8.47 (dt, J=8.0, 1.0 Hz, 1H), 7.83 (dd, J=7.7, 1.3 Hz, 1H), 7.48-7.33 (m, 2H).

### 5-bromo-4-chloro-N-(2-nitrophenyl)pyrimidin-2-amine (2c)

5-bromo-4-chloropyrimidin-2-amine 1c (1.0 g, 4.8 mmol), 1-fluoro-2-nitrobenzene (0.81 g, 0.61 mL, 5.8 mmol, 1.2 equiv.), and NaH (0.38 g, 60% Wt, 9.6 mmol, 2 equiv.) were reacted according to general procedure A for 5 min in the ice/water bath followed by 20 minutes at 60° C. to give 2c (1.4 g, 4.2 mmol, 89%). ¹H NMR (500 MHz, CDCl₃) δ 10.51 (s, 1H), 8.79 (d, J=8.6 Hz, 1H), 8.53 (s, 1H), 8.24 (dt, J=8.5, 1.2 Hz, 1H), 7.65 (t, J=7.9 Hz, 1H), 7.16-7.08 (m, 1H)

### N1-(5-bromo-4-chloropyrimidin-2-yl)benzene-1,2-diamine (3c)

5-bromo-4-chloro-N-(2-nitrophenyl)pyrimidin-2-amine 2c (1.40 g, 4.25 mmol), iron (2.37 g, 42.5 mmol, 10 equiv.), and ammonium chloride (2.27 g, 42.5 mmol, 10 equiv.) were reacted according to general procedure B at 80° C. for 1 h to yield 3c (917 mg, 3.06 mmol, 72.1%). ¹H NMR (500 MHz, CD₃OD) δ 8.38 (s, 1H), 7.24 (d, J=7.8 Hz, 1H), 7.04 (t, J=7.6 Hz, 1H), 6.87 (d, J=7.9 Hz, 1H), 6.74 (t, J=7.6 Hz, 1H).

### 1-(5-bromo-4-chloropyrimidin-2-yl)-1H-benzo[d]imidazole (4c)

N1-(5-bromo-4-chloropyrimidin-2-yl)benzene-1,2-diamine 3c (917 mg, 3.06 mmol), trimethyl orthoformate (6.50 g, 6.70 mL, 61.2 mmol, 20 equiv.) and p-TsOH (116 mg, 0.61 mmol, 0.2 equiv.) were reacted according to general procedure C at 60° C. for 1 h to give 4c (650 mg, 2.10 mmol, 68.6%). ¹H NMR (500 MHz, CDCl₃) δ 8.97 (s, 1H), 8.79 (s, 1H), 8.47 (d, J=8.0 Hz, 1H), 7.83 (d, J=7.8 Hz, 1H), 7.46-7.35 (m, 2H).

### 4-chloro-N-(2-nitrophenyl)-5-(trifluoromethyl)pyrimidin-2-amine (2d)

4-chloro-5-(trifluoromethyl)pyrimidin-2-amine 1d (1 g, 5.06 mmol), 1-fluoro-2-nitrobenzene (857 mg, 641 μL, 6.07 mmol, 1.2 equiv.) and NaH (405 mg, 60% Wt, 10.12 mmol, 2 equiv.) were reacted according to general procedure A for 5 min in the ice/water bath followed by 60° C. for 1.5 h to yield 2d (1.29 g, 4.05 mmol, 80.0%). ¹H NMR (500 MHz, CDCl₃) δ 10.62 (s, 1H), 8.81 (dd, J=8.6, 1.3 Hz, 1H), 8.67 (s, 1H), 8.26 (dd, J=8.4, 1.6 Hz, 1H), 7.69 (ddd, J=8.7, 7.2, 1.6 Hz, 1H), 7.20 (td, J=7.9, 7.2, 1.3 Hz, 1H).

### N1-(4-chloro-5-(trifluoromethyl)pyrimidin-2-yl)benzene-1,2-diamine (3d)

4-chloro-N-(2-nitrophenyl)-5-(trifluoromethyl)pyrimidin-2-amine 2d (1.3 g, 4.1 mmol), iron (2.3 g, 41 mmol, 10 equiv.) and ammonium chloride (2.2 g, 41 mmol, 10 equiv.) were reacted according to general procedure B at 50° C. for 1 h to afford 3d (700 mg, 2.42 mmol, 59%). ¹H NMR (500 MHz, CD₃OD) δ 6.99 (s, 1H), 5.73-5.67 (m, 1H), 5.56 (td, J=7.7, 1.5 Hz, 1H), 5.36 (dd, J=8.1, 1.4 Hz, 1H), 5.22 (td, J=7.6, 1.4 Hz, 1H).

### 1-(4-chloro-5-(trifluoromethyl)pyrimidin-2-yl)-1H-benzo[d]imidazole (4d)

N1-(4-chloro-5-(trifluoromethyl)pyrimidin-2-yl)benzene-1,2-diamine (730 mg, 2.53 mmol), trimethyl orthoformate (5.4 g, 5.5 mL, 50.6 mmol, 20 equiv.) and p-TsOH (96 mg, 0.51 mmol, 0.2 equiv.) were reacted according to general procedure C at 60° C. for 1 h. For this intermediate, solids did not form spontaneously upon completion of the reaction therefore a work-up was performed by diluting the reaction mixture with dichloromethane and saturated aqueous sodium bicarbonate solution. Organics were washed with brine, filtered through an isolute phase separator, and concentrated to give solids which were further triturated from hexanes to give 4d (534 mg, 1.79 mmol, 70.7%). ¹H NMR (500 MHz, CDCl₃) δ 9.03 (s, 1H), 8.94 (s, 1H), 8.51 (d, J=8.0 Hz, 1H), 7.84 (d, J=7.7 Hz, 1H), 7.49-7.38 (m, 2H).

### 4-chloro-2-((2-nitrophenyl)amino)pyrimidine-5-carbonitrile (2e)

2-amino-4-chloropyrimidine-5-carbonitrile 1e (1.0 g, 6.5 mmol), 1-fluoro-2-nitrobenzene (1.1 g, 0.82 mL 7.8 mmol, 1.2 equiv.) and NaH (0.52 g, 60% Wt, 13 mmol, 2 equiv.) were reacted according to general procedure A for 5 min in the ice water bath followed by 60° C. for 1 h to yield 2e (1.25 g, 4.53 mmol, 70%). ¹H NMR (500 MHz, DMSO-d₆) δ 11.07 (s, 1H), 8.88 (s, 1H), 8.05 (dd, J=8.2, 1.5 Hz, 1H), 7.77 (td, J=7.7, 1.5 Hz, 1H), 7.64 (dd, J=8.1, 1.3 Hz, 1H), 7.51-7.45 (m, 1H).

### 2-((2-aminophenyl)amino)-4-chloropyrimidine-5-carbonitrile (3e)

4-chloro-2-((2-nitrophenyl)amino)pyrimidine-5-carbonitrile 2e (1.25 g, 4.53 mmol), iron (2.53 g, 45.3 mmol, 10 equiv.) and ammonium chloride (2.43 g, 45.3 mmol, 10 equiv.) were reacted according to general procedure B at 50° C. for 1 h to yield 3e (850 mg, 3.46 mmol, 76.3%). ¹H NMR (500 MHz, CD₃OD) δ 8.57 (br s, 1H), 7.19 (d, J=7.9 Hz, 1H), 7.08 (t, J=7.7 Hz, 1H), 6.87 (d, J=8.0 Hz, 1H), 6.73 (t, J=7.6 Hz, 1H).

### 2-(1H-benzo[d]imidazol-1-yl)-4-chloropyrimidine-5-carbonitrile (4e)

2-((2-aminophenyl)amino)-4-chloropyrimidine-5-carbonitrile 3e (850 mg, 3.46 mmol), trimethyl orthoformate (7.34 g, 7.57 mL, 69.2 mmol, 20 equiv.) and p-TsOH (132 mg, 0.69 mmol, 0.2 equiv.) were reacted according to general procedure C at 60° C. for 15 min to yield 4e (510 mg, 1.99 mmol, 57.7%). ¹H NMR (500 MHz, CDCl₃) δ 9.00 (s, 1H), 8.91 (s, 1H), 8.46 (dd, J=8.1, 1.3 Hz, 1H), 7.88-7.82 (m, 1H), 7.44 (tt, J=8.7, 6.7 Hz, 2H).

### General Procedure D: Synthesis of Pure Amines

To a vial containing the appropriate intermediate in acetonitrile (0.2 M) was added the relevant amine (2 equiv.) and DIPEA (2 equiv.) after which the reaction was stirred at 50° C. for 16 h after which it was concentrated. If a Boc group was not present, the residue was concentrated then dissolved in 1:1 acetonitrile:water (DMSO added as needed) and purified by reverse phase preparative HPLC with a Phenomenex Gemini-NX C18 column (110 Å, 150×21.2 mm; 5 μm), a gradient of 5% to 80% acetonitrile in water (both solvents contained 0.1% TFA), collection wavelength of 254 nm, and flow rate of 20 mL/min. Relevant fractions were frozen in a dry ice/acetone bath and lyophilized. Unless otherwise noted, compounds purified in this fashion were determined to be 2.0 TFA salts by adding a known amount of internal standard 1,3,5-trimethylbenzene and performing a relative integration.

If a Boc group was present, after the SNAr reaction with the appropriate amine and DIPEA, the reaction was concentrated to a residue then taken up in DCM (0.2 M) and treated with TFA (40 equiv.). After stirring at room temperature for 16 h, the residue was taken up in 1:1 acetonitrile:water (DMSO added as needed) and purified by prep HPLC using the same conditions as those described above. Compounds purified in this fashion were also determined to be 2.0 TFA salts by adding a known amount of internal standard 1,3,5-trimethylbenzene and performing a relative integration.

### (1s,3s)-3-((2-(1H-benzo[d]imidazol-1-yl)-5-chloropyrimidin-4-yl)amino)cyclobutan-1-ol (II-6a)

1-(4,5-dichloropyrimidin-2-yl)-1H-benzo[d]imidazole 4b (50.0 mg, 89 mol), (1s,3s)-3-aminocyclobutan-1-ol, HCl (46.6 mg, 2 equiv., 377 mol) and DIPEA (48.8 mg, 65.7 μL, 2 equiv., 377 mol) were reacted according to general procedure D. The compound was neither treated with TFA nor purified by prep HPLC since it precipitated out of solution upon completion of the reaction. Solids were washed with water and dried to yield II-6a (36 mg, 0.11 mmol, 60%). ¹H NMR (500 MHz, DMSO-d₆) δ 9.04 (s, 1H), 8.47 (d, J=8.1 Hz, 1H), 8.33 (s, 1H), 7.95 (d, J=6.7 Hz, 1H), 7.77 (d, J=7.9 Hz, 1H), 7.42 (t, J=7.7 Hz, 1H), 7.35 (t, J=7.6 Hz, 1H), 5.17 (s, 1H), 4.21-4.10 (m, 1H), 4.01 (p, J=7.4 Hz, 1H), 2.72 (dddd, J=12.1, 9.7, 6.4, 2.9 Hz, 2H), 2.11 (qd, J=8.5, 3.2 Hz, 2H). ¹³C NMR (126 MHz, DMSO-d₆) δ 157.84, 153.69, 153.51, 144.85, 142.62, 131.90, 124.61, 123.65, 120.28, 115.58, 110.38, 59.96, 40.74, 38.50. HRMS (ESI+): m/z calcd for C₁₅H₁₅ClN₅O: 316.0960 [M+H]⁺; found: 316.0960 [M+H]⁺.

### (1r,3r)-3-((2-(1H-benzo[d]imidazol-1-yl)-5-chloropyrimidin-4-yl)amino)cyclobutan-1-ol (II-6b)

1-(4,5-dichloropyrimidin-2-yl)-1H-benzo[d]imidazole 4b (50.0 mg, 189 mol), (1r,3r)-3-aminocyclobutan-1-ol, HCl (35.0 mg, 1.5 equiv., 283 mol) and DIPEA (48.8 mg, 65.7 μL, 2 equiv., 377 mol) were reacted according to general procedure D to give the SNAr product which was purified by prep HPLC to yield II-6b as a 2.0 TFA salt (25 mg, 46 mol, 24%). ¹H NMR (500 MHz, CD₃OD) δ 9.61 (s, 1H), 8.70 (d, J=8.3 Hz, 1H), 8.29 (s, 1H), 7.82 (d, J=8.0 Hz, 1H), 7.61 (t, J=7.7 Hz, 1H), 7.55 (t, J=7.7 Hz, 1H), 4.85 (td, J=7.5, 3.8 Hz, 1H), 4.53 (td, J=6.4, 3.3 Hz, 1H), 2.58-2.45 (m, 5H). ¹³C NMR (126 MHz, CD₃OD) δ 158.32, 152.85, 152.76, 141.35, 137.70, 130.47, 125.99, 125.38, 116.71, 116.42, 112.29, 63.84, 43.44, 38.62. HRMS (ESI+): m/z calcd for C₁₅H₁₅ClN₅O: 316.0960 [M+H]⁺; found: 316.0959 [M+H]⁺.

### (S)-2-(1H-benzo[d]imidazol-1-yl)-5-chloro-N-(pyrrolidin-3-yl)pyrimidin-4-amine (II-6c)

1-(4,5-dichloropyrimidin-2-yl)-1H-benzo[d]imidazole 4b (100.0 mg, 377.2 mol), tert-butyl (S)-3-aminopyrrolidine-1-carboxylate (105.4 mg, 103.1 μL, 1.5 equiv., 565.8 mol) and DIPEA (97.51 mg, 131 μL, 2 equiv., 754.4 mol) were reacted according to general procedure D to give the SNAr product which was subjected to Boc deprotection conditions. The residue was purified by prep HPLC to yield II-6c as a 2.0 TFA salt (45 mg, 83 mol, 22%). ¹H NMR (500 MHz, CD₃OD) δ 9.61 (s, 1H), 8.65 (d, J=8.2 Hz, 1H), 8.39 (d, J=1.4 Hz, 1H), 7.83 (d, J=7.9 Hz, 1H), 7.57 (dt, J=22.4, 7.5 Hz, 2H), 5.09-5.01 (m, 1H), 3.79 (dd, J=12.3, 6.9 Hz, 1H), 3.61 (dt, J=11.6, 7.6 Hz, 1H), 3.50 (ddt, J=21.8, 12.4, 6.1 Hz, 2H), 2.56 (dq, J=14.8, 7.5 Hz, 1H), 2.32 (dq, J=13.5, 6.6 Hz, 1H). ¹³C NMR (126 MHz, CD₃OD) δ 158.55, 153.51, 152.75, 141.58, 138.44, 130.57, 125.93, 125.26, 117.07, 116.23, 112.59, 50.60, 49.48, 44.24, 29.60. HRMS (ESI+): m/z calcd for C₁₅H₁₆ClN₆: 315.1120 [M+H]⁺; found: 315.1123 [M+H]⁺.

### (R)-2-(1H-benzo[d]imidazol-1-yl)-5-chloro-N-(pyrrolidin-3-yl)pyrimidin-4-amine (II-6d)

1-(4,5-dichloropyrimidin-2-yl)-1H-benzo[d]imidazole 4b (100.0 mg, 377.2 mol), tert-butyl (R)-3-aminopyrrolidine-1-carboxylate (105.4 mg, 103.1 μL, 1.5 equiv., 565.8 mol) and DIPEA (97.51 mg, 131 μL, 2 equiv., 754.4 mol) were reacted according to general procedure D to give the SNAr product which was subjected to Boc deprotection conditions. The residue was purified by prep HPLC to give II-6d as a 2.0 TFA salt (53 mg, 98 mol, 41%). ¹H NMR (500 MHz, CD₃OD) δ 9.57 (s, 1H), 8.61 (dd, J=8.0, 1.3 Hz, 1H), 8.35 (s, 1H), 7.83-7.78 (m, 1H), 7.54 (dtd, J=20.8, 7.3, 1.3 Hz, 2H), 5.09-4.97 (m, 1H), 3.79 (dd, J=12.4, 6.9 Hz, 1H), 3.61 (dt, J=11.8, 7.6 Hz, 1H), 3.56-3.43 (m, 2H), 2.61-2.50 (m, 1H), 2.36-2.26 (m, 1H). ¹³C NMR (126 MHz, CD₃OD) δ 158.48, 153.43, 152.67, 141.52, 138.41, 130.51, 125.90, 125.22, 117.05, 116.20, 112.55, 50.58, 49.44, 44.20, 29.60. HRMS (ESI+): m/z calcd for C₁₅H₁₆ClN₆: 315.1120 [M+H]⁺; found: 315.1119 [M+H]⁺.

### (S)-2-(1H-benzo[d]imidazol-1-yl)-5-chloro-N-(tetrahydrofuran-3-yl)pyrimidin-4-amine (II-6j)

1-(4,5-dichloropyrimidin-2-yl)-1H-benzo[d]imidazole 4b (20.0 mg, 75.4 mol), (S)-tetrahydrofuran-3-amine (13.1 mg, 2 equiv., 151 mol) and DIPEA (19.5 mg, 26.3 μL, 2 equiv., 151 mol) were reacted according to general procedure D to give the SNAr product which was purified by prep HPLC to give II-6j as a 2.0 TFA salt (13.8 mg, 25.5 mol, 33.8%). ¹H NMR (500 MHz, DMSO-d₆) δ 9.06 (s, 1H), 8.47 (d, J=8.1 Hz, 1H), 8.38 (s, 1H), 7.80 (dd, J=18.0, 7.2 Hz, 2H), 7.47-7.40 (m, 1H), 7.39-7.32 (m, 1H), 4.81 (dtt, J=9.0, 6.6, 4.5 Hz, 1H), 4.03 (dd, J=9.0, 6.4 Hz, 1H), 3.94 (td, J=8.0, 6.5 Hz, 1H), 3.79 (qd, J=8.3, 5.1 Hz, 2H), 2.32 (dtd, J=12.8, 8.0, 6.4 Hz, 1H), 2.19-2.09 (m, 1H). ¹³C NMR (126 MHz, DMSO-d₆) δ 158.38, 153.70, 153.57, 144.86, 142.68, 131.88, 124.66, 123.71, 120.31, 115.55, 110.72, 72.24, 67.09, 52.39, 31.96. HRMS (ESI+): m/z calcd for C₁₅H₁₅ClN₅O: 316.0960 [M+H]⁺; found: 316.0956 [M+H]⁺.

### (R)-1-(2-(1H-benzo[d]imidazol-1-yl)-5-chloropyrimidin-4-yl)pyrrolidin-3-amine (II-6p-R)

1-(4,5-dichloropyrimidin-2-yl)-1H-benzo[d]imidazole 4b (20.0 mg, 75.4 mol), tert-butyl (R)-pyrrolidin-3-ylcarbamate (28.1 mg, 2 equiv., 151 mol) and DIPEA (19.5 mg, 26.3 μL, 2 equiv., 151 mol) were reacted according to general procedure D to give the SNAr product which was subjected to Boc deprotection conditions. The residue was purified by prep HPLC to give II-6p-(R) as a 2.0 TFA salt (30.5 mg, 56.4 mol, 74.8%). ¹H NMR (500 MHz, DMSO-d₆) δ 9.03 (s, 1H), 8.43 (d, J=8.0 Hz, 1H), 8.39 (s, 1H), 8.25-8.21 (m, 3H), 7.81-7.75 (m, 1H), 7.39 (dtd, J=25.9, 7.3, 1.2 Hz, 2H), 4.07 (dddd, J=40.2, 20.7, 10.8, 5.5 Hz, 6H), 2.33 (dtd, J=14.1, 8.4, 6.0 Hz, 1H), 2.14 (ddt, J=12.4, 7.9, 4.5 Hz, 1H). ¹³C NMR (126 MHz, DMSO-d₆) δ 156.83, 153.04, 144.70, 142.56, 131.76, 124.78, 123.89, 120.34, 115.46, 109.84, 53.17, 49.53, 47.61, 29.20. One aromatic carbon is missing/possibly overlapped with a different peak. HRMS (ESI+): m/z calcd for C₁₅H₁₆ClN₆: 315.1120 [M+H]⁺; found: 315.1120 [M+H]⁺.

### (S)-1-(2-(1H-benzo[d]imidazol-1-yl)-5-chloropyrimidin-4-yl)pyrrolidin-3-amine (II-6p-S)

1-(4,5-dichloropyrimidin-2-yl)-1H-benzo[d]imidazole 4b (20.0 mg, 75.4 mol), tert-butyl (S)-pyrrolidin-3-ylcarbamate (28.1 mg, 2 equiv., 151 mol) and DIPEA (19.5 mg, 26.3 μL, 2 equiv., 151 mol) were reacted according to general procedure D to give the SNAr product which was subjected to Boc deprotection conditions. The residue was purified by prep HPLC to give II-6p-(S) as a 2.0 TFA salt (31.5 mg, 58.2 mol, 77.2%). ¹H NMR (500 MHz, DMSO-d₆) δ 9.03 (s, 1H), 8.45-8.40 (m, 1H), 8.38 (s, 1H), 8.26-8.22 (m, 3H), 7.78 (dd, J=7.6, 1.3 Hz, 1H), 7.42 (td, J=7.7, 1.3 Hz, 1H), 7.37 (td, J=7.5, 1.3 Hz, 1H), 4.26-3.92 (m, 6H), 2.33 (dtd, J=14.1, 8.3, 5.9 Hz, 1H), 2.14 (ddt, J=12.4, 7.9, 4.5 Hz, 1H). ¹³C NMR (126 MHz, DMSO-d₆) δ 156.83, 153.04, 144.70, 142.55, 131.75, 124.78, 123.89, 120.34, 115.46, 109.84, 53.17, 49.52, 47.61, 29.18. One aromatic carbon is missing/possibly overlapped with a different peak. HRMS (ESI+): m/z calcd for C₁₅H₁₆ClN₆: 315.1120 [M+H]⁺; found: 315.1122 [M+H]⁺.

### (R)-2-(1H-benzo[d]imidazol-1-yl)-5-bromo-N-(pyrrolidin-3-yl)pyrimidin-4-amine (II-7d)

1-(5-bromo-4-chloropyrimidin-2-yl)-1H-benzo[d]imidazole 4c (20.0 mg, 64.6 mol), tert-butyl (R)-3-aminopyrrolidine-1-carboxylate (24.1 mg, 2 equiv., 129 mol) and DIPEA (16.7 mg, 22.5 μL, 2 equiv., 129 mol) were reacted according to general procedure D to give the SNAr product which was subjected to Boc deprotection conditions. The residue was purified by prep HPLC to give II-7d as a 2.0 TFA salt (18 mg, 31 mol, 48%). ¹H NMR (500 MHz, CD₃OD) δ 9.17 (s, 1H), 8.60-8.52 (m, 1H), 8.48 (s, 1H), 7.77 (dt, J=7.9, 0.9 Hz, 1H), 7.47 (ddd, J=8.3, 7.3, 1.3 Hz, 1H), 7.42 (ddd, J=8.6, 7.4, 1.3 Hz, 1H), 5.05 (tt, J=7.0, 5.2 Hz, 1H), 3.79 (dd, J=12.3, 6.9 Hz, 1H), 3.60 (dt, J=11.9, 7.6 Hz, 1H), 3.55-3.43 (m, 2H), 2.62-2.51 (m, 1H), 2.32 (ddt, J=13.8, 7.8, 6.1 Hz, 1H). ¹³C NMR (126 MHz, CD₃OD) δ 159.16, 156.73, 154.01, 142.99, 141.94, 131.39, 124.75, 123.91, 118.80, 115.54, 100.31, 50.71, 49.57, 44.29, 29.63. HRMS (ESI+): m/z calcd for C₁₅H₁₆BrN₆: 359.0615 [M+H]⁺; found: 359.0615 [M+H]⁺.

### (R)-1-(2-(1H-benzo[d]imidazol-1-yl)-5-bromopyrimidin-4-yl)pyrrolidin-3-amine (II-7p-R)

1-(5-bromo-4-chloropyrimidin-2-yl)-1H-benzo[d]imidazole 4c (20.0 mg, 1 equiv, 64.6 mol), tert-butyl (R)-pyrrolidin-3-ylcarbamate (24.1 mg, 2 equiv. 129 mol) and DIPEA (16.7 mg, 22.5 μL, 2 equiv., 129 mol) were reacted according to general procedure D to give the SNAr product which was subjected to Boc deprotection conditions. The residue was purified by prep HPLC to give II-7p-(R) as a 2.0 TFA salt (25 mg, 43 mol, 66%). ¹H NMR (500 MHz, DMSO-d₆) δ 9.04 (s, 1H), 8.52 (s, 1H), 8.44 (d, J=8.0 Hz, 1H), 8.25-8.20 (m, 3H), 7.82-7.77 (m, 1H), 7.47-7.40 (m, 1H), 7.38 (td, J=7.6, 1.3 Hz, 1H), 4.22-4.10 (m, 2H), 4.04 (ddt, J=27.8, 10.0, 4.2 Hz, 3H), 2.34 (dtd, J=14.1, 8.3, 6.0 Hz, 1H), 2.15 (ddt, J=12.5, 8.0, 4.7 Hz, 1H). ¹³C NMR (126 MHz, DMSO-d₆) δ 159.89, 157.58, 153.35, 144.73, 142.52, 131.74, 124.78, 123.89, 120.35, 115.47, 97.44, 53.45, 49.59, 47.90, 29.25. HRMS (ESI+): m/z calcd for C₁₅H₁₆BrN₆: 359.0615 [M+H]⁺; found: 359.0612 [M+H]⁺.

### (S)-1-(2-(1H-benzo[d]imidazol-1-yl)-5-bromopyrimidin-4-yl)pyrrolidin-3-amine (II-7p-S)

1-(5-bromo-4-chloropyrimidin-2-yl)-1H-benzo[d]imidazole 4c (20.0 mg, 64.6 mol), tert-butyl (S)-pyrrolidin-3-ylcarbamate (24.1 mg, 2 equiv., 129 mol), and DIPEA (16.7 mg, 22.5 μL, 2 equiv., 129 mol) were reacted according to general procedure D to give the SNAr product which was subjected to Boc deprotection conditions. The residue was purified by prep HPLC to give II-7p-(S) as a 2.0 TFA salt (22 mg, 38 mol, 58%). ¹H NMR (500 MHz, DMSO-d₆) δ 9.04 (s, 1H), 8.52 (s, 1H), 8.44 (d, J=8.0 Hz, 1H), 8.23 (s, 3H), 7.79 (d, J=7.9 Hz, 1H), 7.43 (td, J=7.7, 1.3 Hz, 1H), 7.38 (td, J=7.5, 1.3 Hz, 1H), 4.10 (dddd, J=49.4, 27.7, 11.0, 5.5 Hz, 6H), 2.34 (dtd, J=14.1, 8.2, 5.9 Hz, 1H), 2.15 (ddt, J=12.4, 7.9, 4.7 Hz, 1H). ¹³C NMR (126 MHz, DMSO-d₆) δ 159.88, 157.58, 153.35, 144.74, 142.52, 131.74, 124.77, 123.89, 120.35, 115.47, 97.44, 53.45, 49.59, 47.90, 29.25. HRMS (ESI+): m/z calcd for C₁₅H₁₆BrN₆: 359.0615 [M+H]⁺; found: 359.0618 [M+H]⁺.

### (S)-2-(1H-benzo[d]imidazol-1-yl)-4-(pyrrolidin-3-ylamino)pyrimidine-5-carbonitrile (II-9c)

2-(1H-benzo[d]imidazol-1-yl)-4-chloropyrimidine-5-carbonitrile 4e (20.0 mg, 78.2 mol), tert-butyl (S)-3-aminopyrrolidine-1-carboxylate (29.1 mg, 2 equiv., 156 mol), and DIPEA (20.2 mg, 27.3 μL, 2 equiv., 156 mol) were reacted according to general procedure D to give the SNAr product which was subjected to Boc deprotection conditions. The residue was purified by prep HPLC to give II-9c as a 2.0 TFA salt (18 mg, 34 mol, 43%). ¹H NMR (500 MHz, DMSO-d₆) δ 9.17 (s, 1H), 9.07-8.89 (m, 3H), 8.87 (d, J=2.0 Hz, 1H), 8.57 (d, J=6.4 Hz, 1H), 8.47 (d, J=8.0 Hz, 1H), 7.81 (dd, J=7.9, 1.1 Hz, 1H), 7.47 (td, J=7.8, 1.3 Hz, 1H), 7.41 (td, J=7.6, 1.3 Hz, 1H), 5.05-4.95 (m, 1H), 3.63 (dd, J=12.3, 6.8 Hz, 1H), 3.45 (d, J=9.5 Hz, 1H), 2.37 (dq, J=13.4, 7.5 Hz, 1H), 2.20 (ddt, J=13.4, 7.8, 5.8 Hz, 1H). ¹³C NMR (126 MHz, DMSO-d₆) δ 163.42, 161.33, 156.16, 145.03, 143.05, 131.71, 125.20, 124.40, 120.56, 116.04, 115.41, 88.80, 50.74, 49.38, 44.46, 29.77. HRMS (ESI+): m/z calcd for C₁₆H₁₆N₇: 306.1462 [M+H]⁺; found: 306.1460 [M+H]⁺.

### (R)-2-(1H-benzo[d]imidazol-1-yl)-4-(pyrrolidin-3-ylamino)pyrimidine-5-carbonitrile (II-9d, NUCC-0231068)

2-(1H-benzo[d]imidazol-1-yl)-4-chloropyrimidine-5-carbonitrile 4e (50.0 mg, 196 mol), tert-butyl (R)-3-aminopyrrolidine-1-carboxylate (72.9 mg, 2 equiv., 391 mol) and DIPEA (50.6 mg, 68.1 μL, 2 equiv., 391 mol) were reacted according to general procedure D to give the SNAr product which was subjected to Boc deprotection conditions. The residue was purified by prep HPLC to give II-9d (NUCC-0231068) as a 2.0 TFA salt (57 mg, 0.11 mmol, 55%). ¹H NMR (500 MHz, DMSO-d₆) δ 9.17 (s, 1H), 9.00 (d, J=48.5 Hz, 3H), 8.87 (s, 1H), 8.57 (d, J=6.4 Hz, 1H), 8.47 (d, J=8.1 Hz, 1H), 7.81 (d, J=7.9 Hz, 1H), 7.47 (td, J=7.7, 1.3 Hz, 1H), 7.41 (td, J=7.6, 1.3 Hz, 1H), 5.05-4.95 (m, 1H), 3.64 (dd, J=12.0, 6.9 Hz, 1H), 3.34 (dt, J=12.2, 5.3 Hz, 3H), 2.42-2.32 (m, 1H), 2.25-2.15 (m, 1H). ¹³C NMR (126 MHz, DMSO-d₆) δ 163.41, 161.32, 156.15, 145.03, 143.04, 131.70, 125.19, 124.38, 120.55, 116.03, 115.39, 88.79, 50.73, 49.34, 44.43, 29.77. HRMS (ESI+): m/z calcd for C₁₆H₁₆N₇: 306.1462 [M+H]⁺; found: 306.1462 [M+H]⁺.

### 4-(((1s,3s)-3-(aminomethyl)cyclobutyl)amino)-2-(1H-benzo[d]imidazol-1-yl)pyrimidine-5-carbonitrile (II-9g)

2-(1H-benzo[d]imidazol-1-yl)-4-chloropyrimidine-5-carbonitrile 4e (20.0 mg, 78.2 mol), tert-butyl (((1s,3s)-3-aminocyclobutyl)methyl)carbamate (31.3 mg, 2 equiv., 156 mol) and DIPEA (20.2 mg, 27.3 μL, 2 equiv., 156 mol) were reacted according to general procedure D to give the SNAr product which was subjected to Boc deprotection conditions. The residue was purified by prep HPLC to give II-9g as a 2.0 TFA salt (19.7 mg, 36.1 mol, 46.2%). ¹H NMR (500 MHz, CD₃OD) δ 9.08 (s, 1H), 8.60 (s, 1H), 8.56-8.50 (m, 1H), 7.75 (dt, J=7.6, 0.9 Hz, 1H), 7.48-7.36 (m, 2H), 4.73 (tt, J=9.0, 7.6 Hz, 1H), 3.08 (d, J=7.4 Hz, 2H), 2.83-2.72 (m, 2H), 2.49 (tt, J=9.5, 7.5 Hz, 1H), 2.02 (qd, J=9.3, 2.8 Hz, 2H). ¹³C NMR (126 MHz, CD₃OD) δ 162.03, 160.80, 156.35, 143.73, 142.20, 131.37, 124.81, 124.12, 119.21, 115.71, 114.00, 88.61, 44.42, 42.68, 33.60, 26.46. HRMS (ESI+): m/z calcd for C₁₇H₁₈N₇: 320.1618 [M+H]⁺; found: 320.1621 [M+H]⁺.

### 4-(((1r,3r)-3-(aminomethyl)cyclobutyl)amino)-2-(1H-benzo[d]imidazol-1-yl)pyrimidine-5-carbonitrile (II-9k)

2-(1H-benzo[d]imidazol-1-yl)-4-chloropyrimidine-5-carbonitrile 4e (20.0 mg, 78.2 mol), tert-butyl (((1r,3r)-3-aminocyclobutyl)methyl)carbamate (31.3 mg, 2 equiv., 156 mol) and DIPEA (20.2 mg, 27.3 μL, 2 equiv., 156 mol) were reacted according to general procedure D to give the SNAr product which was subjected to Boc deprotection conditions. The residue was purified by prep HPLC to give 11-9k as a 2.0 TFA salt (24.2 mg, 44.4 mol, 56.7%). ¹H NMR (500 MHz, DMSO-d₆) δ 9.15 (s, 1H), 8.79 (s, 1H), 8.75 (d, J=6.3 Hz, 1H), 8.49 (d, J=8.1 Hz, 1H), 7.87 (s, 3H), 7.80 (d, J=7.5 Hz, 1H), 7.50-7.43 (m, 1H), 7.40 (td, J=7.6, 1.2 Hz, 1H), 4.83 (h, J=7.6 Hz, 1H), 3.16-3.09 (m, 2H), 2.47-2.38 (m, 2H), 2.33 (td, J=8.5, 4.2 Hz, 2H). ¹³C NMR (126 MHz, DMSO-d₆) δ 163.26, 160.58, 156.28, 144.99, 142.97, 131.77, 125.08, 124.25, 120.50, 116.09, 115.51, 88.35, 44.40, 43.40, 31.72, 27.23. HRMS (ESI+): m/z calcd for C₁₇H₁₈N₇: 320.1618 [M+H]⁺; found: 320.1620 [M+H]⁺.

### (R)-4-(3-aminopyrrolidin-1-yl)-2-(1H-benzo[d]imidazol-1-yl)pyrimidine-5-carbonitrile (II-9p-R)

2-(1H-benzo[d]imidazol-1-yl)-4-chloropyrimidine-5-carbonitrile 4e (20.0 mg, 78.2 mol), tert-butyl (R)-pyrrolidin-3-ylcarbamate (29.1 mg, 2 equiv., 156 mol), and DIPEA (20.2 mg, 27.3 L, 2 equiv., 156 mol) were reacted according to general procedure D to give the SNAr product which was subjected to Boc deprotection conditions. The residue was purified by prep HPLC after which the ¹³C NMR spectrum had broad peaks in the aliphatic region presumably due to protonation. The purified material was converted to its free base by gently stirring it in a solution of 1:1 ACN:water in the presence Amberlyst A21 free base for 10 min, filtering the mixture, and lyophilizing the filtrate to yield II-9p-(R) (6.5 mg, 21 mol, 27%). ¹H NMR (500 MHz, CD₃OD) δ 9.14 (s, 1H), 8.70 (s, 1H), 8.54 (d, J=8.3 Hz, 1H), 7.80-7.74 (m, 1H), 7.45 (dtd, J=25.8, 7.4, 1.3 Hz, 2H), 5.13-5.04 (m, 1H), 3.77 (dd, J=12.3, 6.9 Hz, 1H), 3.57 (dt, J=11.8, 7.6 Hz, 1H), 3.53-3.39 (m, 2H), 2.61-2.50 (m, 1H), 2.30 (ddt, J=13.7, 7.8, 5.8 Hz, 1H). ¹³C NMR (126 MHz, CD₃OD) δ 162.38, 161.61, 156.27, 144.01, 142.31, 131.43, 124.94, 124.18, 119.36, 115.72, 113.81, 89.36, 50.76, 49.61, 44.35, 29.69. HRMS (ESI+): m/z calcd for C₁₆H₁₆N₇: 306.1462 [M+H]⁺; found: 306.1463 [M+H]⁺.

### (S)-4-(3-aminopyrrolidin-1-yl)-2-(1H-benzo[d]imidazol-1-yl)pyrimidine-5-carbonitrile (II-9p-S)

2-(1H-benzo[d]imidazol-1-yl)-4-chloropyrimidine-5-carbonitrile 4e (20.0 mg, 78.2 mol), tert-butyl (S)-pyrrolidin-3-ylcarbamate (29.1 mg, 27.3 μL, 2 equiv., 156 mol) and DIPEA (20.2 mg, 27.3 μL, 2 equiv., 156 mol) were reacted according to general procedure D to give the SNAr product which was subjected to Boc deprotection conditions. The residue was purified by prep HPLC after which the ¹³C NMR spectrum had broad peaks in the aliphatic region presumably due to protonation. The purified material was converted to its free base by gently stirring it in a solution of 1:1 ACN:water in the presence Amberlyst A21 free base for 10 min, filtering the mixture, and lyophilizing the filtrate to yield II-9p-(S) (16.2 mg, 53.1 mol, 67.8%). ¹H NMR (500 MHz, CD₃OD) δ 9.15 (s, 1H), 8.72 (s, 1H), 8.55 (dd, J=7.9, 1.2 Hz, 1H), 7.77 (dd, J=7.6, 1.3 Hz, 1H), 7.46 (dtd, J=25.8, 7.4, 1.3 Hz, 2H), 5.10 (tt, J=7.0, 5.1 Hz, 1H), 3.78 (dd, J=12.4, 6.9 Hz, 1H), 3.57 (dt, J=11.8, 7.6 Hz, 1H), 3.54-3.38 (m, 2H), 2.61-2.50 (m, 1H), 2.31 (ddt, J=13.7, 7.8, 5.9 Hz, 1H). ¹³C NMR (126 MHz, CD₃OD) δ 162.40, 161.64, 156.29, 144.03, 142.31, 131.44, 124.94, 124.19, 119.37, 115.73, 113.80, 89.39, 50.73, 49.60, 44.36, 29.64. HRMS (ESI+): m/z calcd for C₁₆H₁₆N₇: 306.1462 [M+H]⁺; found: 306.1464 [M+H]⁺.

### General Procedure E: Synthesis of Pure Ethers

To a vial containing the appropriate intermediate in acetonitrile (0.2 M) was added the relevant alcohol (2 equiv.) and cesium carbonate (2 equiv.) after which the reaction was stirred at 40° C. for 16 h after which it was concentrated. If a Boc group was not present, the residue was concentrated then dissolved in 1:1 acetonitrile:water (DMSO added as needed) and purified by reverse phase preparative HPLC with a Phenomenex Gemini-NX C18 column (110 Å, 150×21.2 mm; 5 μm), a gradient of 5% to 80% acetonitrile in water (both solvents contained 0.1% TFA), collection wavelength of 254 nm, and flow rate of 20 mL/min. Relevant fractions were frozen in a dry ice/acetone bath and lyophilized. Compounds purified in this fashion were determined to be 2.0 TFA salts by adding a known amount of internal standard 1,3,5-trimethylbenzene and performing a relative integration.

If a Boc group was present, after the SNAr reaction with the appropriate alcohol and cesium carbonate, the reaction was concentrated to a residue then taken up in DCM (0.2 M) and treated with TFA (40 equiv.). After stirring at room temperature for 16 h, the residue was taken up in 1:1 acetonitrile:water (DMSO added as needed) and purified by prep HPLC in the same conditions as those described above. Unless otherwise noted, compounds purified in this fashion were also determined to be 2.0 TFA salts by adding a known amount of internal standard 1,3,5-trimethylbenzene and performing a relative integration.

### (S)-1-(5-chloro-4-(pyrrolidin-3-yloxy)pyrimidin-2-yl)-1H-benzo[d]imidazole (II-11a)

1-(4,5-dichloropyrimidin-2-yl)-1H-benzo[d]imidazole 4b (20 mg, 75 mol), Cs₂CO₃ (49 mg, 2 equiv., 0.15 mmol) and tert-butyl (S)-3-hydroxypyrrolidine-1-carboxylate (28 mg, 2 equiv., 0.15 mmol) were reacted according to general procedure E to give the SNAr product which was subjected to Boc deprotection conditions. The residue was purified by prep HPLC to give II-11a as a 2.0 TFA salt (11 mg, 20 mol, 27%). ¹H NMR (500 MHz, CD₃OD) δ 9.23 (s, 1H), 8.74 (d, J=0.8 Hz, 1H), 8.60-8.54 (m, 1H), 7.80 (dt, J=7.9, 1.0 Hz, 1H), 7.52 (ddd, J=8.3, 7.3, 1.3 Hz, 1H), 7.46 (ddd, J=8.4, 7.3, 1.2 Hz, 1H), 6.09 (dp, J=4.4, 1.9 Hz, 1H), 3.87-3.76 (m, 2H), 3.65-3.56 (m, 2H), 2.58 (ddd, J=7.2, 4.2, 1.7 Hz, 2H). ¹³C NMR (126 MHz, CD₃OD) δ 164.05, 157.25, 152.63, 142.38, 141.94, 131.03, 125.20, 124.40, 118.75, 115.43, 114.42, 77.14, 50.42, 44.09, 30.56. HRMS (ESI+): m/z calcd for C₁₅H₁₅ClN₅O: 316.0960 [M+H]⁺; found: 316.0960 [M+H]⁺.

### 1-(5-chloro-4-(2-(pyridin-4-yl)ethoxy)pyrimidin-2-yl)-1H-benzo[d]imidazole (II-11b)

1-(4,5-dichloropyrimidin-2-yl)-1H-benzo[d]imidazole 4b (20.0 mg, 75.4 mol), 2-(pyridin-4-yl)ethan-1-ol (18.6 mg, 2 equiv., 151 mol) and Cs₂CO₃ (49.2 mg, 2 equiv., 151 mol) were reacted according to general procedure E to give the SNAr product which was purified by prep HPLC to give II-11b as a 2.0 TFA salt (16 mg, 28 mol, 37%). ¹H NMR (500 MHz, CDCl₃) δ 9.04 (s, 1H), 8.76 (d, J=6.1 Hz, 2H), 8.50 (s, 1H), 8.47-8.41 (m, 1H), 7.87-7.82 (m, 1H), 7.73 (d, J=6.2 Hz, 2H), 7.46-7.36 (m, 2H), 4.87 (t, J=6.0 Hz, 2H), 3.42 (t, J=5.9 Hz, 2H). ¹³C NMR (126 MHz, CDCl₃) δ 164.63, 157.01, 154.42, 153.07, 143.91, 143.50, 141.54, 131.34, 126.66, 125.28, 124.56, 120.24, 115.26, 113.91, 66.65, 34.87. HRMS (ESI+): m/z calcd for C₁₈H₁₅ClN₅O: 352.0960 [M+H]⁺; found: 352.0960 [M+H]⁺.

### (1S,3R)-3-((2-(1H-benzo[d]imidazol-1-yl)-5-chloropyrimidin-4-yl)oxy)cyclopentan-1-amine (II-11f)

1-(4,5-dichloropyrimidin-2-yl)-1H-benzo[d]imidazole 4b (50.0 mg, 189 mol), Cs₂CO₃ (123 mg, 2 equiv., 377 mol) and tert-butyl ((1S,3R)-3-hydroxycyclopentyl)carbamate (75.9 mg, 2 equiv., 377 mol) were reacted according to general procedure E to give the SNAr product which was subjected to Boc deprotection conditions. The residue was purified by prep HPLC to give II-11f as a 2.0 TFA salt (55 mg, 99 mol, 52%). ¹H NMR (500 MHz, CD₃OD) δ 1H NMR (500 MHz, CD3OD) δ 9.28 (s, 1H), 8.61 (s, 1H), 8.53 (d, J=8.1 Hz, 1H), 7.80-7.75 (m, 1H), 7.48 (dtd, J=23.0, 7.5, 1.2 Hz, 2H), 5.72 (tt, J=6.1, 3.0 Hz, 1H), 3.80 (p, J=7.1 Hz, 1H), 2.86 (ddd, J=14.8, 8.1, 6.6 Hz, 1H), 2.37-2.22 (m, 3H), 2.15-1.92 (m, 2H). ¹³C NMR (126 MHz, CD₃OD) δ 164.64, 156.68, 152.64, 141.79, 141.48, 130.88, 125.35, 124.58, 118.38, 115.54, 114.70, 79.45, 50.49, 37.01, 30.56, 28.81. HRMS (ESI+): m/z calcd for C₁₆H₁₇ClN₅O: 330.1116 [M+H]⁺; found: 330.1116 [M+H]⁺.

### (1s,3s)-3-((2-(1H-benzo[d]imidazol-1-yl)-5-chloropyrimidin-4-yl)oxy)cyclobutan-1-amine (II-11h)

1-(4,5-dichloropyrimidin-2-yl)-1H-benzo[d]imidazole 4b (20.0 mg, 1 equiv., 75.4 mol), tert-butyl ((1s,3s)-3-hydroxycyclobutyl)carbamate (28.3 mg, 2 equiv., 151 mol), and Cs₂CO₃ (49.2 mg, 2 equiv., 151 mol) were reacted according to general procedure E to give the SNAr product which was subjected to Boc deprotection conditions. The residue was purified by prep HPLC to give II-11 h as a 2.0 TFA salt (20 mg, 37 mol, 49%). ¹H NMR (500 MHz, CD₃OD) δ 9.24 (s, 1H), 8.62 (s, 1H), 8.52 (d, J=8.1 Hz, 1H), 7.77 (d, J=7.9 Hz, 1H), 7.54-7.47 (m, 1H), 7.45 (td, J=7.7, 1.2 Hz, 1H), 5.38 (p, J=7.1 Hz, 1H), 3.73 (p, J=8.0 Hz, 1H), 3.16 (tdd, J=9.2, 6.2, 2.6 Hz, 2H), 2.56-2.46 (m, 2H). ¹³C NMR (126 MHz, CD₃OD) δ 164.17, 156.91, 152.71, 141.77, 141.62, 130.89, 125.33, 124.55, 118.43, 115.52, 114.22, 66.12, 38.15, 35.34. HRMS (ESI⁺): m/z calcd for C₁₅H₁₅ClN₅O: 316.0960 [M+H]⁺; found: 316.0961 [M+H]⁺.

### (1S,3R)-3-((2-(1H-benzo[d]imidazol-1-yl)-5-chloropyrimidin-4-yl)oxy)-N-methylcyclopentan-1-amine (II-11n)

1-(4,5-dichloropyrimidin-2-yl)-1H-benzo[d]imidazole 4b (50 mg, 0.19 mmol), tert-butyl ((1S,3R)-3-hydroxycyclopentyl)(methyl)carbamate (61 mg, 1.5 equiv., 0.28 mmol) and Cs₂CO₃ (0.12 g, 2 equiv., 0.38 mmol) were reacted according to general procedure E to give the SNAr product which was subjected to Boc deprotection conditions. The residue was purified by prep HPLC to give II-11n as a 2.0 TFA salt (63 mg, 0.11 mmol, 59%). ¹H NMR (500 MHz, CD₃OD) δ 9.28 (s, 1H), 8.62 (s, 1H), 8.53 (d, J=8.1 Hz, 1H), 7.78 (d, J=7.7 Hz, 1H), 7.48 (dtd, J=23.3, 7.5, 1.2 Hz, 2H), 5.73 (dq, J=6.4, 3.2 Hz, 1H), 3.79-3.70 (m, 1H), 2.89 (ddd, J=15.1, 8.4, 6.5 Hz, 1H), 2.78 (s, 3H), 2.38-2.21 (m, 3H), 2.16-1.99 (m, 2H). ¹³C NMR (126 MHz, CD₃OD) δ 164.59, 156.73, 152.68, 141.83, 141.65, 130.93, 125.32, 124.55, 118.45, 115.52, 114.65, 79.05, 58.75, 35.44, 31.08, 30.45, 27.13. HRMS (ESI+): m/z calcd for C₁₇H₁₉ClN₅O: 344.1273 [M+H]⁺; found: 344.1276 [M+H]⁺.

### (S)-1-(5-bromo-4-(pyrrolidin-3-yloxy)pyrimidin-2-yl)-1H-benzo[d]imidazole (II-12a)

1-(5-bromo-4-chloropyrimidin-2-yl)-1H-benzo[d]imidazole 4c (20 mg, 65 mol), Cs₂CO₃ (42 mg, 2 equiv., 0.13 mmol) and tert-butyl (S)-3-hydroxypyrrolidine-1-carboxylate (24 mg, 2 equiv., 0.13 mmol) were reacted according to general procedure E to give the SNAr product which was subjected to Boc deprotection conditions. The residue was purified by prep HPLC to give II-12a as a 2.0 TFA salt (15 mg, 26 mol, 40%). ¹H NMR (500 MHz, CD₃OD) δ 9.19 (s, 1H), 8.78 (s, 1H), 8.54-8.48 (m, 1H), 7.80-7.75 (m, 1H), 7.47 (dtd, J=25.2, 7.4, 1.3 Hz, 2H), 6.03 (dq, J=4.7, 2.9, 2.3 Hz, 1H), 3.88-3.74 (m, 2H), 3.67-3.53 (m, 2H), 2.64-2.50 (m, 2H). ¹³C NMR (126 MHz, CD₃OD) δ 164.88, 160.08, 153.57, 143.01, 141.96, 131.16, 125.07, 124.26, 119.00, 115.40, 102.30, 77.14, 50.44, 44.17, 30.60. HRMS (ESI+): m/z calcd for C₁₅H₁₅BrN₅O: 360.0455 [M+H]⁺; found: 360.0455 [M+H]⁺.

### 1-(5-bromo-4-(2-(pyridin-4-yl)ethoxy)pyrimidin-2-yl)-1H-benzo[d]imidazole (II-12b)

1-(5-bromo-4-chloropyrimidin-2-yl)-1H-benzo[d]imidazole 4c (20 mg, 65 mol), 2-(pyridin-4-yl)ethan-1-ol (16 mg, 2 equiv., 0.13 mmol) and Cs₂CO₃ (42 mg, 2 equiv., 0.13 mmol) were reacted according to general procedure E to give the SNAr product which was purified by prep HPLC to give II-12b as a 2.0 TFA salt (14 mg, 22 mol, 35%). ¹H NMR (500 MHz, CD₃OD) δ 9.13 (s, 1H), 8.83-8.78 (m, 2H), 8.69 (s, 1H), 8.53-8.48 (m, 1H), 8.16-8.11 (m, 2H), 7.77-7.72 (m, 1H), 7.43 (dtd, J=23.2, 7.4, 1.3 Hz, 2H), 4.99 (t, J=5.9 Hz, 2H), 3.58 (t, J=5.9 Hz, 2H). ¹³C NMR (126 MHz, CD₃OD) δ 165.59, 159.56, 159.50, 153.82, 143.33, 141.94, 141.76, 131.20, 127.76, 124.91, 124.09, 119.08, 115.40, 101.81, 66.88, 34.50. HRMS (ESI+): m/z calcd for C₁₈H₁₅BrN₅O: 396.0455 [M+H]⁺; found: 396.0452 [M+H]⁺.

### (1s,3s)-3-((2-(1H-benzo[d]imidazol-1-yl)-5-bromopyrimidin-4-yl)oxy)-N-methylcyclobutan-1-amine (II-12d)

1-(5-bromo-4-chloropyrimidin-2-yl)-1H-benzo[d]imidazole 4c (20.0 mg, 64.6 mol), Cs₂CO₃ (42.1 mg, 2 equiv., 129 mol), and tert-butyl ((1s,3s)-3-hydroxycyclobutyl)(methyl)carbamate (26.0 mg, 2 equiv., 129 mol) were reacted according to general procedure E to give the SNAr product which was subjected to Boc deprotection conditions. The residue was purified by prep HPLC to give II-12d as a 2.0 TFA salt (17 mg, 28 mol, 44%). ¹H NMR (500 MHz, CD₃OD) δ 9.22 (s, 1H), 8.76 (s, 1H), 8.54 (dt, J=8.2, 1.1 Hz, 1H), 7.81-7.75 (m, 1H), 7.48 (dddd, J=26.8, 8.4, 7.3, 1.2 Hz, 2H), 5.41 (p, J=7.1 Hz, 1H), 3.72-3.62 (m, 1H), 3.18 (dtt, J=9.1, 7.1, 2.4 Hz, 2H), 2.71 (s, 3H), 2.52 (dddd, J=10.5, 8.5, 7.2, 3.0 Hz, 2H). ¹³C NMR (126 MHz, CD₃OD) δ 164.97, 159.82, 153.68, 142.32, 141.82, 131.06, 125.21, 124.41, 118.71, 115.50, 102.09, 65.68, 45.85, 34.07, 29.78. HRMS (ESI+): m/z calcd for C₁₆H₁₇BrN₅O: 374.0611 [M+H]⁺; found: 374.0615 [M+H]⁺.

### ((1s,3s)-3-((2-(1H-benzo[d]imidazol-1-yl)-5-bromopyrimidin-4-yl)oxy)cyclobutyl)methanamine (II-12e)

1-(5-bromo-4-chloropyrimidin-2-yl)-1H-benzo[d]imidazole 4c (20.0 mg, 64.6 mol), Cs₂CO₃ (42.1 mg, 2 equiv., 129 mol), and tert-butyl (((1s,3s)-3-hydroxycyclobutyl)methyl)carbamate (26.0 mg, 2 equiv., 129 mol) were reacted according to general procedure E to give the SNAr product which was subjected to Boc deprotection conditions. The residue was purified by prep HPLC to give II-12e as a 2.0 TFA salt (20 mg, 33 mol, 52%). ¹H NMR (500 MHz, CD₃OD) δ 9.23 (s, 1H), 8.76 (s, 1H), 8.57 (dd, J=8.0, 1.2 Hz, 1H), 7.82-7.76 (m, 1H), 7.51 (ddd, J=8.3, 7.2, 1.3 Hz, 1H), 7.46 (td, J=7.7, 1.3 Hz, 1H), 5.48 (p, J=7.3 Hz, 1H), 3.13 (d, J=7.4 Hz, 2H), 2.92 (dtt, J=9.3, 7.1, 2.4 Hz, 2H), 2.48 (tt, J=9.3, 7.4 Hz, 1H), 2.14 (tdd, J=9.6, 7.5, 2.8 Hz, 2H). ¹³C NMR (126 MHz, CD₃OD) δ 165.27, 159.59, 153.75, 142.23, 141.81, 131.09, 125.17, 124.42, 118.68, 115.50, 102.24, 69.03, 44.36, 33.82, 25.01. HRMS (ESI⁺): m/z calcd for C₁₆H₁₇BrN₅O: 374.0611 [M+H]⁺; found: 374.0613 [M+H]⁺.

### (1S,3R)-3-((2-(1H-benzo[d]imidazol-1-yl)-5-bromopyrimidin-4-yl)oxy)cyclopentan-1-amine (II-12f)

1-(5-bromo-4-chloropyrimidin-2-yl)-1H-benzo[d]imidazole 4c (50.0 mg, 162 mol), Cs₂CO₃ (105 mg, 2 equiv., 323 mol) and tert-butyl ((1S,3R)-3-hydroxycyclopentyl)carbamate (65.0 mg, 2 equiv., 323 mol) were reacted according to general procedure E to give the SNAr product which was subjected to Boc deprotection conditions. The residue was purified by prep HPLC to give II-12f as a 2.0 TFA salt (60 mg, 0.10 mmol, 62%). ¹H NMR (500 MHz, CD₃OD) δ 9.17 (s, 1H), 8.71 (d, J=0.6 Hz, 1H), 8.55-8.49 (m, 1H), 7.79-7.74 (m, 1H), 7.45 (dtd, J=23.6, 7.4, 1.3 Hz, 2H), 5.72 (tt, J=6.2, 3.1 Hz, 1H), 3.80 (p, J=7.2 Hz, 1H), 2.91-2.83 (m, 1H), 2.39-2.20 (m, 3H), 2.10-1.95 (m, 2H). ¹³C NMR (126 MHz, CD₃OD) δ 163.59, 159.51, 153.66, 142.77, 141.85, 131.12, 125.04, 124.23, 118.88, 115.41, 102.53, 79.39, 50.53, 37.06, 30.57, 28.81. HRMS (ESI+): m/z calcd for C₁₆H₁₇BrN₅O: 374.0611 [M+H]⁺; found: 374.0612 [M+H]⁺.

### (R)-1-(5-bromo-4-(pyrrolidin-3-yloxy)pyrimidin-2-yl)-1H-benzo[d]imidazole (II-12k)

1-(5-bromo-4-chloropyrimidin-2-yl)-1H-benzo[d]imidazole 4c (20.0 mg, 64.6 mol), Cs₂CO₃ (42.1 mg, 2 equiv., 129 mol), tert-butyl (R)-3-hydroxypyrrolidine-1-carboxylate (24.2 mg, 2 equiv., 129 mol) were reacted according to general procedure E to give the SNAr product which was subjected to Boc deprotection conditions. The residue was purified by prep HPLC to give II-12k as a 2.0 TFA salt (14 mg, 24 mol, 37%). ¹H NMR (500 MHz, CD₃OD) δ 9.29-9.24 (m, 1H), 8.85-8.80 (m, 1H), 8.59-8.53 (m, 1H), 7.82-7.77 (m, 1H), 7.49 (dtd, J=26.1, 7.4, 1.2 Hz, 2H), 6.06 (tq, J=5.7, 2.2, 1.8 Hz, 1H), 3.94-3.72 (m, 2H), 3.67-3.53 (m, 2H), 2.64-2.51 (m, 2H). ¹³C NMR (126 MHz, CD₃OD) δ 164.95, 160.15, 153.58, 141.96, 131.13, 125.20, 124.41, 118.84, 115.47, 102.44, 77.18, 50.46, 44.19, 30.61. One aromatic carbon is missing/possibly overlapped with a different peak. HRMS (ESI+): m/z calcd for C₁₅H₁₅BrN₅O: 360.0454 [M+H]⁺; found: 360.0453 [M+H]⁺.

### (1R,3R)-3-((2-(1H-benzo[d]imidazol-1-yl)-5-bromopyrimidin-4-yl)oxy)-N-methylcyclopentan-1-amine (II-12m)

1-(5-bromo-4-chloropyrimidin-2-yl)-1H-benzo[d]imidazole 4c (20.0 mg, 64.6 mol), Cs₂CO3 (42.1 mg, 2 equiv., 129 mol), and tert-butyl ((1R,3R)-3-hydroxycyclopentyl)(methyl)carbamate (27.8 mg, 2 equiv., 129 mol) were reacted according to general procedure E to give the SNAr product which was subjected to Boc deprotection conditions. The residue was purified by prep HPLC to give II-12m as a 2.0 TFA salt (14 mg, 23 mol, 35%). ¹H NMR (500 MHz, CD₃OD) δ 9.24 (s, 1H), 8.75 (d, J=0.8 Hz, 1H), 8.56 (dd, J=8.0, 1.2 Hz, 1H), 7.79 (dt, J=7.9, 1.0 Hz, 1H), 7.54-7.42 (m, 2H), 5.87 (dq, J=5.5, 2.6 Hz, 1H), 3.88 (p, J=7.6 Hz, 1H), 2.79 (s, 3H), 2.61 (ddt, J=14.8, 7.7, 2.0 Hz, 1H), 2.55-2.40 (m, 2H), 2.28 (ddd, J=14.5, 8.5, 5.9 Hz, 1H), 2.22-2.12 (m, 1H), 1.99-1.87 (m, 1H). ¹³C NMR (126 MHz, CD₃OD) δ 165.35, 159.62, 153.66, 142.42, 141.87, 131.11, 125.15, 124.37, 118.75, 115.48, 102.64, 79.38, 58.59, 35.75, 31.04, 30.16, 26.84. HRMS (ESI+): m/z calcd for C₁₇H₁₉BrN₅O: 388.0767 [M+H]⁺; found: 388.0767 [M+H]⁺.

### (1S,3R)-3-((2-(1H-benzo[d]imidazol-1-yl)-5-bromopyrimidin-4-yl)oxy)-N-methylcyclopentan-1-amine (II-12n)

1-(5-bromo-4-chloropyrimidin-2-yl)-1H-benzo[d]imidazole 4c (50.0 mg, 162 mol), tert-butyl ((1S,3R)-3-hydroxycyclopentyl)(methyl)carbamate (69.5 mg, 2 equiv., 323 mol) and Cs₂CO₃ (105 mg, 2 equiv., 323 mol) were reacted according to general procedure E to give the SNAr product which was subjected to Boc deprotection conditions. The residue was purified by prep HPLC to give II-12n as a 2.0 TFA salt (43 mg, 70 mol, 43%). ¹H NMR (500 MHz, DMSO-d₆) δ 9.13 (s, 1H), 8.95-8.86 (m, 2H), 8.84 (d, J=1.0 Hz, 1H), 8.45 (d, J=8.1 Hz, 1H), 7.80 (d, J=7.9 Hz, 1H), 7.49-7.42 (m, 1H), 7.39 (td, J=7.6, 7.1, 1.2 Hz, 1H), 5.70-5.57 (m, 1H), 3.62 (p, J=7.0 Hz, 1H), 2.82 (dt, J=14.7, 7.5 Hz, 1H), 2.63 (t, J=5.4 Hz, 3H), 2.25-2.04 (m, 3H), 1.92 (dddd, J=19.9, 17.2, 9.8, 6.6 Hz, 2H). ¹³C NMR (126 MHz, DMSO-d₆) δ 165.34, 159.96, 153.87, 144.69, 142.70, 131.62, 125.11, 124.22, 120.43, 115.59, 102.34, 79.03, 57.93, 35.50, 31.68, 30.55, 27.27. HRMS (ESI+): m/z calcd for C₁₇H₁₉BrN₅O: 388.0767 [M+H]⁺; found: 388.0766 [M+H]⁺.

### (1S,3R)-3-((2-(1H-benzo[d]imidazol-1-yl)-5-(trifluoromethyl)pyrimidin-4-yl)oxy)cyclopentan-1-amine (II-13f)

1-(4-chloro-5-(trifluoromethyl)pyrimidin-2-yl)-1H-benzo[d]imidazole 4d (20.0 mg, 67.0 μmol), Cs₂CO₃ (43.6 mg, 2 equiv., 134 mol), and tert-butyl ((1S,3R)-3-hydroxycyclopentyl)carbamate (27.0 mg, 2 equiv., 134 mol) were reacted according to general procedure E to give the SNAr product which was subjected to Boc deprotection conditions. The residue was purified by prep HPLC to give II-13f as a 2.0 TFA salt (13 mg, 22 mol, 33%). ¹H NMR (500 MHz, CD₃OD) δ 9.23 (s, 1H), 8.90 (d, J=1.1 Hz, 1H), 8.59 (dt, J=8.3, 0.9 Hz, 1H), 7.78 (dd, J=7.9, 1.2 Hz, 1H), 7.51 (ddd, J=8.3, 7.3, 1.3 Hz, 1H), 7.45 (td, J=7.6, 1.2 Hz, 1H), 5.83 (tt, J=6.6, 3.7 Hz, 1H), 3.76 (q, J=7.6 Hz, 1H), 2.93 (dt, J=14.8, 7.5 Hz, 1H), 2.41-2.20 (m, 3H), 2.07-1.90 (m, 2H). ¹³C NMR (126 MHz, CD₃OD) δ 166.55, 157.23 (q, J=5.0 Hz), 157.09, 143.39, 142.19, 131.25, 125.22, 124.49, 122.59 (q, J=268.9 Hz), 119.20, 115.62, 108.81 (q, J=34 Hz), 79.22, 50.32, 36.95, 30.49, 28.76. HRMS (ESI+): m/z calcd for C₁₇H₁₇F3N₅O: 364.1380 [M+H]⁺; found: 364.1381 [M+H]⁺.

### (1S,3R)-3-((2-(1H-benzo[d]imidazol-1-yl)-5-(trifluoromethyl)pyrimidin-4-yl)oxy)-N-methylcyclopentan-1-amine (II-13n)

1-(4-chloro-5-(trifluoromethyl)pyrimidin-2-yl)-1H-benzo[d]imidazole 4d (20.0 mg, 67.0 mol), Cs₂CO₃ (43.6 mg, 2 equiv., 134 mol), and tert-butyl ((1S,3R)-3-hydroxycyclopentyl)(methyl)carbamate (28.8 mg, 2 equiv., 134 mol) were reacted according to general procedure E to give the SNAr product which was subjected to Boc deprotection conditions. The residue was purified by prep HPLC to give II-13n as a 2.0 TFA salt (11 mg, 18 mol, 27%). ¹H NMR (500 MHz, CD₃OD) δ 9.23 (s, 1H), 8.94-8.90 (m, 1H), 8.63-8.57 (m, 1H), 7.79 (dd, J=8.0, 1.1 Hz, 1H), 7.51 (ddd, J=8.4, 7.4, 1.3 Hz, 1H), 7.46 (td, J=7.7, 1.2 Hz, 1H), 5.85 (dt, J=6.0, 2.9 Hz, 1H), 3.78-3.68 (m, 1H), 2.97 (ddd, J=15.0, 8.3, 6.7 Hz, 1H), 2.77 (s, 3H), 2.31 (dddd, J=21.8, 15.0, 8.3, 3.7 Hz, 3H), 2.10-1.94 (m, 2H). ¹³C NMR (126 MHz, CD₃OD) δ 166.52, 157.30 (q, J=5.0 Hz), 157.13, 143.53, 142.22, 131.29, 125.20, 124.47, 122.6 (q, J=268.8 Hz), 119.26, 115.61, 108.78 (q, J=33.6 Hz), 78.83, 58.49, 35.31, 30.96, 30.40, 27.02. HRMS (ESI+): m/z calcd for C₁₈H₁₉F3N₅O: 378.1536 [M+H]⁺; found: 378.1539 [M+H]⁺.

### General Procedure F: Synthesis of Pure CN Containing Ethers II-14f, II-14k, and II-14n

To intermediate 4e in acetonitrile (0.2 M) was added the appropriate alcohol (1.5 equiv.) and phosphazene base P₁-t-Bu-tris(tetramethylene) (BTPP) (2 equiv.) was added. The reaction was stirred vigorously at 40° C. for 1 min after which it was removed from the stir plate and immediately (and slowly) acidified with TFA (40 equiv.) then stirred at room temperature for 2 h and monitored by LCMS for completion of the Boc deprotection. The reaction mixture was concentrated, taken up in 1:1 aceonitrile:water (and DMSO if needed) and purified in the same fashion as that described above for prep HPLC. Compounds purified in this fashion were also determined to be 2.0 TFA salts by adding a known amount of internal standard 1,3,5-trimethylbenzene and performing a relative integration.

### 4-(((1R,3S)-3-aminocyclopentyl)oxy)-2-(1H-benzo[d]imidazol-1-yl)pyrimidine-5-carbonitrile (II-14f)

2-(1H-benzo[d]imidazol-1-yl)-4-chloropyrimidine-5-carbonitrile 4e (50.0 mg, 196 mol), tert-butyl ((1S,3R)-3-hydroxycyclopentyl)carbamate (59.0 mg, 1.5 equiv., 293 mol), and BTPP (122 mg, 120 μL, 2 equiv., 391 mol) were reacted according to general procedure F to give the SNAr product which was subjected to Boc deprotection conditions. The residue was purified by prep HPLC to give II-14f as a 2.0 TFA salt (28 mg, 51 mol, 26%). ¹H NMR (500 MHz, CD₃OD) δ 9.16 (s, 1H), 8.97 (t, J=1.1 Hz, 1H), 8.56-8.51 (m, 1H), 7.78 (dd, J=7.8, 1.2 Hz, 1H), 7.53-7.46 (m, 1H), 7.45 (td, J=7.6, 1.2 Hz, 1H), 5.83 (dp, J=6.1, 2.9 Hz, 1H), 3.81 (p, J=7.3 Hz, 1H), 2.89 (ddd, J=15.0, 8.1, 6.6 Hz, 1H), 2.41-2.24 (m, 3H), 2.16-1.90 (m, 2H). ¹³C NMR (126 MHz, CD₃OD) δ 169.15, 163.53, 156.15, 143.74, 142.27, 131.16, 125.30, 124.64, 119.44, 115.65, 112.64, 93.51, 80.14, 50.46, 36.92, 30.60, 28.83. HRMS (ESI+): m/z calcd for C₁₇H₁₇N₆O: 321.1459 [M+H]⁺; found: 321.1463 [M+H]⁺.

### (R)-2-(1H-benzo[d]imidazol-1-yl)-4-(pyrrolidin-3-yloxy)pyrimidine-5-carbonitrile (II-14k)

2-(1H-benzo[d]imidazol-1-yl)-4-chloropyrimidine-5-carbonitrile 4e (50.0 mg, 196 mol), BTPP (122 mg, 120 μL, 2 equiv., 391 mol), and tert-butyl (R)-3-hydroxypyrrolidine-1-carboxylate (54.9 mg, 1.5 equiv., 293 mol) were reacted according to general procedure F to give the SNAr product which was subjected to Boc deprotection conditions. The residue was purified by prep HPLC to give 11-14k as a 2.0 TFA salt (25 mg, 47 mol, 24%). ¹H NMR (500 MHz, CD₃OD) δ 9.21 (s, 1H), 9.03 (s, 1H), 8.53 (dt, J=8.2, 0.9 Hz, 1H), 7.82-7.76 (m, 1H), 7.51 (ddd, J=8.3, 7.3, 1.3 Hz, 1H), 7.46 (td, J=7.6, 1.3 Hz, 1H), 6.13 (tt, J=4.5, 1.9 Hz, 1H), 3.90-3.78 (m, 2H), 3.61 (qdd, J=11.8, 8.9, 6.1 Hz, 2H), 2.68-2.51 (m, 2H). ¹³C NMR (126 MHz, CD₃OD) δ 168.60, 163.92, 156.00, 143.77, 142.34, 131.15, 125.38, 124.72, 119.47, 115.68, 112.29, 93.50, 77.79, 50.38, 44.14, 30.53. HRMS (ESI+): m/z calcd for C₁₆H₁₅N₆O: 307.1302 [M+H]⁺; found: 307.1303 [M+H]⁺.

### 2-(1H-benzo[d]imidazol-1-yl)-4-(((R,3S)-3-(methylamino)cyclopentyl)oxy)pyrimidine-5-carbonitrile (II-14n)

2-(1H-benzo[d]imidazol-1-yl)-4-chloropyrimidine-5-carbonitrile 4e (50.0 mg, 196 mol), BTPP (122 mg, 120 μL, 2 equiv., 391 mol), and tert-butyl ((1S,3R)-3-hydroxycyclopentyl)(methyl)carbamate (63.2 mg, 1.5 equiv., 293 mol) were reacted according to general procedure F to give the SNAr product which was subjected to Boc deprotection conditions. The residue was purified by prep HPLC to give II-14n as a 2.0 TFA salt (40 mg, 71 mol, 36%). ¹H NMR (500 MHz, CD₃OD) δ 9.19 (s, 1H), 8.98 (s, 1H), 8.54 (dt, J=8.3, 1.0 Hz, 1H), 7.82-7.76 (m, 1H), 7.54-7.42 (m, 2H), 5.87-5.80 (m, 1H), 3.76 (tt, J=8.5, 6.4 Hz, 1H), 2.91 (ddd, J=15.1, 8.6, 6.5 Hz, 1H), 2.79 (s, 3H), 2.40-2.27 (m, 3H), 2.15 (ddd, J=15.1, 6.7, 3.3 Hz, 1H), 2.11-2.00 (m, 1H). ¹³C NMR (126 MHz, CD₃OD) δ 169.10, 163.55, 156.15, 143.67, 142.29, 131.16, 125.32, 124.66, 119.41, 115.66, 112.64, 93.53, 79.81, 58.73, 35.33, 31.08, 30.47, 27.16. HRMS (ESI+): m/z calcd for C₁₈H₁₉N₆O: 335.1615 [M+H]⁺; found: 335.1619 [M+H]⁺.

The remaining compounds of II-5a-II-5p, II-6a-II-6p, II-7a-II-7p, II-8a-II-8p, II-9a-II-9p, II-10a-II-10p, II-11a-II-11p, II-12a-II-12p, II-13a-II-13p, and II-14a-II-14p may be synthesized according to the method disclosed in General procedure D-F using appropriate starting materials.

Phospho-eIF4E in-Cell Western Assay

The MNK activity was assessed in a cell-based assay using the phosphorylation level of Ser209 on its downstream substrate protein eIF4E. The culture media for LN229 cells was DMEM (ThermoFisher Scientific, Cat No. 11965092) with 10% fetal bovine serum (ThermoFisher Scientific, Cat No. A5670701) and 50 U/mL penicillin-strep (ThermoFisher Scientific, Cat No. 15070063; 1:100 dilution in the final media from the 5,000 U/ml stock). Stock solutions were prepared in DMSO at an initial concentration of 30 mM, which were transferred to 384-well Low Dead Volume Microplates (Labcyte Cat No. LP-0200) manually. A three-fold serial dilution was performed to generate a range of 11 concentrations for the assay. 10K cells were plated 40 μl per well in a 384-well assay plates (Greiner, Cat. No. 781080). After overnight incubation of the cell assay plates, compound solutions of different concentrations were transferred to the cell assay plates using an Echo® 550 liquid transfer system. The duration of the treatment was 2 hours. The cells were then fixed with 1.5% PFA followed by permeabilization with cold MeOH. The phosphorylation of eIF4E was detected using Ser209 anti-phospho-eIF4E antibody (Abcam, 183301), followed by binding with an anti-rabbit secondary antibody conjugated with horse radish peroxidase (HRP). The chemiluminescent intensity from HRP was detected using SuperSignal ELISA Femto Substrate (Thermo Scientific, 37074) and was measured by luminescence plate reader Synergy Neo2 (BioTek).

### Western Blots

LN229 cells were seeded at 1.5×10⁶ cells per 10 cm dish. The next day, cells were treated with the indicated MNK inhibitors at 10 nM and 100 nM or DMSO as vehicle control for 2h. SDS-PAGE and Immunoblots were executed as previously described,⁴² with the exception that cells were lysed in NP-40 lysis buffer (50 mM Tris, pH 7.5, 150 mM NaCl, 5 mM EDTA, 0.5% NP-40, 1 mM DTT and protease- and phosphatase-inhibitors from Roche). The polyclonal rabbit antibody against phosphorylated eIF4E at Ser209 was purchased from Cell Signaling Technology (#9741) and monoclonal mouse antibodies against eIF4E (#sc-9976) and HSP90 (#sc-13119) were from Santa Cruz Biotechnology. Secondary HRP-conjugated and AF488-conjugated antibodies were applied simultaneously, followed by visualization using a ChemiDoc MP imager (Bio-Rad).

### Biochemical MNK1 and MNK2 Assays.

Compounds were tested at Eurofins Scientific SE and were tested against MNK1 (71-002KP) and MNK2 (14-664KP) in a radiometric biochemical assay each using [ATP]=Kₘ for that kinase isoform.

MNK1: MNK1 (human) is incubated with 8 mM MOPS pH 7.0, 0.2 mM EDTA, 250 μM KKLRRTLSFAEPG peptide, 10 mM Magnesium acetate and [γ-33P]-ATP]. The reaction is initiated by the addition of the Mg/ATP mix (200 μM ATP). After incubation for 40 minutes at room temperature, the reaction is stopped by the addition of phosphoric acid to a concentration of 0.5%. 8 μl of the stopped reaction is spotted onto KPF filter and washed four times for 4 minutes in 0.425% phosphoric acid and once in ethanol prior to drying and scintillation counting.

MNK2: Mnk2 (human) is incubated with 8 mM MOPS pH 7.0, 0.2 mM EDTA, 0.33 mg/mL myelin basic protein, 10 mM Magnesium acetate and [γ-33P]-ATP. The reaction is initiated by the addition of the Mg/ATP mix (120 μM ATP). After incubation for 40 minutes at room temperature, the reaction is stopped by the addition of phosphoric acid to a concentration of 0.5%. An aliquot of the reaction is then spotted onto a filter and washed four times for 4 minutes in 0.425% phosphoric acid and once in ethanol prior to drying and scintillation counting.

### REFERENCES

(24) Vagadia, P. P.; Izquierdo-Ferrer, J.; Mazewski, C.; Blyth, G.; Beauchamp, E. M.; Clutter, M. R.; Stern, C. L.; Mishra, R. K.; Nahotko, D.; Small, S.; Eckerdt, F.; Platanias, L. C.; Schiltz, G. E. Discovery of Potent and Selective MNK Kinase Inhibitors for the Treatment of Leukemia. J. Med. Chem. 2025, 68 (5), 5824-5844. DOI: 10.1021/acs.jmedchem.4c03158
(42) Beauchamp, E. M.; Kosciuczuk, E. M.; Serrano, R.; Nanavati, D.; Swindell, E. P.; Viollet, B.; O'Halloran, T. V.; Altman, J. K.; Platanias, L. C. Direct binding of arsenic trioxide to AMPK and generation of inhibitory effects on acute myeloid leukemia precursors. Mol. Cancer Ther. 2015, 14 (1), 202-212. DOI: 10.1158/1535-7163.MCT-14-0665-T

I/We claim:

1. A compound of formula (I), or a pharmaceutically acceptable salt, hydrate, or solvate thereof, 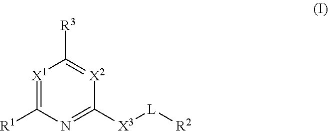
wherein
(i) X³ is NR⁹, X¹ is N, X² is CR⁸;
R³ is —H;
R⁸ is —CN, —H, halogen,- or —CF₃;
R⁹ is —H, or R⁹ together with the nitrogen atom it is attached to, L, and R² form a monocyclic or bicyclic heterocycloalkyl substituted with 0-1 hydroxyl or —NH₂;
R¹ is benzimidazolyl substituted with 0-1 —NH₂;
L is absent or an alkylene, wherein L is substituted with 0-1 hydroxyl; and
R² is heterocycloalkyl, -4-pyridyl, phenyl, or cycloalkyl; wherein R² is substituted with 0-1 hydroxyl, halogen, —NH₂, or alkyl optionally substituted with amino;
or
(ii) X³ is O, X¹ is N, X² is CR⁴;
R³ is —H, alkyl or cycloalkyl;
R⁴ is halogen, —H, —CN, or haloalkyl;
R¹ is heteroaryl, wherein R¹ is substituted with 0-1 —NH₂, halogen, —CN, or —NO₂;
L is absent or an alkylene; and
R² is pyridyl, heterocycloalkyl, cycloalkyl, or phenyl, wherein R² is substituted with 0-1 amino, alkyl, oxo, halogen, —NH—C(O)-cycloalkyl, —C(O)—O-alkyl, —C(O)—OH, or alkyl optionally substituted with amino;
or
(iii) X³ is O, X¹ is CR⁵, X² is N;
R³ is —H;
R⁵ is —H or halogen;
R¹ is a heteroaryl, wherein R¹ is substituted with 0-1 —NH₂;
L is absent; and
R² is piperidinyl, wherein R² is substituted with 0-1 alkyl;
or
(iv) X³ is NR⁶, X¹ is CR⁷, X² is N;
R³ is —H;
R⁶ is —H, or R⁶ together with the nitrogen atom it is attached to, L, and R² form a bicyclic heterocycloalkyl;
R⁷ is —H, —Cl, or —Br;
R¹ is benzimidazolyl, pyrazolyl, or imidazolyl, wherein R¹ is substituted with 0-1 —NH₂, halogen, —CN, methyl, —C₆H₅, or —C(O)—NH₂; and
-L-R² is 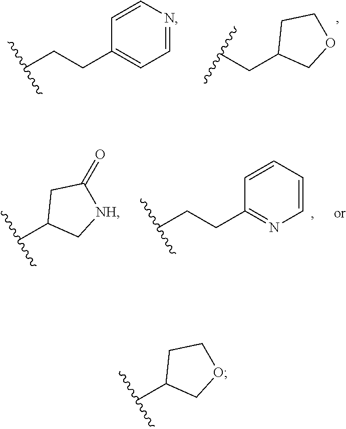 [SMILES: *CCc1ccncc1 ; *CC1CCOC1 ; *C1CNC(=O)C1 ; *CCc1ccccn1 ; *C1CCOC1]
with the proviso that the compound is not

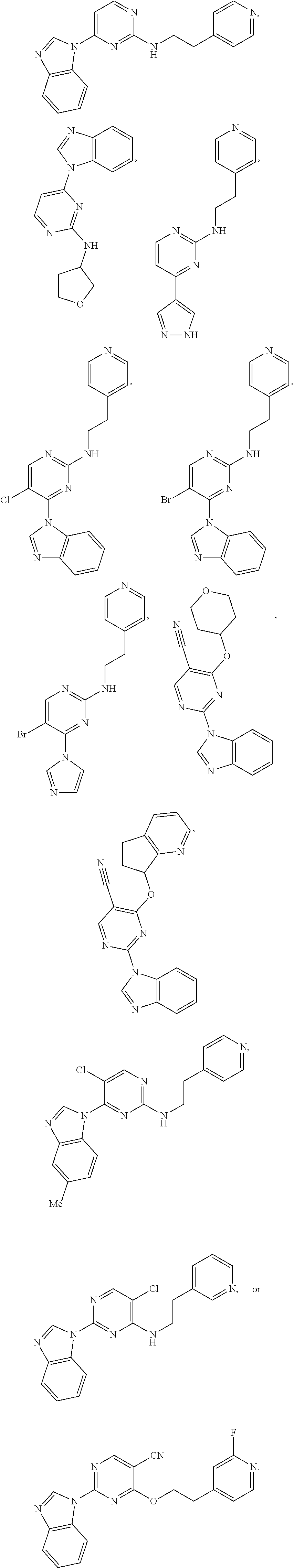

2. The compound of claim 1, wherein X³ is NR⁹, X¹ is N, and X² is CR⁸.

3. The compound of claim 2, wherein R⁸ is —CN, —H, —Cl, —Br, or —CF₃.

4. The compound of any one of claims 2-3, wherein L is absent.

5. The compound of any one of claims 2-3, wherein L is ethylene substituted with 0-1 hydroxyl.

6. The compound of any one of claims 2-5, wherein R⁹ is —H.

7. The compound of any one of claims 2-5, wherein R⁹ together with the nitrogen atom it is attached to, L, and R² form

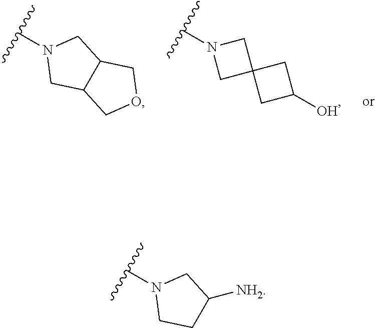 [SMILES: *N1CC2COCC2C1 ; *N1CC2(CC(O)C2)C1 ; *N1CCC(N)C1]

8. The compound of any one of claims 2-7, wherein R² is heterocycloalkyl or cycloalkyl, wherein R² is substituted with 0-1 hydroxyl, —NH₂, or —CH₂—NH₂.

9. The compound of claim 8, wherein R² is

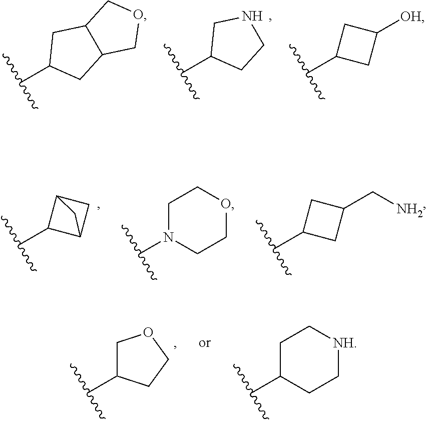

10. The compound of any one of claims 2-7, wherein R² is pyridyl or phenyl substituted with 0-1 —F.

11. The compound of claim 1, wherein X³ is O, X¹ is N, and X² is CR⁴.

12. The compound of claim 11, wherein R¹ is benzimidazolyl or imidazopyridinyl substituted with 0-1 —NH₂, halogen, —CN, or —NO₂.

13. The compound of claim 11 or claim 12, wherein R¹ is benzimidazolyl substituted with 0-1 —NH₂, —CN, and —Br.

14. The compound of any one of claims 11-13, wherein R³ is H.

15. The compound of any one of claims 11-13, wherein R³ is methyl or cyclopropyl.

16. The compound of any one of claims 11-15, wherein R⁴ is —Cl, —F, —Br, —CF₃, or —CN.

17. The compound of any one of claims 11-16, wherein L is C₁-C₆ alkylene.

18. The compound of claim 17, wherein L is ethylene.

19. The compound of any one of claims 11-16, wherein L is absent.

20. The compound of any one of claims 11-19, wherein R² is heterocycloalkyl substituted with 0-1 methyl, oxo, or —C(O)—O-tBu.

21. The compound of claim 20, wherein R² is

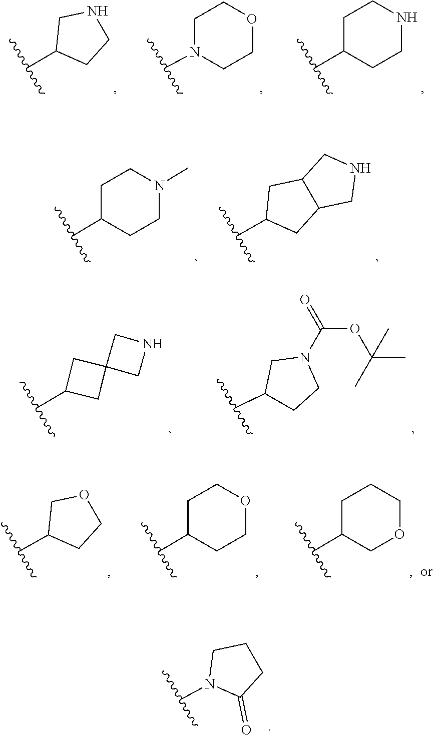

22. The compound of any one of claims 11-19, wherein R² is pyridyl substituted with 0-1 —F.

23. The compound of any one of claims 11-19, wherein R² is cycloalkyl substituted with 0-1 —NH₂, —NH—CH₃, —NH—C(O)-cyclopropyl, —CH₂—NH₂, or —C(O)—OH.

24. The compound of claim 23, wherein R² is

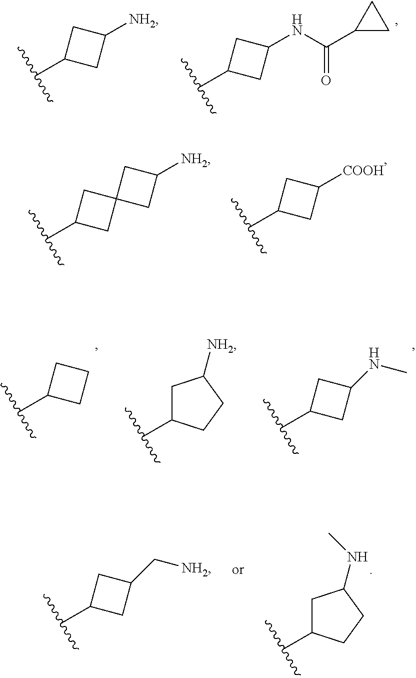

25. The compound of any one of claims 11-19, wherein R² is phenyl substituted with 0-1 —F, —Cl, or —NH₂.

26. The compound of claim 1, wherein X³ is O, X¹ is CR⁵, and X² is N.

27. The compound of claim 26, wherein R⁵ is —Cl.

28. The compound of any one of claims 26-27, wherein R¹ is benzimidazolyl substituted with 0-1 —NH₂.

29. The compound of any one of claims 26-28, wherein R² is piperidinyl substituted with 0-1 methyl.

30. The compound of claim 29, wherein R² is

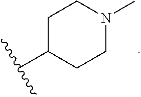 [SMILES: *C1CCN(C)CC1]

31. The compound of claim 1, wherein X³ is NR⁶, X¹ is CR⁷, and X² is N.

32. The compound of claim 31, wherein R⁶ is H.

33. The compound of any one of claim 31-32, wherein R¹ is pyrazolyl substituted with 0-1 methyl or —C₆H₅.

34. The compound of any one of claim 31-32, wherein R¹ is benzimidazolyl substituted with 0-1 —C(O)—NH₂, —CN, —NH₂, or —Br.

35. The compound of any one of claim 31-32, wherein R¹ is imidazolyl.

36. The compound of claim 1, or a pharmaceutically acceptable salt, hydrate, or solvate thereof, wherein the compound is

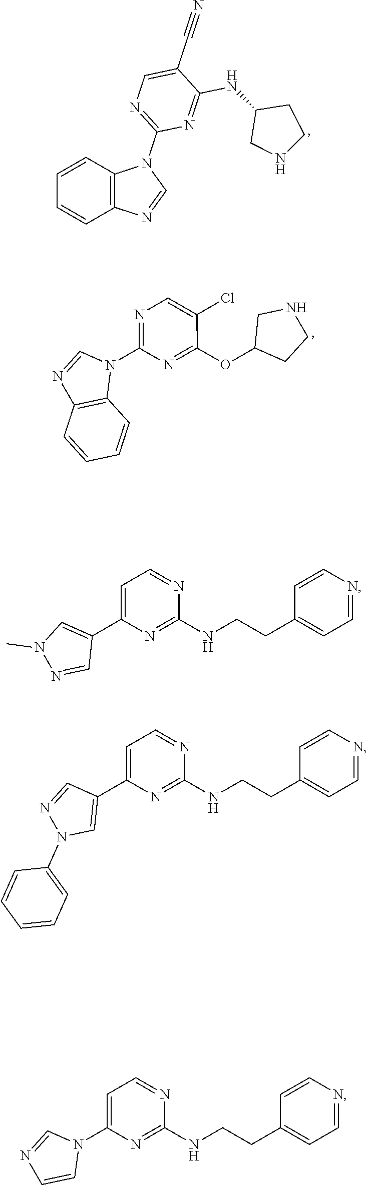 [SMILES: N#Cc1cnc(-n2cnc3ccccc32)nc1N[C@@H]1CCNC1 ; Clc1cnc(-n2cnc3ccccc32)nc1OC1CCNC1 ; Cn1cc(-c2ccnc(NCCc3ccncc3)n2)cn1 ; c1ccc(-n2cc(-c3ccnc(NCCc4ccncc4)n3)cn2)cc1 ; c1cc(CCNc2nccc(-n3ccnc3)n2)ccn1]

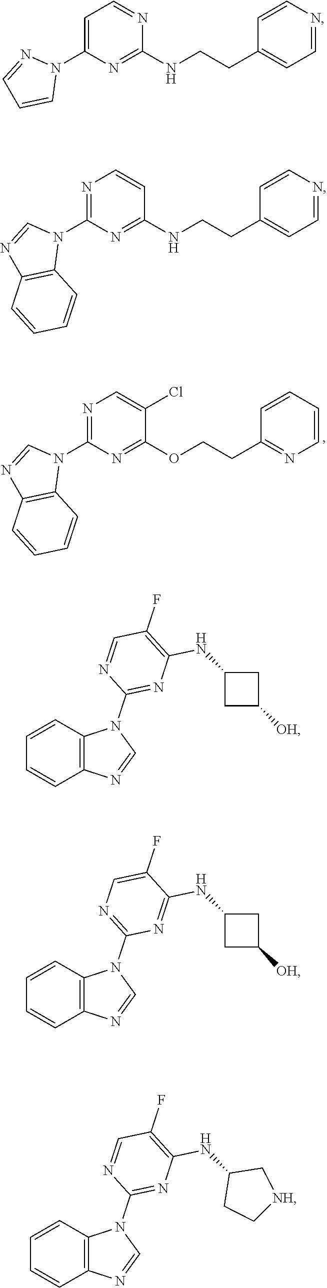 [SMILES: c1cnn(-c2ccnc(NCCc3ccncc3)n2)c1 ; c1ccc2c(c1)ncn2-c1nccc(NCCc2ccncc2)n1 ; Clc1cnc(-n2cnc3ccccc32)nc1OCCc1ccccn1 ; O[C@H]1C[C@@H](Nc2nc(-n3cnc4ccccc43)ncc2F)C1 ; O[C@H]1C[C@H](Nc2nc(-n3cnc4ccccc43)ncc2F)C1 ; Fc1cnc(-n2cnc3ccccc32)nc1N[C@H]1CCNC1]

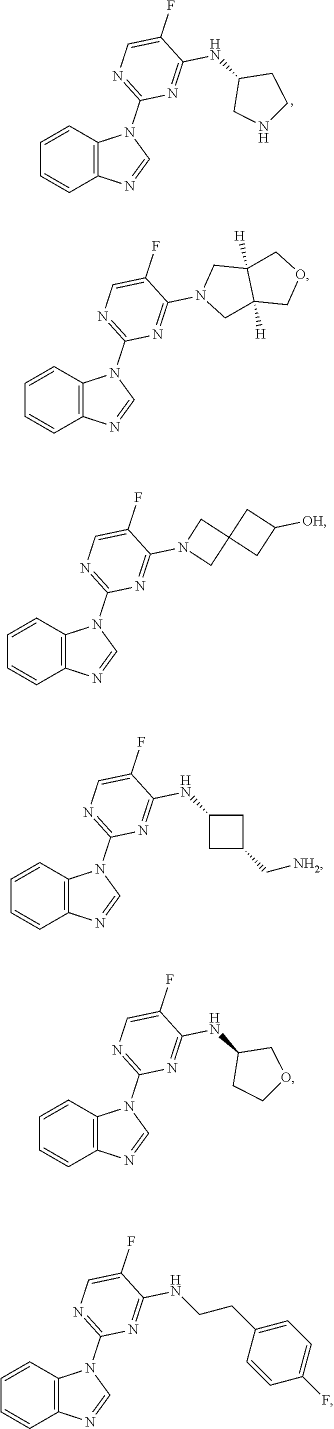 [SMILES: Fc1cnc(-n2cnc3ccccc32)nc1N[C@@H]1CCNC1 ; Fc1cnc(-n2cnc3ccccc32)nc1N1C[C@H]2COC[C@H]2C1 ; OC1CC2(C1)CN(c1nc(-n3cnc4ccccc43)ncc1F)C2 ; NC[C@H]1C[C@@H](Nc2nc(-n3cnc4ccccc43)ncc2F)C1 ; Fc1cnc(-n2cnc3ccccc32)nc1N[C@@H]1CCOC1 ; Fc1ccc(CCNc2nc(-n3cnc4ccccc43)ncc2F)cc1]

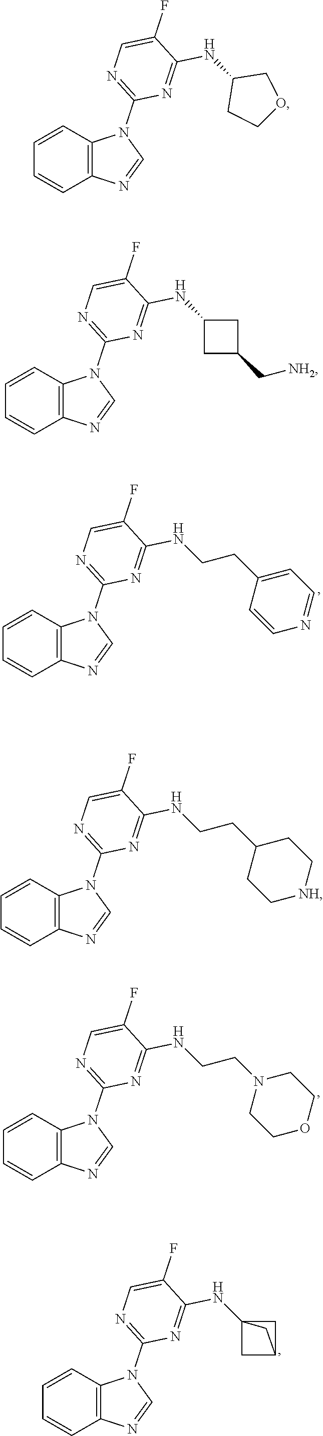 [SMILES: Fc1cnc(-n2cnc3ccccc32)nc1N[C@H]1CCOC1 ; NC[C@H]1C[C@H](Nc2nc(-n3cnc4ccccc43)ncc2F)C1 ; Fc1cnc(-n2cnc3ccccc32)nc1NCCc1ccncc1 ; Fc1cnc(-n2cnc3ccccc32)nc1NCCC1CCNCC1 ; Fc1cnc(-n2cnc3ccccc32)nc1NCCN1CCOCC1 ; Fc1cnc(-n2cnc3ccccc32)nc1NC12CC(C1)C2]

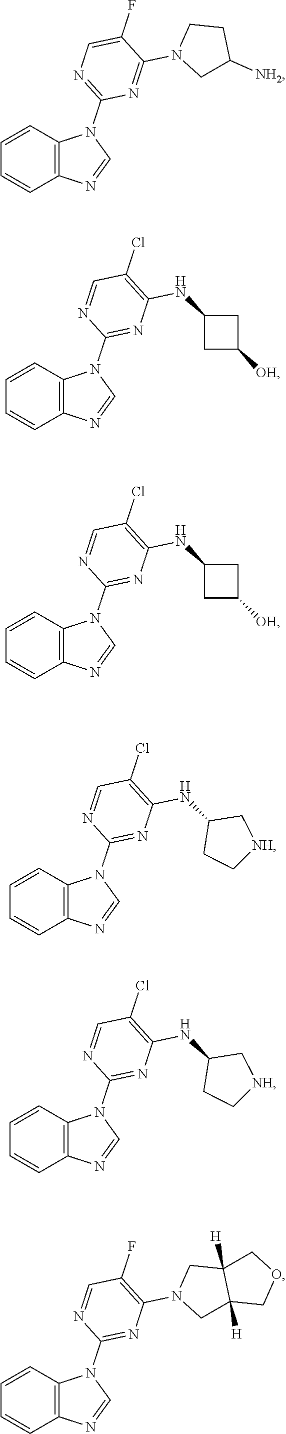 [SMILES: NC1CCN(c2nc(-n3cnc4ccccc43)ncc2F)C1 ; O[C@H]1C[C@@H](Nc2nc(-n3cnc4ccccc43)ncc2Cl)C1 ; O[C@H]1C[C@H](Nc2nc(-n3cnc4ccccc43)ncc2Cl)C1 ; Clc1cnc(-n2cnc3ccccc32)nc1N[C@H]1CCNC1 ; Clc1cnc(-n2cnc3ccccc32)nc1N[C@@H]1CCNC1 ; Fc1cnc(-n2cnc3ccccc32)nc1N1C[C@H]2COC[C@H]2C1]

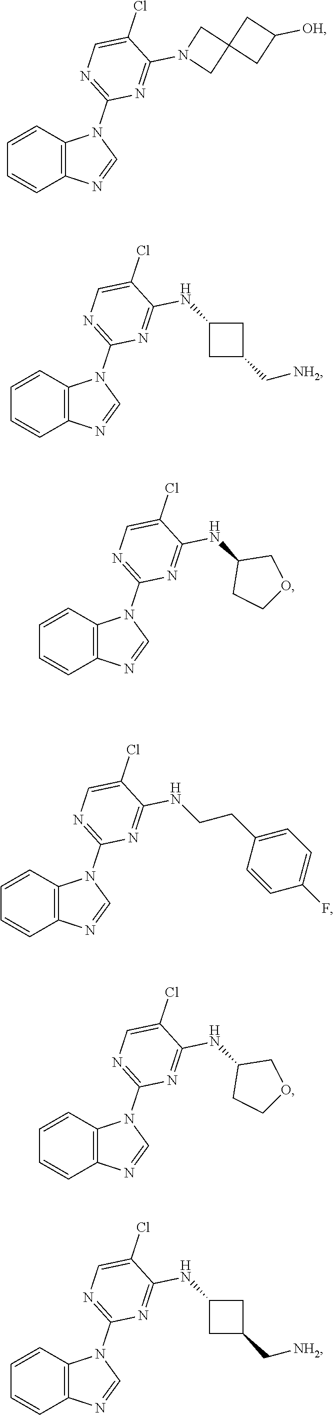 [SMILES: OC1CC2(C1)CN(c1nc(-n3cnc4ccccc43)ncc1Cl)C2 ; NC[C@H]1C[C@@H](Nc2nc(-n3cnc4ccccc43)ncc2Cl)C1 ; Clc1cnc(-n2cnc3ccccc32)nc1N[C@@H]1CCOC1 ; Fc1ccc(CCNc2nc(-n3cnc4ccccc43)ncc2Cl)cc1 ; Clc1cnc(-n2cnc3ccccc32)nc1N[C@H]1CCOC1 ; NC[C@H]1C[C@H](Nc2nc(-n3cnc4ccccc43)ncc2Cl)C1]

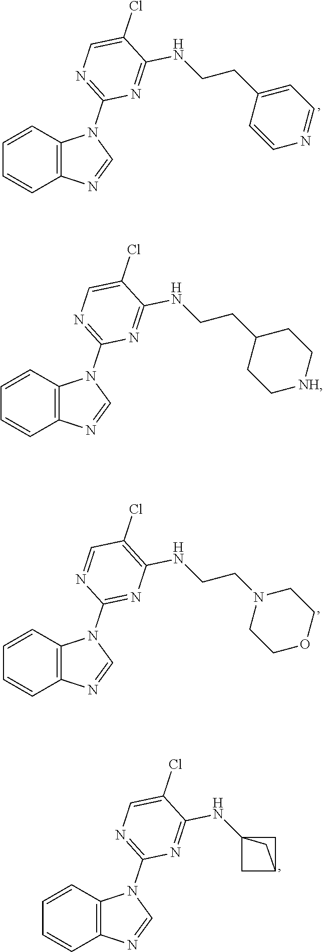 [SMILES: Clc1cnc(-n2cnc3ccccc32)nc1NCCc1ccncc1 ; Clc1cnc(-n2cnc3ccccc32)nc1NCCC1CCNCC1 ; Clc1cnc(-n2cnc3ccccc32)nc1NCCN1CCOCC1 ; Clc1cnc(-n2cnc3ccccc32)nc1NC12CC(C1)C2]

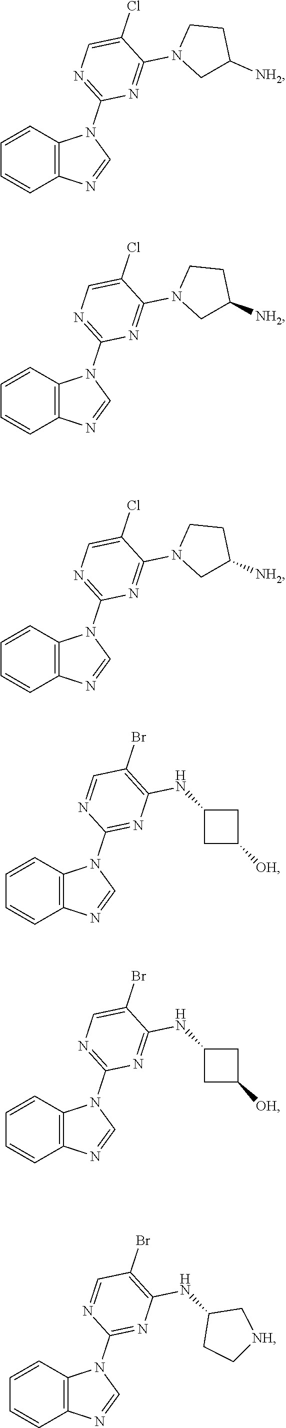 [SMILES: NC1CCN(c2nc(-n3cnc4ccccc43)ncc2Cl)C1 ; N[C@@H]1CCN(c2nc(-n3cnc4ccccc43)ncc2Cl)C1 ; N[C@H]1CCN(c2nc(-n3cnc4ccccc43)ncc2Cl)C1 ; O[C@H]1C[C@@H](Nc2nc(-n3cnc4ccccc43)ncc2Br)C1 ; O[C@H]1C[C@H](Nc2nc(-n3cnc4ccccc43)ncc2Br)C1 ; Brc1cnc(-n2cnc3ccccc32)nc1N[C@H]1CCNC1]

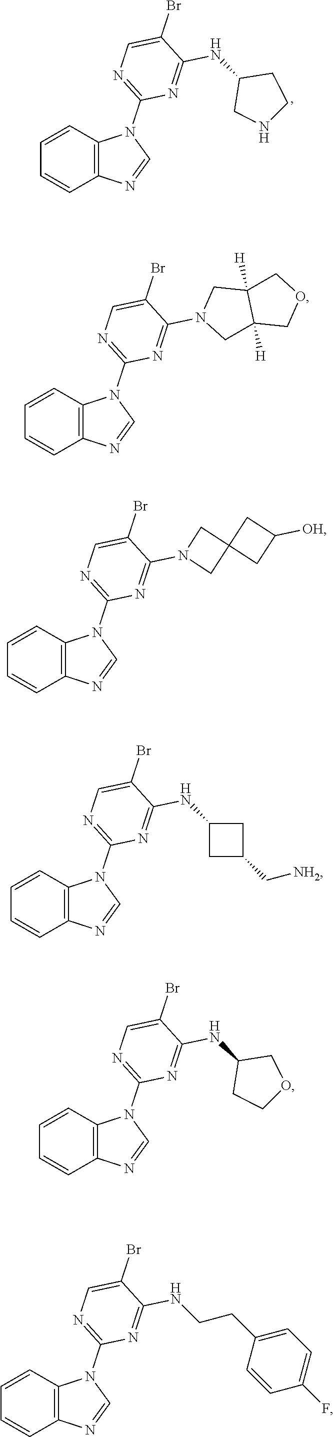 [SMILES: Brc1cnc(-n2cnc3ccccc32)nc1N[C@@H]1CCNC1 ; Brc1cnc(-n2cnc3ccccc32)nc1N1C[C@H]2COC[C@H]2C1 ; OC1CC2(C1)CN(c1nc(-n3cnc4ccccc43)ncc1Br)C2 ; NC[C@H]1C[C@@H](Nc2nc(-n3cnc4ccccc43)ncc2Br)C1 ; Brc1cnc(-n2cnc3ccccc32)nc1N[C@@H]1CCOC1 ; Fc1ccc(CCNc2nc(-n3cnc4ccccc43)ncc2Br)cc1]

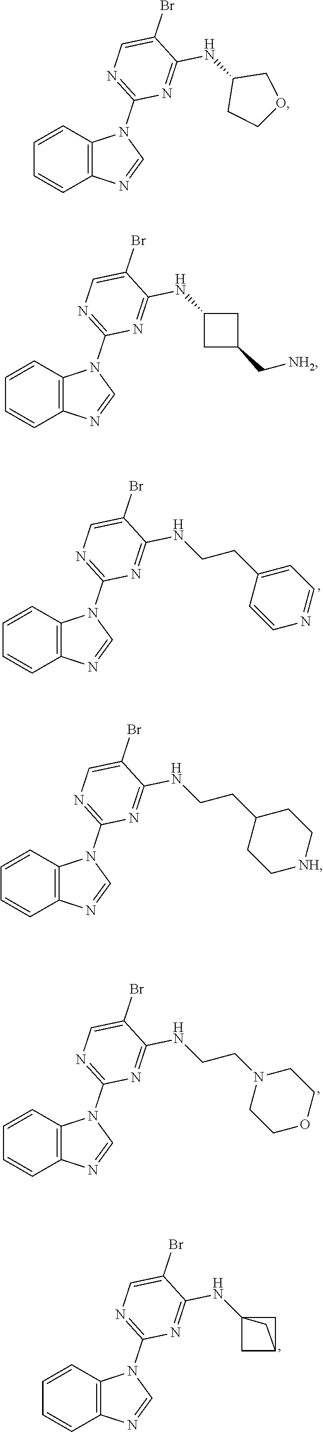 [SMILES: Brc1cnc(-n2cnc3ccccc32)nc1N[C@H]1CCOC1 ; NC[C@H]1C[C@H](Nc2nc(-n3cnc4ccccc43)ncc2Br)C1 ; Brc1cnc(-n2cnc3ccccc32)nc1NCCc1ccncc1 ; Brc1cnc(-n2cnc3ccccc32)nc1NCCC1CCNCC1 ; Brc1cnc(-n2cnc3ccccc32)nc1NCCN1CCOCC1 ; Brc1cnc(-n2cnc3ccccc32)nc1NC12CC(C1)C2]

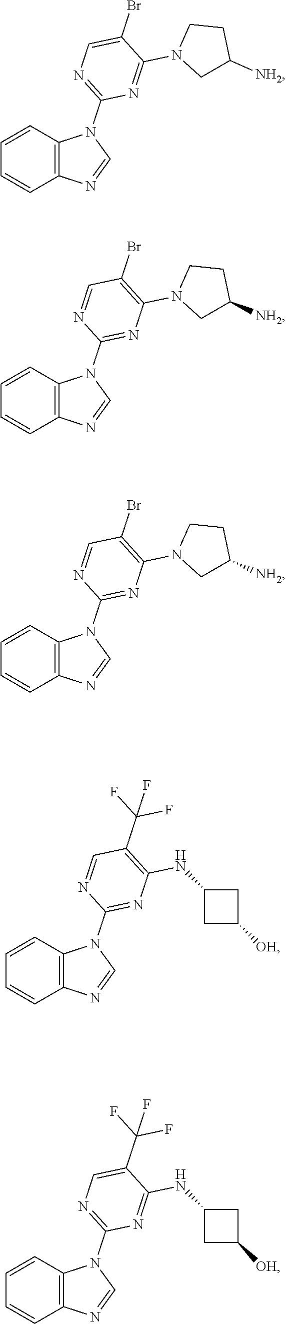 [SMILES: NC1CCN(c2nc(-n3cnc4ccccc43)ncc2Br)C1 ; N[C@@H]1CCN(c2nc(-n3cnc4ccccc43)ncc2Br)C1 ; N[C@H]1CCN(c2nc(-n3cnc4ccccc43)ncc2Br)C1 ; O[C@H]1C[C@@H](Nc2nc(-n3cnc4ccccc43)ncc2C(F)(F)F)C1 ; O[C@H]1C[C@H](Nc2nc(-n3cnc4ccccc43)ncc2C(F)(F)F)C1]

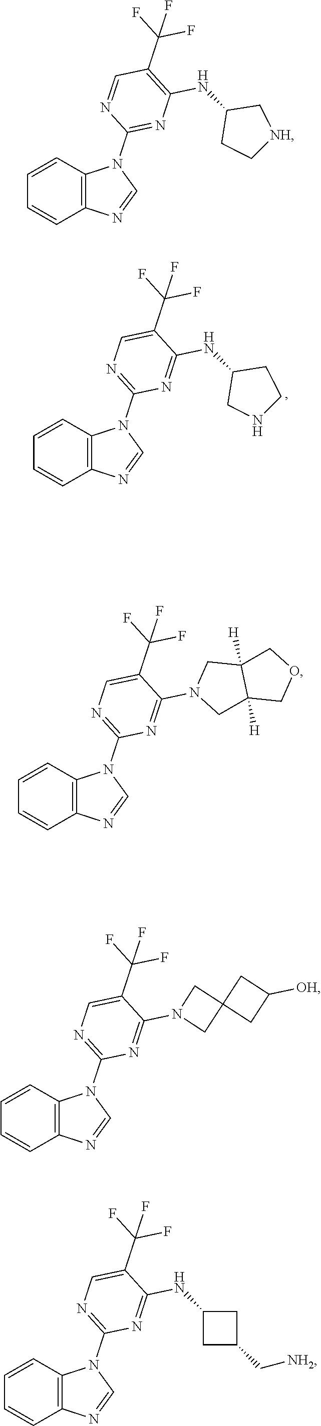 [SMILES: FC(F)(F)c1cnc(-n2cnc3ccccc32)nc1N[C@H]1CCNC1 ; FC(F)(F)c1cnc(-n2cnc3ccccc32)nc1N[C@@H]1CCNC1 ; FC(F)(F)c1cnc(-n2cnc3ccccc32)nc1N1C[C@H]2COC[C@H]2C1 ; OC1CC2(C1)CN(c1nc(-n3cnc4ccccc43)ncc1C(F)(F)F)C2 ; NC[C@H]1C[C@@H](Nc2nc(-n3cnc4ccccc43)ncc2C(F)(F)F)C1]

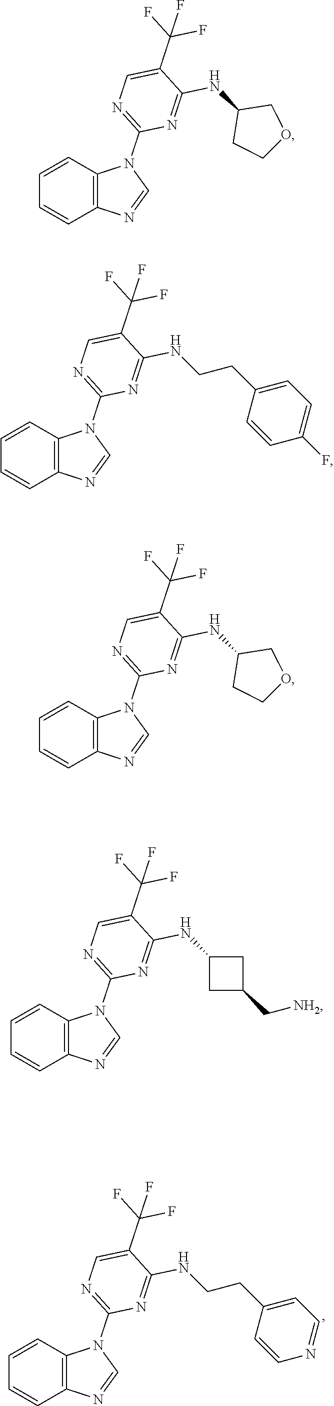 [SMILES: FC(F)(F)c1cnc(-n2cnc3ccccc32)nc1N[C@@H]1CCOC1 ; Fc1ccc(CCNc2nc(-n3cnc4ccccc43)ncc2C(F)(F)F)cc1 ; FC(F)(F)c1cnc(-n2cnc3ccccc32)nc1N[C@H]1CCOC1 ; NC[C@H]1C[C@H](Nc2nc(-n3cnc4ccccc43)ncc2C(F)(F)F)C1 ; FC(F)(F)c1cnc(-n2cnc3ccccc32)nc1NCCc1ccncc1]

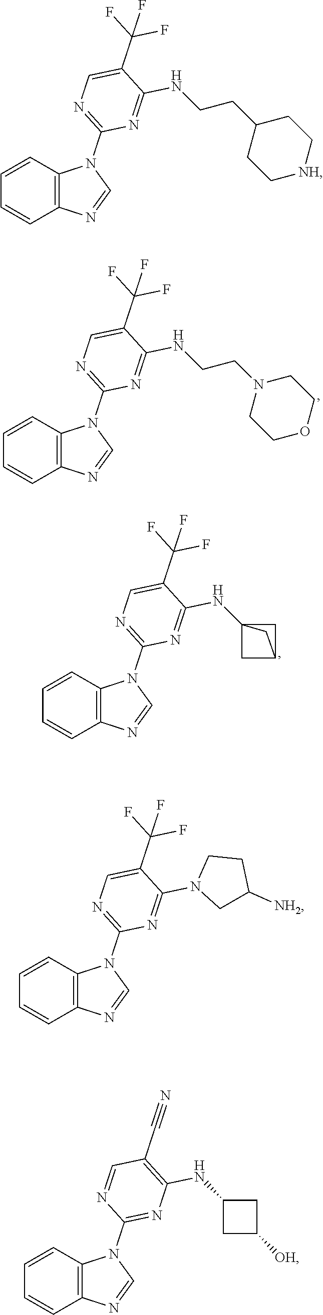 [SMILES: FC(F)(F)c1cnc(-n2cnc3ccccc32)nc1NCCC1CCNCC1 ; FC(F)(F)c1cnc(-n2cnc3ccccc32)nc1NCCN1CCOCC1 ; FC(F)(F)c1cnc(-n2cnc3ccccc32)nc1NC12CC(C1)C2 ; NC1CCN(c2nc(-n3cnc4ccccc43)ncc2C(F)(F)F)C1 ; N#Cc1cnc(-n2cnc3ccccc32)nc1N[C@H]1C[C@@H](O)C1]

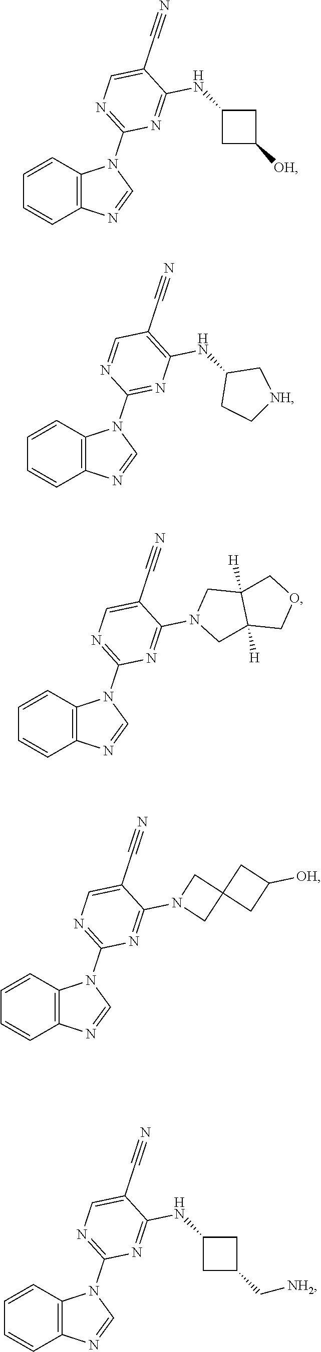 [SMILES: N#Cc1cnc(-n2cnc3ccccc32)nc1N[C@H]1C[C@H](O)C1 ; N#Cc1cnc(-n2cnc3ccccc32)nc1N[C@H]1CCNC1 ; N#Cc1cnc(-n2cnc3ccccc32)nc1N1C[C@H]2COC[C@H]2C1 ; N#Cc1cnc(-n2cnc3ccccc32)nc1N1CC2(CC(O)C2)C1 ; N#Cc1cnc(-n2cnc3ccccc32)nc1N[C@H]1C[C@@H](CN)C1]

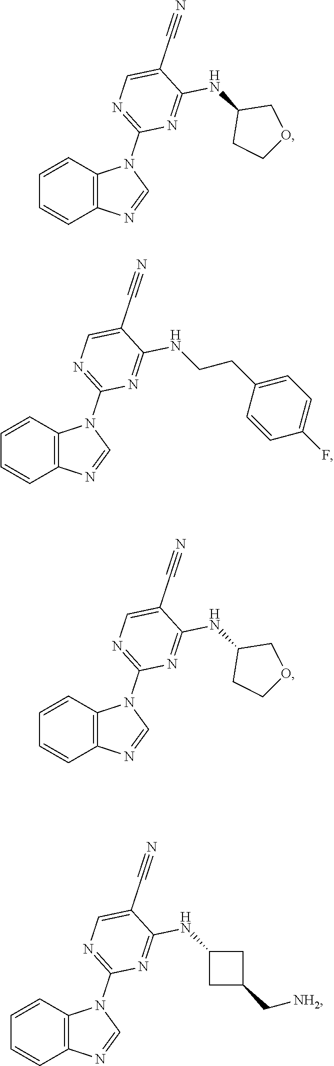 [SMILES: N#Cc1cnc(-n2cnc3ccccc32)nc1N[C@@H]1CCOC1 ; N#Cc1cnc(-n2cnc3ccccc32)nc1NCCc1ccc(F)cc1 ; N#Cc1cnc(-n2cnc3ccccc32)nc1N[C@H]1CCOC1 ; N#Cc1cnc(-n2cnc3ccccc32)nc1N[C@H]1C[C@H](CN)C1]

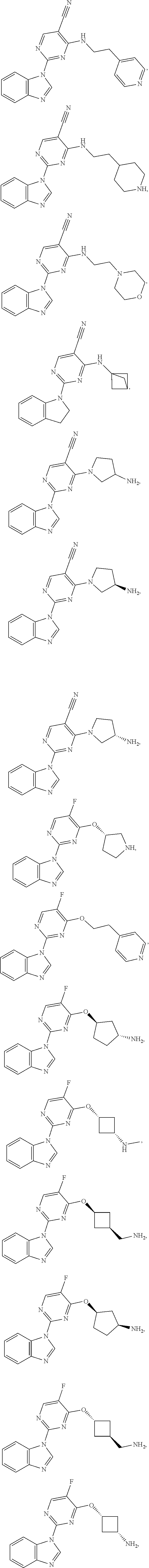

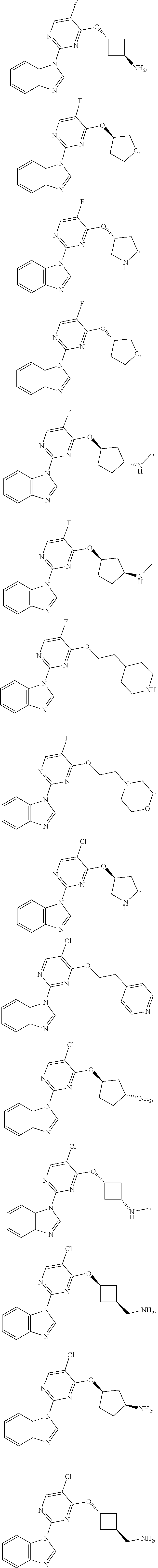

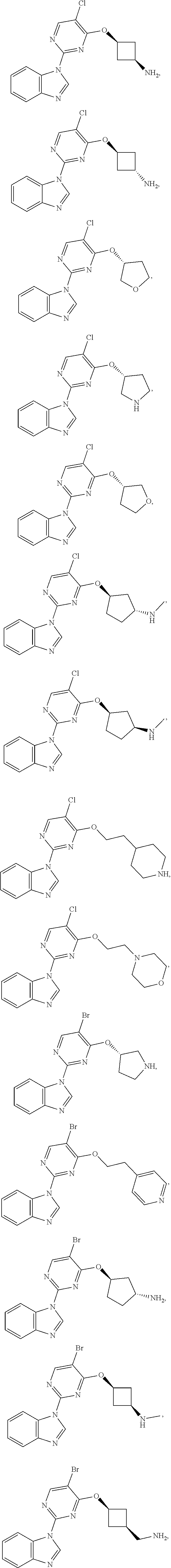

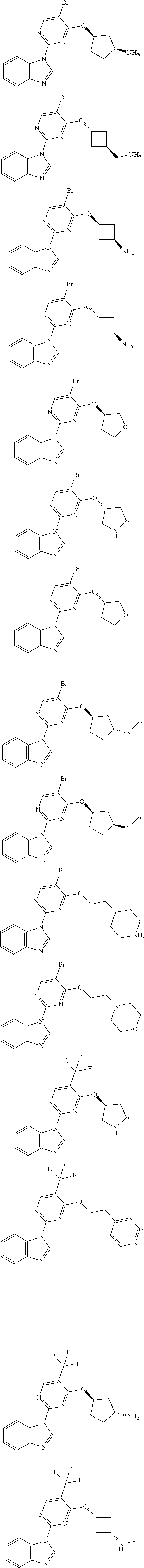

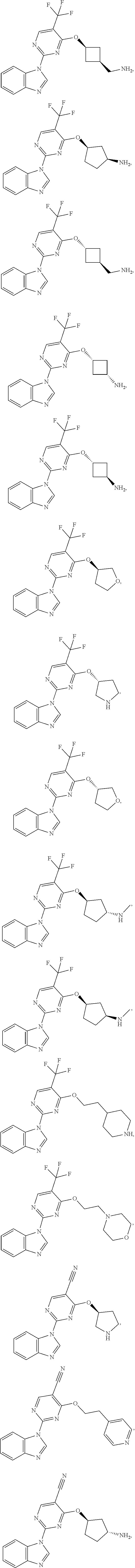

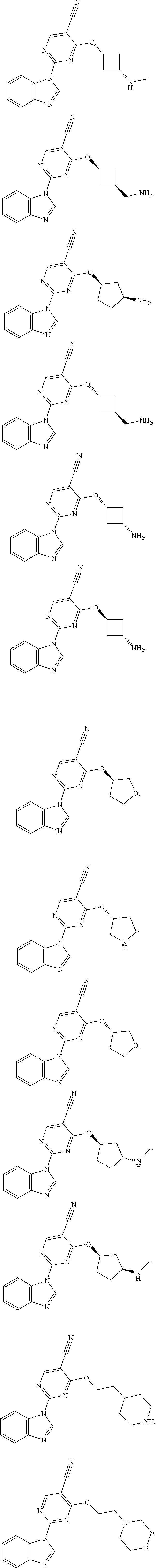

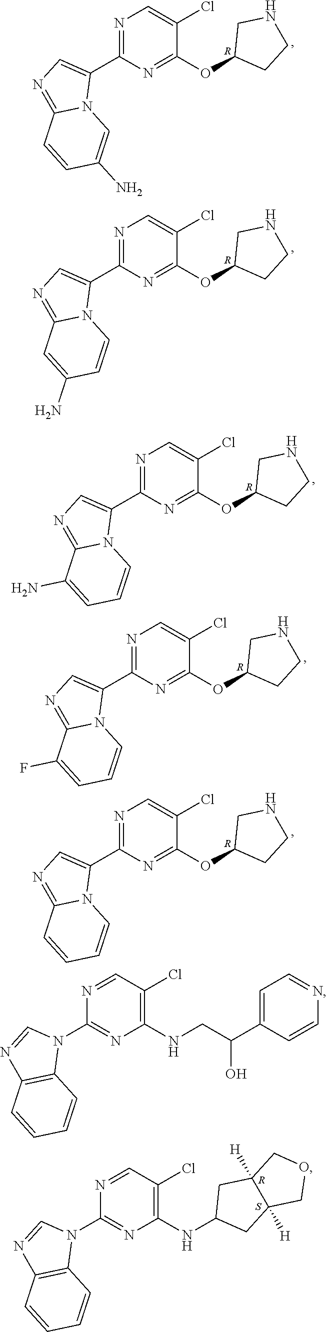

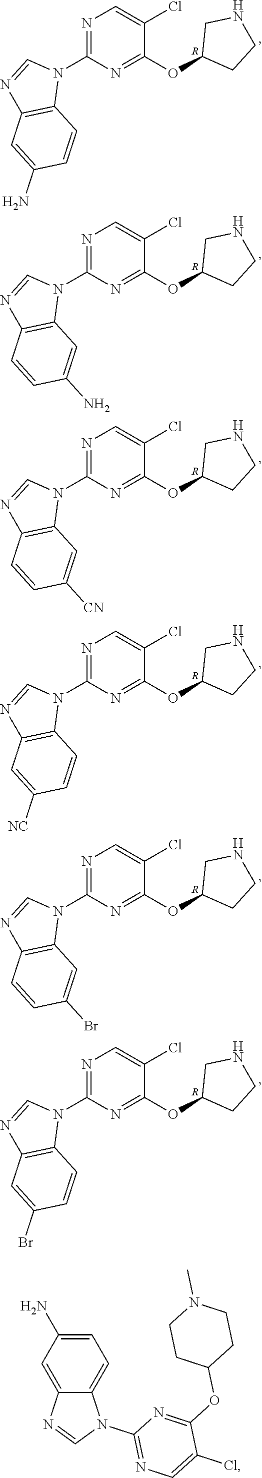

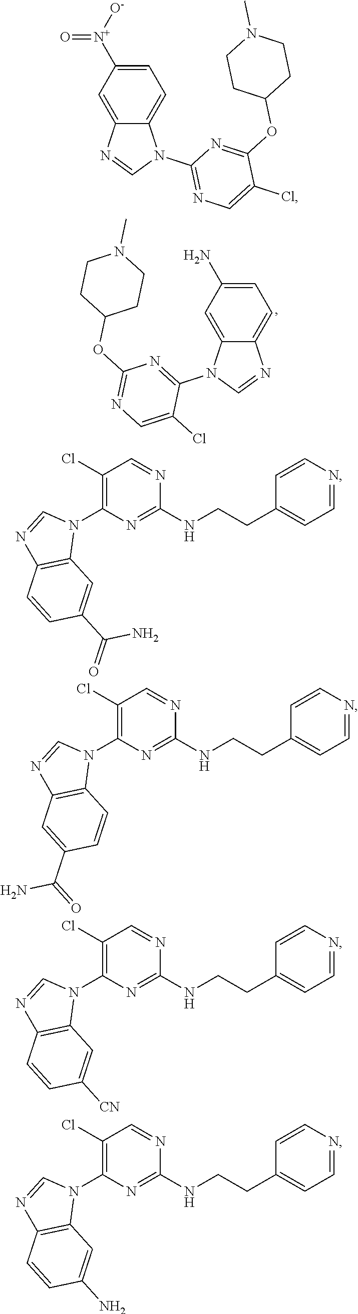

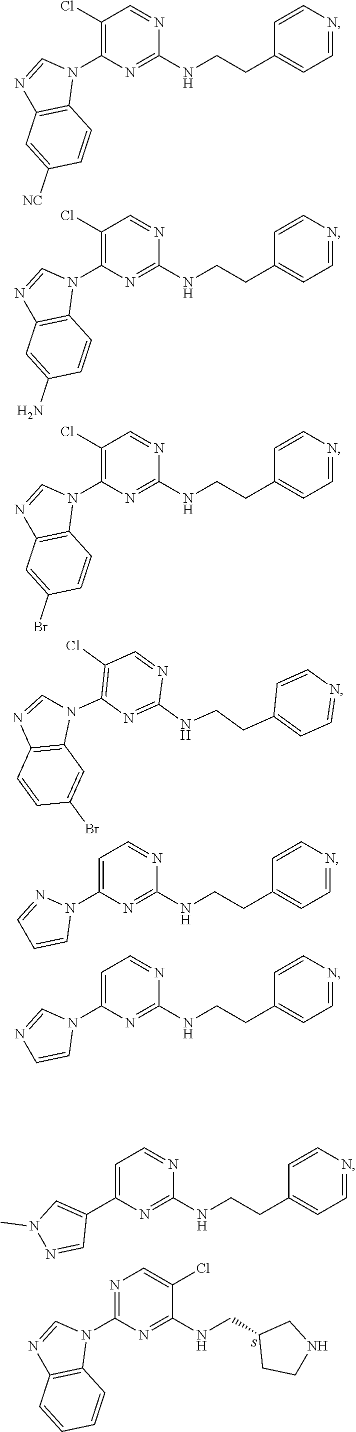

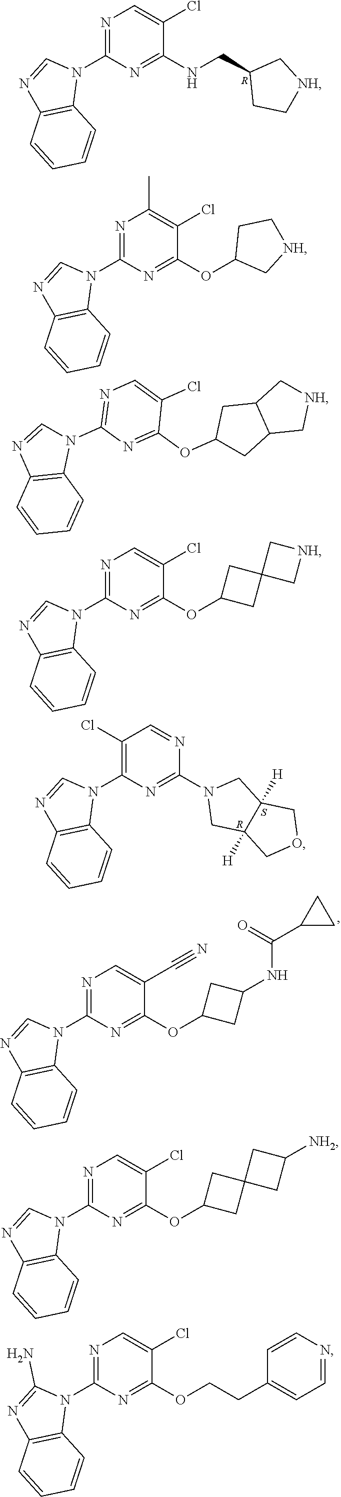

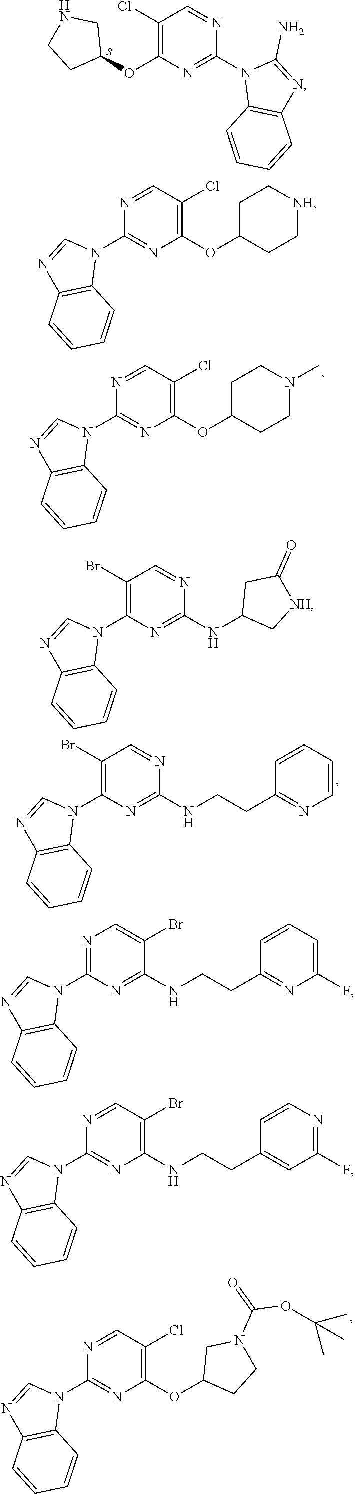

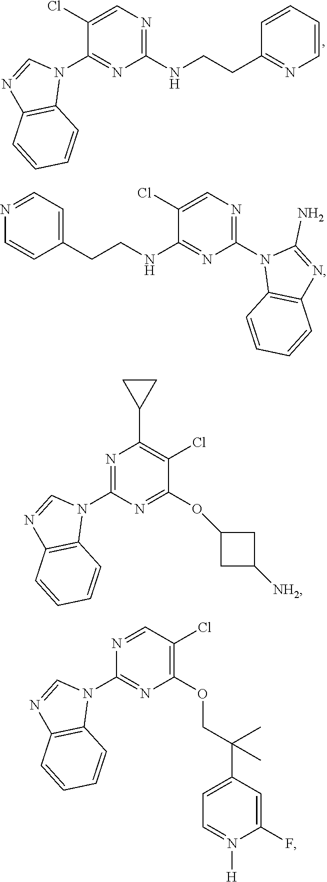

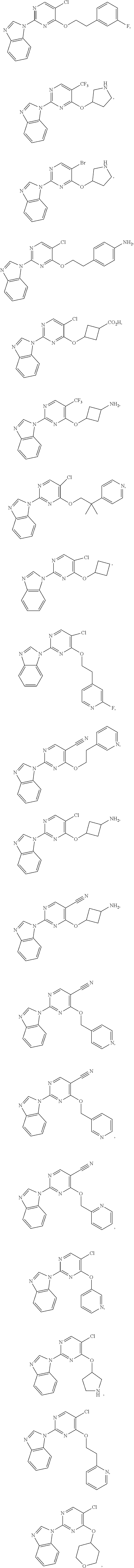

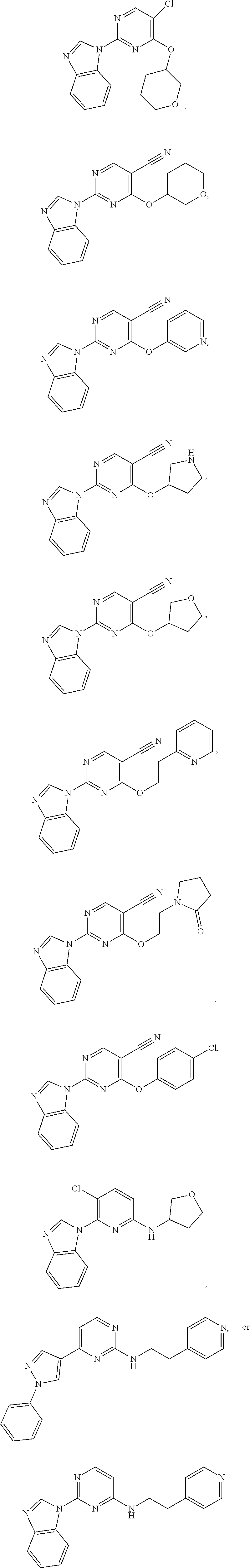

37. The compound of claim 1, or a pharmaceutically acceptable salt, hydrate, or solvate thereof, wherein the compound is

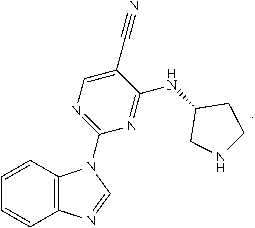 [SMILES: N#Cc1cnc(-n2cnc3ccccc32)nc1N[C@@H]1CCNC1]

38. The compound of claim 1, or a pharmaceutically acceptable salt, hydrate, or solvate thereof, wherein the compound is

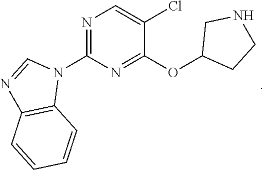 [SMILES: Clc1cnc(-n2cnc3ccccc32)nc1OC1CCNC1]

39. The compound of any one of claims 1-38, wherein the compound decreases phosphorylation of eIF4E at Ser209 in AML cells or glioblastoma cells.

40. The compound of any one of claims 1-39, wherein the compound decreases AML cell viability.

41. The compound of any one of claims 1-40, wherein the compound induces apoptosis in AML cells.

42. The compound of any one of claims 1-41, wherein the compound achieves a total brain/plasma (B/P) ratio of at least 3.5 in a subject after administering the compound to the subject.

43. A pharmaceutical composition comprising the compound according to any one of claims 1-42 and a pharmaceutically acceptable excipient, carrier, or diluent.

44. A method for treating a disease or disorder associated with mitogen-activated protein kinase interacting kinase 1 and/or 2 (MNK1 and/or MNK2) activity in a subject in need thereof, the method comprising administering an effective amount of the compound of any one of claims 1-42, or a pharmaceutically acceptable salt, hydrate, or solvate thereof, or the pharmaceutical composition of claim 43 to the subject.

45. The method of claim 44, wherein the disease or disorder is cancer or a cell proliferative disorder.

46. The method of claim 45, wherein the cancer or the cell proliferative disorder is a hematological malignancy.

47. The method of claim 46, wherein the hematological malignancy is acute myeloid leukemia (AML).

48. The method of any one of claims 44-47, further comprising administering a hypomethylating agent.

49. The method of claim 48, wherein the hypomethylating agent is 5-azacytidine.

50. The method of claim 44, wherein the disease or disorder is a central nervous system (CNS) disease.

51. The method of claim 50, wherein the disease or disorder is amyotrophic lateral sclerosis (ALS).

52. The method of claim 44, wherein the disease or disorder is glioblastoma.
